(19) United States
(12) Patent Application Publication
(10) Pub. No.: US 2023/0242544 A1
(43) Pub. Date: Aug. 3, 2023

(54) QUINAZOLINE COMPOUNDS, PREPARATION METHODS AND USES THEREOF

(71) Applicant: INVENTISBIO CO., LTD., Shanghai (CN)

(72) Inventors: Xing DAI, Short Hills, NJ (US); Yaolin WANG, Shanghai (CN); Yueheng JIANG, Shanghai (CN); Haotao NIU, Shanghai (CN); Yanqin LIU, Shanghai (CN); Hong YANG, Shanghai (CN); Zixing HAN, Shanghai (CN); Zhenwu WANG, Shanghai (CN); Liangshan TAO, Shanghai (CN); Qiang ZHANG, Shanghai (CN); Zhe SHI, Shanghai (CN); Jifang WENG, Shanghai (CN)

(21) Appl. No.: 18/011,138

(22) PCT Filed: Jun. 30, 2021
(86) PCT No.: PCT/CN2021/103372

§ 371 Date: Dec. 16, 2022

(30) Foreign Application Priority Data
Jun. 30, 2020 (WO) PCT/CN2020/099104
Feb. 7, 2021 (WO) PCT/CN2021/075828

(51) Int. Cl. C07D 487/08; C07D 403/12; C07D 403/14; C07D 487/04; C07D 487/10; C07D 519/00; C07D 417/14; A61K 45/06

(52) U.S. Cl. CPC C07D 487/08; C07D 403/12; C07D 403/14; C07D 487/04; C07D 487/10; C07D 519/00; C07D 417/14; A61K 45/06

(57) ABSTRACT

Provided herein are novel compounds, for example, compounds having a Formula (I), Formula (II), or Formula (III), or a pharmaceutically acceptable salt thereof. Also provided herein are methods of preparing the compounds and methods of using the compounds, for example, in inhibiting KRAS^{G12D} in a cancer cell, and/or in treating various cancer such as pancreatic cancer, colorectal cancer, lung cancer or endometrial cancer.

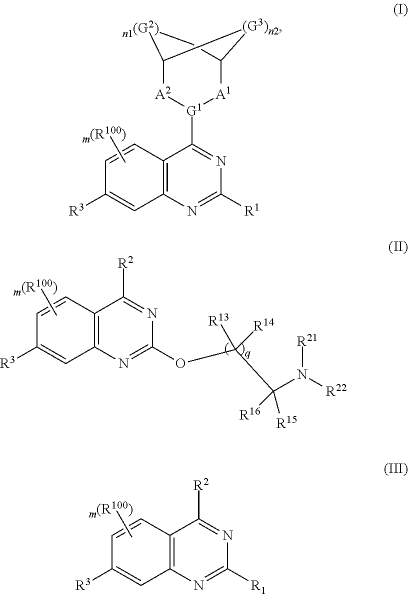

### CROSS REFERENCE TO RELATED APPLICATIONS

This application claims priority of International Application Nos. PCT/CN2020/099104, filed Jun. 30, 2020, and PCT/CN2021/075828, filed Feb. 7, 2021, the entire contents of each of which are incorporated herein by reference.

### BACKGROUND

### Field of the Disclosure

In various embodiments, the present disclosure generally relates to novel quinazoline compounds, compositions of the same, methods of preparing and methods of using the same, e.g., for inhibiting RAS and/or for treating a number of diseases or disorders, such as cancers.

### Background

RAS (KRAS, NRAS and HRAS) proteins regulate key cellular pathway transmitting signal received from cellular membrane receptor to downstream molecules such as Raf, MEK, ERK and PI3K, which are crucial for cell proliferation and survival. RAS cycles between the inactive GDP-bound form and active GTP-bound form. RAS is frequently mutated in cancers with KRAS accounted for ˜80% of all RAS mutations. KRAS mutation occurs in approximately 86% of pancreatic cancer, 41% of colorectal cancer, 36% of lung adenocarcinoma and 20% of endometrial carcinoma (F. McCormick, 2017, Clin Cancer Res 21: 1797-1801. Cancer Genome Atlas Network, 2017, Cancer Cell 32: 185-203). The RAS hot-spot mutations occur at codons 12, 13 and 61, with 75% of KRAS mutations occurs at codon 12 (Glycine) (D. K. Simanshu, D. V. Nissley and F. McCormick, 2017, Cell, 170: 17-33). KRAS^{G12D} (change of glycine at codon 12 to aspartic acid) is frequently mutated in pancreatic adenocarcinoma, colon adenocarcinoma and lung adenocarcinoma. However, targeting the KRAS^{G12D} mutation with small molecule is a challenge due to its shallow pocket.

There is a huge unmet medical need for therapeutic intervention of cancer patients with RAS mutations.

### BRIEF SUMMARY

In various embodiments, the present disclosure provides novel compounds, pharmaceutical compositions, methods of preparing and using the same. Typically, the compounds herein are RAS inhibitors, such as mutant KRAS (e.g., G12C, G12D, G12V, or G12A, more particularly G12D) inhibitors. The compounds and compositions herein are useful for treating various diseases or disorders, such as cancer or cancer metastasis.

In some embodiments, the present disclosure provides a compound of Formula I, Formula II, or Formula III, or a pharmaceutically acceptable salt thereof:

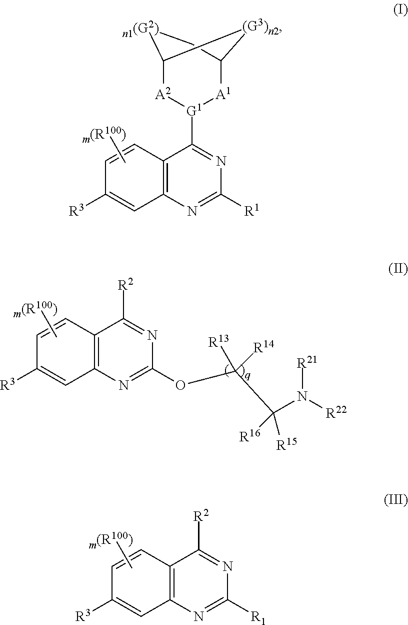

wherein R¹, R², R³, R¹³, R¹⁴, R¹⁵, R¹⁶, R²¹, R²², G¹, A¹, A², G², G³, R¹⁰⁰, m, n1, n2 and q are defined herein.

Certain embodiments of the present disclosure are directed to a pharmaceutical composition comprising one or more of the compounds of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) and optionally a pharmaceutically acceptable excipient. The pharmaceutical composition described herein can be formulated for different routes of administration, such as oral administration, parenteral administration, or inhalation etc.

Certain embodiments are directed to a method of treating a disease or disorder associated with RAS, e.g., KRAS G12D. In some embodiments, the method comprises administering to a subject in need thereof a therapeutically effective amount of a compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a therapeutically effective amount of a pharmaceutical composition described herein. Diseases or disorders associated with RAS, e.g., KRAS G12D, suitable to be treated with the method include those described herein.

In some embodiments, a method of treating cancer is provided. In some embodiments, the method comprises administering to a subject in need thereof a therapeutically effective amount of a compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a therapeutically effective amount of a pharmaceutical composition described herein. In various embodiments, the cancer can be pancreatic cancer, endometrial cancer, colorectal cancer or lung cancer (e.g., non-small cell lung cancer). In some embodiments, the cancer is a hematological cancer (e.g., described herein). In some embodiments, the cancer can be appendix cancer, cholangiocarcinoma, bladder urothelial cancer, ovarian cancer, gastric cancer, breast cancer, or bile duct cancer.

In some embodiments, a method of treating cancer metastasis or tumor metastasis is provided. In some embodiments, the method comprises administering to a subject in need thereof a therapeutically effective amount of a compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a therapeutically effective amount of a pharmaceutical composition described herein.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally.

The compounds of the present disclosure can be used as a monotherapy or in a combination therapy. In some embodiments, the combination therapy includes treating the subject with a targeted therapeutic agent, chemotherapeutic agent, therapeutic antibody, radiation, cell therapy, or immunotherapy.

It is to be understood that both the foregoing summary and the following detailed description are exemplary and explanatory only, and are not restrictive of the invention herein.

### DETAILED DESCRIPTION

In various embodiments, provided herein are novel compounds, pharmaceutical compositions, methods of preparation and methods of use.

### Compounds

Some embodiments of the present disclosure are directed to novel compounds. The compounds herein typically can be an inhibitor of a KRAS protein, particularly, a KRAS G12D mutant protein, and useful for treating various diseases or disorders, such as those described herein, e.g., cancer.

In some embodiments, the present disclosure provides a compound of Formula I, or a pharmaceutically acceptable salt thereof:

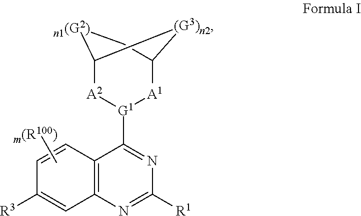

wherein:
G¹ is CR¹⁰ or N;
each occurrence of G² and G³ is independently CR¹¹R¹², O, or NR²⁰, provided that at least one instance of G² and G³ is NR²⁰;
n1 and n2 are each independently an integer of 1, 2, 3, or 4;
A¹ and A² are each independently a bond, CR¹¹R¹², O, or NR²⁰, provided that at least one of A¹ and A² is not O or NR²⁰.
R¹ is hydrogen, -(L¹)ⱼ₁-OR³⁰, halogen, -(L¹)ⱼ₁-NR²¹R²², or an optionally substituted heterocyclic or heteroaryl ring;
R³ is an optionally substituted aryl or an optionally substituted heteroaryl,
R¹⁰⁰ at each occurrence is independently F, Cl, Br, I, CN, —OH, —C(O)NH₂, —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), cyclopropyl, cyclobutyl, optionally substituted C₁₋₄ alkoxy (e.g., methoxy, ethoxy, —O—CH₂-cyclopropyl), cyclopropoxy, cyclobutoxy, S—R^{A}, S(O)R^{A}, or S(O)₂R^{A}; wherein R^{A} at each occurrence is independently hydrogen, optionally substituted C₁₋₄ alkyl (e.g., methyl, ethyl, CF₃, etc.), cyclopropyl, or cyclobutyl, and
m is 0, 1, 2, or 3;
wherein:
   j1 is 0 or 1, and when j1 is 1, L¹ is an optionally substituted alkylene, an optionally substituted carbocyclylene, an optionally substituted heterocyclylene; each occurrence of R¹⁰, R¹¹, or R¹² is independently hydrogen, F, —OH, or an optionally substituted C₁₋₆ alkyl, or R¹¹ and R¹² together with the carbon they are both attached to are joined to form an oxo or imino group or a ring;
   R²⁰ at each occurrence is independently hydrogen, a nitrogen protecting group, or an optionally substituted C₁₋₆ alkyl;
   R²¹ and R²² are independently hydrogen, a nitrogen protecting group, an optionally substituted C₁₋₆ alkyl, an optionally substituted carbocyclic ring, or an optionally substituted heterocyclic ring; or R²¹ and R²² are joined to form an optionally substituted heterocyclic or heteroaryl ring; and
   R³⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring.

The compound of Formula I (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula I (including any of the applicable sub-formulae as described herein) can exist as a mixture of atropisomers in any ratio, including about 1:1. In some embodiments, when applicable, the compound of Formula I (including any of the applicable sub-formulae as described herein) can exist as an isolated individual atropisomer substantially free (e.g., with less than 20%, less than 10%, less than 5%, less than 1%, by weight, by HPLC area, or both, or with a non-detectable amount) of the other atropisomer(s).

In some embodiments, G¹ in Formula I is N.

In some embodiments, G¹ in Formula I is CR¹⁰. In some embodiments, R¹⁰ can be hydrogen, F, —OH, or C₁₋₆ alkyl (such as methyl, ethyl, etc.) which can be optionally substituted, for example, with F, —OH, methoxy, etc. Typically, when G¹ is CR¹⁰, R¹⁰ is hydrogen.

A¹ and A² in Formula I can independently be a bond, a carbon-based linker, oxygen, or a nitrogen-based linker. Typically, A¹ and A² in Formula I can independently be a bond or CR¹¹R¹². In some embodiments, one of A¹ and A² is a bond. In some embodiments, both A¹ and A² are a bond, thus, both of the bridging points are directly connected to G¹. In some embodiments, one of A¹ and A² is CR¹¹R¹², wherein R¹¹ and R¹² can be independently hydrogen, F, —OH, or C₁₋₆ alkyl (such as methyl, ethyl, etc.) which can be optionally substituted, for example, with F, —OH, methoxy, etc. In some embodiments, one of A¹ and A² is CR¹¹R¹², wherein R¹¹ and R¹² together with the carbon they are both attached to are joined to form an oxo or imino group or a ring (e.g., cyclopropyl), for example, A¹ can be C═O, C═NH, etc. In some embodiments, both A¹ and A² are independently selected CR¹¹R¹², wherein R¹¹ and R¹² are defined herein in. For example, in some embodiments, both A¹ and A² are CH₂. In some embodiments, one of A¹ and A² is CH₂ and the other of A¹ and A² is C═O or C═NH. In some embodiments, both A¹ and A² are C═O.

In some embodiments, each occurrence of G² can be independently CR¹¹R¹². In such embodiments, at least one instance of G³ is NR²⁰. In some embodiments, each occurrence of G² can be the same. In some embodiments, each occurrence of G² can also be different from each other, or some of the G² are the same whereas others are different. In some embodiments, each occurrence of G² can be independently CR¹¹R¹², wherein R¹¹ and R¹² can be independently hydrogen, F, —OH, or C₁₋₆ alkyl (such as methyl, ethyl, etc.) which can be optionally substituted, for example, with F, —OH, methoxy, etc. In some embodiments, one or two instances of G² can be CR¹¹R¹², wherein R¹¹ and R¹² together with the carbon they are both attached to are joined to form an oxo or imino group or a ring (e.g., cyclopropyl). For example, in some embodiments, one instance of G² can be C═O or C═NH.

In some embodiments, one or two instances of G² can be O or NR²⁰. Typically, at most one of G² is heteroatom based moiety, such as O or NR²⁰, and the other instances of G² are independently CR¹¹R¹².

In some embodiments, each occurrence of G³ can be independently CR¹¹R¹². In such embodiments, at least one instance of G² is NR²⁰. In some embodiments, each occurrence of G³ can be the same. In some embodiments, each occurrence of G³ can also be different from each other, or some of the G³ are the same whereas others are different. In some embodiments, each occurrence of G³ can be independently CR¹¹R¹², wherein R¹¹ and R¹² can be independently hydrogen, F, —OH, or C₁₋₆ alkyl (such as methyl, ethyl, etc.) which can be optionally substituted, for example, with F, —OH, methoxy, etc. In some embodiments, one or two instances of G³ can be CR¹¹R¹², wherein R¹¹ and R¹² together with the carbon they are both attached to are joined to form an oxo or imino group or a ring (e.g., cyclopropyl). For example, in some embodiments, one instance of G³ can be C═O or C═NH.

In some embodiments, one or two instances of G³ can be O or NR²⁰. Typically, at most one of G³ is heteroatom based moiety, such as O or NR²⁰, and the other instances of G³ are independently CR¹¹R¹².

Typically, Formula I includes 1, 2, or 3 G² (as defined herein), i.e., n1 is 1, 2 or 3. In some embodiments, Formula I includes 1, 2, or 3 G³ (as defined herein), i.e., n2 is 1, 2 or 3.

As described herein, at least one instance out of all G² and G³ is NR²⁰. In some embodiments, one instance out of all G² and G³, i.e., one G² or one G³ among all G² and G³, is NR²⁰. For example, in some embodiments, among all G² and G³, one G² or one G³ is NR²⁰, wherein R²⁰ is hydrogen or C₁₋₄ alkyl (e.g., methyl). In some embodiments, R²⁰ at each occurrence can be independently hydrogen, a nitrogen protecting group (e.g., described herein), or a C₁₋₆ alkyl (e.g., methyl, ethyl, isopropyl, etc.), which can be optionally substituted, for example, with 1, 2, or 3 substituents independently selected from F, —OH, protected hydroxyl, oxo, NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, cyclopropyl, fluoro-substituted C₁₋₄ alkyl (e.g., CF₃), C₁₋₄ alkoxy, and fluoro-substituted C₁₋₄ alkoxy.

In some embodiments, the compound of Formula I can be characterized as having Formula I-1, I-2, or I-3:

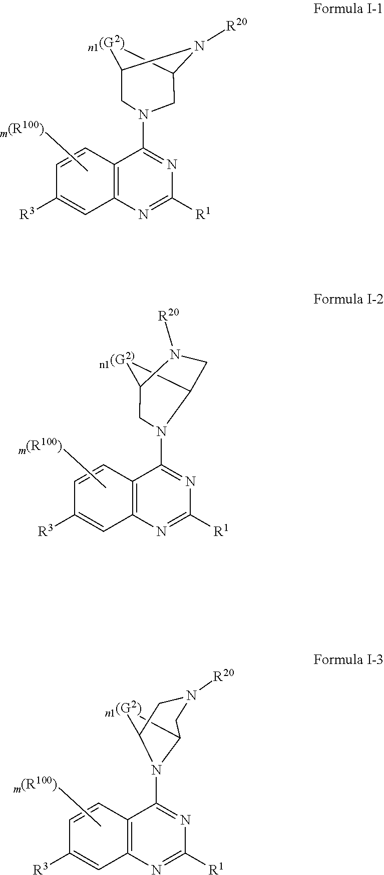

wherein the variables R¹, R³, R¹⁰⁰, R²⁰, G², and n1 are defined herein. For example, in some embodiments, n1 is 1, 2, or 3, and each G² can be CH₂. In some embodiments, R²⁰ can be hydrogen.

In some specific embodiments, the moiety

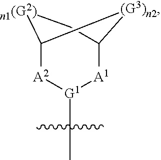

in Formula I is selected from the following:

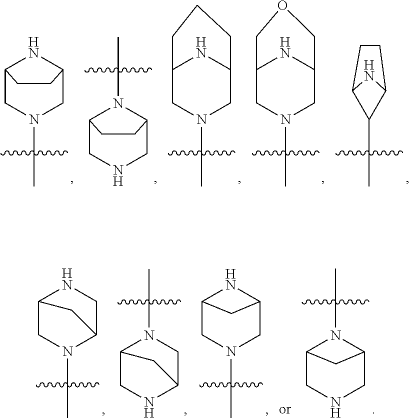

For example, in some embodiments, the compound of Formula I can be characterized as having Formula I-1-A, I-2-A, or I-3-A:

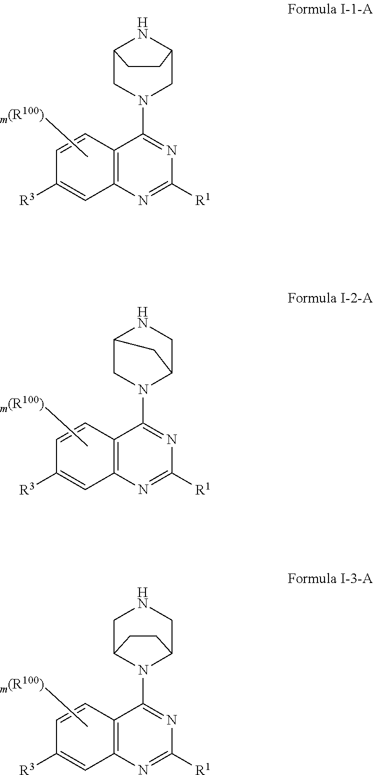

wherein the variables R¹, R³, R¹⁰⁰, and m are defined herein.

Various groups are suitable as R¹ in Formula I. In some embodiments, R¹ in Formula I (e.g., sub-formulae I-1, I-2, I-3, I-1-A, I-2-A, I-3-A) can be hydrogen. In some embodiments, R¹ in Formula I (e.g., sub-formulae I-1, I-2, I-3, I-1-A, I-2-A, I-3-A) can be a halogen, such as F or Cl. Various R¹ suitable for Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) are exemplified herein in the specific examples.

In some embodiments, R¹ in Formula I (e.g., sub-formulae I-1, I-2, I-3, I-1-A, I-2-A, I-3-A) can be -(L¹)ⱼ₁-OR³⁰. In some embodiments, j1 is 0, i.e., R¹ is —OR³⁰. In some embodiments, R³⁰ can be an optionally substituted C₁₋₆ alkyl, for example, in some embodiments, R³⁰ can be methyl. In some embodiments, j1 is 1, and L¹ can be an optionally substituted C₁₋₄ alkylene, an optionally substituted C₃₋₆ carbocyclylene, an optionally substituted 3-7 membered heterocyclylene. For example, in some embodiments, j1 is 1, and L can be a C₁₋₄ alkylene such as —CH₂—, —CH₂—CH₂—, or —CH₂—CH₂—CH₂—.

In some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) is —OR³⁰, wherein R³⁰ is a —C₁₋₆ alkylene-R¹⁰¹, wherein R¹⁰¹ is NR²³R²⁴ or an optionally substituted 4-10 membered heterocyclic ring, wherein the C₁₋₆ alkylene is optionally substituted, e.g., with one or more substituents independently selected from F, OH, NR²⁵R²⁶, and C₁₋₄ alkyl optionally substituted with 1-3 fluorine, or two substituents of the alkylene group are joined to form a ring; R²³ and R²⁴ are independently hydrogen, a nitrogen protecting group, an optionally substituted C₁₋₆ alkyl, an optionally substituted carbocyclic ring, or an optionally substituted heterocyclic ring; or R²³ and R²⁴ are joined to form an optionally substituted heterocyclic or heteroaryl ring; and R²⁵ and R²⁶ are independently hydrogen, a nitrogen protecting group, an optionally substituted C₁₋₆ alkyl, an optionally substituted carbocyclic ring, or an optionally substituted heterocyclic ring; or R²⁵ and R²⁶ are joined to form an optionally substituted heterocyclic or heteroaryl ring. In some embodiments, the —C₁₋₆ alkylene-unit in R³⁰ is unsubstituted C₁₋₄ alkylene (straight chain or branched). In some embodiments, the —C₁₋₆ alkylene-unit in R³⁰ is a C₁₋₄ alkylene optionally substituted with 1, 2, or 3 substituents, preferably 1 or 2 substituents, independently selected from F, —OH, methyl, ethyl, and CF₃. In some embodiments, the —C₁₋₆ alkylene-unit in R³⁰ is a C₁₋₄ alkylene, wherein two substituents (e.g., of the same carbon) are joined to form a cyclopropyl, cyclobutyl, or a 5-6 membered heterocyclic ring such as pyrrolidine, piperidine, tetrahydrofurane, tetrahydropyrane ring, which ring may be optionally substituted with substituents such as F, —OH, methyl, ethyl, and CF₃. In some embodiments, the —C₁₋₆ alkylene-unit in R³⁰ is selected from —CH₂—, —CH₂—CH₂—, —CH₂—CH₂—CH₂—,

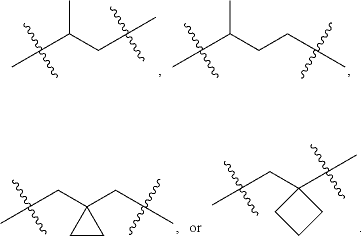

In some embodiments, R³⁰ is —CH₂—R¹⁰¹, —CH₂—CH₂—R¹⁰¹, —CH₂—CH₂—CH₂—R¹⁰¹,

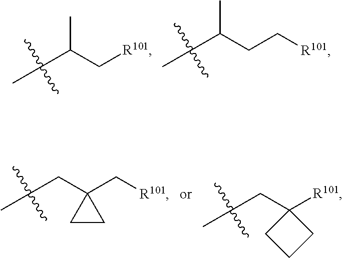

wherein R¹⁰¹ is defined herein.

R¹⁰¹ is typically NR²³R²⁴ or an optionally substituted 4-10 membered heterocyclic ring having 1-3 ring heteroatoms independently selected from O, S, and N.

In some embodiments, R¹⁰¹ is NR²³R²⁴, wherein R²³ and R²⁴ are independently hydrogen or an optionally substituted C₁₋₄ alkyl, such as methyl, ethyl, isopropyl, etc. For example, in some embodiments, R¹⁰¹ is NH₂, NH(C₁₋₄ alkyl), or N(C₁₋₄ alkyl)(C₁₋₄ alkyl). As used herein, the two C₁₋₄ alkyl in N(C₁₋₄ alkyl)(C₁₋₄ alkyl) can be the same or different, for example, it includes N(CH₃)₂ and N(CH₃)(C₂H₅), etc. Other similar expressions should be understood similarly. In some embodiments, R¹⁰¹ is NR²³R²⁴, wherein one of R²³ and R²⁴ is hydrogen or an optionally substituted C₃₋₆ cycloalkyl, and the other of R²³ and R²⁴ is defined herein, for example, in some embodiments, the other of R²³ and R²⁴ is hydrogen, an optionally substituted C₃₋₆ cycloalkyl, or a C₁₋₄ alkyl such as methyl. In some embodiments, R¹⁰¹ is NR²³R²⁴, wherein one of R²³ and R²⁴ is hydrogen or an optionally substituted 4-8 membered heterocyclic ring such as those having 1 or 2 heteroatoms independently selected from O and N, preferably, the ring has at most one oxygen, and the other of R²³ and R²⁴ is defined herein, for example, in some embodiments, the other of R²³ and R²⁴ is hydrogen or a C₁₋₄ alkyl such as methyl.

In some embodiments, R¹⁰¹ is NR²³R²⁴, wherein R²³ and R²⁴ together with the N they are both attached to are joined to form an optionally substituted 4-8 membered monocyclic heterocyclic ring having one or two ring heteroatoms, e.g., one ring nitrogen atom, two ring nitrogen atoms, one ring nitrogen atom and one ring sulfur atom, or one ring nitrogen atom and one ring oxygen atom, etc. For example, in some embodiments, R¹⁰¹ is NR²³R²⁴, wherein R²³ and R²⁴ together with the N they are both attached to are joined to form a ring selected from

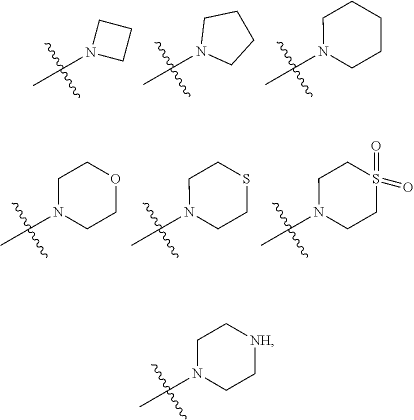

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. The substituents can be attached to any available positions in the ring, including for example an available ring nitrogen atom. Though not prohibited, for ring nitrogen substitutions, it is generally preferred not to form a quaternary salt, in other words, only one substituent is typically attached to a ring nitrogen (if substituted).

In some embodiments, R¹⁰¹ can be a monocyclic 4-8 membered heterocyclic ring having 1 or 2 ring heteroatoms independently selected from N, O, and S, or a fused or spiro bicyclic 6-10 membered heterocyclic ring having one to three ring heteroatoms independently selected from N, O, and S, wherein the monocyclic or bicyclic ring is optionally substituted. The monocyclic or bicyclic ring can be attached to the —C₁₋₆ alkylene-moiety via any available position to form a R³⁰. For the bicyclic ring, the attaching point can be on either of the two rings.

For example, in some embodiments, R¹⁰¹ can be a monocyclic ring selected from the following:

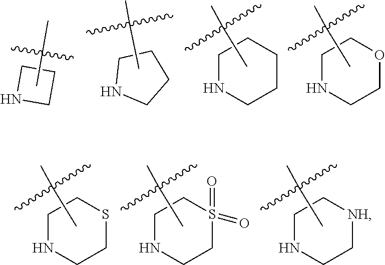

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

In some embodiments, R¹⁰¹ can be a bicyclic ring selected from the following:

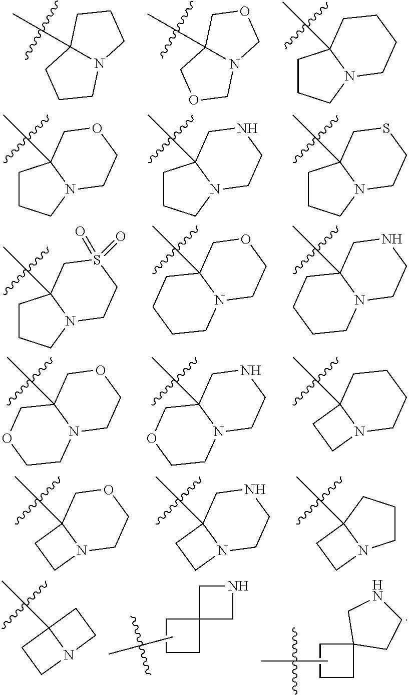

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. To be clear, the attaching point of the two spiro-bicyclic structure above can be a ring atom from either the cyclobutyl ring or the azetidine or pyrrolidine ring. In some embodiments, the attaching point is a ring atom from the cyclobutyl ring, e.g., on the carbon that's not adjacent to the spiro center.

Any of the R¹⁰¹ can be combined with any of the —C₁₋₆ alkylene-moiety described herein to form a R³⁰ suitable for Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12), wherein R¹ is —OR³⁰. For example, in some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can be selected from:

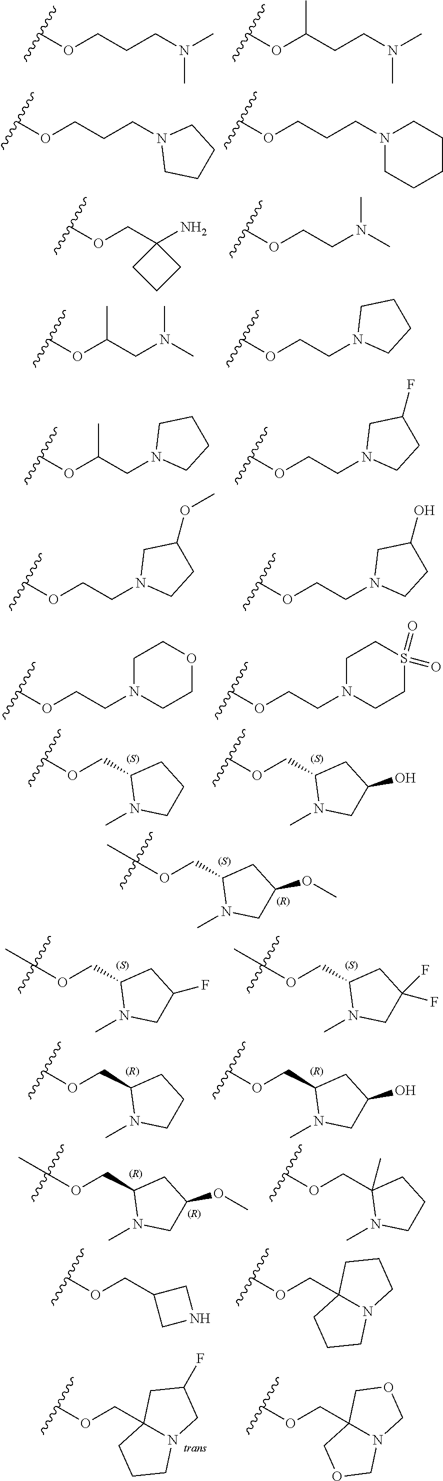

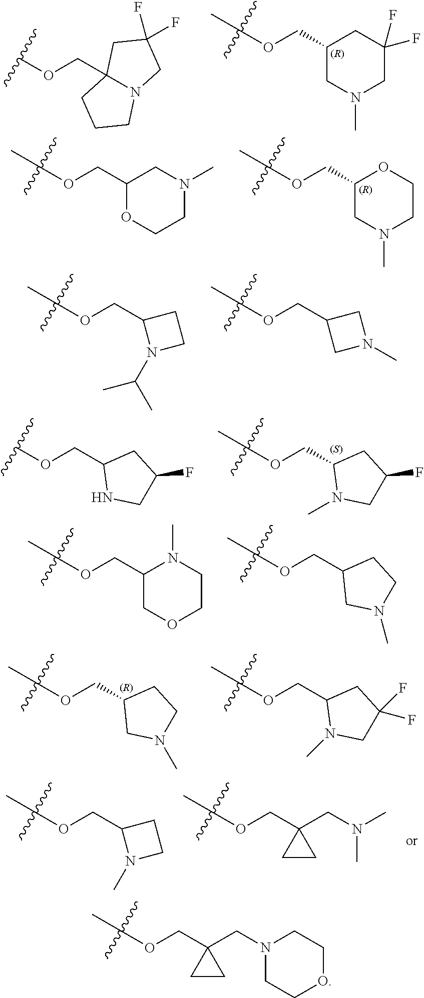

In some embodiments, the compound of Formula I can be characterized as having a Formula I-1-A-1, I-1-A-2, or I-1-A-3:

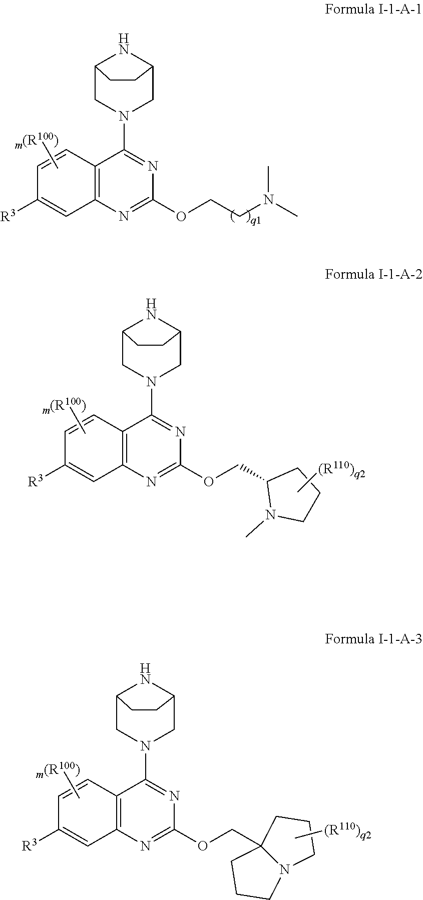

wherein R³, R¹⁰⁰, and m are defined herein, q1 is 1 or 2, q2 is 0, 1, or 2, R¹¹⁰ at each occurrence is independently F or hydroxyl. In some embodiments, q2 in Formula I-1-A-2 or I-1-A-3 is 0. In some embodiments, q2 in Formula I-1-A-2 is 1, and R¹¹⁰ is F or hydroxyl. In some embodiments, q2 in Formula I-1-A-3 is 1, and R¹¹⁰ is F. In some embodiments, q2 in Formula I-1-A-2 or I-1-A-3 is 2, and both R¹¹⁰ are F. In some embodiments, the compound of Formula I can be characterized as having a Formula I-1-A-4 or I-1-A-5:

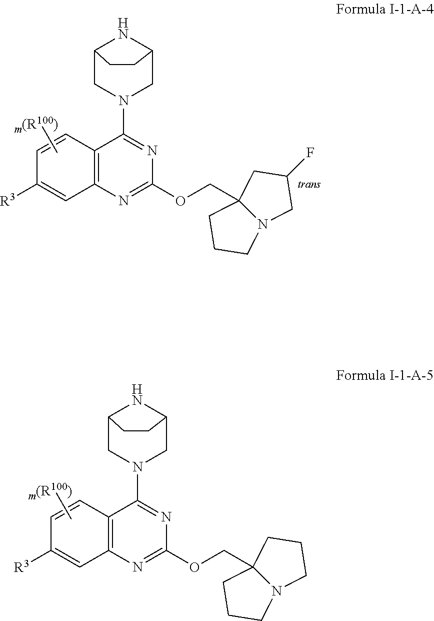

wherein R³, R¹⁰⁰, and m are defined herein. The “trans” designation in Formula I-1-A-4 indicates that the F substitution is trans to the quinazoline-linked moiety. For the avoidance of doubt, Formula I-1-A-4 includes individual stereoisomers (enantiomers etc.) and mixtures of stereoisomers in any ratio (including racemic mixtures). In some embodiments, the compound of Formula I-1-A-4 can have a formula according to I-1-A-4-E1 or I-1-A-4-E2:

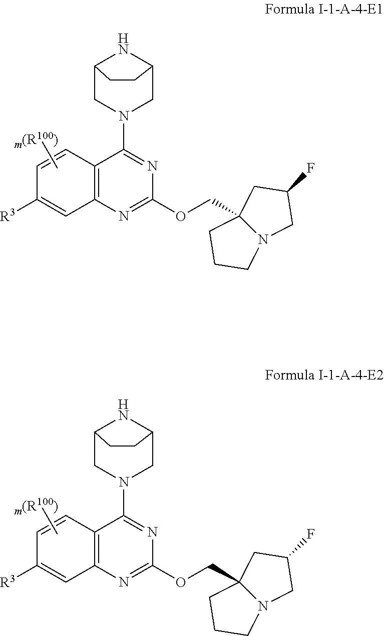

wherein R³, R¹⁰⁰, and m are defined herein. In some embodiments, compounds of Formula I-1-A-4-E1 or I-1-A-4-E2 can exist predominantly as the as-drawn stereoisomer (with respect to the two chiral centers showing stereochemical drawings), such as with less than 20%, less than 10%, less than 5%, less than 1%, by weight, by HPLC area, or both, or with a non-detectable amount of the other stereoisomer(s). The stereoisomers can be typically separated through chiral HPLC, e.g., as exemplified herein.

In some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can also be —OR³⁰ wherein R³⁰ is an optionally substituted C₃₋₆ carbocyclic ring or 4-10 membered heterocyclic ring. The oxygen can be connected with the carbocyclic or heterocyclic ring via any available attaching point, however, typically not through a heteroatom or a carbon atom adjacent to a heteroatom. In some embodiments, R³⁰ is a monocyclic 4-8 membered heterocyclic ring having 1 or 2 ring heteroatoms independently selected from N, O, and S, or a fused or spiro bicyclic 6-10 membered heterocyclic ring having one to three ring heteroatoms independently selected from N, O, and S, wherein the monocyclic or bicyclic ring is optionally substituted.

In some embodiments, R³⁰ is a 4-8 membered monocyclic saturated ring having one ring heteroatom, a ring nitrogen. For example, in some embodiments, R³⁰ is a monocyclic saturated ring selected from the following:

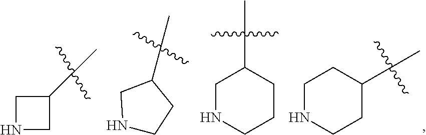

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, tetrahydropyranyl, —N(CH₃)₂, —OH, and —OCH₃.

In some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can also be —OR³⁰ wherein R³⁰ is an optionally substituted aryl or heteroaryl ring.

In some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can be selected from the following:

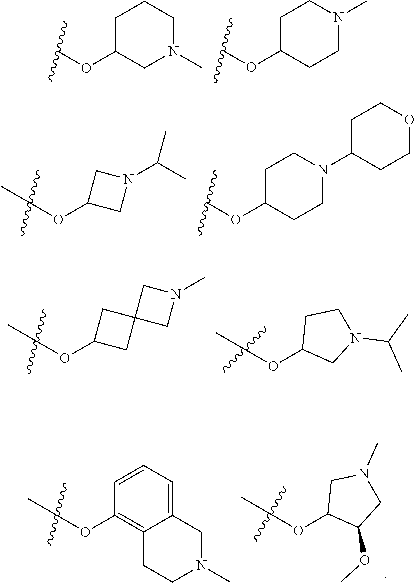

In some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can also be -(L¹)ⱼ₁-NR²¹R²². In some embodiments, j1 is 0, i.e., R¹ is NR²R². In some embodiments, j1 is 1, and L¹ can be an optionally substituted C₁₋₆ alkylene, an optionally substituted C₃₋₆ carbocyclylene, an optionally substituted 3-7 membered heterocyclylene. For example, in some embodiments, j1 is 1, and L¹ can be a C₁₋₄ alkylene such as —CH₂—, —CH₂—CH₂—, or —CH₂—CH₂—CH₂—.

For example, in some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can be NR²¹R²² or —C₁₋₆ alkylene-NR²¹R²². In some embodiments, R²¹ and R²² are independently hydrogen, an optionally substituted C₁₋₆ alkyl, or an optionally substituted heterocyclic ring; or R²¹ and R²² together with the N they are both attached to are joined to form an optionally substituted heterocyclic ring having one or two ring heteroatoms. In some embodiments, one of R¹¹ and R¹² is an optionally substituted 4-8 membered monocyclic saturated heterocyclic ring such as those having 1 or 2 heteroatoms independently selected from O and N, preferably, the ring has at most one oxygen. In some embodiments, the 4-8 membered monocyclic saturated heterocyclic ring is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. In some embodiments, the 4-8 membered monocyclic saturated heterocyclic ring has one ring heteroatom, which is a ring nitrogen atom (e.g., azetidine, pyrrolidine, piperazine, etc.). Typically, the attaching point is not the ring nitrogen atom or a carbon atom adjacent to the ring nitrogen. In some embodiments, the other of R²¹ and R²² is hydrogen or an optionally substituted C₁₋₆ alkyl, such as C₁₋₄ alkyl, e.g., methyl, ethyl, or isopropyl.

In some embodiments, R²¹ and R²² together with the N they are both attached to are joined to form a ring selected from

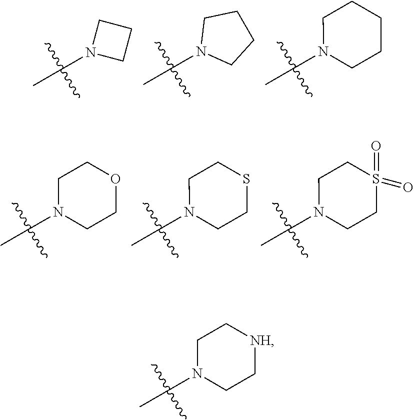

each of which is optionally substituted, for example, optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

In some specific embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can be

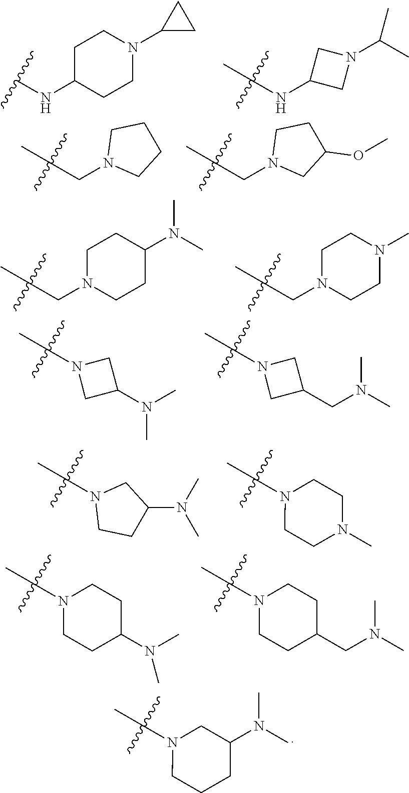

In some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can also be an optionally substituted heterocyclic or heteroaryl ring. In some embodiments, R¹ is an optionally substituted heterocyclic ring, preferably, a monocyclic 4-8 membered heterocyclic ring having 1 or 2 ring heteroatoms independently selected from N, O, and S, or a fused or spiro bicyclic 6-10 membered heterocyclic ring having one to three ring heteroatoms independently selected from N, O, and S, wherein the monocyclic or bicyclic ring is optionally substituted. In some embodiments, R¹ is an optionally substituted 4-8 membered monocyclic saturated heterocyclic ring such as those having 1 or 2 heteroatoms independently selected from O and N, preferably, the ring has at most one oxygen. In some embodiments, the 4-8 membered monocyclic saturated heterocyclic ring is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. In some embodiments, the 4-8 membered monocyclic saturated heterocyclic ring has one ring heteroatom, which is a ring nitrogen atom (e.g., azetidine, pyrrolidine, piperazine, etc.).

In some embodiments, R¹ in Formula I (e.g., sub-formulae I-1, I-2, I-3, I-1-A, I-2-A, I-3-A, I-1-A-6, I-1-A-7, I-1-A-8, I-1-A-9, I-1-A-10, I-1-A-11, or I-1-A-12) can be an optionally substituted fused or spiro bicyclic 6-10 membered heterocyclic ring having one to three ring heteroatoms independently selected from N, O, and S. For example, in some embodiments, R¹ is selected from

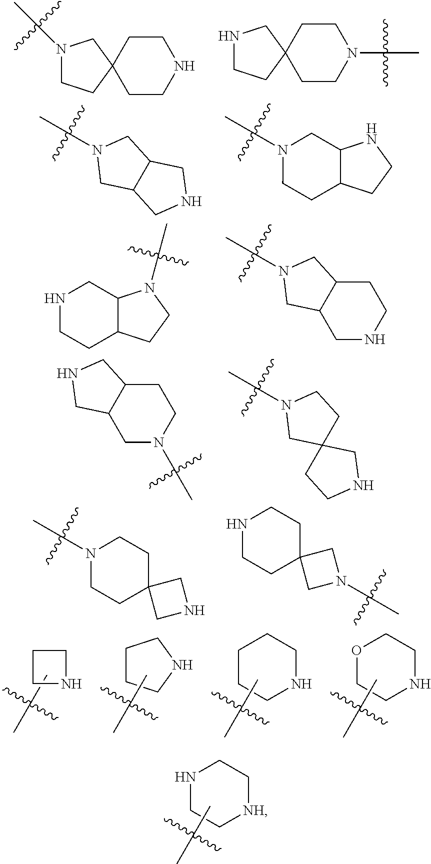

each of which is optionally substituted, for example, optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃. For example, in some embodiments, R¹ can be selected from

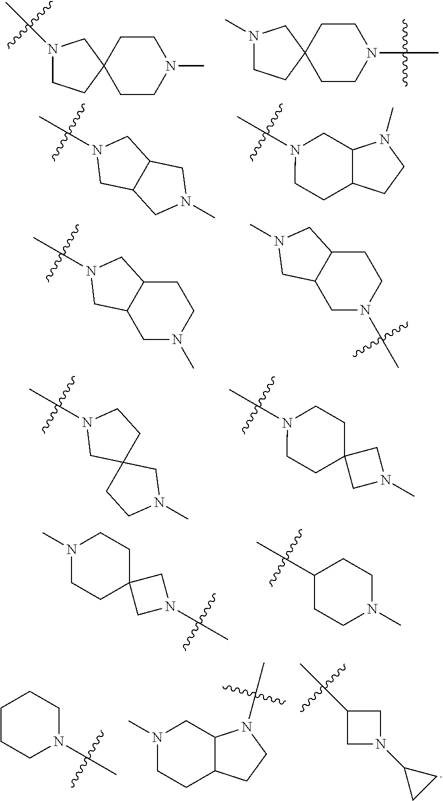

Typically, one or two R¹⁰⁰ are present in Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5), i.e., m is 1 or 2. Various groups are suitable for R¹⁰⁰. In some embodiments, R¹⁰⁰ at each occurrence is independently F, Cl, —CN, —OH, methoxy, ethoxy, —O—CH₂-cyclopropyl, —C(O)NHMe, CF₃, SCF₃, methyl, ethyl, isopropyl, or cyclopropyl. When two R¹⁰⁰ are present, they are both preferably ortho to the R³ group, such as shown in F-4:

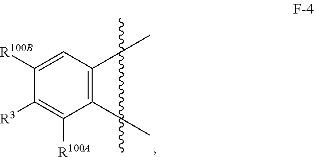

the remainder of Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5) is not shown in F-4, wherein each of R^{100A} and R^{100B} is independently a R¹⁰⁰ as defined herein. In some embodiments, R^{100A} in F-4 is F, and R^{100B} in F-4 is F, Cl, —CN, —OH, methoxy, ethoxy, —O—CH₂-cyclopropyl, —C(O)NHMe, CF₃, SCF₃, methyl, ethyl, isopropyl, or cyclopropyl. In some preferred embodiments, R^{100A} in F-4 is F, and R^{100B} in F-4 is Cl or CN. In some preferred embodiments, R^{100A} in F-4 is F, and R^{100B} in F-4 is F. In some preferred embodiments, R^{100A} in F-4 is F, and R^{100B} in F-4 is methoxy or ethoxy. In some embodiments, when two R¹⁰⁰ are present, one of them is ortho to the R³ group and the other is meta to the R³ group, such as shown in F-5:

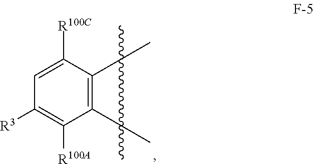

the remainder of Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5) is not shown in F-5, wherein each of R^{100A} and R^{100C} is independently a R¹⁰⁰ as defined herein. In some embodiments, R^{100A} in F-5 is F, and R^{100C} in F-5 is F, Cl, —CN, —OH, C₁₋₄ alkyl or C₁₋₄ alkoxy (such as methoxy, ethoxy, or isopropoxy). In some embodiments, R^{100A} in F-5 is F, and R^{100C} in F-5 is F, Cl, methoxy, ethoxy, or isopropoxy.

Various selections and combinations of R¹⁰⁰ suitable for Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5) are exemplified herein in the specific examples. In some specific embodiments, the compound of Formula I can be characterized as having a formula I-1-A-6, I-1-A-7, I-1-A-8, I-1-A-9, I-1-A-10, I-1-A-11, or I-1-A-12:

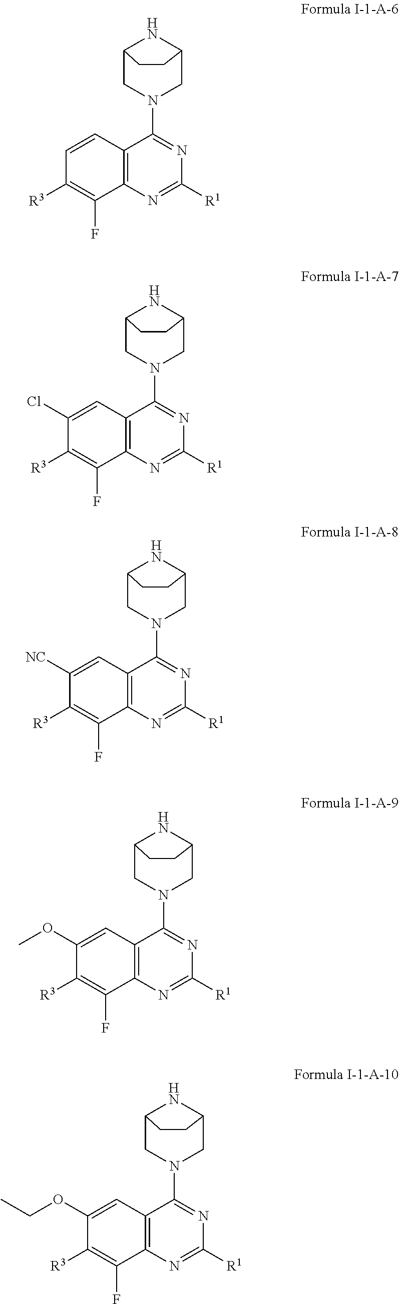

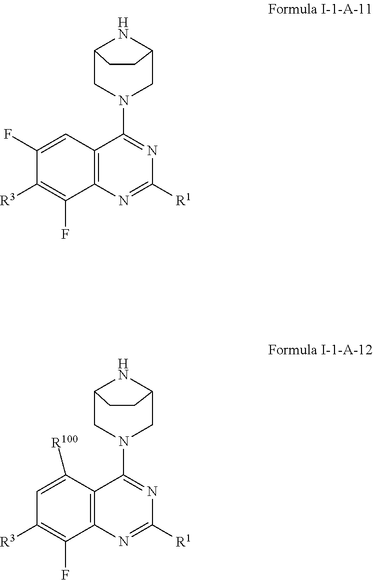

wherein R¹ and R³ and R¹⁰⁰ are defined herein. For example, in some embodiments, R¹⁰⁰ in Formula I-1-A-12 is F, Cl, —CN, —OH, or C₁₋₄ alkoxy (such as methoxy, ethoxy, or isopropoxy).

In some embodiments, R³ in Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12) can be a phenyl or 5 or 6 membered heteroaryl, such as pyridyl, which is optionally substituted. In some embodiments, R³ is a phenyl substituted with 1-3 substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, R³ is a pyridyl substituted with 1-3 substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, at most one of the substituents is OH, —NH₂, protected —OH, or a protected —NH₂.

In some embodiments, R³ in Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12) can be a naphthyl, which is optionally substituted, for example, with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂. In some embodiments, at most one of the substituents is OH, —NH₂, protected —OH, or a protected —NH₂. In some embodiments, R³ is

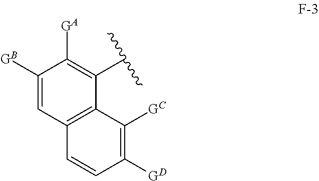

wherein:
1) G^{B} is OH, G^{A} is H, and G^{C} and GD are independently H, F, Cl, CN, C₁₋₄ alkyl optionally substituted with 1-3 fluorine, such as methyl, ethyl, or CF₃, preferably, G^{D} is H, F, or methyl;
2) G^{C} is Cl, methyl, ethyl, ethynyl, or CN, G^{A} is H, G^{B} is H or OH, and G^{D} is H, F, Cl, CN, C₁₋₄ alkyl optionally substituted with 1-3 fluorine, such as methyl, ethyl, or CF₃, preferably, G^{D} is H, F, or methyl; or
3) GA is Cl, G^{B} is H, F, or methyl, G^{C} and G^{D} are independently H, F, Cl, CN, C₁₋₄ alkyl optionally substituted with 1-3 fluorine, such as methyl, ethyl, or CF₃, preferably, G^{C} and G^{D} are independently H, F, or methyl.

In some embodiments, R³ in Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12) can be an optionally substituted naphthyl, such as a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, at most one of the substituents is OH, —NH₂, protected —OH, or a protected —NH₂. In some embodiments, R³ is

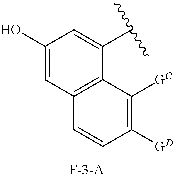

wherein G^{C} and G^{D} are independently H, F, Cl, CN, C₁₋₄ alkyl optionally substituted with 1-3 fluorine, such as methyl, ethyl, or CF₃, cyclopropyl, or C₂₋₄ alkynyl (e.g., ethynyl), preferably, G is H, F, or methyl. In some embodiments, in F-3-A, GC is Cl, methyl, ethyl, ethynyl, or CN, and G is H, F, Cl, CN, C₁₋₄ alkyl optionally substituted with 1-3 fluorine, such as methyl, ethyl, or CF₃. In some embodiments, in F-3-A, G^{C} is Cl, methyl, ethyl, ethynyl, or CN, and G^{D} is H or F. In some embodiments, R³ is

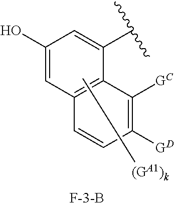

wherein G^{C} and G^{D} are independently H, F, Cl, CN, C₁₋₄ alkyl optionally substituted with 1-3 fluorine, such as methyl, ethyl, or CF₃, cyclopropyl, or C₂₋₄ alkynyl (e.g., ethynyl), preferably, G is H, F, or methyl, wherein G^{A} at each occurrence is independently a halo (e.g., F, or Cl), OH, CN, cyclopropyl, optionally substituted C₁₋₄ alkyl, or optionally substituted C₁₋₄ alkoxy, and k is 1, 2, or 3. It should be noted that the G^{A1} in F-3-B can be substituted at any available position of the naphthyl ring, although preferably, one or two G^{A1} is/are ortho to the OH group. In some embodiments, in F-3-B, G^{C} is Cl, methyl, ethyl, ethynyl, or CN, and G^{D} is H, F, Cl, CN, C₁₋₄ alkyl optionally substituted with 1-3 fluorine, such as methyl, ethyl, or CF₃. In some embodiments, in F-3-B, G^{C} is Cl, methyl, ethyl, ethynyl, or CN, and G^{D} is H or F. In some embodiments, k is 1, G^{A1} is ortho to the OH group, and G^{A1} is F, Cl, CN, or C₁₋₄ alkyl optionally substituted with 1-3 fluorine. In some embodiments, k is 2, both G^{A1} are ortho to the OH group, and each G^{A1} is independently F, Cl, CN, or C₁₋₄ alkyl optionally substituted with 1-3 fluorine.

In some embodiments, R³ in Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12) can be a bicyclic heteroaryl (e.g., benzothiazolyl, indazolyl, or isoquinolinyl), which is optionally substituted, for example, with 1-3 substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, at most one of the substituents is OH, —NH₂, protected —OH, or a protected —NH₂. For example, in some embodiments, R³ is

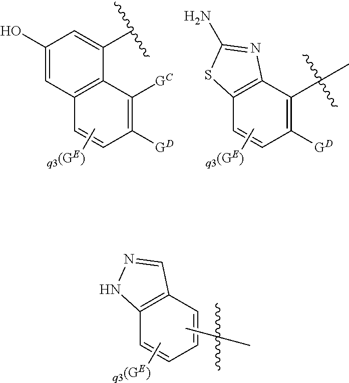

wherein: q3 is 0, 1, or 2, and G^{E} at each occurrence is independently F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, q3 is 0, 1, or 2, and G^{E} at each occurrence is F, Cl, C₁₋₄ alkyl (e.g., methyl, ethyl, propyl, isopropyl, tert-butyl), C₂₋₄ alkenyl, C₂₋₄ alkynyl (e.g., ethynyl), cyclopropyl, CH₂CH₂—CN, CF₂H, CF₃, or —CN.

Various selections of R³ suitable for Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12) are exemplified herein in the specific examples. In some embodiments, R³ in Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12) can be selected from:

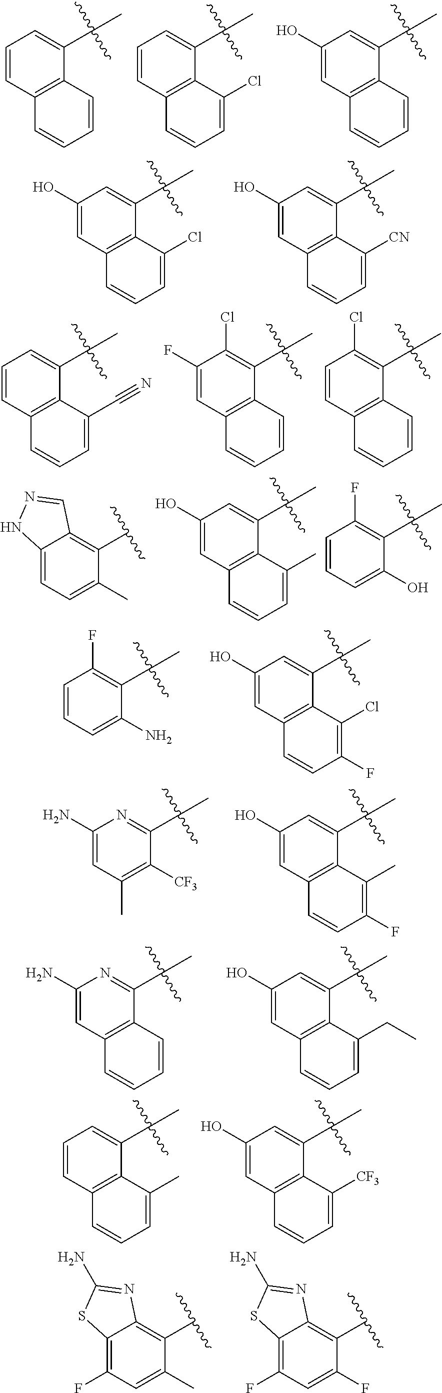

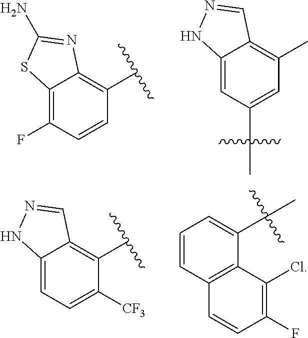 [SMILES: *c1ccc(F)c2sc(N)nc12 ; *c1cc(C)c2cn[nH]c2c1 ; *c1c(C(F)(F)F)ccc2[nH]ncc12 ; *c1cccc2ccc(F)c(Cl)c12]

In some embodiments, R³ in Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12) can be selected from:

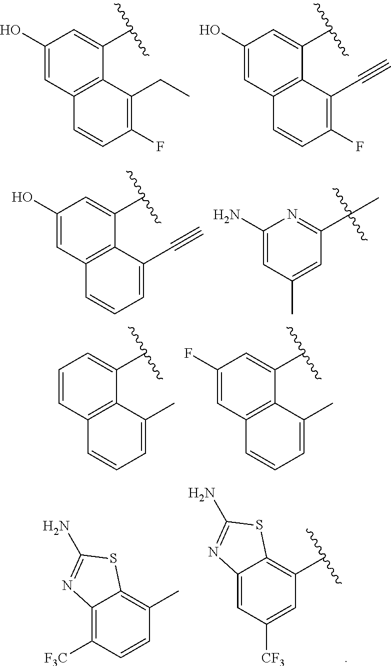

In some preferred embodiments, R³ in Formula I (e.g., sub-formulae I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12) can be selected from:

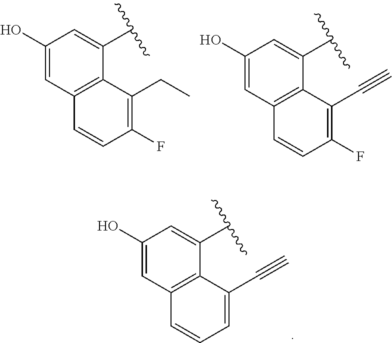 [SMILES: *c1cc(O)cc2ccc(F)c(CC)c12 ; *c1cc(O)cc2ccc(F)c(C#C)c12 ; *c1cc(O)cc2cccc(C#C)c12]

In some embodiments, the present disclosure provides a compound of Formula II, or a pharmaceutically acceptable salt thereof:

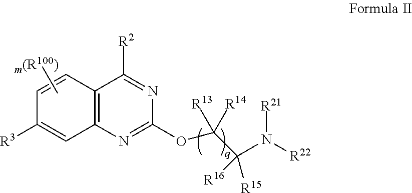

wherein:
R¹³ and R¹⁴ at each occurrence are independently hydrogen or a C₁₋₄ alkyl,
q is an integer of 0-6,
R¹⁵, R¹⁶, R²¹, and R²², together with the intervening carbon and nitrogen atoms, form an optionally substituted 6-10 membered fused bicyclic ring,
R² is a ring or ring-chain structure, e.g., having a pKa of about 6 or higher,
R³ is an optionally substituted aryl or an optionally substituted heteroaryl,
R¹⁰⁰ at each occurrence is independently F, Cl, Br, I, —CN, —OH, —C(O)NH₂, —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), cyclopropyl, cyclobutyl, optionally substituted C₁₋₄ alkoxy (e.g., methoxy, ethoxy, —O—CH₂-cyclopropyl), cyclopropoxy, cyclobutoxy, or S—R^{A}, S(O)R^{A}, or S(O)₂R^{A}; wherein R^{A} at each occurrence is independently hydrogen, optionally substituted C₁₋₄ alkyl (e.g., methyl, ethyl, CF₃, etc.), cyclopropyl, or cyclobutyl; and
m is 0, 1, 2, or 3.

The compound of Formula II (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula II (including any of the applicable sub-formulae as described herein) can exist as a mixture of atropisomers in any ratio, including about 1:1. In some embodiments, when applicable, the compound of Formula II (including any of the applicable sub-formulae as described herein) can exist as an isolated individual atropisomer substantially free (e.g., with less than 20%, less than 10%, less than 5%, less than 1%, by weight, by HPLC area, or both, or with a non-detectable amount) of the other atropisomer(s).

Typically, in Formula II, q is 1-3. In some embodiments, q is 1. In some embodiments, q is 2. R¹³ and R¹⁴ in Formula II are typically hydrogen or methyl. For example, in some embodiments, R¹³ and R¹⁴ at each occurrence are independently hydrogen or methyl.

In some embodiments, R¹, R, R², and R²², together with the intervening carbon and nitrogen atoms, form an optionally substituted 6-10 membered fused bicyclic ring selected from:

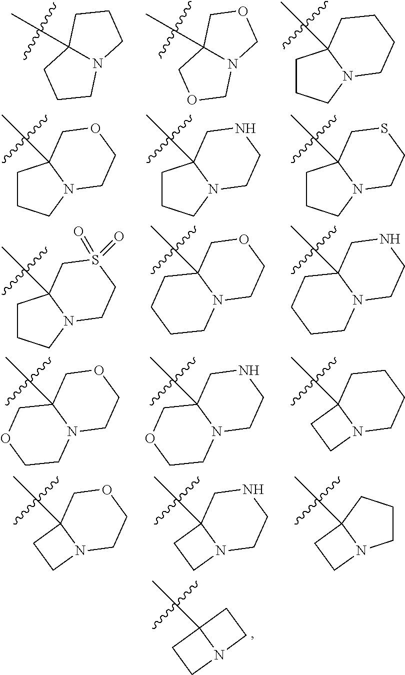

each of which is optionally substituted, for example, optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

In some embodiments, R¹⁵, R¹⁶, R²¹, and R²², together with the intervening carbon and nitrogen atoms, form

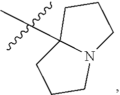 [SMILES: *C12CCCN1CCC2]

which is optionally substituted, on one or both rings. In some embodiments, the

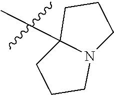 [SMILES: *C12CCCN1CCC2]

is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃. In some embodiments, only one of the pyrrolidine ring is substituted, e.g., with one fluorine.

In some specific embodiments, the compound of Formula II can be characterized as having a formula II-1:

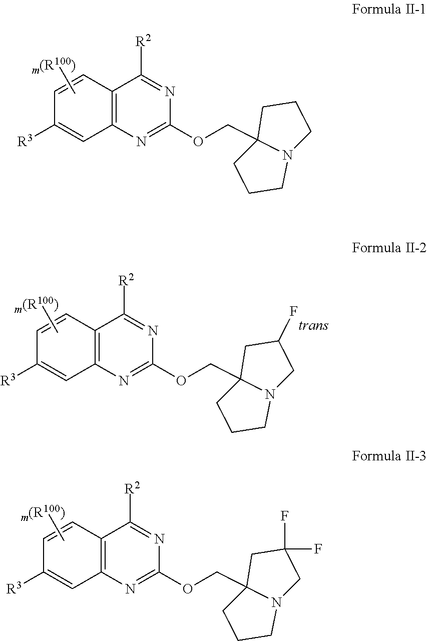

wherein R², R³, R¹⁰⁰, and m are defined herein. The “trans” designation in Formula II-2 indicates that the F substitution is trans to the quinazoline-linked moiety. For the avoidance of doubt, Formula II-2 includes individual stereoisomers (enantiomers etc.) and mixtures of stereoisomers in any ratio (including racemic mixtures). In some embodiments, the compound of Formula II-2 can have a formula according to II-2-E1 or II-2-E2:

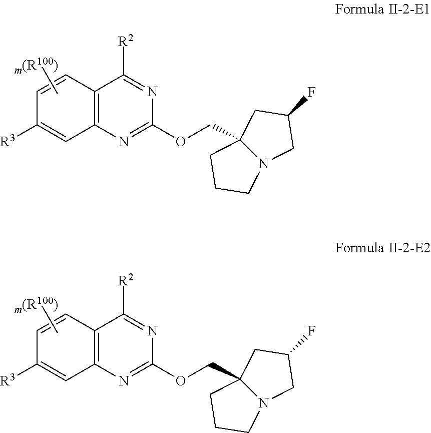

wherein R², R³, R¹⁰⁰, and m are defined herein. In some embodiments, compounds of Formula II-2-E1 or II-2-E2 can exist predominantly as the as-drawn enantiomer (with respect to the two chiral centers showing stereochemical drawings), such as with less than 20%, less than 10%, less than 5%, less than 1%, by weight, by HPLC area, or both, or with a non-detectable amount of the other enantiomer. The enantiomers can be typically separated through chiral HPLC, e.g., as exemplified herein.

Various groups are suitable as R² for Formula II, some of which are also exemplified in the specific compounds herein. In some embodiments, R² can be represented by -(L²)ⱼ₂-R¹⁰², wherein j2 is 0-3, typically 0 or 1, and when j2 is not 0, for example, j2 is 1, L² at each occurrence is independently CH₂, O, NH, or NCH₃, R¹⁰² is an optionally substituted 4-10 membered heterocyclic ring or a heteroaryl ring, e.g., those heterocyclic or heteroaryl rings having one or two ring nitrogen atoms. To be clear, when it is said that the heterocyclic or heteroaryl rings have one or two ring nitrogen atoms, the heterocyclic or heteroaryl rings may contain additional ring heteroatoms such as ring oxygen or ring sulfur atom(s). However, in some embodiments, the heterocyclic or heteroaryl rings only have the ring nitrogen atoms as ring heteroatoms. In some embodiments, j2 is 0. In some embodiments, j2 is 1.

In some embodiments, j2 is 0, and R¹⁰² is an optionally substituted 4-10 membered heterocyclic ring having one or two ring nitrogen atoms. For example, in some embodiments, R¹⁰² is selected from the following ring structures:

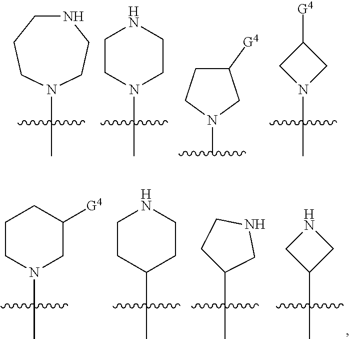

each of which is optionally substituted,
wherein G⁴ is -(L³)ⱼ₃-NH₂, -(L³)ⱼ₃-NH(C₁₋₄ alkyl), wherein j3 is 0 or 1, and when j3 is 1,
L³ is C₁₋₄ alkylene (e.g., methylene, ethylene, propylene, isopropylene, etc.), or G⁴ and one substituent on the ring are joined together to form a 4-6 membered heterocyclic ring having one or two ring nitrogen atoms. In some embodiments, each of the ring structures drawn above is optionally substituted with 1-3 (typically 1 or 2) substituents independently selected from C₁₋₄ alkyl (e.g., methyl, ethyl, etc.), fluorine substituted C₁₋₄ alkyl (e.g., CF₃), hydroxyl substituted C₁₋₄ alkyl, alkoxy substituted C₁₋₄ alkyl, cyano substituted C₁₋₄ alkyl, and CONH₂, or two substituents are combined to form an oxo, imino, or a ring structure. The substitution can occur on any available position of the rings, including the ring nitrogen atoms.

In some preferred embodiments, in Formula II (e.g., subformulae II-1, II-2, II-2-E1, II-2-E2, or II-3), R² is selected from:

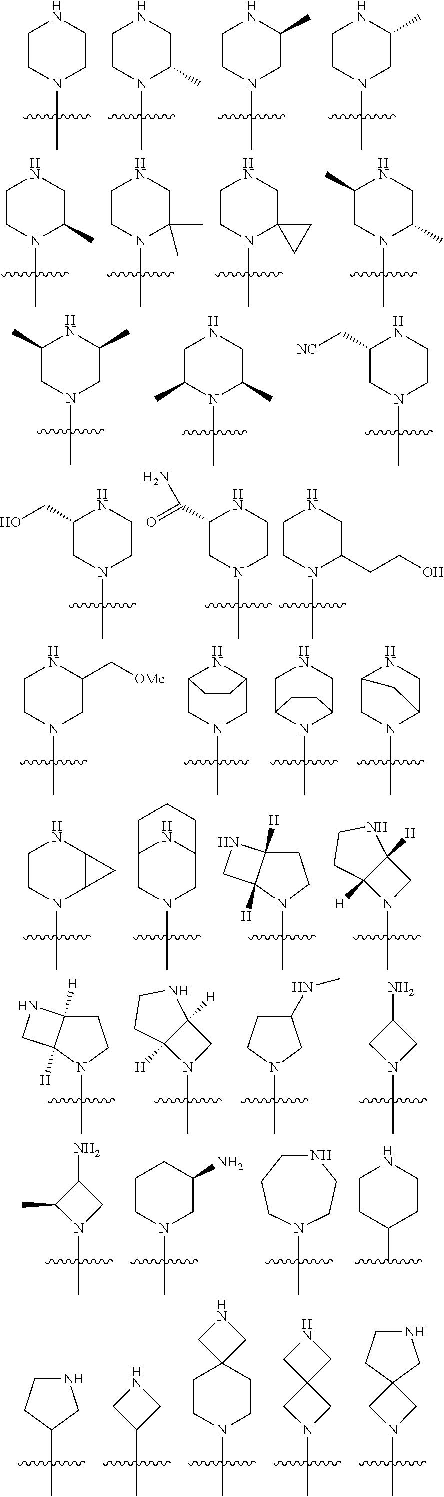

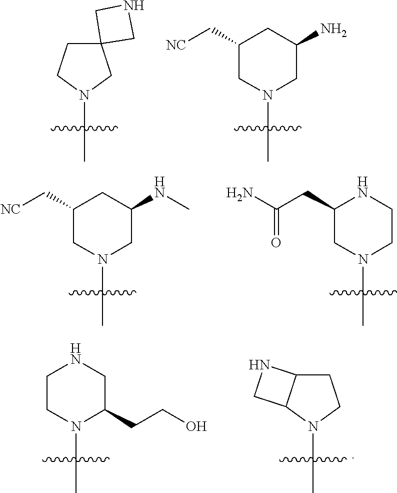 [SMILES: *N1CCC2(CNC2)C1 ; *N1C[C@H](N)C[C@@H](CC#N)C1 ; *N1C[C@H](CC#N)C[C@@H](NC)C1 ; *N1CCN[C@H](CC(N)=O)C1 ; *N1CCNC[C@H]1CCO ; *N1CCC2NCC21]

In some preferred embodiments, in Formula II (e.g., subformulae II-1, II-2, II-2-E1, II-2-E2, or II-3), R² is

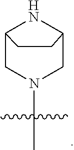 [SMILES: *N1CC2CCC(C1)N2]

In some embodiments, j2 is 1, L² is CH₂ or NH, and R¹⁰² is an optionally substituted 4-10 membered heterocyclic ring having one or two ring nitrogen atoms. For example, in some embodiments, j2 is 1, L² is CH₂ or NH, and R¹⁰² is an optionally substituted 4-8 membered heterocyclic ring, e.g., a monocyclic saturated 4-8 membered ring, which is optionally substituted. For example, in some embodiments, j2 is 1, L² is CH₂ or NH, and R¹⁰² is selected from:

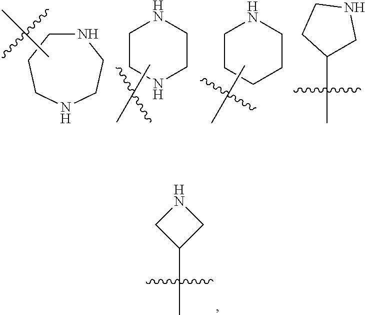

each of which is optionally substituted, for example, optionally substituted with 1-3 (typically 1 or 2) substituents independently selected from C₁₋₄ alkyl (e.g., methyl, ethyl, etc.), fluorine substituted C₁₋₄ alkyl (e.g., CF₃), hydroxyl substituted C₁₋₄ alkyl, alkoxy substituted C₁₋₄ alkyl, cyano substituted C₁₋₄ alkyl, and CONH₂, or two substituents are combined to form an oxo, imino, or a ring structure. The substitution can occur on any available position of the rings, including the ring nitrogen atoms.

In some embodiments, in Formula II (e.g., subformulae II-1, II-2, II-2-E1, II-2-E2, or II-3), R² is selected from:

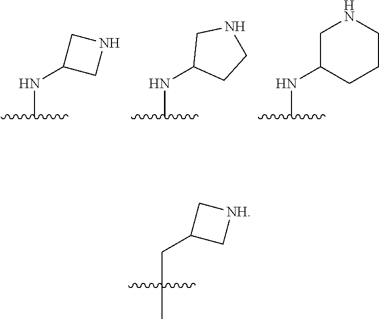 [SMILES: *NC1CNC1 ; *NC1CCNC1 ; *NC1CCCNC1 ; *CC1CNC1]

In some embodiments, in Formula II (e.g., subformulae II-1, II-2, II-2-E1, II-2-E2, or II-3), R² can also be a C₃₋₇ carbocyclic, phenyl, or 5 or 6 membered heteroaryl ring, each of which has at least one nitrogen containing substituent, e.g., a basic nitrogen containing substituent, such as NH₂, NH(C₁₋₄ alkyl), or NH(C₁₋₄ alkyl)(C₁₋₄ alkyl). For example, in some embodiments, R² is selected from

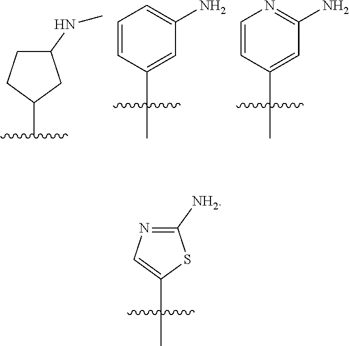

Typically, one or two R¹⁰⁰ are present in Formula II (e.g., subformulae II-1, II-2, II-2-E1, II-2-E2, or II-3), i.e., m is 1 or 2. Various groups are suitable for R¹⁰⁰. In some embodiments, R¹⁰⁰ at each occurrence is independently F, Cl, —CN, —OH, methoxy, ethoxy, —O—CH₂-cyclopropyl, —C(O)NHMe, CF₃, methyl, ethyl, isopropyl, or cyclopropyl. When two R¹⁰⁰ are present, they are both preferably ortho to the R³ group, such as shown in F-4:

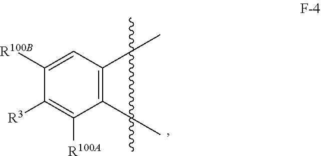

the remainder of Formula II (e.g., subformulae II-1, II-2, II-2-E1, II-2-E2, or II-3) is not shown in F-4, wherein each of R^{100A} and R^{100B} is independently a R¹⁰⁰ as defined herein. In some embodiments, R^{100A} in F-4 is F, and R^{100B} in F-4 is F, Cl, —CN, —OH, methoxy, ethoxy, —O—CH₂-cyclopropyl, —C(O)NHMe, CF₃, SCF₃, methyl, ethyl, isopropyl, or cyclopropyl. In some preferred embodiments, R^{100A} in F-4 is F, and R^{100B} in F-4 is Cl or CN. In some preferred embodiments, R^{100A} in F-4 is F, and R^{100B} in F-4 is F. In some preferred embodiments, R^{100A} in F-4 is F, and R^{100B} in F-4 is methoxy or ethoxy. In some embodiments, when two R¹⁰⁰ are present, one of them is ortho to the R³ group and the other is meta to the R³ group, such as shown in F-5:

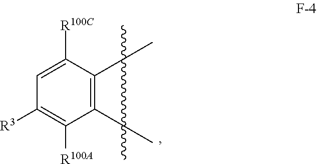

the remainder of Formula II (e.g., subformulae II-1, II-2, II-2-E1, II-2-E2, or II-3) is not shown in F-5, wherein each of R^{100A} and R^{100C} is independently a R¹⁰⁰ as defined herein. In some embodiments, R^{100A} in F-5 is F, and R^{100C} in F-5 is F, Cl, —CN, —OH, C₁₋₄ alkyl or C₁₋₄ alkoxy (such as methoxy, ethoxy, or isopropoxy). In some embodiments, R^{100A} in F-5 is F, and R^{100C} in F-5 is F, Cl, methoxy, ethoxy, or isopropoxy.

Various selections and combinations of R¹⁰⁰ suitable for Formula II (e.g., (e.g., subformulae II-1, II-2, II-2-E1, II-2-E2, or II-3) are exemplified herein in the specific examples. In some specific embodiments, the compound of Formula II can be characterized as having a formula II-1-A, II-1-B, II-1-C, II-2-A, II-2-B, or II-2-C:

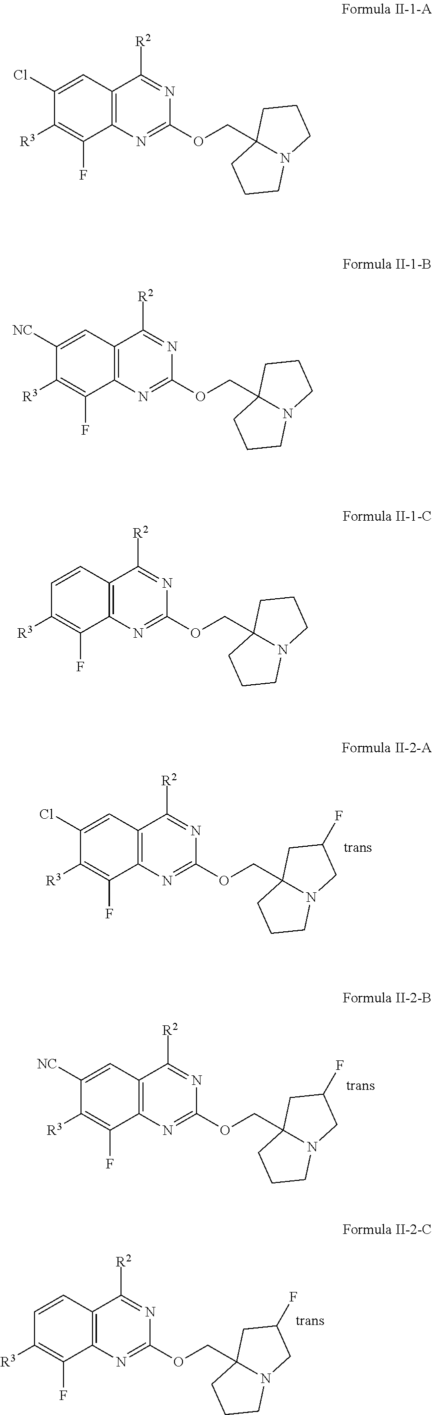

wherein R² and R³ are defined herein. In some embodiments, the compound of Formula II can be characterized as having Formula II-2-A-E1, II-2-B-E1, II-2-C-E1, II-2-A-E2, II-2-B-E2, or II-2-C-E2:

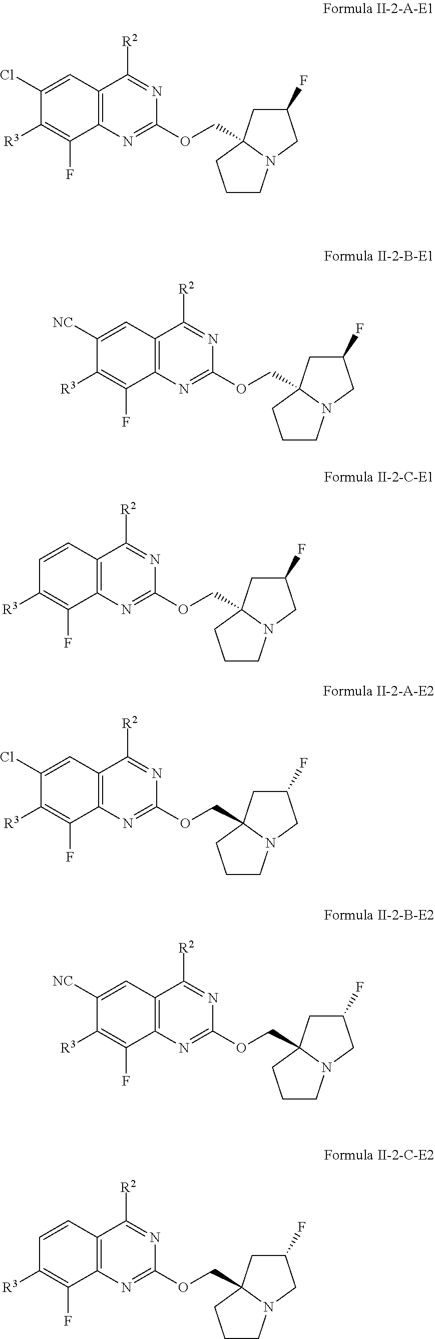

wherein R² and R³ are defined herein. In some embodiments, compounds of Formula II-2-A-E1, II-2-B-E1, II-2-C-E1, II-2-A-E2, II-2-B-E2, or II-2-C-E2 can exist predominantly as the as-drawn stereoisomer (with respect to the two chiral centers showing stereochemical drawings), such as with less than 20%, less than 10%, less than 5%, less than 1%, by weight, by HPLC area, or both, or with a non-detectable amount of the other stereoisomer(s). The stereoisomers can be typically separated through chiral HPLC, e.g., as exemplified herein.

In some embodiments, R³ in Formula II (e.g., subformulae II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2) can be a phenyl or 5 or 6 membered heteroaryl, such as pyridyl, which is optionally substituted. In some embodiments, R³ is a phenyl substituted with 1-3 substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, R³ is a pyridyl substituted with 1-3 substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, at most one of the substituents is OH, —NH₂, protected —OH, or a protected —NH₂.

In some embodiments, R³ in Formula II (e.g., subformulae II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2) can be a naphthyl, which is optionally substituted, for example, with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂. In some embodiments, at most one of the substituents is OH, —NH₂, protected —OH, or a protected —NH₂. In some embodiments, R³ is

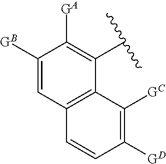

wherein:
1) G^{B} is OH, G^{A} is H, and G^{C} and G^{D} are independently H, F, Cl, CN, C₁₋₄ alkyl optionally substituted with 1-3 fluorine, such as methyl, ethyl, or CF₃, preferably, G^{D} is H, F, or methyl;
2) G^{C} is Cl, methyl, ethyl, ethynyl, or CN, G^{A} is H, G^{B} is H or OH, and G^{D} is H, F, Cl, CN, C₁₋₄ alkyl optionally substituted with 1-3 fluorine, such as methyl, ethyl, or CF₃, preferably, G^{D} is H, F, or methyl; or
3) G^{A} is Cl, G^{B} is H, F, or methyl, G^{C} and G^{D} are independently H, F, Cl, CN, C₁₋₄ alkyl optionally substituted with 1-3 fluorine, such as methyl, ethyl, or CF₃, preferably, G^{C} and G^{D} are independently H, F, or methyl.

In some embodiments, R³ in Formula II (e.g., subformulae II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2) can be an optionally substituted naphthyl, such as a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, at most one of the substituents is OH, —NH₂, protected —OH, or a protected —NH₂. In some embodiments, R³ is

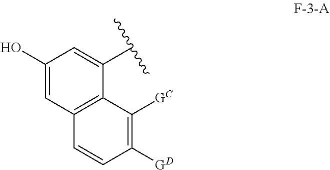

wherein G^{C} and G^{D} are independently H, F, Cl, CN, C₁₋₄ alkyl optionally substituted with 1-3 fluorine, such as methyl, ethyl, or CF₃, cyclopropyl, or C₂₋₄ alkynyl (e.g., ethynyl), preferably, G^{D} is H, F, or methyl. In some embodiments, in F-3-A, G^{C} is Cl, methyl, ethyl, ethynyl, or CN, and G is H, F, Cl, CN, C₁₋₄ alkyl optionally substituted with 1-3 fluorine, such as methyl, ethyl, or CF₃. In some embodiments, in F-3-A, G^{C} is Cl, methyl, ethyl, ethynyl, or CN, and G^{D} is H or F. In some embodiments, R³ is

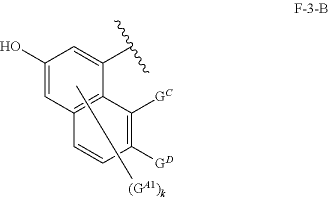

wherein G^{C} and G^{D} are independently H, F, Cl, CN, C₁₋₄ alkyl optionally substituted with 1-3 fluorine, such as methyl, ethyl, or CF₃, cyclopropyl, or C₂₋₄ alkynyl (e.g., ethynyl), preferably, G^{D} is H, F, or methyl, wherein G^{A1} at each occurrence is independently a halo (e.g., F, or Cl), OH, CN, cyclopropyl, optionally substituted C₁₋₄ alkyl, or optionally substituted C₁₋₄ alkoxy, and k is 1, 2, or 3. It should be noted that the G^{A1} in F-3-B can be substituted at any available position of the naphthyl ring, although preferably, one or two G^{A1} is/are ortho to the OH group. In some embodiments, in F-3-B, G^{C} is Cl, methyl, ethyl, ethynyl, or CN, and G^{D} is H, F, Cl, CN, C₁₋₄ alkyl optionally substituted with 1-3 fluorine, such as methyl, ethyl, or CF₃. In some embodiments, in F-3-B, G^{C} is Cl, methyl, ethyl, ethynyl, or CN, and G^{D} is H or F. In some embodiments, k is 1, G^{A1} is ortho to the OH group, and G^{A1} is F, Cl, CN, or C₁₋₄ alkyl optionally substituted with 1-3 fluorine. In some embodiments, k is 2, both G^{A1} are ortho to the OH group, and each G^{A1} is independently F, Cl, CN, or C₁₋₄ alkyl optionally substituted with 1-3 fluorine.

In some embodiments, R³ in Formula II (e.g., subformulae II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2) can be a bicyclic heteroaryl (e.g., benzothiazolyl, indazolyl, or isoquinolinyl), which is optionally substituted, for example, with 1-3 substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, at most one of the substituents is OH, —NH₂, protected —OH, or a protected —NH₂. For example, in some embodiments, R³ is

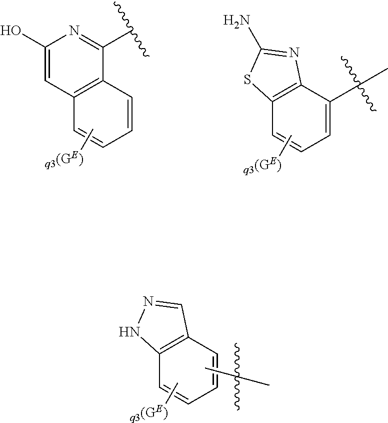

wherein: q3 is 0, 1, or 2, and G^{E} at each occurrence is independently F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂. In some embodiments, q3 is 0, 1, or 2, and G^{E} at each occurrence is F, Cl, C₁₋₄ alkyl (e.g., methyl, ethyl, propyl, isopropyl, tert-butyl), C₂₋₄ alkenyl, C₂₋₄ alkynyl (e.g., ethynyl), cyclopropyl, CH₂CH₂—CN, CF₂H, CF₃, or —CN.

Various selections of R³ suitable for Formula II (e.g., subformulae II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2) are exemplified herein in the specific examples. In some embodiments, R³ in Formula II (e.g., subformulae II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2) can be selected from:

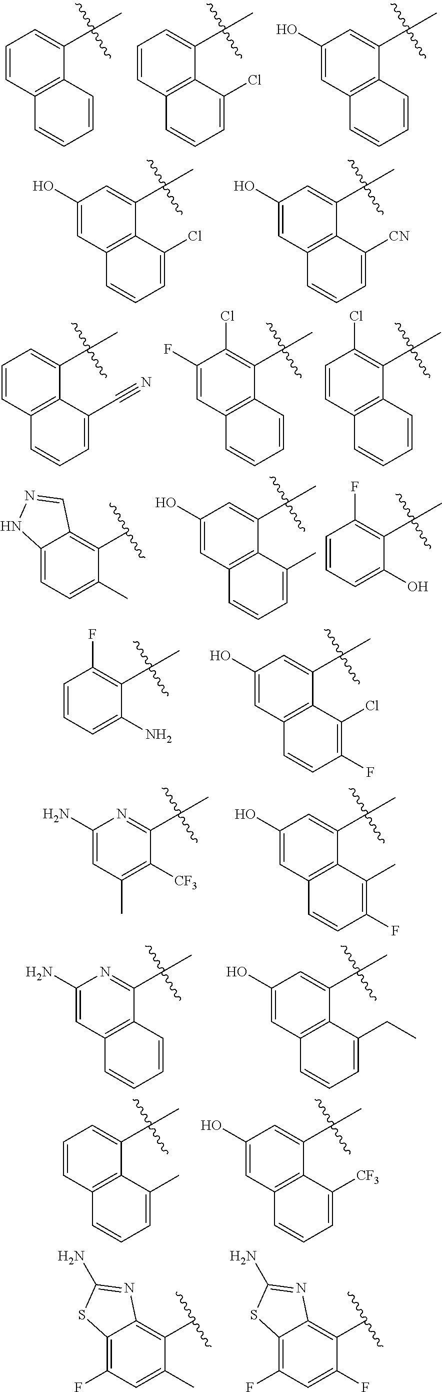

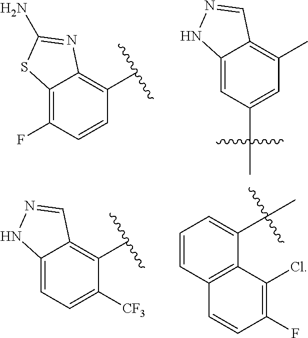 [SMILES: *c1ccc(F)c2sc(N)nc12 ; *c1cc(C)c2cn[nH]c2c1 ; *c1c(C(F)(F)F)ccc2[nH]ncc12 ; *c1cccc2ccc(F)c(Cl)c12]

In some embodiments, R³ in Formula II (e.g., subformulae II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2) can be selected from:

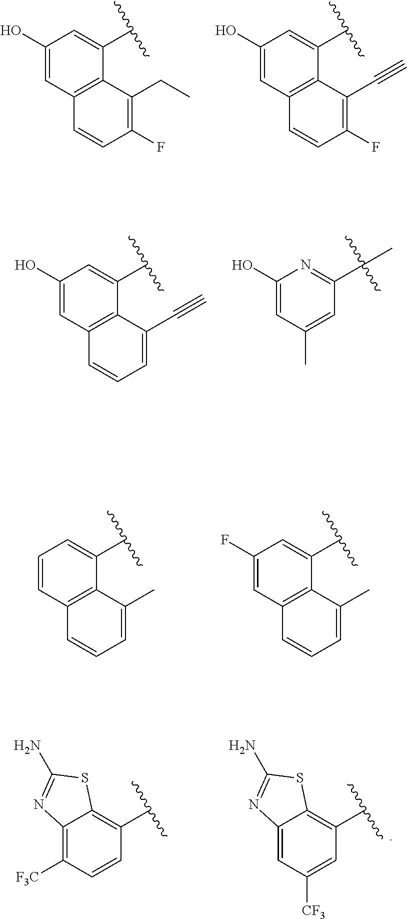

In some preferred embodiments, R³ in Formula II (e.g., subformulae II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2) can be selected from:

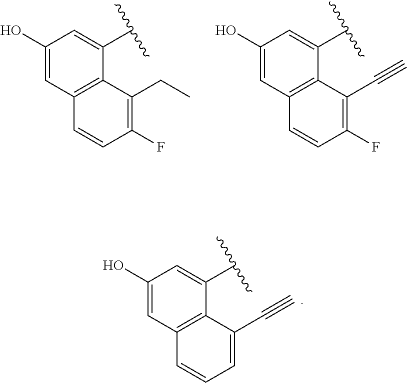 [SMILES: *c1cc(O)cc2ccc(F)c(CC)c12 ; *c1cc(O)cc2ccc(F)c(C#C)c12 ; *c1cc(O)cc2cccc(C#C)c12]

In some embodiments, the present disclosure also provides a compound of Formula III, or a pharmaceutically acceptable salt thereof:

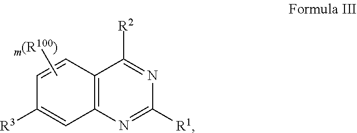

wherein:
R¹ is hydrogen, -(L¹)ⱼ₁-OR³⁰, halogen, -(L¹)ⱼ₁-NR²¹R²², or an optionally substituted heterocyclic or heteroaryl ring;
R² is a ring or ring-chain structure, e.g., having a pKa of about 6 or higher,
R³ is an optionally substituted aryl or an optionally substituted heteroaryl,
R¹⁰⁰ at each occurrence is independently F, Cl, Br, I, CN, —OH, —C(O)NH₂, —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), cyclopropyl, cyclobutyl, optionally substituted C₁₋₄ alkoxy (e.g., methoxy, ethoxy, —O—CH₂-cyclopropyl), cyclopropoxy, cyclobutoxy, or S—R^{A}, S(O)R^{A}, or S(O)₂R^{A}; wherein R^{A} at each occurrence is independently hydrogen, optionally substituted C₁₋₄ alkyl (e.g., methyl, ethyl, CF₃, etc.), cyclopropyl, or cyclobutyl; and
m is 0, 1, 2, or 3;
wherein:
   j1 is 0 or 1, and when j1 is 1, L¹ is an optionally substituted alkylene, an optionally substituted carbocyclylene, an optionally substituted heterocyclylene;
   R²¹ and R²² are independently hydrogen, a nitrogen protecting group, an optionally substituted C₁₋₆ alkyl, an optionally substituted carbocyclic ring, or an optionally substituted heterocyclic ring; or R²¹ and R²² are joined to form an optionally substituted heterocyclic or heteroaryl ring; and
   R³⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring.

The compound of Formula III (including any of the applicable sub-formulae as described herein) can exist in the form of an individual enantiomer, diastereomer, atropisomer, and/or geometric isomer, as applicable, or a mixture of stereoisomers, including racemic mixtures and mixtures enriched in one or more stereoisomers. In some embodiments, when applicable, the compound of Formula III (including any of the applicable sub-formulae as described herein) can exist as a mixture of atropisomers in any ratio, including about 1:1. In some embodiments, when applicable, the compound of Formula III (including any of the applicable sub-formulae as described herein) can exist as an isolated individual atropisomer substantially free (e.g., with less than 20%, less than 10%, less than 5%, less than 1%, by weight, by HPLC area, or both, or with a non-detectable amount) of the other atropisomer(s).

Suitable R¹, R², and R³ groups for Formula III include any of those described herein in connection with Formula I (e.g., its subformulae) and/or Formula II (e.g., its subformulae) in any combination. Suitable R¹⁰⁰ and m definitions for Formula III also include any of those described herein in connection with Formula I (or its subformulae) and/or Formula II (or its subformulae) in any combination. For example, in some embodiments, one or two R¹⁰⁰ are present in Formula III, i.e., m is 1 or 2. In some embodiments, R¹⁰⁰ at each occurrence is independently F, Cl, —CN, —OH, methoxy, ethoxy, —O—CH₂-cyclopropyl, —C(O)NHMe, CF₃, methyl, ethyl, isopropyl, or cyclopropyl. In some embodiments, two R¹⁰⁰ are present, and they are both ortho to the R³ group. In some embodiments, one of R¹⁰⁰ is F and the other of R¹⁰⁰ is Cl or CN. In some embodiments, the compound of Formula III can have a formula III-1, III-2, III-3, III-4, III-5, III-6, III-7, III-8, or III-9:

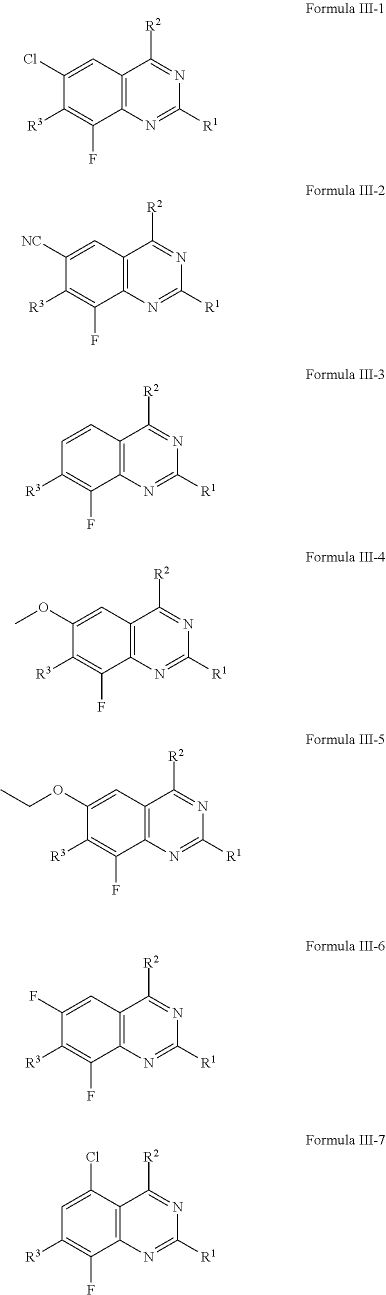

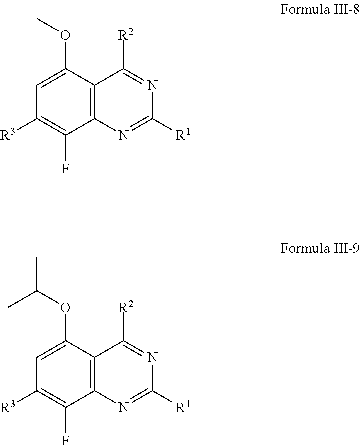

wherein R¹, R², and R³ are defined herein.

For example, in some embodiments, R¹ in Formula III (e.g., subformulae III-1, III-2, III-3, III-4, III-5, III-6, III-7, III-8, or III-9) can be selected from:

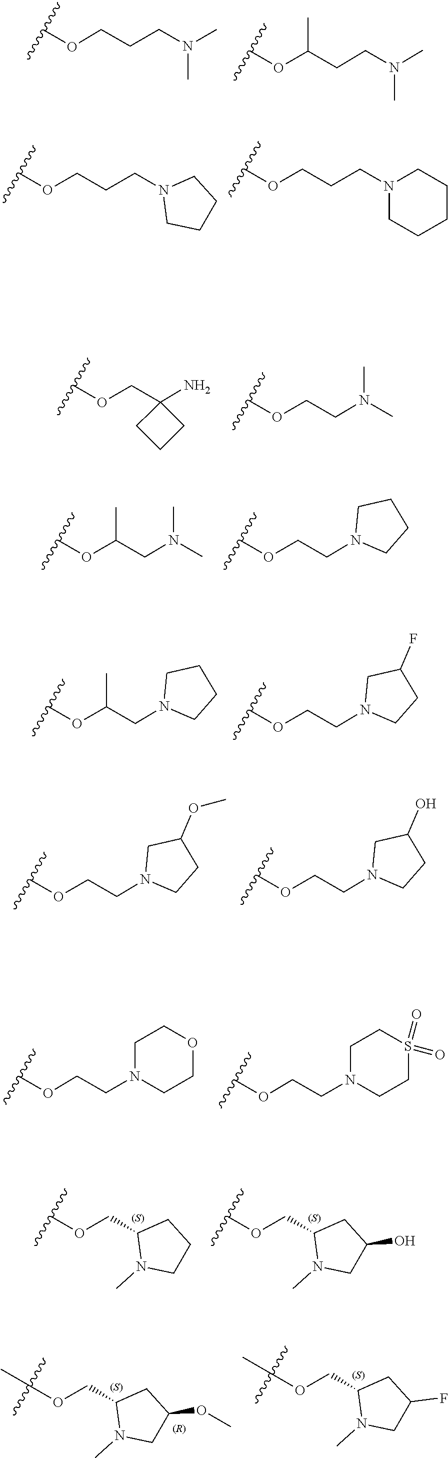

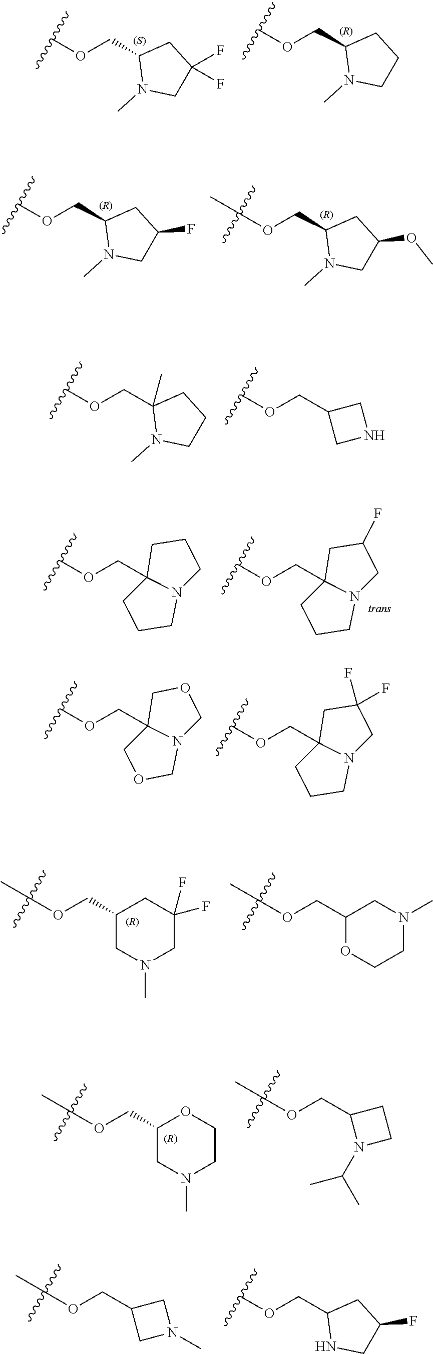

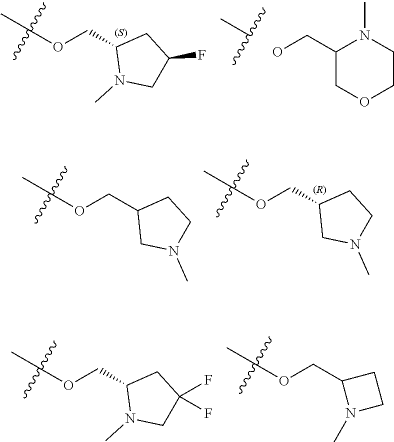

or R¹ can be hydrogen, methoxy,

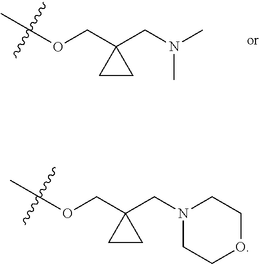

In some embodiments, R¹ in Formula III (e.g., subformulae III-1, III-2, III-3, III-4, III-5, III-6, III-7, III-8, or III-9) can be selected from:

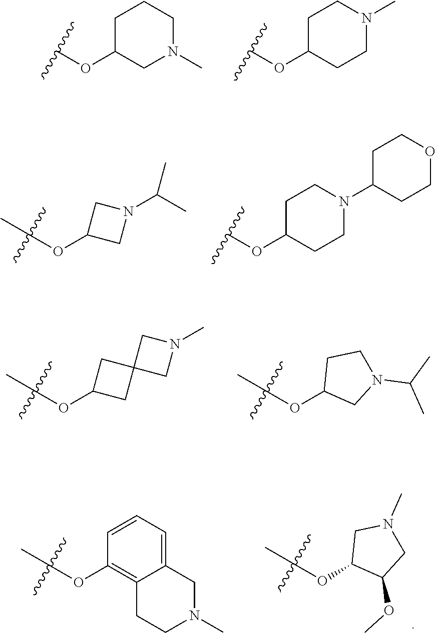

In some embodiments, R¹ in Formula III (e.g., subformulae III-1, III-2, III-3, III-4, III-5, III-6, III-7, III-8, or III-9) can be selected from:

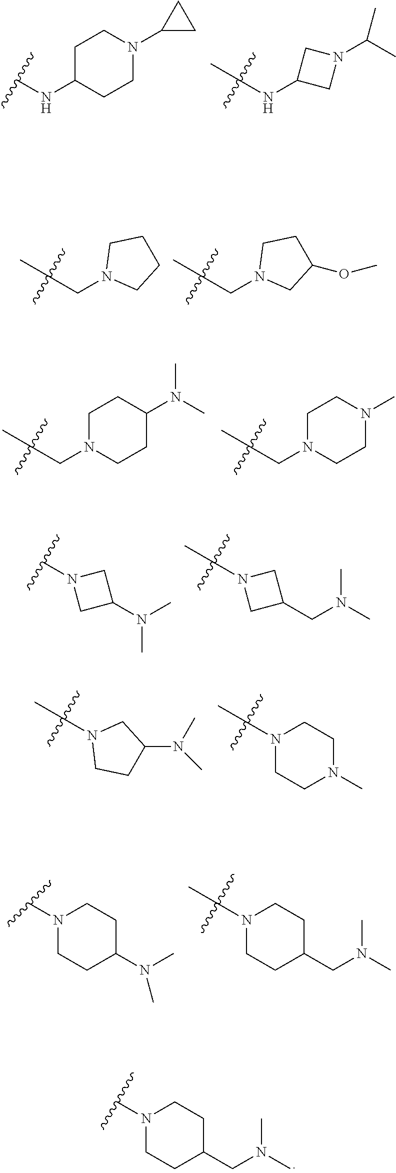

In some embodiments, R¹ in Formula III (e.g., subformulae III-1, III-2, III-3, III-4, III-5, III-6, III-7, III-8, or III-9) can be selected from:

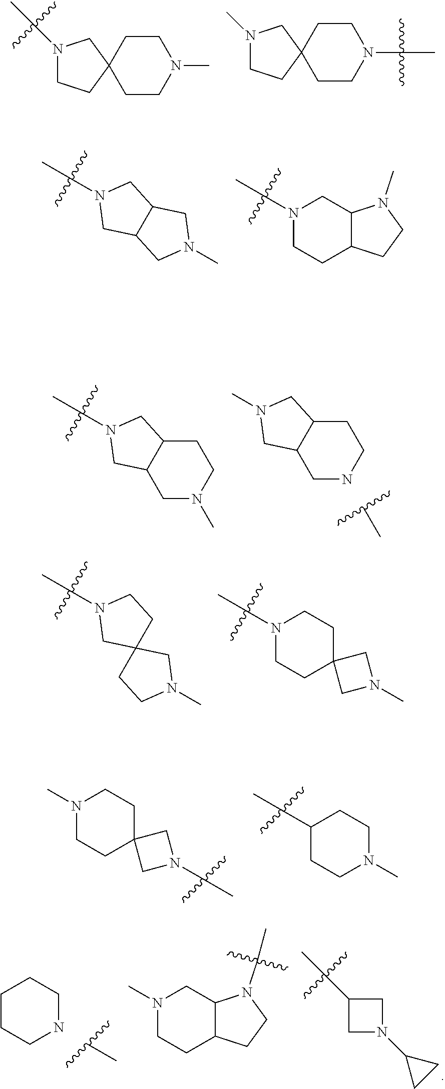

In some embodiments, R² in Formula III (e.g., subformulae III-1, III-2, III-3, III-4, III-5, III-6, III-7, III-8, or III-9) can be selected from:

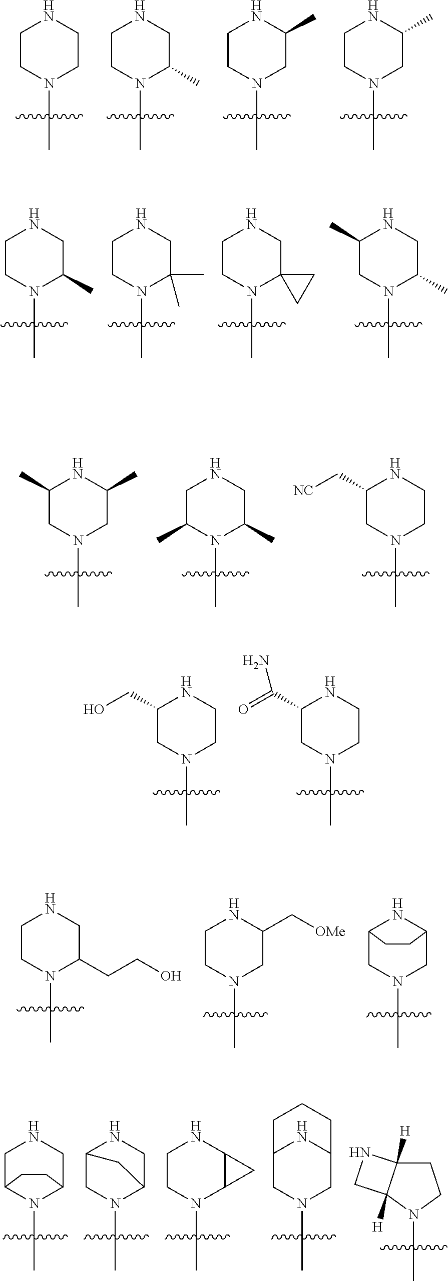

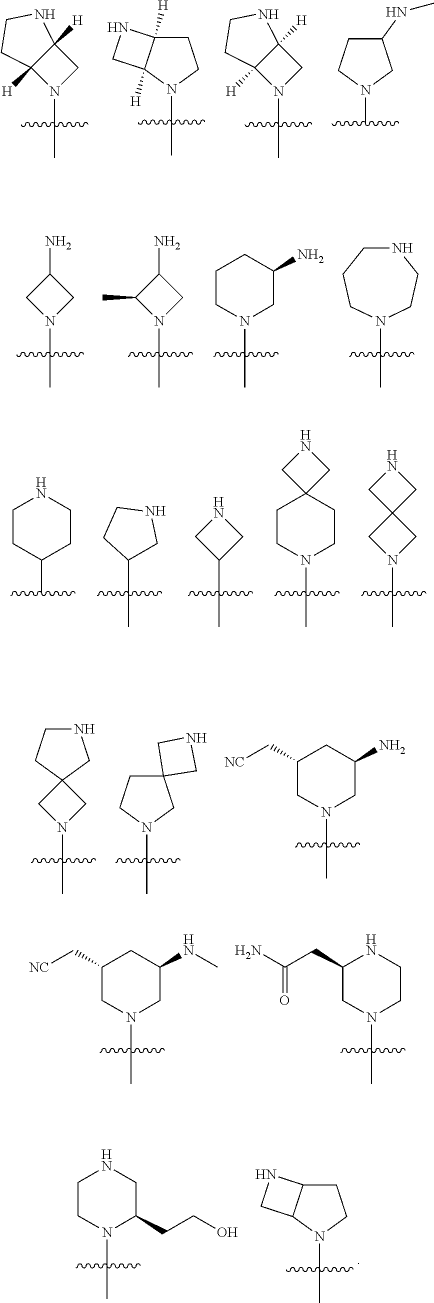

In some embodiments, R³ in Formula III (e.g., subformulae III-1, III-2, III-3, III-4, III-5, III-6, III-7, III-8, or III-9) can be selected from:

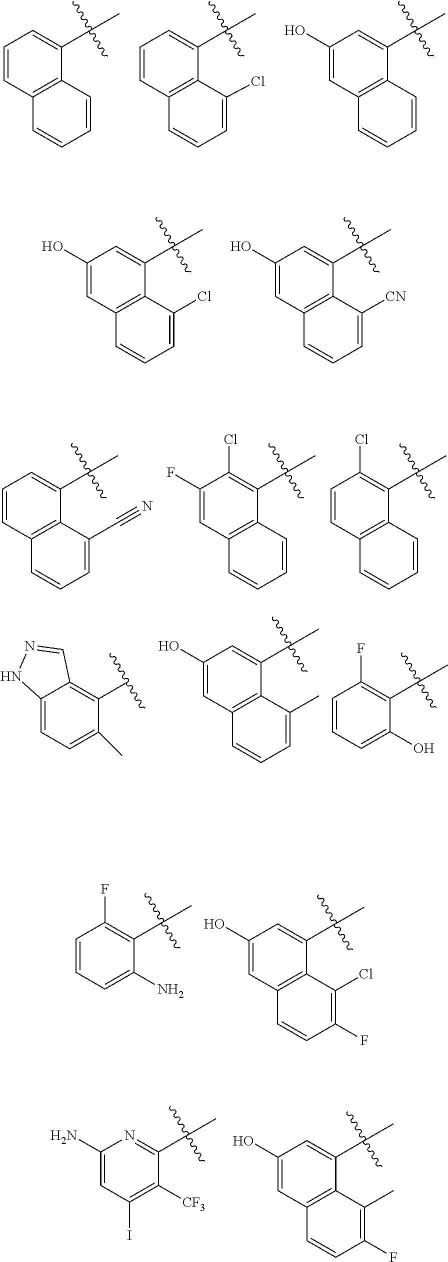

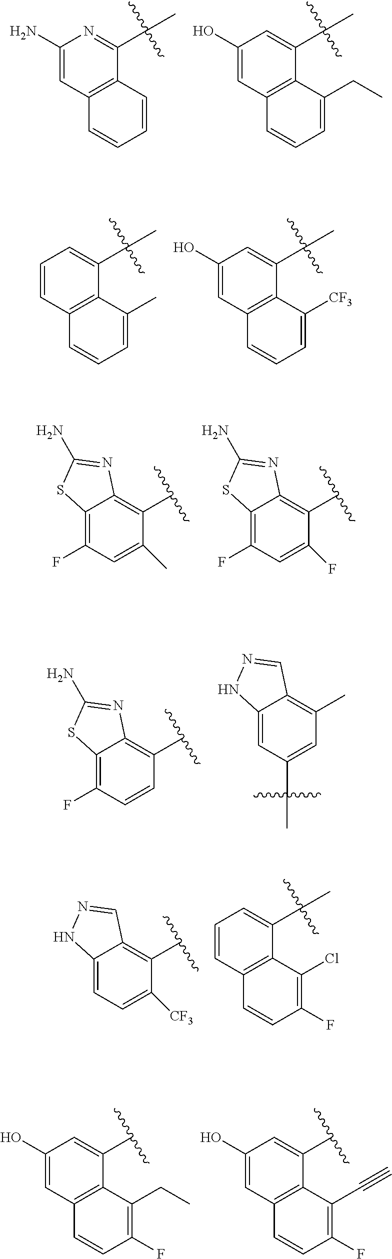

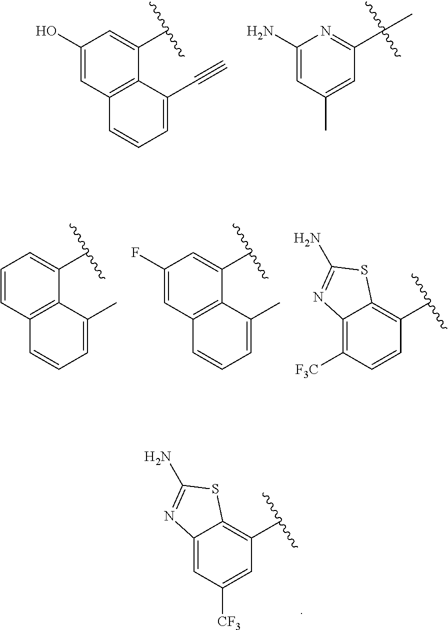

Other suitable definitions of R¹, R², and R³ for Formula III (e.g., subformulae III-1, III-2, III-3, III-4, III-5, III-6, III-7, III-8, or III-9) include any of those defined herein for the respective variables in connection with Formula I (or its subformulae) and/or Formula II (or its subformulae) in any combinations.

In some embodiments, the present disclosure also provides a compound selected from the compounds listed in Table A below, or a pharmaceutically acceptable salt thereof:

TABLE A
List of Compounds

| | |
|---|---|
| 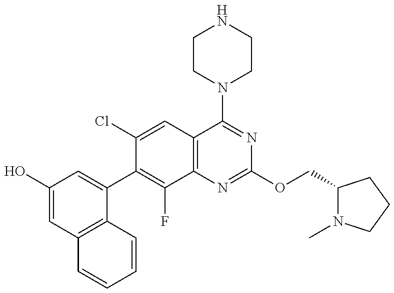 [SMILES: CN1CCC[C@H]1COc1nc(N2CCNCC2)c2cc(Cl)c(-c3cc(O)cc4ccccc34)c(F)c2n1] | 1 |
| 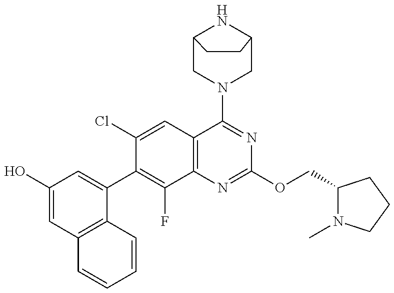 [SMILES: CN1CCC[C@H]1COc1nc(N2CC3CCC(C2)N3)c2cc(Cl)c(-c3cc(O)cc4ccccc34)c(F)c2n1] | 2 |
| 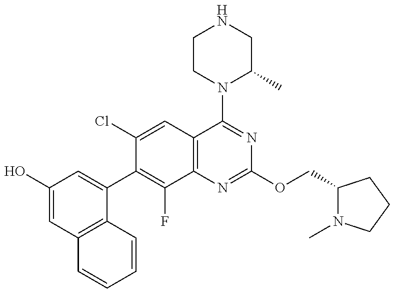 [SMILES: C[C@H]1CNCCN1c1nc(OC[C@@H]2CCCN2C)nc2c(F)c(-c3cc(O)cc4ccccc34)c(Cl)cc12] | 3 |
| 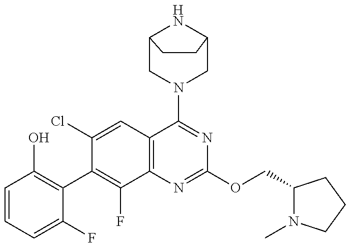 [SMILES: CN1CCC[C@H]1COc1nc(N2CC3CCC(C2)N3)c2cc(Cl)c(-c3c(O)cccc3F)c(F)c2n1] | 4 |
| 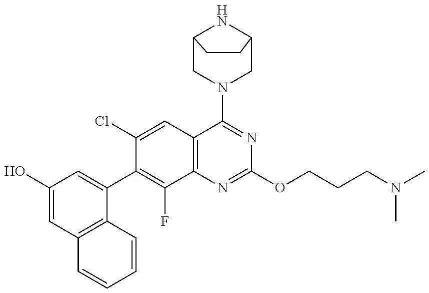 [SMILES: CN(C)CCCOc1nc(N2CC3CCC(C2)N3)c2cc(Cl)c(-c3cc(O)cc4ccccc34)c(F)c2n1] | 5 |
| 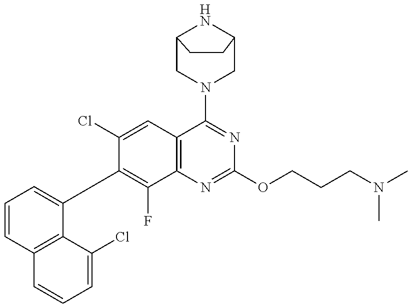 [SMILES: CN(C)CCCOc1nc(N2CC3CCC(C2)N3)c2cc(Cl)c(-c3cccc4cccc(Cl)c34)c(F)c2n1] | 6 |
| 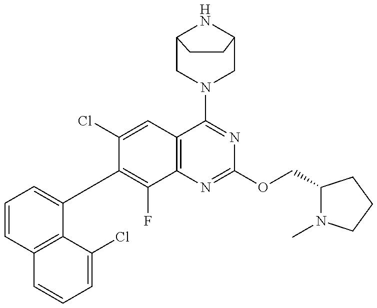 [SMILES: CN1CCC[C@H]1COc1nc(N2CC3CCC(C2)N3)c2cc(Cl)c(-c3cccc4cccc(Cl)c34)c(F)c2n1] | 7 |
| 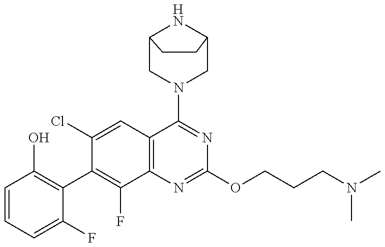 [SMILES: CN(C)CCCOc1nc(N2CC3CCC(C2)N3)c2cc(Cl)c(-c3c(O)cccc3F)c(F)c2n1] | 8 |
| 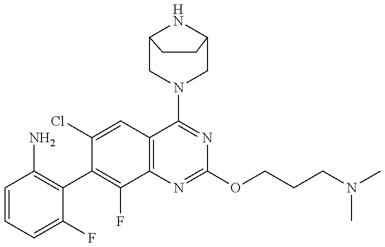 [SMILES: CN(C)CCCOc1nc(N2CC3CCC(C2)N3)c2cc(Cl)c(-c3c(N)cccc3F)c(F)c2n1] | 9 |
| 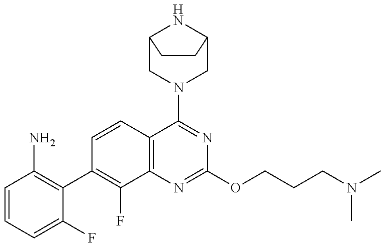 [SMILES: CN(C)CCCOc1nc(N2CC3CCC(C2)N3)c2ccc(-c3c(N)cccc3F)c(F)c2n1] | 10 |
| 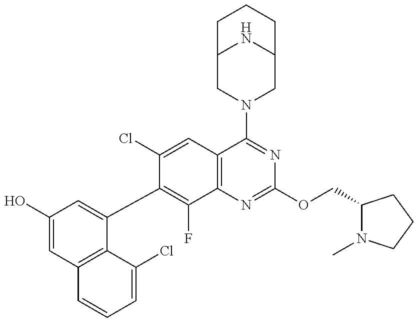 [SMILES: CN1CCC[C@H]1COc1nc(N2CC3CCCC(C2)N3)c2cc(Cl)c(-c3cc(O)cc4cccc(Cl)c34)c(F)c2n1] | 11 |
| 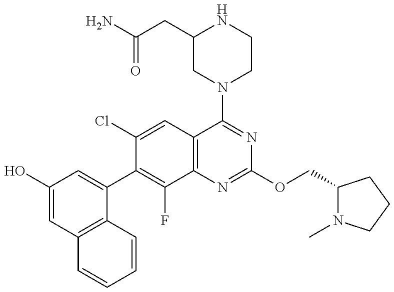 [SMILES: CN1CCC[C@H]1COc1nc(N2CCNC(CC(N)=O)C2)c2cc(Cl)c(-c3cc(O)cc4ccccc34)c(F)c2n1] | 12 |
| 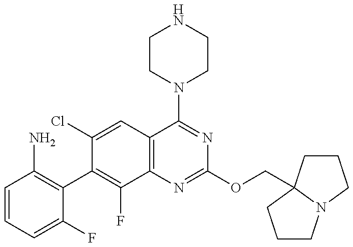 [SMILES: Nc1cccc(F)c1-c1c(Cl)cc2c(N3CCNCC3)nc(OCC34CCCN3CCC4)nc2c1F] | 14 |
| 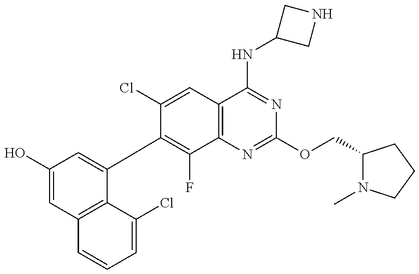 [SMILES: CN1CCC[C@H]1COc1nc(NC2CNC2)c2cc(Cl)c(-c3cc(O)cc4cccc(Cl)c34)c(F)c2n1] | 15 |
| 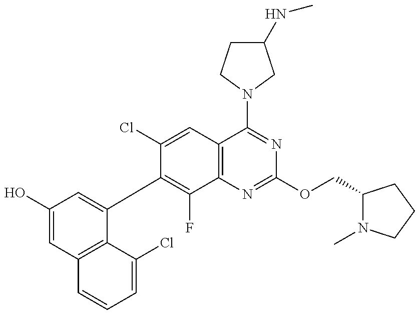 [SMILES: CNC1CCN(c2nc(OC[C@@H]3CCCN3C)nc3c(F)c(-c4cc(O)cc5cccc(Cl)c45)c(Cl)cc23)C1] | 16 |
| 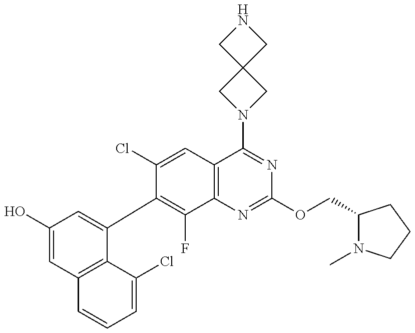 [SMILES: CN1CCC[C@H]1COc1nc(N2CC3(CNC3)C2)c2cc(Cl)c(-c3cc(O)cc4cccc(Cl)c34)c(F)c2n1] | 17 |
| 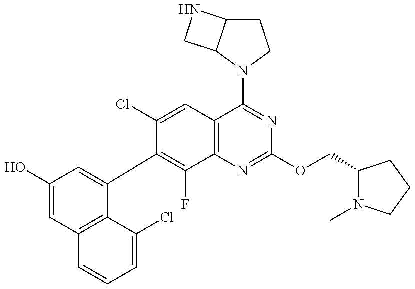 [SMILES: CN1CCC[C@H]1COc1nc(N2CCC3NCC32)c2cc(Cl)c(-c3cc(O)cc4cccc(Cl)c34)c(F)c2n1] | 18 |
| 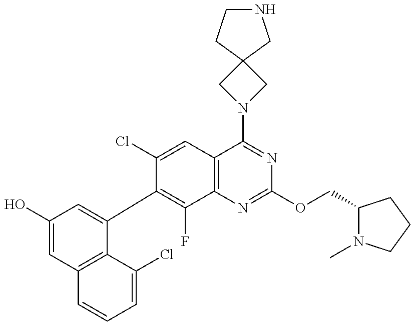 [SMILES: CN1CCC[C@H]1COc1nc(N2CC3(CCNC3)C2)c2cc(Cl)c(-c3cc(O)cc4cccc(Cl)c34)c(F)c2n1] | 19 |
| 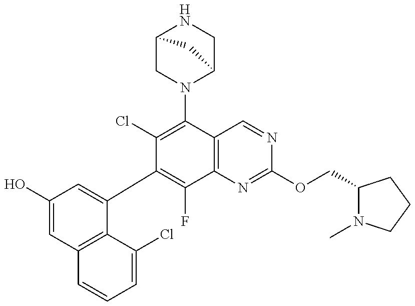 [SMILES: CN1CCC[C@H]1COc1ncc2c(N3C[C@@H]4C[C@H]3CN4)c(Cl)c(-c3cc(O)cc4cccc(Cl)c34)c(F)c2n1] | 20 |
| 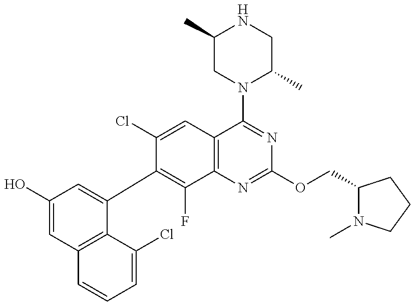 [SMILES: C[C@@H]1CN(c2nc(OC[C@@H]3CCCN3C)nc3c(F)c(-c4cc(O)cc5cccc(Cl)c45)c(Cl)cc23)[C@@H](C)CN1] | 21 |
| 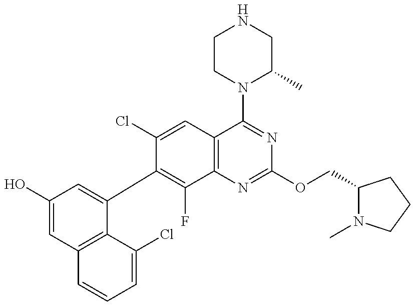 [SMILES: C[C@H]1CNCCN1c1nc(OC[C@@H]2CCCN2C)nc2c(F)c(-c3cc(O)cc4cccc(Cl)c34)c(Cl)cc12] | 22 |
| 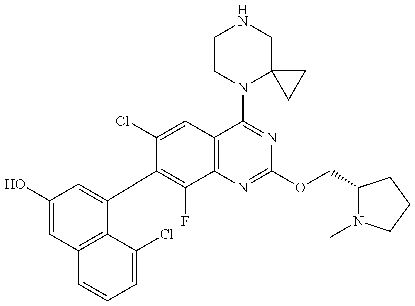 [SMILES: CN1CCC[C@H]1COc1nc(N2CCNCC23CC3)c2cc(Cl)c(-c3cc(O)cc4cccc(Cl)c34)c(F)c2n1] | 23 |
| 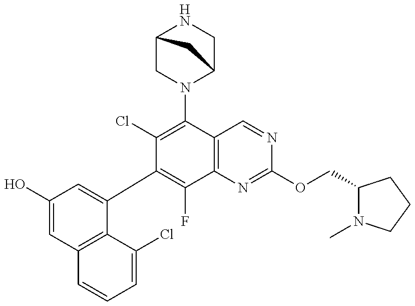 [SMILES: CN1CCC[C@H]1COc1ncc2c(N3C[C@H]4C[C@@H]3CN4)c(Cl)c(-c3cc(O)cc4cccc(Cl)c34)c(F)c2n1] | 24 |
| 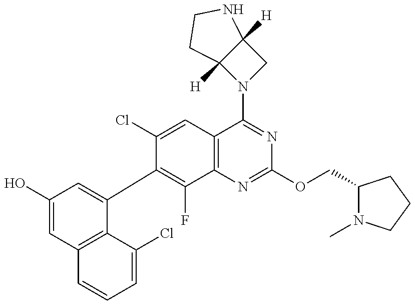 [SMILES: CN1CCC[C@H]1COc1nc(N2C[C@H]3NCC[C@H]32)c2cc(Cl)c(-c3cc(O)cc4cccc(Cl)c34)c(F)c2n1] | 25 |
| 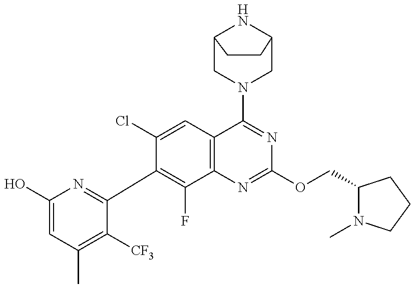 [SMILES: Cc1cc(O)nc(-c2c(Cl)cc3c(N4CC5CCC(C4)N5)nc(OC[C@@H]4CCCN4C)nc3c2F)c1C(F)(F)F] | 26 |
| 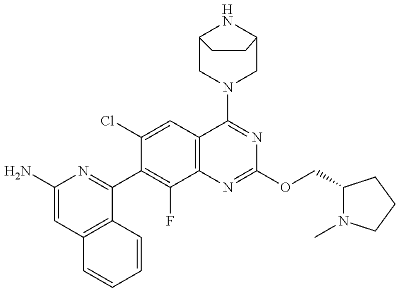 [SMILES: CN1CCC[C@H]1COc1nc(N2CC3CCC(C2)N3)c2cc(Cl)c(-c3nc(N)cc4ccccc34)c(F)c2n1] | 27 |
| 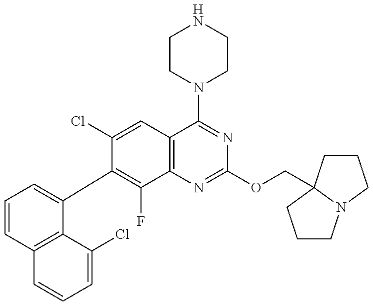 [SMILES: Fc1c(-c2cccc3cccc(Cl)c23)c(Cl)cc2c(N3CCNCC3)nc(OCC34CCCN3CCC4)nc12] | 28 |
| 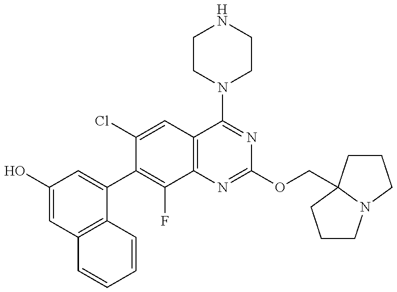 [SMILES: Oc1cc(-c2c(Cl)cc3c(N4CCNCC4)nc(OCC45CCCN4CCC5)nc3c2F)c2ccccc2c1] | 29 |
| 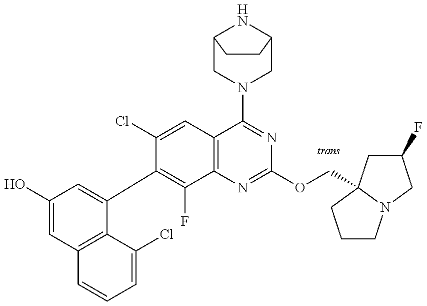 [SMILES: Oc1cc(-c2c(Cl)cc3c(N4CC5CCC(C4)N5)nc(OC[C@@]45CCCN4C[C@H](F)C5)nc3c2F)c2c(Cl)cccc2c1] | 30 |
| 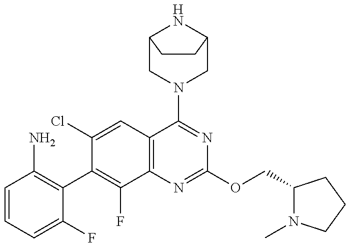 [SMILES: CN1CCC[C@H]1COc1nc(N2CC3CCC(C2)N3)c2cc(Cl)c(-c3c(N)cccc3F)c(F)c2n1] | 31 |
| 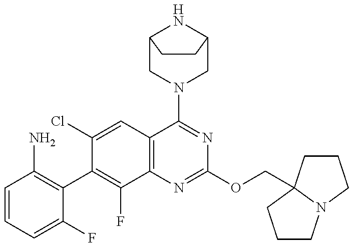 [SMILES: Nc1cccc(F)c1-c1c(Cl)cc2c(N3CC4CCC(C3)N4)nc(OCC34CCCN3CCC4)nc2c1F] | 32 |
| 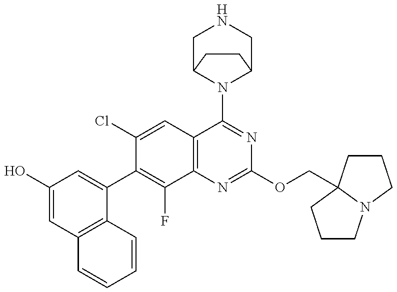 [SMILES: Oc1cc(-c2c(Cl)cc3c(N4C5CCC4CNC5)nc(OCC45CCCN4CCC5)nc3c2F)c2ccccc2c1] | 33 |
| 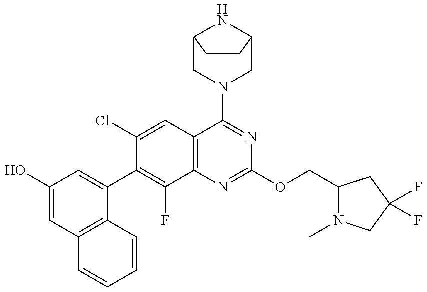 [SMILES: CN1CC(F)(F)CC1COc1nc(N2CC3CCC(C2)N3)c2cc(Cl)c(-c3cc(O)cc4ccccc34)c(F)c2n1] | 34 |
| 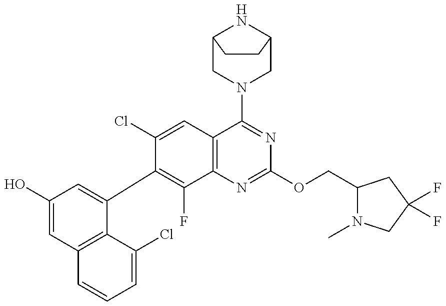 [SMILES: CN1CC(F)(F)CC1COc1nc(N2CC3CCC(C2)N3)c2cc(Cl)c(-c3cc(O)cc4cccc(Cl)c34)c(F)c2n1] | 35 |
| 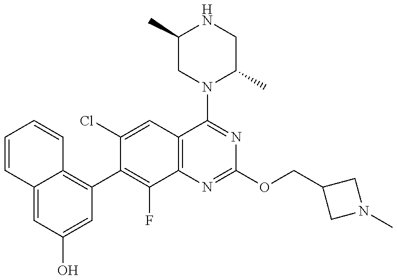 [SMILES: C[C@@H]1CN(c2nc(OCC3CN(C)C3)nc3c(F)c(-c4cc(O)cc5ccccc45)c(Cl)cc23)[C@@H](C)CN1] | 36 |
| 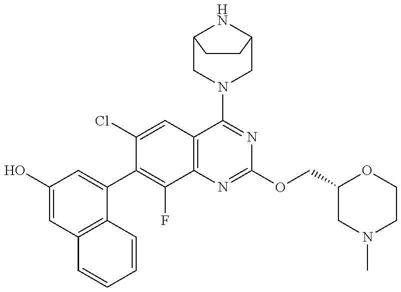 [SMILES: CN1CCO[C@@H](COc2nc(N3CC4CCC(C3)N4)c3cc(Cl)c(-c4cc(O)cc5ccccc45)c(F)c3n2)C1] | 37 |
| 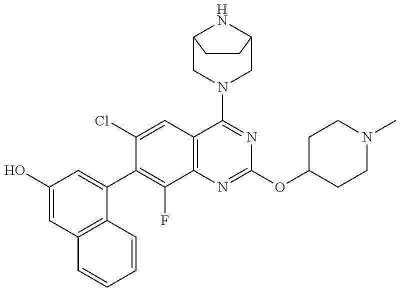 [SMILES: CN1CCC(Oc2nc(N3CC4CCC(C3)N4)c3cc(Cl)c(-c4cc(O)cc5ccccc45)c(F)c3n2)CC1] | 38 |
| 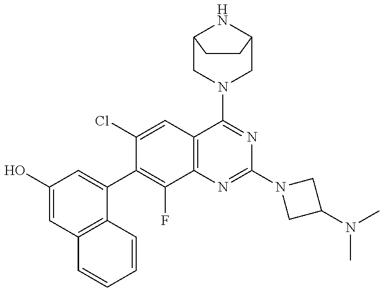 [SMILES: CN(C)C1CN(c2nc(N3CC4CCC(C3)N4)c3cc(Cl)c(-c4cc(O)cc5ccccc45)c(F)c3n2)C1] | 39 |
| 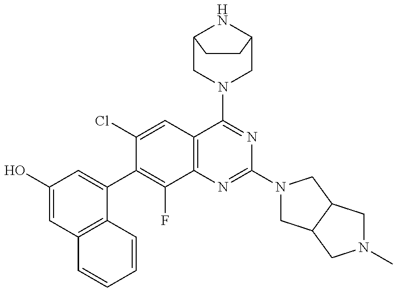 [SMILES: CN1CC2CN(c3nc(N4CC5CCC(C4)N5)c4cc(Cl)c(-c5cc(O)cc6ccccc56)c(F)c4n3)CC2C1] | 40 |
| 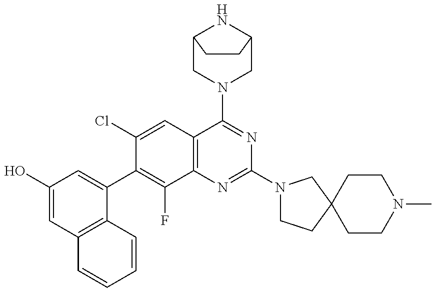 [SMILES: CN1CCC2(CC1)CCN(c1nc(N3CC4CCC(C3)N4)c3cc(Cl)c(-c4cc(O)cc5ccccc45)c(F)c3n1)C2] | 41 |
| 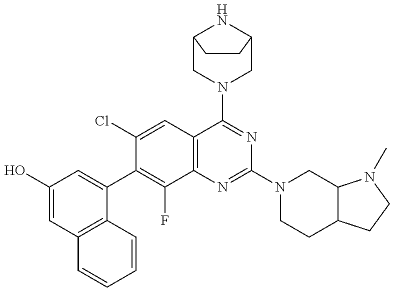 [SMILES: CN1CCC2CCN(c3nc(N4CC5CCC(C4)N5)c4cc(Cl)c(-c5cc(O)cc6ccccc56)c(F)c4n3)CC21] | 42 |
| 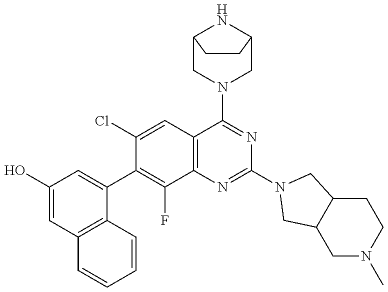 [SMILES: CN1CCC2CN(c3nc(N4CC5CCC(C4)N5)c4cc(Cl)c(-c5cc(O)cc6ccccc56)c(F)c4n3)CC2C1] | 43 |
| 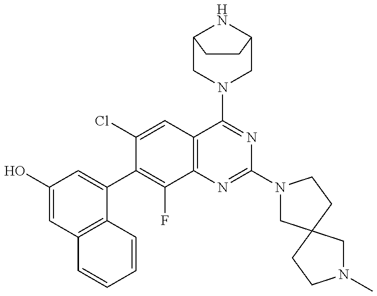 [SMILES: CN1CCC2(CCN(c3nc(N4CC5CCC(C4)N5)c4cc(Cl)c(-c5cc(O)cc6ccccc56)c(F)c4n3)C2)C1] | 44 |
| 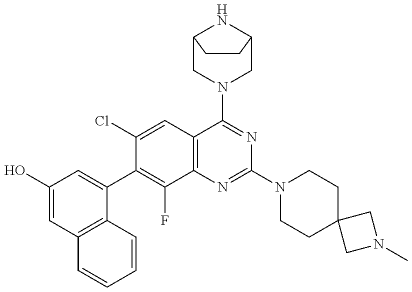 [SMILES: CN1CC2(CCN(c3nc(N4CC5CCC(C4)N5)c4cc(Cl)c(-c5cc(O)cc6ccccc56)c(F)c4n3)CC2)C1] | 45 |
| 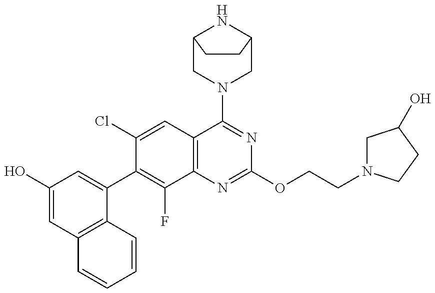 [SMILES: Oc1cc(-c2c(Cl)cc3c(N4CC5CCC(C4)N5)nc(OCCN4CCC(O)C4)nc3c2F)c2ccccc2c1] | 46 |
| 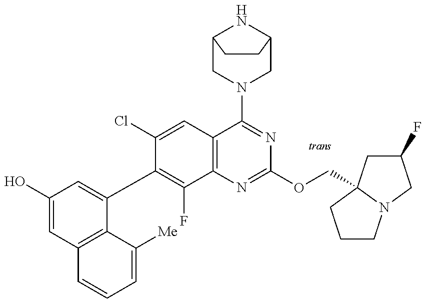 [SMILES: Cc1cccc2cc(O)cc(-c3c(Cl)cc4c(N5CC6CCC(C5)N6)nc(OC[C@@]56CCCN5C[C@H](F)C6)nc4c3F)c12] | 47 |
| 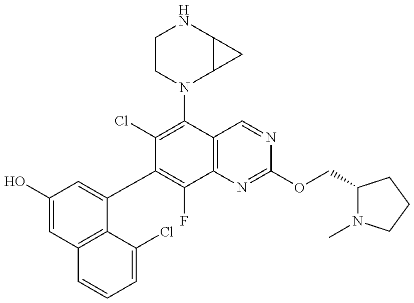 [SMILES: CN1CCC[C@H]1COc1ncc2c(N3CCNC4CC43)c(Cl)c(-c3cc(O)cc4cccc(Cl)c34)c(F)c2n1] | 48 |
| 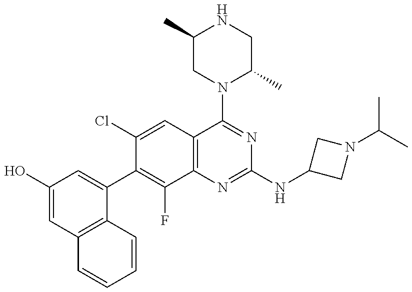 [SMILES: CC(C)N1CC(Nc2nc(N3C[C@@H](C)NC[C@@H]3C)c3cc(Cl)c(-c4cc(O)cc5ccccc45)c(F)c3n2)C1] | 49 |
| 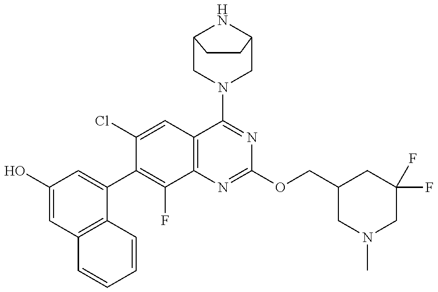 [SMILES: CN1CC(COc2nc(N3CC4CCC(C3)N4)c3cc(Cl)c(-c4cc(O)cc5ccccc45)c(F)c3n2)CC(F)(F)C1] | 50 |
| 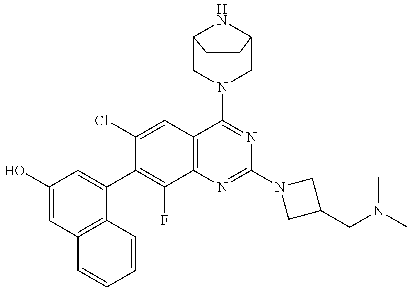 [SMILES: CN(C)CC1CN(c2nc(N3CC4CCC(C3)N4)c3cc(Cl)c(-c4cc(O)cc5ccccc45)c(F)c3n2)C1] | 51 |
| 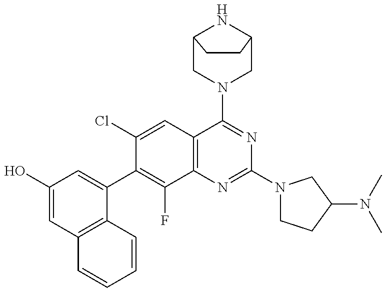 [SMILES: CN(C)C1CCN(c2nc(N3CC4CCC(C3)N4)c3cc(Cl)c(-c4cc(O)cc5ccccc45)c(F)c3n2)C1] | 52 |
| 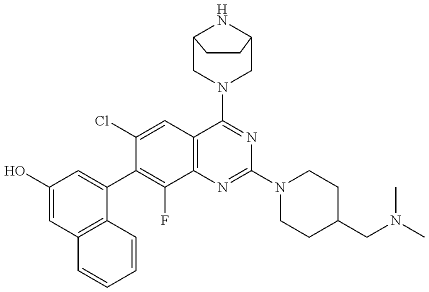 [SMILES: CN(C)CC1CCN(c2nc(N3CC4CCC(C3)N4)c3cc(Cl)c(-c4cc(O)cc5ccccc45)c(F)c3n2)CC1] | 53 |
| 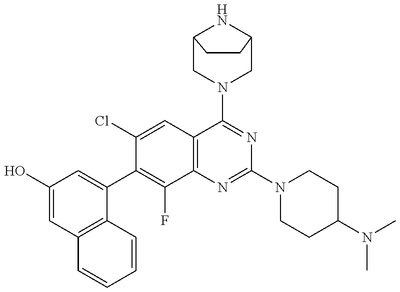 [SMILES: CN(C)C1CCN(c2nc(N3CC4CCC(C3)N4)c3cc(Cl)c(-c4cc(O)cc5ccccc45)c(F)c3n2)CC1] | 54 |
| 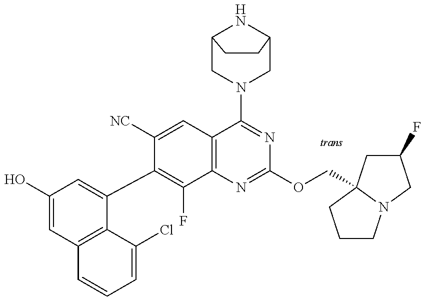 [SMILES: N#Cc1cc2c(N3CC4CCC(C3)N4)nc(OC[C@@]34CCCN3C[C@H](F)C4)nc2c(F)c1-c1cc(O)cc2cccc(Cl)c12] | 55 |
| 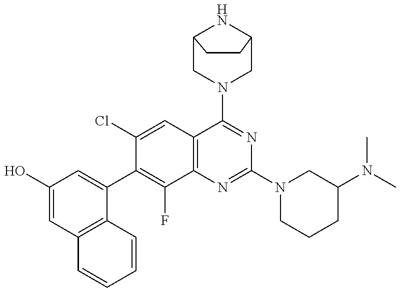 [SMILES: CN(C)C1CCCN(c2nc(N3CC4CCC(C3)N4)c3cc(Cl)c(-c4cc(O)cc5ccccc45)c(F)c3n2)C1] | 56 |
| 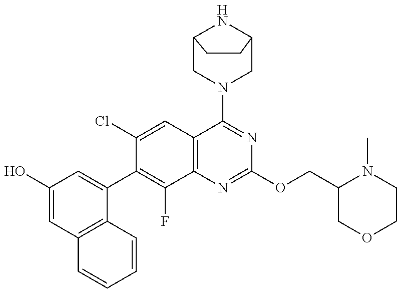 [SMILES: CN1CCOCC1COc1nc(N2CC3CCC(C2)N3)c2cc(Cl)c(-c3cc(O)cc4ccccc34)c(F)c2n1] | 57 |
| 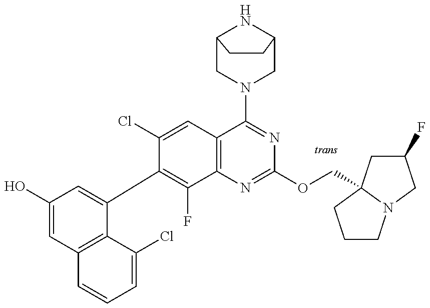 [SMILES: Oc1cc(-c2c(Cl)cc3c(N4CC5CCC(C4)N5)nc(OC[C@@]45CCCN4C[C@H](F)C5)nc3c2F)c2c(Cl)cccc2c1] | 58 |
| 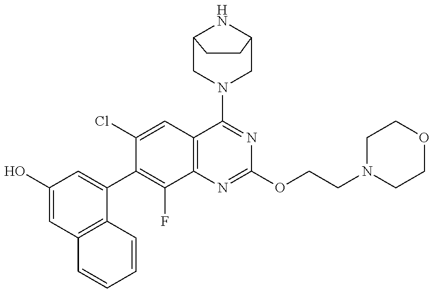 [SMILES: Oc1cc(-c2c(Cl)cc3c(N4CC5CCC(C4)N5)nc(OCCN4CCOCC4)nc3c2F)c2ccccc2c1] | 59 |
| 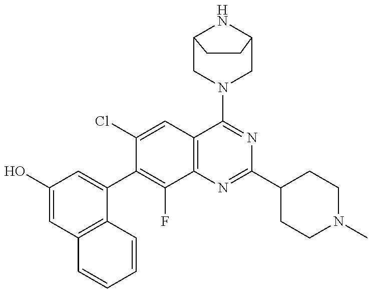 [SMILES: CN1CCC(c2nc(N3CC4CCC(C3)N4)c3cc(Cl)c(-c4cc(O)cc5ccccc45)c(F)c3n2)CC1] | 60 |
| 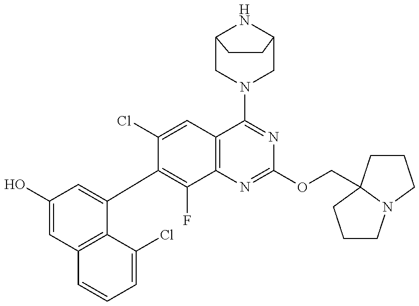 [SMILES: Oc1cc(-c2c(Cl)cc3c(N4CC5CCC(C4)N5)nc(OCC45CCCN4CCC5)nc3c2F)c2c(Cl)cccc2c1] | 61 |
| 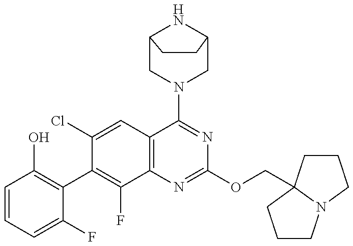 [SMILES: Oc1cccc(F)c1-c1c(Cl)cc2c(N3CC4CCC(C3)N4)nc(OCC34CCCN3CCC4)nc2c1F] | 63 |
| 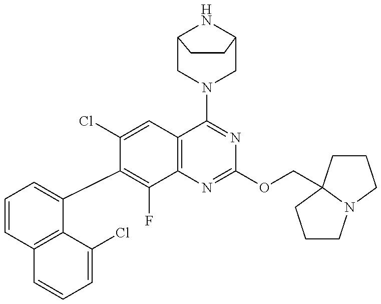 [SMILES: Fc1c(-c2cccc3cccc(Cl)c23)c(Cl)cc2c(N3CC4CCC(C3)N4)nc(OCC34CCCN3CCC4)nc12] | 64 |
| 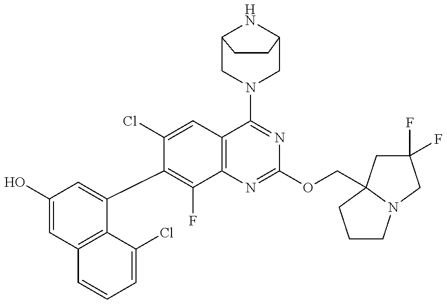 [SMILES: Oc1cc(-c2c(Cl)cc3c(N4CC5CCC(C4)N5)nc(OCC45CCCN4CC(F)(F)C5)nc3c2F)c2c(Cl)cccc2c1] | 65 |
| 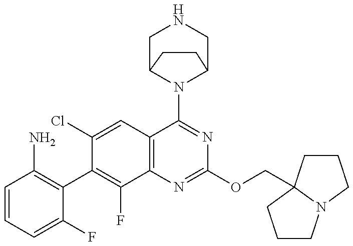 [SMILES: Nc1cccc(F)c1-c1c(Cl)cc2c(N3C4CCC3CNC4)nc(OCC34CCCN3CCC4)nc2c1F] | 66 |
| 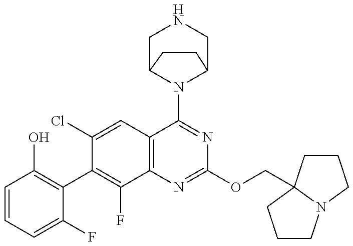 [SMILES: Oc1cccc(F)c1-c1c(Cl)cc2c(N3C4CCC3CNC4)nc(OCC34CCCN3CCC4)nc2c1F] | 67 |
| 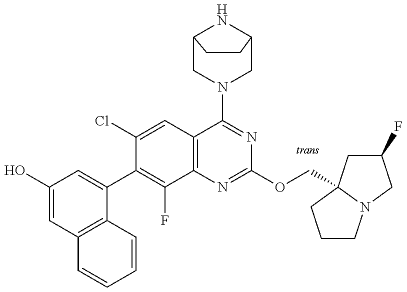 [SMILES: Oc1cc(-c2c(Cl)cc3c(N4CC5CCC(C4)N5)nc(OC[C@@]45CCCN4C[C@H](F)C5)nc3c2F)c2ccccc2c1] | 68 |
| 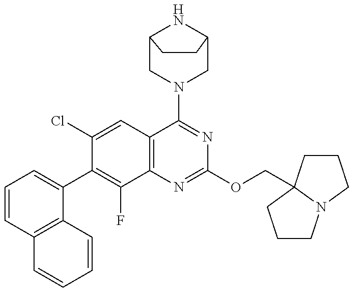 [SMILES: Fc1c(-c2cccc3ccccc23)c(Cl)cc2c(N3CC4CCC(C3)N4)nc(OCC34CCCN3CCC4)nc12] | 69 |
| 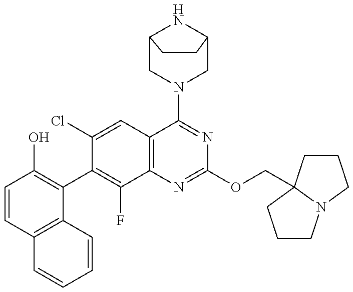 [SMILES: Oc1ccc2ccccc2c1-c1c(Cl)cc2c(N3CC4CCC(C3)N4)nc(OCC34CCCN3CCC4)nc2c1F] | 70 |
| 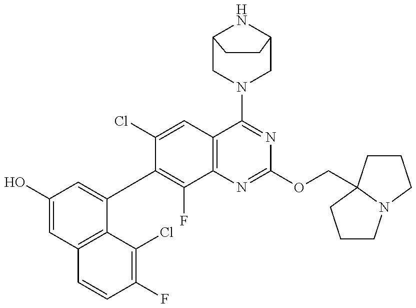 [SMILES: Oc1cc(-c2c(Cl)cc3c(N4CC5CCC(C4)N5)nc(OCC45CCCN4CCC5)nc3c2F)c2c(Cl)c(F)ccc2c1] | 71 |
| 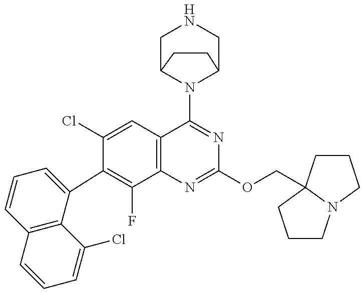 [SMILES: Fc1c(-c2cccc3cccc(Cl)c23)c(Cl)cc2c(N3C4CCC3CNC4)nc(OCC34CCCN3CCC4)nc12] | 72 |
| 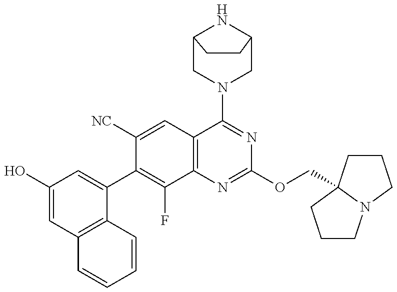 | 73 |
| 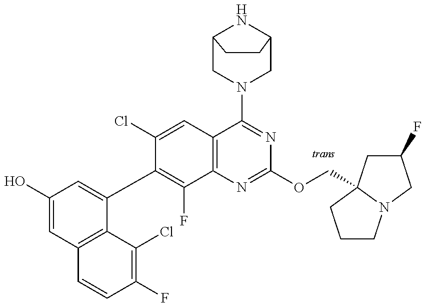 [SMILES: Oc1cc(-c2c(Cl)cc3c(N4CC5CCC(C4)N5)nc(OC[C@@]45CCCN4C[C@H](F)C5)nc3c2F)c2c(Cl)c(F)ccc2c1] | 74 |
| 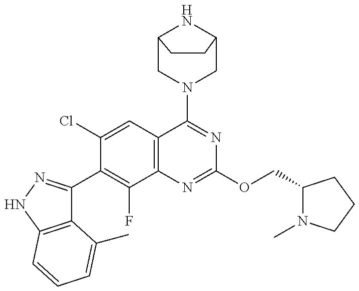 [SMILES: Cc1cccc2[nH]nc(-c3c(Cl)cc4c(N5CC6CCC(C5)N6)nc(OC[C@@H]5CCCN5C)nc4c3F)c12] | 75 |
| 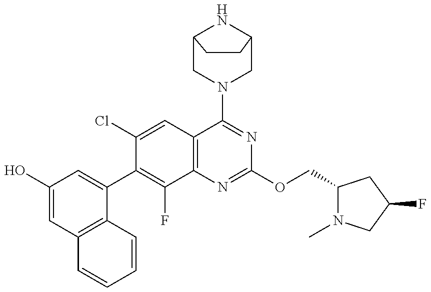 [SMILES: CN1C[C@H](F)C[C@H]1COc1nc(N2CC3CCC(C2)N3)c2cc(Cl)c(-c3cc(O)cc4ccccc34)c(F)c2n1] | 76 |
| 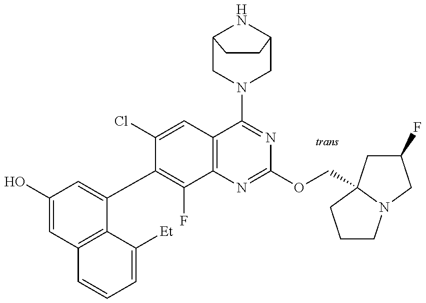 [SMILES: CCc1cccc2cc(O)cc(-c3c(Cl)cc4c(N5CC6CCC(C5)N6)nc(OC[C@@]56CCCN5C[C@H](F)C6)nc4c3F)c12] | 77 |
| 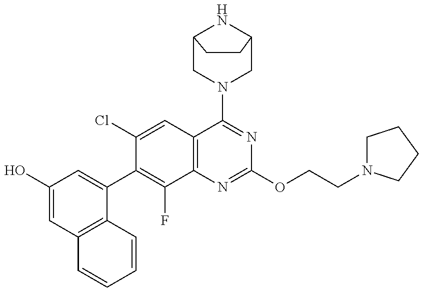 [SMILES: Oc1cc(-c2c(Cl)cc3c(N4CC5CCC(C4)N5)nc(OCCN4CCCC4)nc3c2F)c2ccccc2c1] | 78 |
| 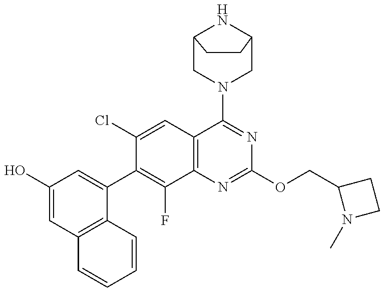 [SMILES: CN1CCC1COc1nc(N2CC3CCC(C2)N3)c2cc(Cl)c(-c3cc(O)cc4ccccc34)c(F)c2n1] | 79 |
| 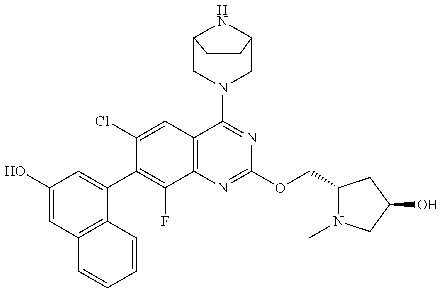 [SMILES: CN1C[C@H](O)C[C@H]1COc1nc(N2CC3CCC(C2)N3)c2cc(Cl)c(-c3cc(O)cc4ccccc34)c(F)c2n1] | 80 |
| 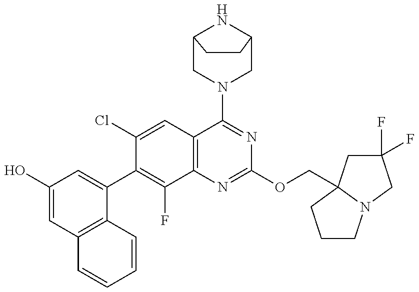 [SMILES: Oc1cc(-c2c(Cl)cc3c(N4CC5CCC(C4)N5)nc(OCC45CCCN4CC(F)(F)C5)nc3c2F)c2ccccc2c1] | 81 |
| 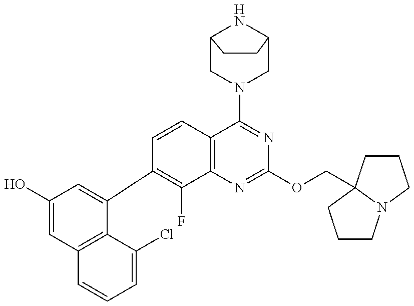 [SMILES: Oc1cc(-c2ccc3c(N4CC5CCC(C4)N5)nc(OCC45CCCN4CCC5)nc3c2F)c2c(Cl)cccc2c1] | 82 |
| 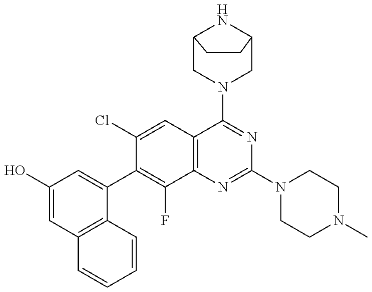 [SMILES: CN1CCN(c2nc(N3CC4CCC(C3)N4)c3cc(Cl)c(-c4cc(O)cc5ccccc45)c(F)c3n2)CC1] | 83 |
| 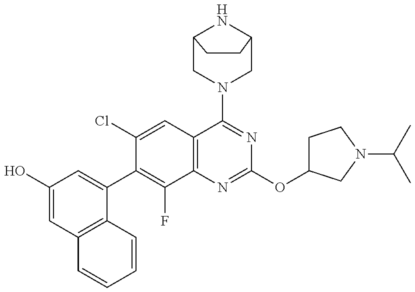 [SMILES: CC(C)N1CCC(Oc2nc(N3CC4CCC(C3)N4)c3cc(Cl)c(-c4cc(O)cc5ccccc45)c(F)c3n2)C1] | 84 |
| 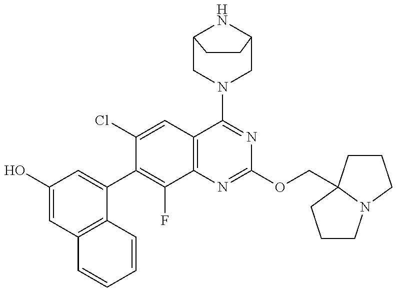 [SMILES: Oc1cc(-c2c(Cl)cc3c(N4CC5CCC(C4)N5)nc(OCC45CCCN4CCC5)nc3c2F)c2ccccc2c1] | 85 |
| 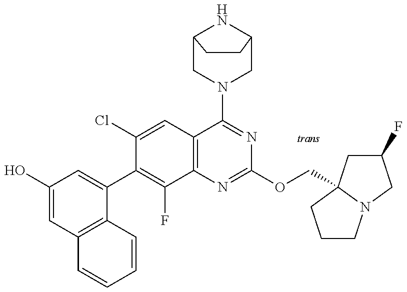 [SMILES: Oc1cc(-c2c(Cl)cc3c(N4CC5CCC(C4)N5)nc(OC[C@@]45CCCN4C[C@H](F)C5)nc3c2F)c2ccccc2c1] | 86 |
| 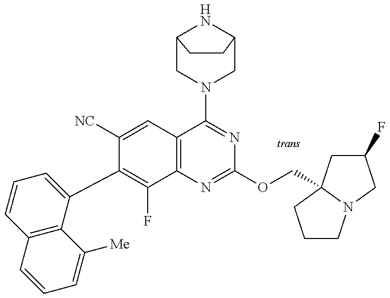 [SMILES: Cc1cccc2cccc(-c3c(C#N)cc4c(N5CC6CCC(C5)N6)nc(OC[C@@]56CCCN5C[C@H](F)C6)nc4c3F)c12] | 87 |
| 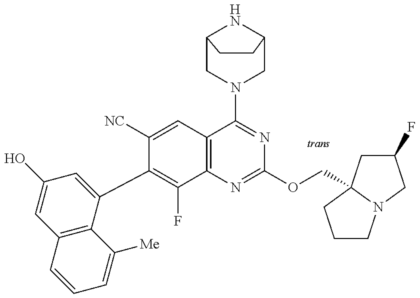 [SMILES: Cc1cccc2cc(O)cc(-c3c(C#N)cc4c(N5CC6CCC(C5)N6)nc(OC[C@@]56CCCN5C[C@H](F)C6)nc4c3F)c12] | 88 |
| 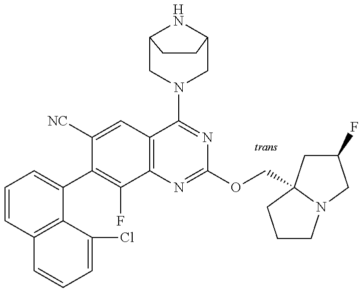 [SMILES: N#Cc1cc2c(N3CC4CCC(C3)N4)nc(OC[C@@]34CCCN3C[C@H](F)C4)nc2c(F)c1-c1cccc2cccc(Cl)c12] | 89 |
| 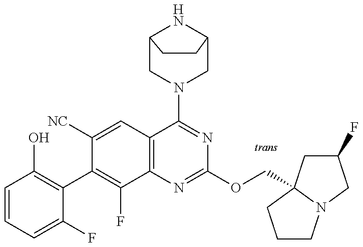 [SMILES: N#Cc1cc2c(N3CC4CCC(C3)N4)nc(OC[C@@]34CCCN3C[C@H](F)C4)nc2c(F)c1-c1c(O)cccc1F] | 90 |
| 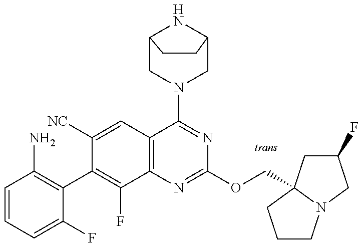 [SMILES: N#Cc1cc2c(N3CC4CCC(C3)N4)nc(OC[C@@]34CCCN3C[C@H](F)C4)nc2c(F)c1-c1c(N)cccc1F] | 91 |
| 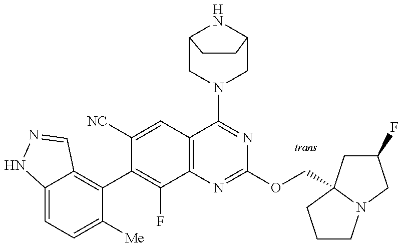 [SMILES: Cc1ccc2[nH]ncc2c1-c1c(C#N)cc2c(N3CC4CCC(C3)N4)nc(OC[C@@]34CCCN3C[C@H](F)C4)nc2c1F] | 92 |
| 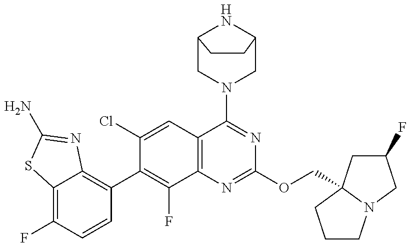 [SMILES: Nc1nc2c(-c3c(Cl)cc4c(N5CC6CCC(C5)N6)nc(OC[C@@]56CCCN5C[C@H](F)C6)nc4c3F)ccc(F)c2s1] | 95 |
| 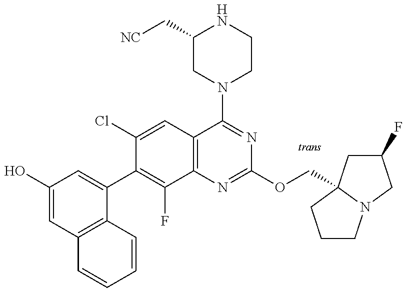 [SMILES: N#CC[C@H]1CN(c2nc(OC[C@@]34CCCN3C[C@H](F)C4)nc3c(F)c(-c4cc(O)cc5ccccc45)c(Cl)cc23)CCN1] | 96 |
| 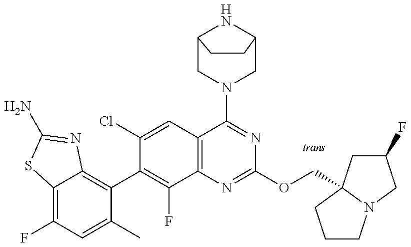 [SMILES: Cc1cc(F)c2sc(N)nc2c1-c1c(Cl)cc2c(N3CC4CCC(C3)N4)nc(OC[C@@]34CCCN3C[C@H](F)C4)nc2c1F] | 100 |
| 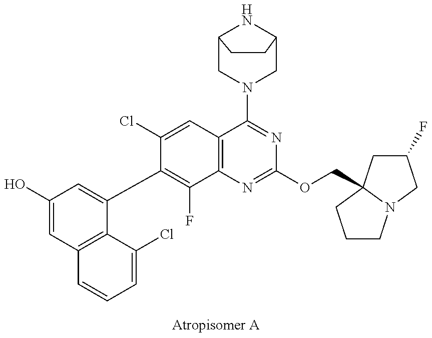 [SMILES: Oc1cc(-c2c(Cl)cc3c(N4CC5CCC(C4)N5)nc(OC[C@]45CCCN4C[C@@H](F)C5)nc3c2F)c2c(Cl)cccc2c1] | 101 |
| 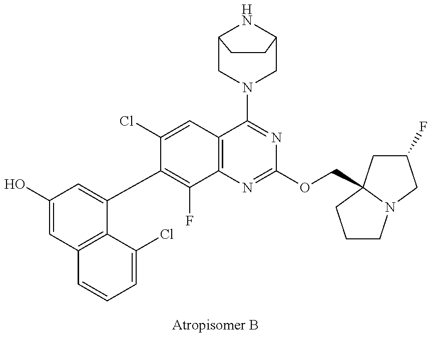 [SMILES: Oc1cc(-c2c(Cl)cc3c(N4CC5CCC(C4)N5)nc(OC[C@]45CCCN4C[C@@H](F)C5)nc3c2F)c2c(Cl)cccc2c1] | 102 |
| 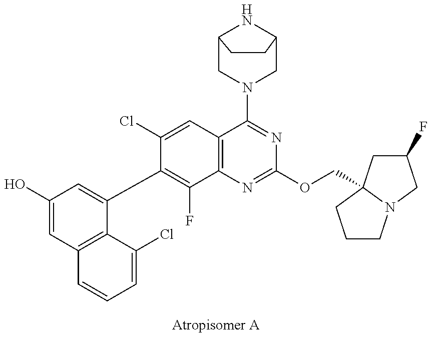 [SMILES: Oc1cc(-c2c(Cl)cc3c(N4CC5CCC(C4)N5)nc(OC[C@@]45CCCN4C[C@H](F)C5)nc3c2F)c2c(Cl)cccc2c1] | 103 |
| 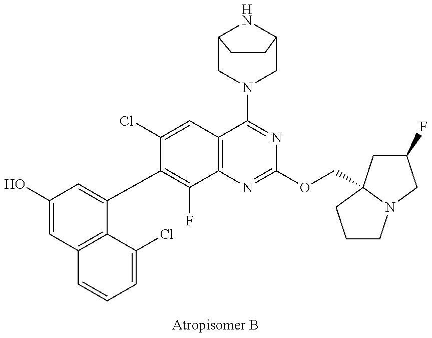 [SMILES: Oc1cc(-c2c(Cl)cc3c(N4CC5CCC(C4)N5)nc(OC[C@@]45CCCN4C[C@H](F)C5)nc3c2F)c2c(Cl)cccc2c1] | 104 |
| 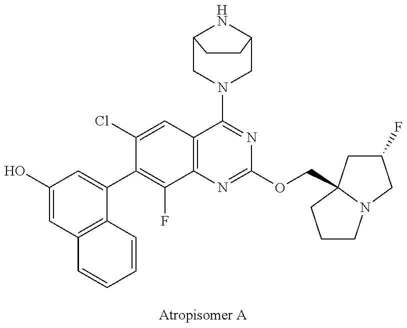 [SMILES: Oc1cc(-c2c(Cl)cc3c(N4CC5CCC(C4)N5)nc(OC[C@]45CCCN4C[C@@H](F)C5)nc3c2F)c2ccccc2c1] | 105 |
| 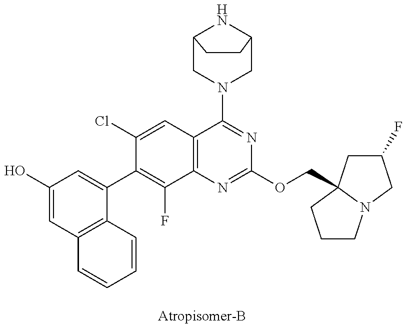 [SMILES: Oc1cc(-c2c(Cl)cc3c(N4CC5CCC(C4)N5)nc(OC[C@]45CCCN4C[C@@H](F)C5)nc3c2F)c2ccccc2c1] | 106 |
| 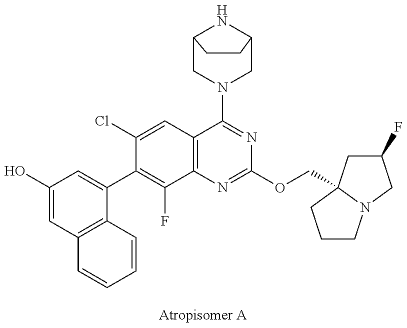 [SMILES: Oc1cc(-c2c(Cl)cc3c(N4CC5CCC(C4)N5)nc(OC[C@@]45CCCN4C[C@H](F)C5)nc3c2F)c2ccccc2c1] | 107 |
| 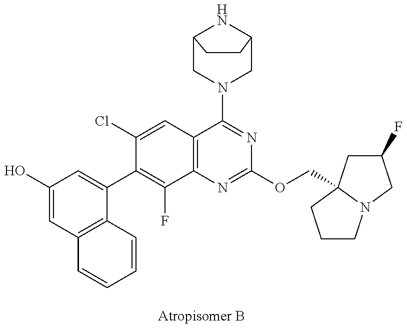 [SMILES: Oc1cc(-c2c(Cl)cc3c(N4CC5CCC(C4)N5)nc(OC[C@@]45CCCN4C[C@H](F)C5)nc3c2F)c2ccccc2c1] | 108 |
| 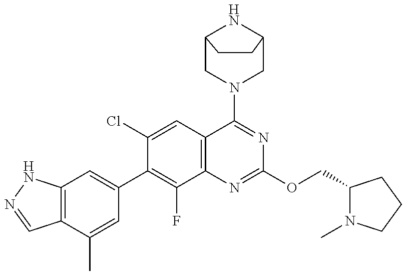 [SMILES: Cc1cc(-c2c(Cl)cc3c(N4CC5CCC(C4)N5)nc(OC[C@@H]4CCCN4C)nc3c2F)cc2[nH]ncc12] | 109 |
| 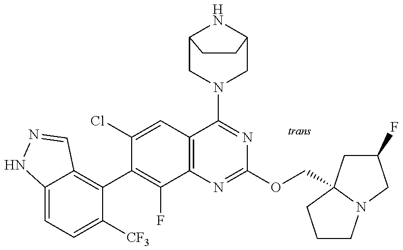 [SMILES: Fc1c(-c2c(C(F)(F)F)ccc3[nH]ncc23)c(Cl)cc2c(N3CC4CCC(C3)N4)nc(OC[C@@]34CCCN3C[C@H](F)C4)nc12] | 111 |
| 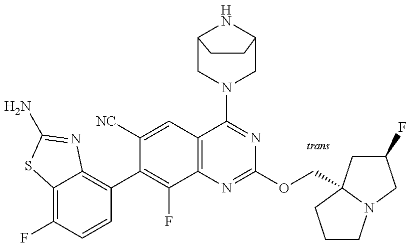 [SMILES: N#Cc1cc2c(N3CC4CCC(C3)N4)nc(OC[C@@]34CCCN3C[C@H](F)C4)nc2c(F)c1-c1ccc(F)c2sc(N)nc12] | 112 |
| 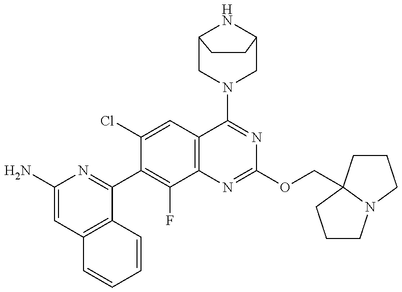 [SMILES: Nc1cc2ccccc2c(-c2c(Cl)cc3c(N4CC5CCC(C4)N5)nc(OCC45CCCN4CCC5)nc3c2F)n1] | 116 |
| 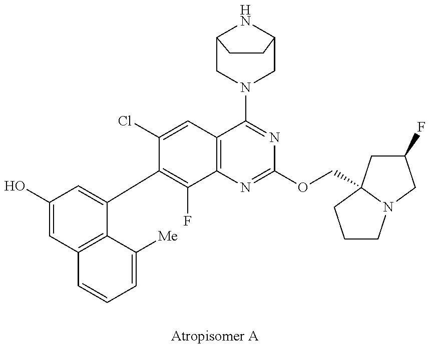 [SMILES: Cc1cccc2cc(O)cc(-c3c(Cl)cc4c(N5CC6CCC(C5)N6)nc(OC[C@@]56CCCN5C[C@H](F)C6)nc4c3F)c12] | 117 |
| 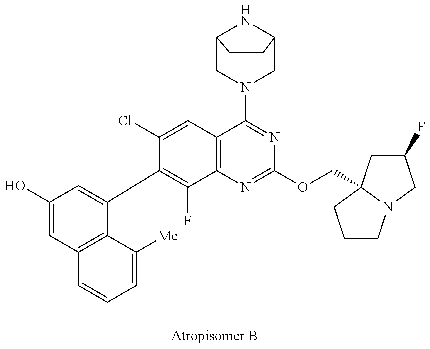 [SMILES: Cc1cccc2cc(O)cc(-c3c(Cl)cc4c(N5CC6CCC(C5)N6)nc(OC[C@@]56CCCN5C[C@H](F)C6)nc4c3F)c12] | 118 |
| 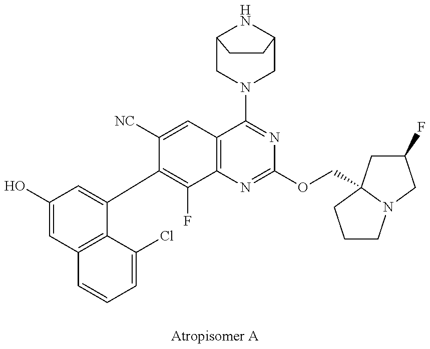 [SMILES: N#Cc1cc2c(N3CC4CCC(C3)N4)nc(OC[C@@]34CCCN3C[C@H](F)C4)nc2c(F)c1-c1cc(O)cc2cccc(Cl)c12] | 119 |
| 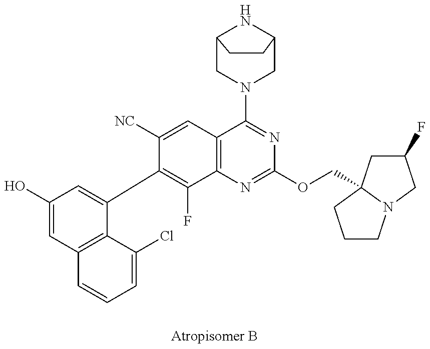 [SMILES: N#Cc1cc2c(N3CC4CCC(C3)N4)nc(OC[C@@]34CCCN3C[C@H](F)C4)nc2c(F)c1-c1cc(O)cc2cccc(Cl)c12] | 120 |
| 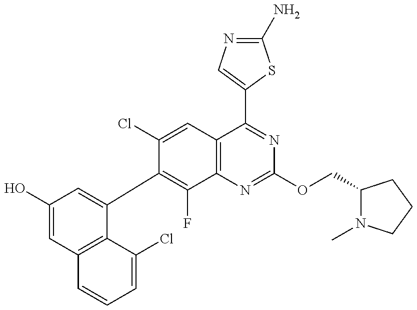 [SMILES: CN1CCC[C@H]1COc1nc(-c2cnc(N)s2)c2cc(Cl)c(-c3cc(O)cc4cccc(Cl)c34)c(F)c2n1] | 121 |
| 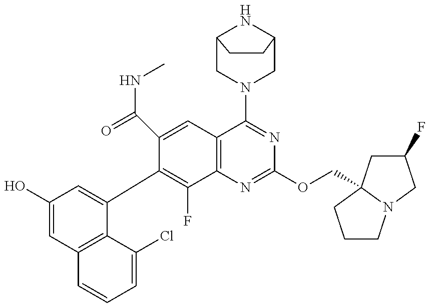 [SMILES: CNC(=O)c1cc2c(N3CC4CCC(C3)N4)nc(OC[C@@]34CCCN3C[C@H](F)C4)nc2c(F)c1-c1cc(O)cc2cccc(Cl)c12] | 122 |
| 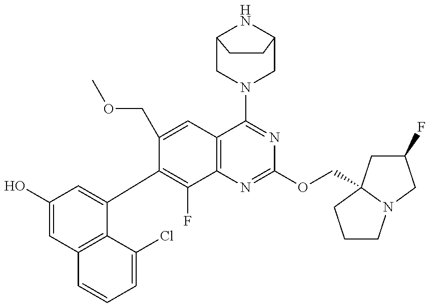 [SMILES: COCc1cc2c(N3CC4CCC(C3)N4)nc(OC[C@@]34CCCN3C[C@H](F)C4)nc2c(F)c1-c1cc(O)cc2cccc(Cl)c12] | 123 |
| 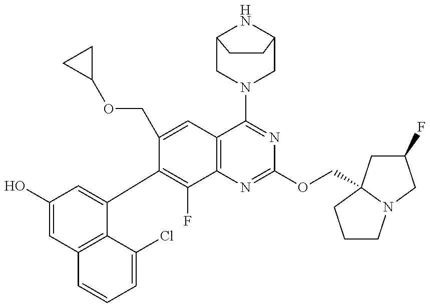 [SMILES: Oc1cc(-c2c(COC3CC3)cc3c(N4CC5CCC(C4)N5)nc(OC[C@@]45CCCN4C[C@H](F)C5)nc3c2F)c2c(Cl)cccc2c1] | 124 |
| 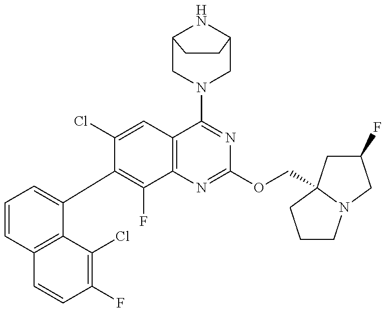 [SMILES: Fc1ccc2cccc(-c3c(Cl)cc4c(N5CC6CCC(C5)N6)nc(OC[C@@]56CCCN5C[C@H](F)C6)nc4c3F)c2c1Cl] | 125 |
| 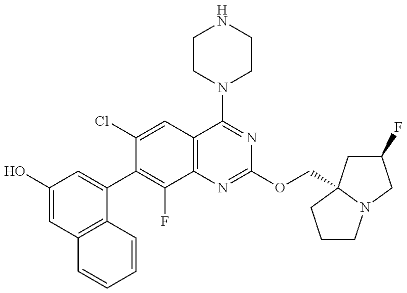 [SMILES: Oc1cc(-c2c(Cl)cc3c(N4CCNCC4)nc(OC[C@@]45CCCN4C[C@H](F)C5)nc3c2F)c2ccccc2c1] | 126 |
| 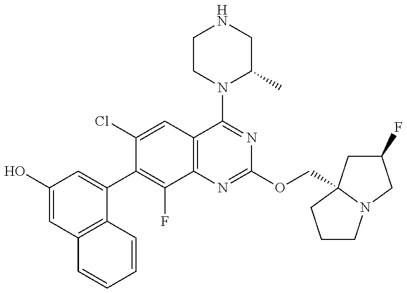 [SMILES: C[C@H]1CNCCN1c1nc(OC[C@@]23CCCN2C[C@H](F)C3)nc2c(F)c(-c3cc(O)cc4ccccc34)c(Cl)cc12] | 127 |
| 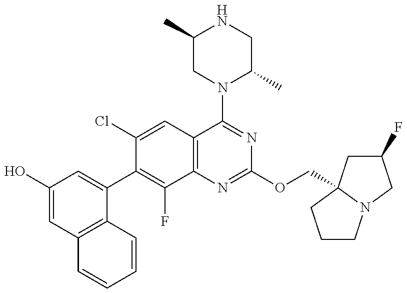 [SMILES: C[C@@H]1CN(c2nc(OC[C@@]34CCCN3C[C@H](F)C4)nc3c(F)c(-c4cc(O)cc5ccccc45)c(Cl)cc23)[C@@H](C)CN1] | 128 |
| 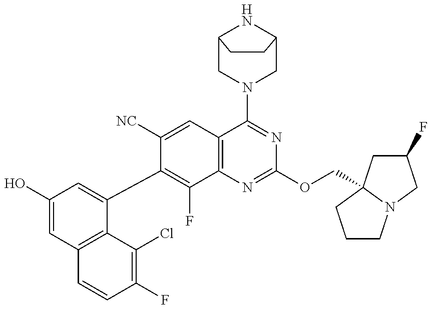 [SMILES: N#Cc1cc2c(N3CC4CCC(C3)N4)nc(OC[C@@]34CCCN3C[C@H](F)C4)nc2c(F)c1-c1cc(O)cc2ccc(F)c(Cl)c12] | 129 |
| 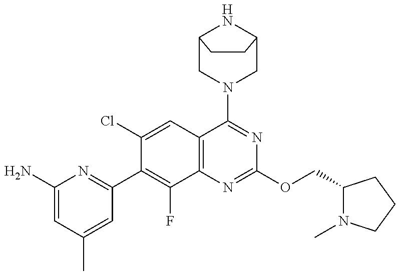 [SMILES: Cc1cc(N)nc(-c2c(Cl)cc3c(N4CC5CCC(C4)N5)nc(OC[C@@H]4CCCN4C)nc3c2F)c1] | 130 |
| 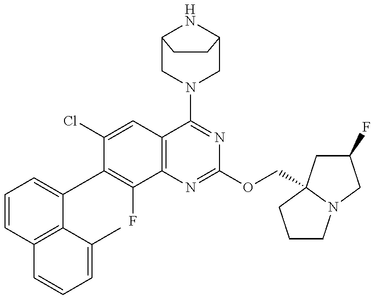 [SMILES: Cc1cccc2cccc(-c3c(Cl)cc4c(N5CC6CCC(C5)N6)nc(OC[C@@]56CCCN5C[C@H](F)C6)nc4c3F)c12] | 131 |
| 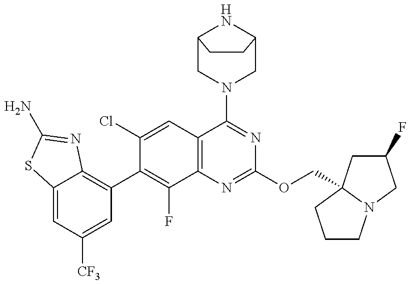 [SMILES: Nc1nc2c(-c3c(Cl)cc4c(N5CC6CCC(C5)N6)nc(OC[C@@]56CCCN5C[C@H](F)C6)nc4c3F)cc(C(F)(F)F)cc2s1] | 132 |
| 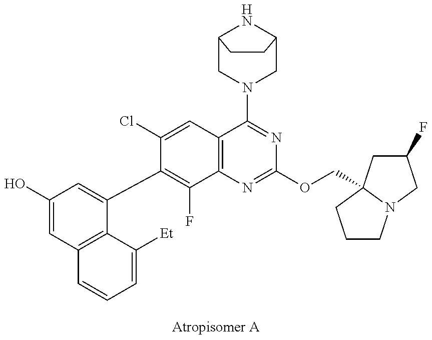 [SMILES: CCc1cccc2cc(O)cc(-c3c(Cl)cc4c(N5CC6CCC(C5)N6)nc(OC[C@@]56CCCN5C[C@H](F)C6)nc4c3F)c12] | 133 |
| 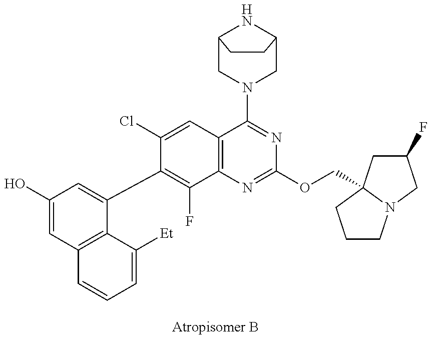 [SMILES: CCc1cccc2cc(O)cc(-c3c(Cl)cc4c(N5CC6CCC(C5)N6)nc(OC[C@@]56CCCN5C[C@H](F)C6)nc4c3F)c12] | 134 |
| 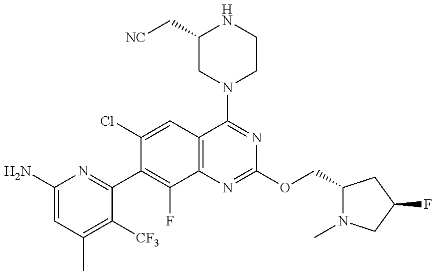 [SMILES: Cc1cc(N)nc(-c2c(Cl)cc3c(N4CCN[C@@H](CC#N)C4)nc(OC[C@@H]4C[C@@H](F)CN4C)nc3c2F)c1C(F)(F)F] | 135 |
| 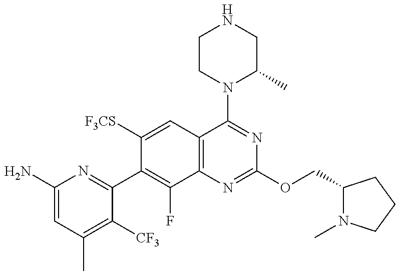 | 136 |
| 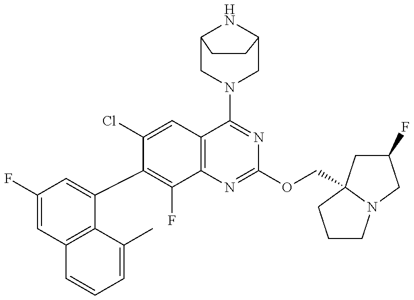 [SMILES: Cc1cccc2cc(F)cc(-c3c(Cl)cc4c(N5CC6CCC(C5)N6)nc(OC[C@@]56CCCN5C[C@H](F)C6)nc4c3F)c12] | 137 |
| 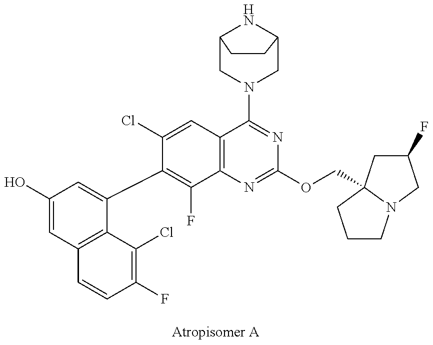 [SMILES: Oc1cc(-c2c(Cl)cc3c(N4CC5CCC(C4)N5)nc(OC[C@@]45CCCN4C[C@H](F)C5)nc3c2F)c2c(Cl)c(F)ccc2c1] | 138 |
| 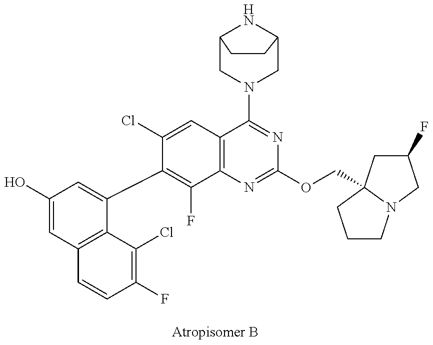 [SMILES: Oc1cc(-c2c(Cl)cc3c(N4CC5CCC(C4)N5)nc(OC[C@@]45CCCN4C[C@H](F)C5)nc3c2F)c2c(Cl)c(F)ccc2c1] | 139 |
| 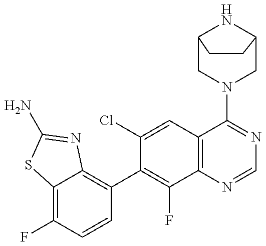 [SMILES: Nc1nc2c(-c3c(Cl)cc4c(N5CC6CCC(C5)N6)ncnc4c3F)ccc(F)c2s1] | 140 |
| 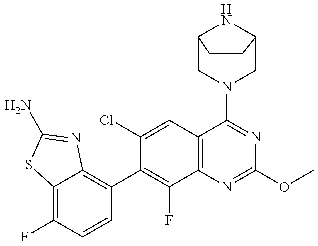 [SMILES: COc1nc(N2CC3CCC(C2)N3)c2cc(Cl)c(-c3ccc(F)c4sc(N)nc34)c(F)c2n1] | 141 |
| 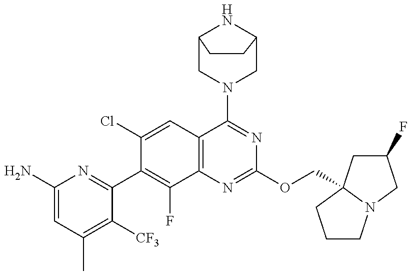 [SMILES: Cc1cc(N)nc(-c2c(Cl)cc3c(N4CC5CCC(C4)N5)nc(OC[C@@]45CCCN4C[C@H](F)C5)nc3c2F)c1C(F)(F)F] | 142 |
| 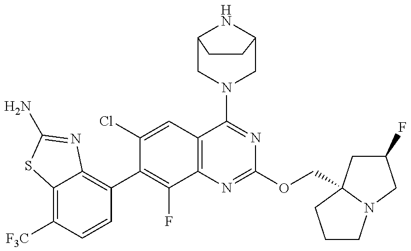 [SMILES: Nc1nc2c(-c3c(Cl)cc4c(N5CC6CCC(C5)N6)nc(OC[C@@]56CCCN5C[C@H](F)C6)nc4c3F)ccc(C(F)(F)F)c2s1] | 143 |
| 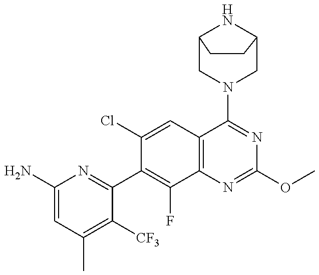 [SMILES: COc1nc(N2CC3CCC(C2)N3)c2cc(Cl)c(-c3nc(N)cc(C)c3C(F)(F)F)c(F)c2n1] | 144 |
| 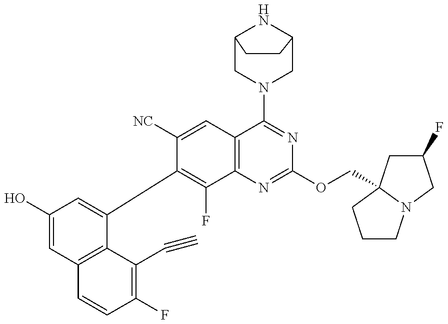 [SMILES: C#Cc1c(F)ccc2cc(O)cc(-c3c(C#N)cc4c(N5CC6CCC(C5)N6)nc(OC[C@@]56CCCN5C[C@H](F)C6)nc4c3F)c12] | 145 |
| 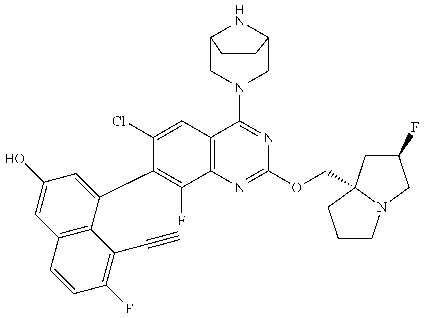 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3F)c12] | 146 |
| 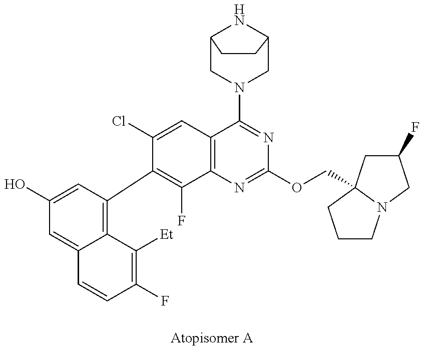 [SMILES: CCc1c(F)ccc2cc(O)cc(-c3c(Cl)cc4c(N5CC6CCC(C5)N6)nc(OC[C@@]56CCCN5C[C@H](F)C6)nc4c3F)c12] | 147 |
| 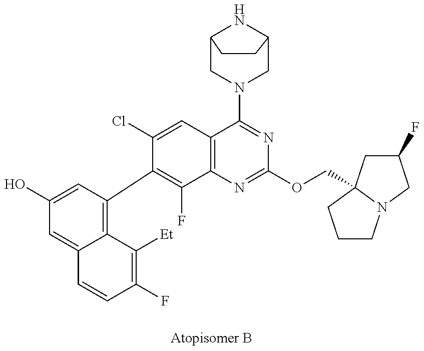 [SMILES: CCc1c(F)ccc2cc(O)cc(-c3c(Cl)cc4c(N5CC6CCC(C5)N6)nc(OC[C@@]56CCCN5C[C@H](F)C6)nc4c3F)c12] | 148 |
| 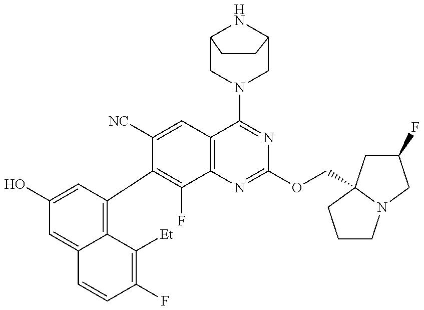 [SMILES: CCc1c(F)ccc2cc(O)cc(-c3c(C#N)cc4c(N5CC6CCC(C5)N6)nc(OC[C@@]56CCCN5C[C@H](F)C6)nc4c3F)c12] | 149 |
| 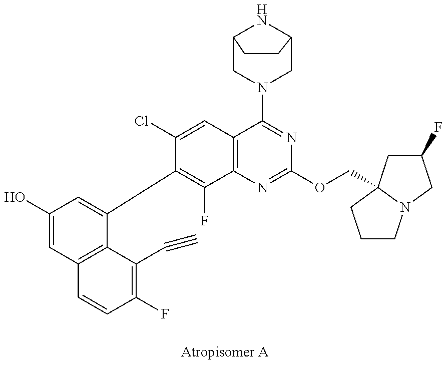 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3F)c12] | 150 |
| 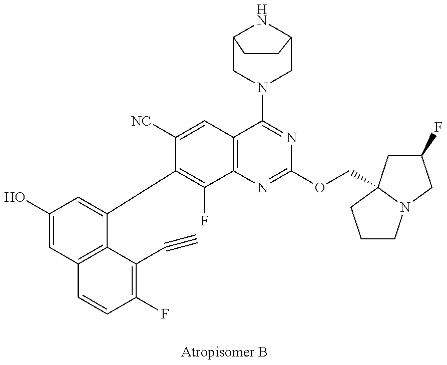 [SMILES: C#Cc1c(F)ccc2cc(O)cc(-c3c(C#N)cc4c(N5CC6CCC(C5)N6)nc(OC[C@@]56CCCN5C[C@H](F)C6)nc4c3F)c12] | 151 |
| 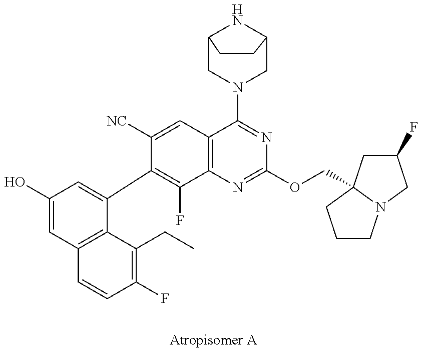 [SMILES: CCc1c(F)ccc2cc(O)cc(-c3c(C#N)cc4c(N5CC6CCC(C5)N6)nc(OC[C@@]56CCCN5C[C@H](F)C6)nc4c3F)c12] | 152 |
| 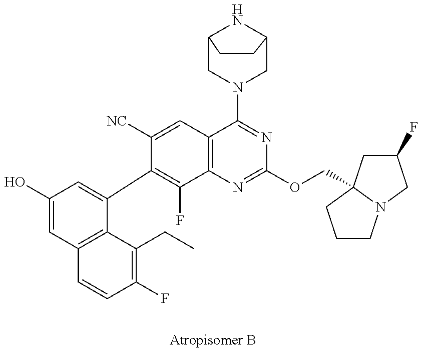 [SMILES: CCc1c(F)ccc2cc(O)cc(-c3c(C#N)cc4c(N5CC6CCC(C5)N6)nc(OC[C@@]56CCCN5C[C@H](F)C6)nc4c3F)c12] | 153 |
| 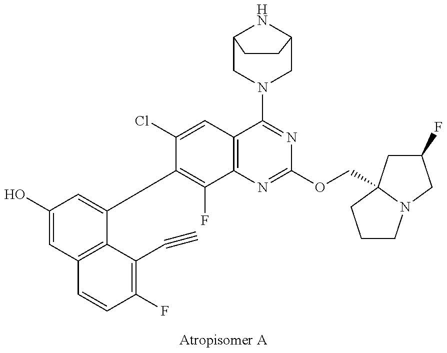 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3F)c12] | 154 |
| 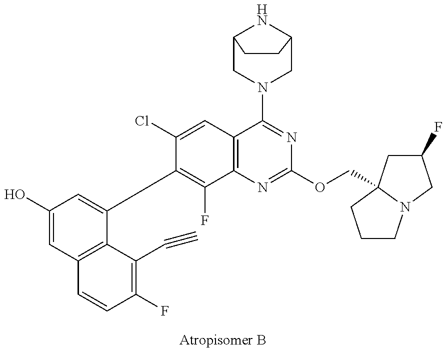 [SMILES: C#Cc1c(F)ccc2cc(O)cc(-c3c(Cl)cc4c(N5CC6CCC(C5)N6)nc(OC[C@@]56CCCN5C[C@H](F)C6)nc4c3F)c12] | 155 |
| 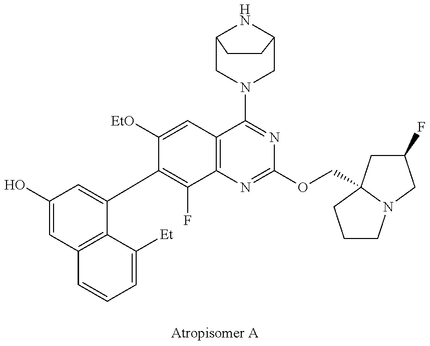 [SMILES: CCOc1cc2c(N3CC4CCC(C3)N4)nc(OC[C@@]34CCCN3C[C@H](F)C4)nc2c(F)c1-c1cc(O)cc2cccc(CC)c12] | 156 |
| 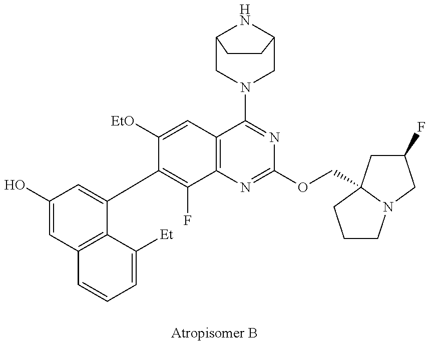 [SMILES: CCOc1cc2c(N3CC4CCC(C3)N4)nc(OC[C@@]34CCCN3C[C@H](F)C4)nc2c(F)c1-c1cc(O)cc2cccc(CC)c12] | 157 |
| 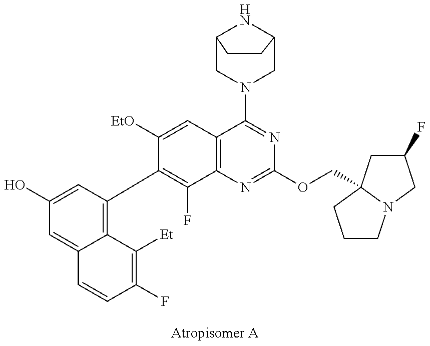 [SMILES: CCOc1cc2c(N3CC4CCC(C3)N4)nc(OC[C@@]34CCCN3C[C@H](F)C4)nc2c(F)c1-c1cc(O)cc2ccc(F)c(CC)c12] | 158 |
| 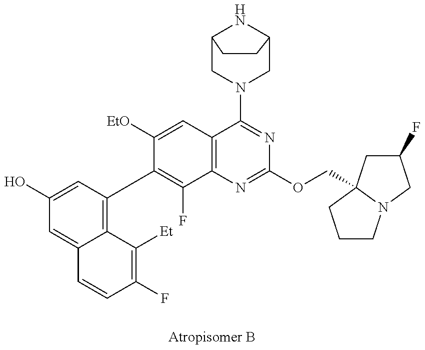 [SMILES: CCOc1cc2c(N3CC4CCC(C3)N4)nc(OC[C@@]34CCCN3C[C@H](F)C4)nc2c(F)c1-c1cc(O)cc2ccc(F)c(CC)c12] | 159 |
| 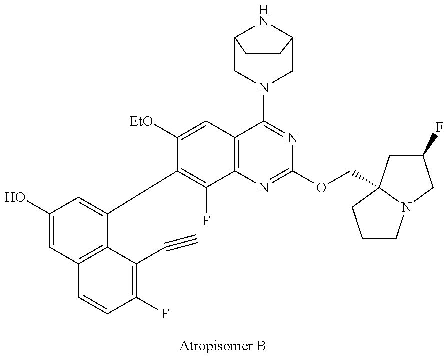 [SMILES: C#Cc1c(F)ccc2cc(O)cc(-c3c(OCC)cc4c(N5CC6CCC(C5)N6)nc(OC[C@@]56CCCN5C[C@H](F)C6)nc4c3F)c12] | 160 |
| 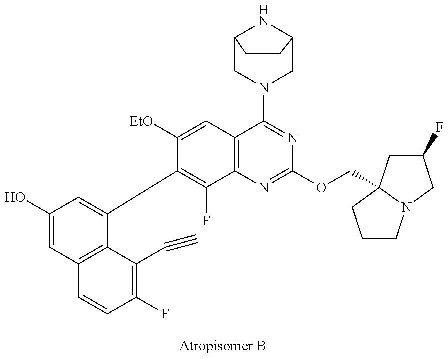 [SMILES: C#Cc1c(F)ccc2cc(O)cc(-c3c(OCC)cc4c(N5CC6CCC(C5)N6)nc(OC[C@@]56CCCN5C[C@H](F)C6)nc4c3F)c12] | 161 |
| 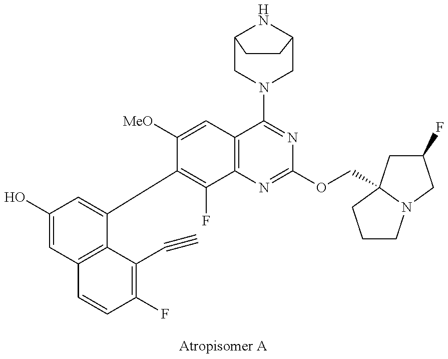 [SMILES: C#Cc1c(F)ccc2cc(O)cc(-c3c(OC)cc4c(N5CC6CCC(C5)N6)nc(OC[C@@]56CCCN5C[C@H](F)C6)nc4c3F)c12] | 162 |
| 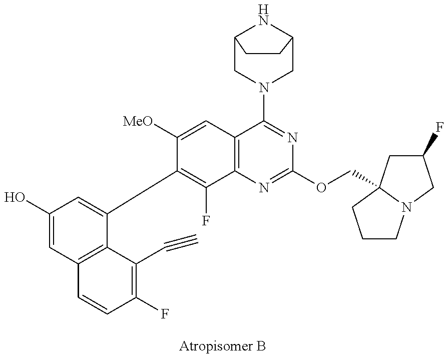 [SMILES: C#Cc1c(F)ccc2cc(O)cc(-c3c(OC)cc4c(N5CC6CCC(C5)N6)nc(OC[C@@]56CCCN5C[C@H](F)C6)nc4c3F)c12] | 163 |
| 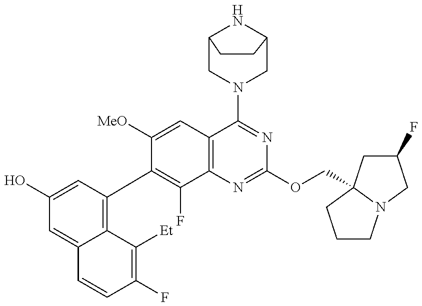 [SMILES: CCc1c(F)ccc2cc(O)cc(-c3c(OC)cc4c(N5CC6CCC(C5)N6)nc(OC[C@@]56CCCN5C[C@H](F)C6)nc4c3F)c12] | 164 |
| 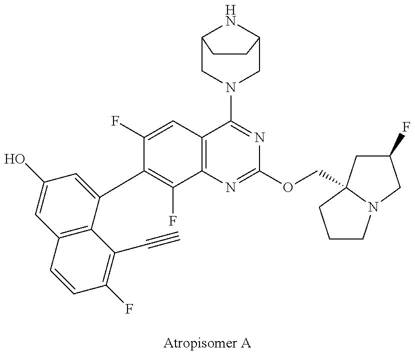 [SMILES: C#Cc1c(F)ccc2cc(O)cc(-c3c(F)cc4c(N5CC6CCC(C5)N6)nc(OC[C@@]56CCCN5C[C@H](F)C6)nc4c3F)c12] | 165 |
| 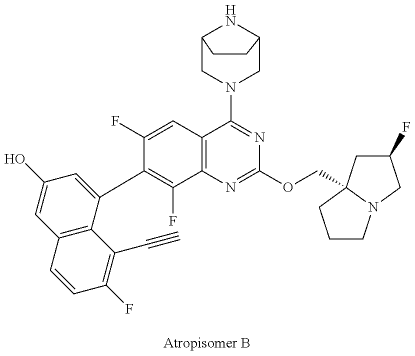 [SMILES: C#Cc1c(F)ccc2cc(O)cc(-c3c(F)cc4c(N5CC6CCC(C5)N6)nc(OC[C@@]56CCCN5C[C@H](F)C6)nc4c3F)c12] | 166 |
| 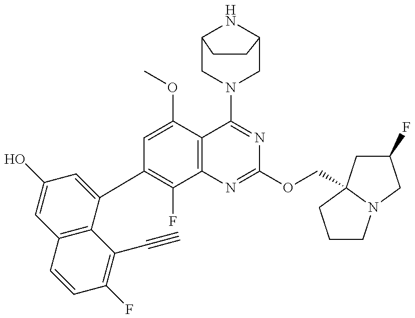 [SMILES: C#Cc1c(F)ccc2cc(O)cc(-c3cc(OC)c4c(N5CC6CCC(C5)N6)nc(OC[C@@]56CCCN5C[C@H](F)C6)nc4c3F)c12] | 167 |
| 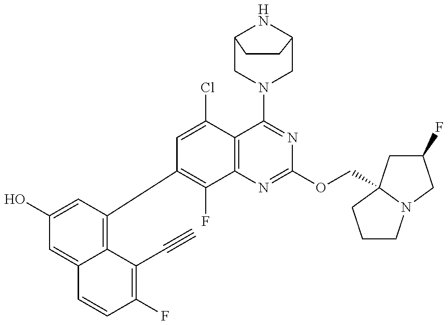 [SMILES: C#Cc1c(F)ccc2cc(O)cc(-c3cc(Cl)c4c(N5CC6CCC(C5)N6)nc(OC[C@@]56CCCN5C[C@H](F)C6)nc4c3F)c12] | 168 |
| 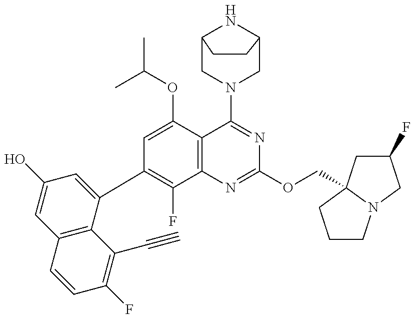 [SMILES: C#Cc1c(F)ccc2cc(O)cc(-c3cc(OC(C)C)c4c(N5CC6CCC(C5)N6)nc(OC[C@@]56CCCN5C[C@H](F)C6)nc4c3F)c12] | 169 |
| 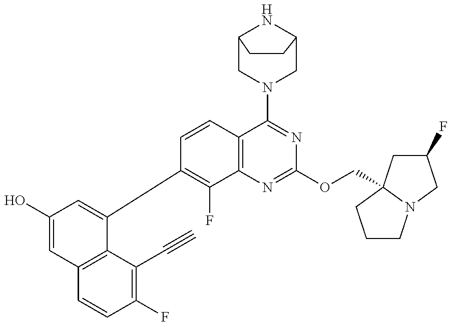 [SMILES: C#Cc1c(F)ccc2cc(O)cc(-c3ccc4c(N5CC6CCC(C5)N6)nc(OC[C@@]56CCCN5C[C@H](F)C6)nc4c3F)c12] | 170 |

In some of the specific compounds in Table A above and the specific compounds in the Examples section below, the structure is labeled as “trans”. Unless obviously contrary from context, such designation should be understood that the specific compound with the “trans” designation is in a racemic form with respect to the pair of chiral centers on the pyrrolizidine ring, which can be separated into two enantiomers. To be clear, the separated/enriched individual enantiomers are also compounds of the present disclosure.

In some embodiments, to the extent applicable, the genus of compounds in the present disclosure also excludes any of the compounds specifically prepared and disclosed prior to this disclosure.

### Method of Synthesis

The compounds of the present disclosure can be readily synthesized by those skilled in the art in view of the present disclosure. Exemplified syntheses are also shown in the Examples section.

The following synthetic process of Formula I is illustrative, which can be applied similarly by those skilled in the art for the synthesis of compounds of Formula II or III, by using a proper synthetic starting material or intermediate. In some embodiments, the present disclosure also provides synthetic methods and synthetic intermediates for preparing the compounds of Formula I, II, or III, as represented by the scheme herein.

As shown in Scheme 1, compounds of Formula I can typically be synthesized through three coupling reactions. In some embodiments, a compound S-1 can be coupled with a R³ donor S-2, wherein M¹ can be hydrogen, a metal (such as Zn²⁺), boronic acid or ester, tributyltin, etc., typically under a transition metal catalyzed coupling reaction, such as a palladium catalyzed coupling reaction as exemplified herein. Lg³ is typically a leaving group described herein, such as a halide or a sulfonate leaving group that are suitable for metal catalyzed coupling reactions. The reaction conditions can be adjusted such that R³ is introduced to replace Lg³. Compound S-3 can then be transformed into S-5 through a second coupling reaction. Depending on the nature of G¹, this coupling can be carried out with or without a transition metal catalyst. In some embodiments, M² can be hydrogen, and G¹-M² in S-4 is N—H, and the bridged ring can replace Lg¹, which can be a leaving group described herein such as halogen (e.g., Cl), to produce compound S-5, typically, under basic conditions in an aprotic polar solvent such as dimethyl sulfoxide. Compound S-5 can then be converted into Formula I by reacting with S-6. R¹-M³ in S-6 typically includes a —OH, or —NH functional group, for example, M³ can be hydrogen, such that it can react with S-5 to replace the leaving group Lg², which can be a halogen or another leaving group described herein such as sulfone, etc. Example 1 shows exemplary reaction conditions for converting a compound of S-1 into a compound of Formula I. The variables R¹, R³, G¹, A¹, A², G², G³, R¹⁰⁰, m, n1, and n2 in formulae of Scheme 1 are defined hereinabove in connection with Formula I.

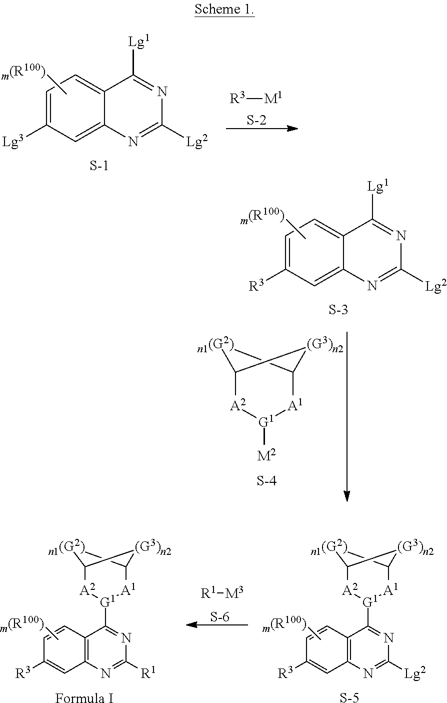

The sequence of coupling shown in Scheme 1 is not absolutely necessary, as one of ordinary skill in the art viewing the present disclosure could prepare compounds of Formula I through a slightly different coupling sequence, for example, by introducing the bridged ring to replace Lg¹ first, then followed by introducing R¹ group, and lastly introduce R³ group.

Suitable coupling partners such as S-1, S-4 or S-6 can be prepared by methods known in the art or methods in view of the present disclosure, see e.g., the Examples section. Also see e.g., US Patent Application Publication No. 2019/0127336.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wis., USA), Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more of the compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) and a pharmaceutically acceptable excipient. Pharmaceutically acceptable excipients are known in the art. Non-limiting suitable excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof), e.g., in a therapeutically effective amount. In any of the embodiments described herein, the pharmaceutical composition can comprise a compound selected from any of the compounds listed in Table A herein, or a pharmaceutically acceptable salt thereof, e.g., in a therapeutically effective amount of.

The pharmaceutical composition can also be formulated for delivery via any of the known routes of delivery, which include but are not limited to oral, parenteral, inhalation, etc.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

In some embodiments, the pharmaceutical composition is formulated for inhalation. The inhalable formulations can be, for example, formulated as a nasal spray, dry powder, or an aerosol administrable through a metered-dose inhaler. Excipients for preparing formulations for inhalation are known in the art. Non-limiting suitable excipients include, for example, lactose, talc, silicic acid, aluminum hydroxide, calcium silicates and polyamide powder, and mixtures of these substances. Sprays can additionally contain propellants, such as chlorofluorohydrocarbons and volatile unsubstituted hydrocarbons, such as butane and propane.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof). In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, which can depend on the recipient of the treatment, the disease or disorder being treated and the severity thereof, the composition containing the compound, the time of administration, the route of administration, the duration of treatment, the compound potency (e.g., for inhibiting KRAS G12D), its rate of clearance and whether or not another drug is co-administered.

For veterinary use, a compound of the present disclosure can be administered as a suitably acceptable formulation in accordance with normal veterinary practice. The veterinarian can readily determine the dosing regimen and route of administration that is most appropriate for a particular animal.

In some embodiments, all the necessary components for the treatment of KRAS-related disorder using a compound of the present disclosure either alone or in combination with another agent or intervention traditionally used for the treatment of such disease can be packaged into a kit. Specifically, in some embodiments, the present invention provides a kit for use in the therapeutic intervention of the disease comprising a packaged set of medicaments that include the compound disclosed herein as well as buffers and other components for preparing deliverable forms of said medicaments, and/or devices for delivering such medicaments, and/or any agents that are used in combination therapy with the compound of the present disclosure, and/or instructions for the treatment of the disease packaged with the medicaments. The instructions may be fixed in any tangible medium, such as printed paper, or a computer readable magnetic or optical medium, or instructions to reference a remote computer data source such as a world wide web page accessible via the internet.

### Method of Treatment

Compounds of the present disclosure are useful as therapeutic active substances for the treatment and/or prophylaxis of diseases or disorders that are associated with RAS, e.g., KRAS^{G12D}.

In some embodiments, the present disclosure provides a method of inhibiting RAS-mediated cell signaling comprising contacting a cell (e.g., a cancer cell) with an effective amount of one or more compounds of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof). Inhibition of RAS-mediated signal transduction can be assessed and demonstrated by a wide variety of ways known in the art. Non-limiting examples include a showing of (a) a decrease in GTPase activity of RAS; (b) a decrease in GTP binding affinity or an increase in GDP binding affinity; (c) an increase in K_{off} of GTP or a decrease in K_{off} of GDP; (d) a decrease in the levels of signaling transduction molecules downstream in the RAS pathway, such as a decrease in pMEK, pERK, or pAKT levels; and/or (e) a decrease in binding of RAS complex to downstream signaling molecules including but not limited to Raf. Kits and commercially available assays can be utilized for determining one or more of the above.

In some embodiments, the present disclosure provides a method of inhibiting KRAS^{G12D}, HRAS^{G12D}, and/or NRAS^{G12D} in a cell, e.g., a cancer cell, the method comprising contacting the cell with an effective amount of one or more compounds of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof).

In some embodiments, the present disclosure provides a method of inhibiting KRAS mutant protein in a cell, e.g., a cancer cell, such as inhibiting KRAS^{G12D} in a cell, the method comprising contacting the cell with an effective amount of one or more compounds of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof).

In some embodiments, the present disclosure provides a method of inhibiting proliferation of a cell population (e.g., a cancer cell population), the method comprising contacting the cell population with an effective amount of one or more compounds of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1. III-2, III-3, III-4, III-5, III-6, III-7, III-8, or III-9), any of the compounds listed in Table A herein, or a pharmaceutically acceptable salt thereof). In some embodiments, the inhibition of proliferation is measured as a decrease in cell viability of the cell population.

In some embodiments, the present disclosure provides a method of treating cancer in a subject, the method comprising administering to the subject a therapeutically effective amount of one or more compounds of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a therapeutically effective amount of a pharmaceutical composition described herein. In some embodiments, the cancer is a pancreatic cancer, lung cancer, colorectal cancer, endometrial cancer, appendix cancer, cholangiocarcinoma, bladder urothelial cancer, ovarian cancer, gastric cancer, breast cancer, bile duct cancer, or a hematologic malignancy. In some embodiments, the subject has a mutation of KRAS^{G12D}, HRAS^{G12D} and/or NRAS^{G12D}.

In some embodiments, the present disclosure provides a method of treating cancer metastasis or tumor metastasis in a subject, the method comprising administering to the subject a therapeutically effective amount of one or more compounds of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a therapeutically effective amount of a pharmaceutical composition described herein.

In some embodiments, the present disclosure provides a method of treating a disease or disorder, e.g., a cancer associated with G12D mutation of KRAS, HRAS and/or NRAS, such as a cancer associated with KRAS^{G12D}, in a subject in need thereof. In some embodiments, the method comprises administering to the subject a therapeutically effective amount of a compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a therapeutically effective amount of a pharmaceutical composition described herein.

In some embodiments, a method treating cancer is provided, the method comprising administering to a subject in need thereof an effective amount of any of the compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a pharmaceutical composition comprising the compound of the present disclosure. In some embodiments, the cancer comprises a G12D mutation of KRAS, HRAS and/or NRAS, e.g., a KRAS-G12D mutation. Determining whether a tumor or cancer comprises a G12D mutation of KRAS, HRAS and/or NRAS is known in the art, either by a PCR kit or using DNA sequencing. In various embodiments, the cancer can be pancreatic, colorectal, lung, or endometrial cancer. In some embodiments, the cancer is appendix cancer, cholangiocarcinoma, bladder urothelial cancer, ovarian cancer, gastric cancer, breast cancer, or bile duct cancer. In some embodiments, the cancer is a hematological malignancy (e.g., acute myeloid leukemia).

In some embodiments the present disclosure provides a method of treating a disease or disorder mediated by a Ras mutant protein (such as K-Ras, H-Ras, and/or N-Ras) in a subject in need thereof, the method comprising: a) determining if the subject has a Ras mutation; and b) if the subject is determined to have the Ras mutation, then administering to the subject a therapeutically effective amount of at least one compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a pharmaceutical composition described herein. In some embodiments, the disease or disorder is cancer, for example, lung cancer (e.g., non-small cell lung cancer), pancreatic cancer, colorectal cancer, endometrial cancer, appendix cancer, cholangiocarcinoma, bladder urothelial cancer, ovarian cancer, gastric cancer, breast cancer, bile duct cancer or hematological malignancy such as acute myeloid leukemia. In some embodiments, the disease or disorder is MYH associated polyposis.

In some embodiments the present disclosure provides a method of treating a disease or disorder (e.g., a cancer described herein) in a subject in need thereof, wherein the method comprises determining if the subject has a G12D mutation of KRAS, HRAS and/or NRAS, e.g., KRAS^{G12D} mutation, and if the subject is determined to have the KRAS, HRAS and/or NRAS^{G12D} mutation, e.g., KRAS G12D mutation, then administering to the subject a therapeutically effective dose of at least one compound of the present disclosure (e.g., a compound of Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, or a pharmaceutically acceptable salt thereof) or a pharmaceutical composition comprising the at least one compound of the present disclosure.

G12D mutation of KRAS, HRAS and/or NRAS has also been identified in hematological malignancies (e.g., cancers that affect blood, bone marrow and/or lymph nodes). Accordingly, certain embodiments are directed to a method of treating hematological malignancy in a subject in need thereof, the method typically comprises administration of a compound of the present disclosure (e.g., in the form of a pharmaceutical composition) to the subject. Such malignancies include, but are not limited to leukemias and lymphomas, such as Acute lymphoblastic leukemia (ALL), Acute myeloid leukemia (AML), Chronic lymphocytic leukemia (CLL), small lymphocytic lymphoma (SLL), Chronic myelogenous leukemia (CML), Acute monocytic leukemia (AMoL) and/or other leukemias. In some embodiments, the hematological malignancy can also include lymphomas such as Hodgkins lymphoma or non-Hodgkins lymphoma, plasma cell malignancies such as multiple myeloma, mantle cell lymphoma, and Waldenstrom's macroglubunemia.

Compounds of the present disclosure can be used as a monotherapy or in a combination therapy. In some embodiments, the combination therapy includes treating the subject with a targeted therapeutic agent, chemotherapeutic agent, therapeutic antibody, radiation, cell therapy, or immunotherapy. In some embodiments, compounds of the present disclosure can also be co-administered with an additional pharmaceutically active compound, either concurrently or sequentially in any order, to a subject in need thereof (e.g., a subject having a cancer associated with KRAS^{G12D} mutation as described herein). In some embodiments, the additional pharmaceutically active compound can be a targeted agent (e.g. MEK inhibitor), a a chemotherapeutic agent (e.g. cisplatin or docetaxel), a therapeutic antibody (e.g. anti-PD-1 antibody), etc. Any of the known therapeutic agents can be used in combination with the compounds of the present disclosure. In some embodiments, compounds of the present disclosure can also be used in combination with a radiation therapy, hormone therapy, cell therapy, surgery and immunotherapy, which therapies are well known to those skilled in the art.

Many chemotherapeutics are presently known in the art and can be used in combination with the compounds of the present disclosure. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), venetoclax, and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; gemcitabine; capecitabine; and pharmaceutically acceptable salts, acids or derivatives of any of the above.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, onapristone, and toremifene (Fareston); and anti-androgens such as flutamide, nilutamide, bicalutamide, leuprolide, and goserelin; chlorambucil; 6-thioguanine; mercaptopurine; methotrexate; pemetrexed; platinum analogs such as cisplatin, carboplatin and oxaliplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

Where desired, the compounds or pharmaceutical composition of the present disclosure can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, Afatinib, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, Zosuquidar.

The compounds of the present disclosure may also be used in combination with an additional pharmaceutically active compound that disrupts or inhibits RAS-RAF-ERK or PI3K-AKT-TOR signaling pathways. In other such combinations, the additional pharmaceutically active compound is a PD-1 and PD-L1 antagonist. The compounds or pharmaceutical compositions of the disclosure can also be used in combination with an amount of one or more substances selected from EGFR inhibitors, CDK inhibitors, MEK inhibitors, PI3K inhibitors, AKT inhibitors, TOR inhibitors, Mcl-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAG1, and anti-OX40 agents, anti-4-1BB (CD137) agonists, anti-GITR agonists, CAR-T cells, and BiTEs.

Exemplary anti-PD-1 or anti-PDL-1 antibodies and methods for their use are described by Goldberg et al., Blood 110(1):186-192 (2007), Thompson et al., Clin. Cancer Res. 13(6):1757-1761 (2007), and Korman et al., International Application No. PCT/JP2006/309606 (publication no. WO 2006/121168 A1), each of which are expressly incorporated by reference herein, include: pembrolizumab (Keytruda®), nivolumab (Opdivo®), Yervoy™ (ipilimumab) or Tremelimumab (to CTLA-4), galiximab (to B7.1), M7824 (a bifunctional anti-PD-L1/TGF-β Trap fusion protein), AMP224 (to B7DC), BMS-936559 (to B7-H1), MPDL3280A (to B7-H1), MEDI-570 (to ICOS), AMG 404, AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), Ipilumumab (to CTLA-4). Immune therapies also include genetically engineered T-cells (e.g., CAR-T cells) and bispecific antibodies (e.g., BiTEs). Non-limiting useful additional agents also include anti-EGFR antibody and small molecule EGFR inhibitors such as cetuximab (Erbitux), panitumumab (Vectibix), zalutumumab, nimotuzumab, matuzumab, gefitinib, erlotinib, lapatinib, osimertinib, etc. Non-limiting useful additional agents also include CDK inhibitors such as CDK4/6 inhibitors, such as palbociclib, abemaciclib, ribociclib, dinaciclib, etc. Non-limiting useful additional agents also include MEK inhibitors such as trametinib and binimetinib. Non-limiting useful additional agents also include SHP2 inhibitors such as TNO155. RMC-4630 and RLY-1971.

The administering herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally.

Dosing regimen including doses can vary and can be adjusted, which can depend on the recipient of the treatment, the disease or disorder being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable atoms or groups for the variables herein are independently selected. The definitions of the variables can be combined. Using Formula I as an example, any of the definitions of one of R¹, R³, G¹, A¹, A², G², G³, R¹⁰⁰, m, n1, and n2 in Formula I can be combined with any of the definitions of the others of R¹, R³, G¹, A¹, A², G², G³, R¹⁰⁰, m, n1, and n2 in Formula I. Such combination is contemplated and within the scope of the present disclosure.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds of the present disclosure can comprise one or more asymmetric centers and/or axial chirality, and thus can exist in various isomeric forms, e.g., enantiomers and/or diastereomers. For example, the compounds described herein can be in the form of an individual enantiomer, diastereomer, atropisomer, or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers including racemic mixtures. In embodiments herein, unless otherwise obviously contrary from context, when a stereochemistry is specifically drawn, it should be understood that with respect to that particular chiral center or axial chirality, the compound can exist predominantly as the as-drawn stereoisomer, such as with less than 20%, less than 10%, less than 5%, less than 1%, by weight, by HPLC area, or both, or with a non-detectable amount of the other stereoisomer(s). The presence and/or amounts of stereoisomers can be determined by those skilled in the art in view of the present disclosure, including through the use of chiral HPLC.

Compounds of the present disclosure can have atropisomers. In any of the embodiments described herein, when applicable, the compound of the present disclosure can exist as a mixture of atropisomers in any ratio. In some embodiments, when applicable, the compound can exist as an isolated individual atropisomer substantially free (e.g., with less than 20%, less than 10%, less than 5%, less than 1%, by weight, by HPLC area, or both, or with a non-detectable amount) of the other atropisomer(s). The Examples section shows some exemplary isolated atropisomers of compounds of the present disclosure. As understood by those skilled in the art, when the rotation is restricted around a single bond, e.g., a biaryl single bond, a compound may exist in a mixture of atropisomers with each individual atropisomer isolable.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂-5, C₂₋₄, C₂-3, C₃₋₆, C₃₋₅, C₃₋₄, C₄₋₆, C₄₋₅, and C₅₋₆.

As used herein, the term “compound(s) of the present disclosure” or “compound(s) of the present invention” refers to any of the compounds described herein according to Formula I (e.g., Formula I-1, I-2, I-3, I-1-A, I-2-A, I-3-A, I-1-A-1, I-1-A-2, I-1-A-3, I-1-A-4, I-1-A-4-E1, I-1-A-4-E2, I-1-A-5, I-1-A-6, I-1-A-7, I-1-A-8, I-1-A-9, I-1-A-10, I-1-A-11, or I-1-A-12), Formula II (e.g., Formula II-1, II-2, II-2-E1, II-2-E2, II-3, II-1-A, II-1-B, II-1-C, II-2-A, II-2-B, II-2-C, II-2-A-E1, II-2-B-E1, II-2-C-E1, II-2-A-E2, II-2-B-E2, or II-2-C-E2), Formula III (e.g., Formula III-1, III-2, III-3, III-4, III-5, III-6, III-7, III-8, or III-9), any of the compounds listed in Table A herein, any of the title compounds in the Examples section or those characterized in Table 1, isotopically labeled compound(s) thereof (such as a deuterated analog wherein one or more of the hydrogen atoms is/are substituted with a deuterium atom with an abundance above its natural abundance), possible stereoisomers thereof (including diastereoisomers, enantiomers, and racemic mixtures), geometric isomers thereof, atropisomers thereof, tautomers thereof, conformational isomers thereof, and/or pharmaceutically acceptable salts thereof (e.g., acid addition salt such as HCl salt or base addition salt such as Na salt). Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₂ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. In one embodiment, the alkyl group is a C₁₋₄ alkyl group selected from methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “heteroalkyl” refers to an alkyl group as defined above, with one or more carbon being replaced with a heteroatom, such as O or N. Those skilled in the art would understand that an O atom will replace a CH₂ unit and an N atom will replace a CH unit. A heteroalkyl can be designated by its number of carbons. For example, a C₁₋₄ heteroalkyl refers to a heteroalkyl group containing 1-4 carbons. Examples of heteroalkyl include but not limited to —O—CH₂CH₂—OCH₃, HO—CH₂CH₂—O—CH₂—, —CH₂CH₂—N(H)—CH₃, —N—(CH₃)₂, —CH(CH₃)(OCH₃), etc. When optionally substituted, either the heteroatom or the carbon atom of the heteroalkyl group can be substituted with a permissible substituent. As used herein, the term “heteroalkylene” as used by itself or as part of another group refers to a divalent radical derived from a heteroalkyl group.

As used herein, the term “alkenyl” as used by itself or as part of another group refers to a straight- or branched-chain aliphatic hydrocarbon containing one or more, such as one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, such as one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₄ haloalkyl group.

“Carbocyclyl” or “carbocyclic” as used by itself or as part of another group refers to a radical of a non-aromatic cyclic hydrocarbon group having from 3 to 10 ring carbon atoms (“C₃₋₁₀ carbocyclyl”) and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”).

“Heterocyclyl” or “heterocyclic” as used by itself or as part of another group refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl).

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

As commonly understood by those skilled in the art, alkylene, alkenylene, alkynylene, carbocyclylene, heterocyclylene, arylene, and heteroarylene refer to the corresponding divalent radicals of alkyl, alkenyl, alkynyl, carbocyclyl, heterocyclyl, aryl, and heteroaryl groups, respectively.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkenyl, alkynyl, carbocyclic, cycloalkyl, alkoxy, cycloalkoxy, or heterocyclic group herein can be unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl or heteroaryl group herein can be unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

Exemplary carbon atom substituents include, but are not limited to, halogen, —CN, —NO₂, —N₃, —SO₂H, —SO₃H, —OH, —OR^{aa}, —ON(R^{bb}), —N(R^{bb})₂, —N(R^{bb})⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)Ra, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(ORC)₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 Rd groups; wherein X is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂—SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rd groups; wherein X⁻ is a counterion;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ee})₂, —OC(═O)N(R^{ff})₂, —NR^{ff}C(═O)R^{ee}, —NR^{ff}CO₂R^{cc}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{bb})N(R^{ee})₂, —OC(═NR^{bb})N(R^{ff})₂, —NR^{bb}C(═NR^{bb})N(R^{ee})₂, —NR^{ff}SO₂R^{ee}, —SO₂N(R^{ff})₂, —SO₂RV, —SO₂OR^{ee}, —OSO₂R^{ee}, —S(═O)R^{ee}, —Si(R^{ff})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee}), —OP(═O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₄ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl), —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₄ alkyl, C₁₋₄ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, BPh₄⁻, Al(OC(CF₃)₃)₄⁻, and a carborane anion (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

“Acyl” refers to a moiety selected from the group consisting of —C(═O)R^{aa}, —CHO, —CO₂R^{aa}, —C(═O)N(R^{bb}), —C(═NR^{bb})R^{aa}, —C(═NR^{bb})OR, —C(═NR^{bb})N(R^{bb}), —C(═O)NR^{bb}SO₂R^{aa}, —C(═S)N(R^{bb})₂, —C(═O)SR^{aa}, or —C(═S)SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{ee}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 Rd groups, and wherein R^{aa}, R^{bb}, R^{cc}, and R^{dd} are as defined above.

In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, —R^{aa}, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂ wherein X⁻, R^{aa}, R^{bb} and R^{cc} are as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, alkyl ethers or substituted alkyl ethers such as methyl, allyl, benzyl, substituted benzyls such as 4-methoxybenzyl, methoxylmethyl (MOM), benzyloxymethyl (BOM), 2-methoxyethoxymethyl (MEM), etc., silyl ethers such as trymethylsilyl (TMS), triethylsilyl (TES), triisopropylsilyl (TIPS), t-butyldimethylsilyl (TBDMS), etc., acetals or ketals, such as tetrahydropyranyl (THP), esters such as formate, acetate, chloroacetate, dichloroacetate, trichloroacetate, trifluoroacetate, methoxyacetate, etc., carbonates, sulfonates such as methanesulfonate (mesylate), benzylsulfonate, and tosylate (Ts), etc.

The term “leaving group” is given its ordinary meaning in the art of synthetic organic chemistry, for example, it can refer to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra.

### Example 1 Synthesis of Compound 2

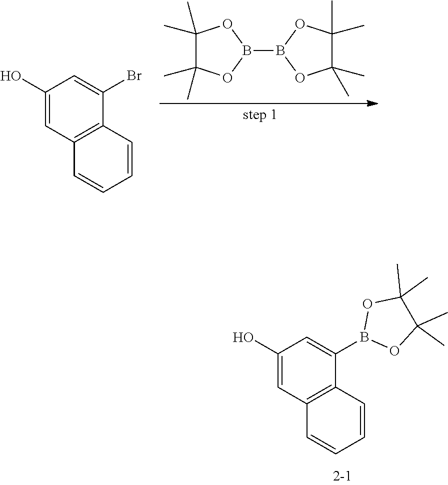

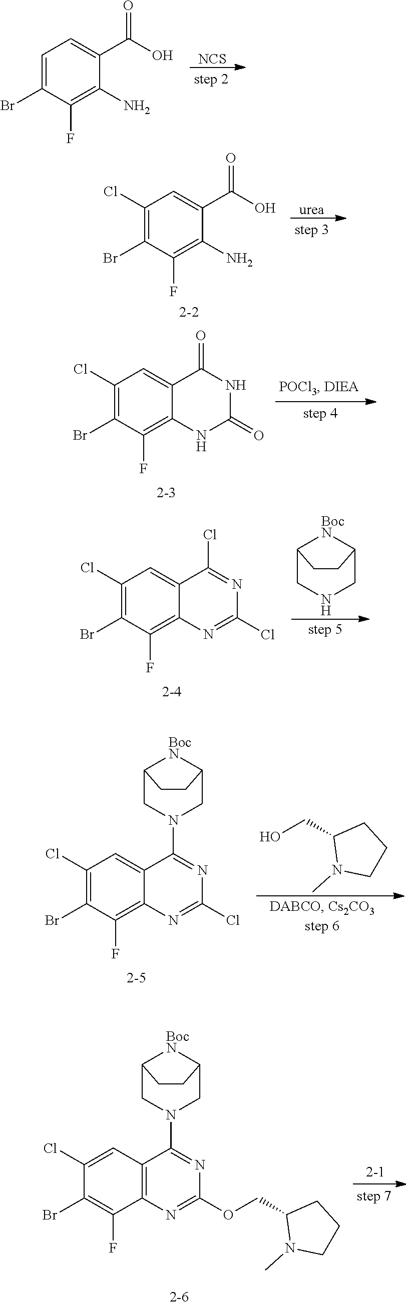

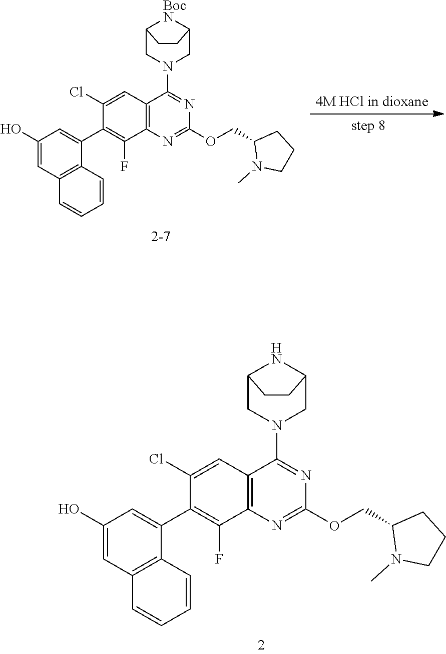 [SMILES: CN1CCC[C@H]1COc1nc(N2CC3CCC(C2)N3C(=O)OC(C)(C)C)c2cc(Cl)c(-c3cc(O)cc4ccccc34)c(F)c2n1 ; CN1CCC[C@H]1COc1nc(N2CC3CCC(C2)N3)c2cc(Cl)c(-c3cc(O)cc4ccccc34)c(F)c2n1]

Step 1: A mixture of 4-bromonaphthalen-2-ol (3.0 g, 13.4 mmol), bis(pinacolato)diboron (4.1 g, 16.1 mmol), Pd(dppf)Cl₂ (0.98 g, 1.35 mmol) and KOAc (3.9 g, 40.3 mmol) in 1,4-dioxane (30 mL) was stirred at 95° C. for 2 h under nitrogen atmosphere. The mixture was cooled and diluted with water. The resulting mixture was extracted with ethyl acetate. The combined organic layers were washed with brine, dried over anhydrous Na₂SO₄ and filtered. The residue was purified by silica gel column chromatography (petroleum ether/ethyl acetate=10/1) to afford 2-1.

Step 2: A mixture of 2-amino-4-bromo-3-fluorobenzoic acid (4.68 g, 20 mmol) and NCS (2.68 g, 20 mmol) in DMF (50 mL) was stirred at 70° C. for 16 h. The mixture was poured into ice-water (200 mL) and stirred for 30 min. The precipitate was collected by filtration and dried to afford 2-2.

Step 3: A mixture of 2-2 (5 g, 18.6 mmol) and urea (9 g, 149 mmol) was heated to 200° C. and stirred for 2 h. The mixture was cooled to room temperature and 200 mL of water was added. The mixture was heated to 100° C. and stirred for 3 h. The precipitate was collected by filtration and dried to afford 2-3.

Step 4: A mixture of 2-3 (5 g, 17 mmol) and N,N-diisopropylethylamine (5 mL) in phosphoryl trichloride (50 mL) was stirred at reflux for 16 h. The mixture was concentrated. The residue was poured into water and then extracted with ethyl acetate. The combined organic layers were washed with brine, dried over Na₂SO₄ and concentrated. The residue was purified by column chromatography on silica gel (petroleum ether/ethyl acetate=4/1) to afford 2-4.

Step 5: To a solution of tert-butyl (1R,5S)-3,8-diazabicyclo [3.2.1] octane-8-carboxylate (970 mg, 4.6 mmol) in DMSO (50 mL) was added N,N-diisopropylethylamine (1.2 g, 9.2 mmol) and 2-4 (1.5 g, 4.6 mmol). The reaction was stirred at room temperature for 2 h. The mixture was extracted with ethyl acetate and washed with water. The organic layer was dried over Na₂SO₄, filtered and concentrated. The residue was purified by column chromatography on silica gel (petroleum ether to petroleum ether/ethyl acetate=4/1) to afford 2-5.

Step 6: A mixture of 2-5 (600 mg, 1.18 mmol), (2S)-1-methylpyrrolidin-2-yl]methanol (409 mg, 3.55 mmol), triethylenediamine (133 mg, 1.18 mmol) and Cs₂CO₃ (1.16 g, 3.5 mmol) in DMF (4 mL) and THE (4 mL) was stirred at room temperature for 4 h. The mixture was extracted with ethyl acetate and washed with water. The combined organic layers were dried over Na₂SO₄ and concentrated. The residue was purified by column chromatography on silica gel (dichloromethane to dichloromethane/methanol=10/1) to afford 2-6.

Step 7: A mixture of 2-6 (140 mg, 0.24 mmol), 2-1 (84 mg, 0.31 mmol), Na₂CO₃ (63 mg, 0.60 mmol) and Pd(PPh₃)₄ (28 mg, 0.024 mmol) in 1,4-dioxane/water (1.5 mL/0.3 mL) was stirred at 95° C. for 4 h under nitrogen atmosphere. The mixture was concentrated and purified by column chromatography on silica gel (dichloromethane/methanol/ammonia=100/10/0.5) to afford 2-7.

Step 8: To a solution of 2-7 (100 mg, 0.15 mmol) in dichloromethane (4 mL) was added trifluoroacetic acid (1 mL). The reaction was stirred for 1 h at room temperature. The mixture was concentrated and purified by prep-HPLC (acetonitrile with 0.1% of formic acid in water: 5% to 25%) to afford 2 as a 0.6 eq of formic acid salt. LCMS (ESI, m/z): [M+H]⁺=548.5; HNMR (300 MHz, DMSO-d₆, ppm): δ 8.30-8.20 (m, 0.6H), 7.94 (s, 1H), 7.81 (d, J=8.4 Hz, 1H), 7.49-7.39 (m, 1H), 7.29 (d, J=2.4 Hz, 1H), 7.22 (d, J=4.2 Hz, 2H), 7.07 (d, J=2.4 Hz, 1H), 4.39-4.35 (m, 3H), 4.20-4.13 (m, 1H), 3.60-3.40 (m, 4H), 2.99-2.91 (m, 1H), 2.62-2.58 (m, 1H), 2.36 (s, 3H), 2.25-2.15 (m, 1H), 2.05-1.87 (m, 1H), 1.74-1.56 (m, 7H). FNMR (282 MHz, DMSO-d₆, ppm): δ −122.46 (1F).

### Example 2 Synthesis of Compound 28

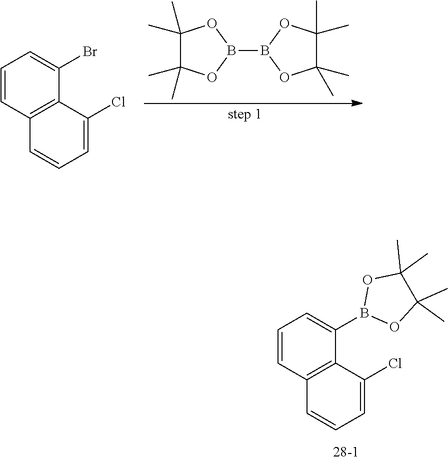

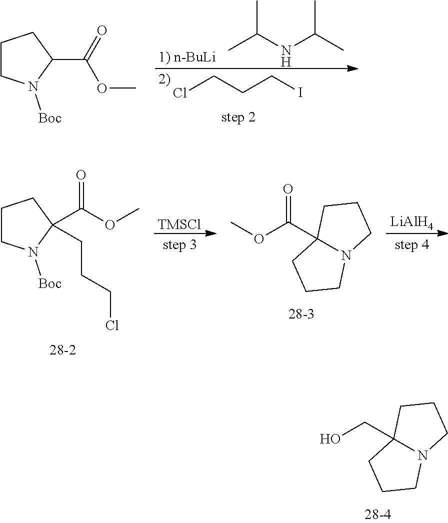

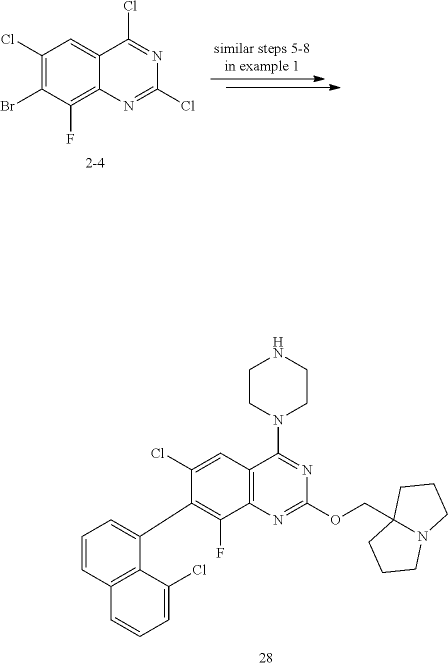 [SMILES: Fc1c(Br)c(Cl)cc2c(Cl)nc(Cl)nc12 ; Fc1c(-c2cccc3cccc(Cl)c23)c(Cl)cc2c(N3CCNCC3)nc(OCC34CCCN3CCC4)nc12]

Step 1: A mixture of 1-bromo-8-chloronaphthalene (5.0 g, 20.7 mmol), bis(pinacolato)diboron (5.8 g, 22.8 mmol), Pd(dppf)Cl₂ (1.5 g, 2.1 mmol) and KOAc (6.1 g, 62.1 mmol) in DMF (120 mL) was stirred at 80° C. for 3 h under nitrogen atmosphere. The mixture was cooled and diluted with water. The resulting mixture was extracted with ethyl acetate. The combined organic layers were washed with brine, dried over anhydrous Na₂SO₄ and filtered. The residue was purified by silica gel column chromatography (petroleum ether/ethyl acetate=20/1) to afford 28-1.

Step 2: To a solution of di-isopropylamine (37.1 g, 366.4 mmol) in THE was added n-BuLi (2.5 M in hexane, 136.0 mL, 340.2 mmol) dropwise at −78° C. under argon atmosphere. The mixture was stirred at −78° C. for 20 min, followed by addition of 1-tert-butyl 2-methyl pyrrolidine-1,2-dicarboxylate (60.0 g, 261.7 mmol) in THF. The resulting mixture was stirred at −78° C. for 1 h before addition of 1-chloro-3-iodopropane (107.0 g, 523.4 mmol) dropwise. The resulting mixture was stirred overnight at room temperature and then quenched with sat. NH₄Cl (aq.). The aqueous layer was extracted with ethyl acetate. The combined organic layers were dried over anhydrous Na₂SO₄, filtered and concentrated. The residue was purified by flash column chromatography on silica gel (petroleum ether/ethyl acetate=10/1) to afford 28-2.

Step 3: To a solution of 28-2 (69.0 g, 225.6 mmol) in methanol (1.4 L) was added TMSCl (122.6 g, 1128.2 mmol) at 0° C. The mixture was stirred overnight at room temperature. The mixture was basified to pH 8 with sat. NaHCO₃ solution. The aqueous layer was extracted with dichloromethane. The combined organic layers were dried over anhydrous Na₂SO₄, filtered and concentrated. The residue was purified by flash column chromatography on silica gel (dichloromethane/methanol=10/1) to afford 28-3.

Step 4: To a solution of 28-3 (20.0 g, 118.2 mmol) in THE (200 mL) was added LiAlH₄ (6.7 g, 177.3 mmol) in portions at 0° C. under nitrogen atmosphere. The resulting mixture was stirred at 0° C. for 30 min. The reaction was quenched by Na₂SO₄·10H₂O (20 g) and then 15% NaOH (5 mL) at 0° C. The mixture was filtered and washed with THF. The combined organic layers were dried over anhydrous Na₂SO₄, filtered and concentrated to afford 28-4.

Compound 28 was prepared following the procedures for the synthesis of compound 2 in example 1 as a formic acid salt. LCMS (ESI, m/z): [M+H]⁺=566.2; HNMR (400 MHz, DMSO-d₆, ppm): δ 8.28 (s, 1H), 8.21 (d, J=8.2 Hz, 1H), 8.11 (d, J=8.1 Hz, 1H), 7.88 (s, 1H), 7.74 (t, J=7.7 Hz, 1H), 7.66 (d, J=7.3 Hz, 1H), 7.61-7.45 (m, 2H), 4.14 (s, 2H), 3.95-3.45 (m, 4H), 3.17-2.93 (m, 6H), 2.75-2.65 (m, 2H), 2.05-1.65 (m, 8H). FNMR (282 MHz, DMSO-d₆, ppm): δ −122.25 (1F).

### Example 3 Synthesis of Compound 11

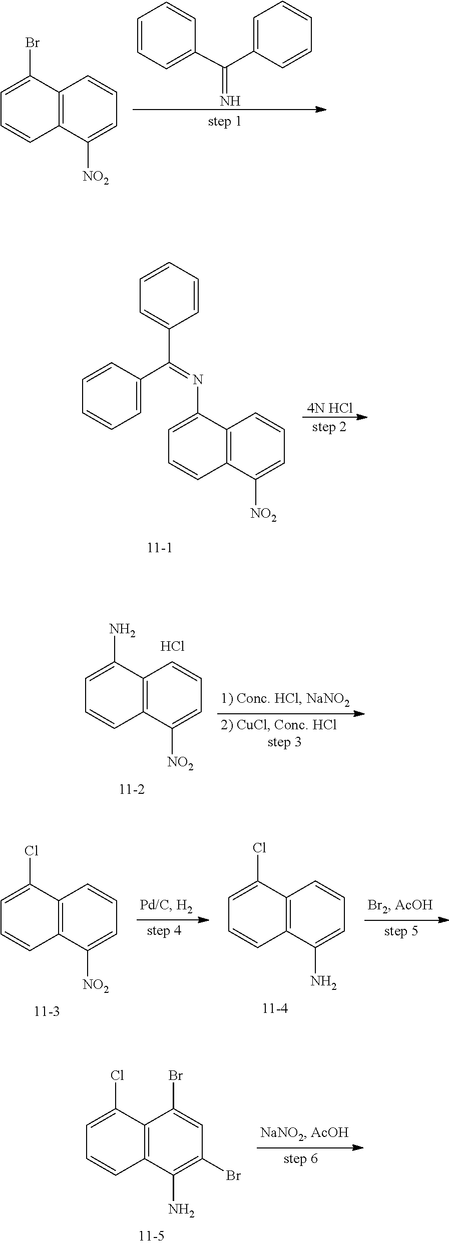

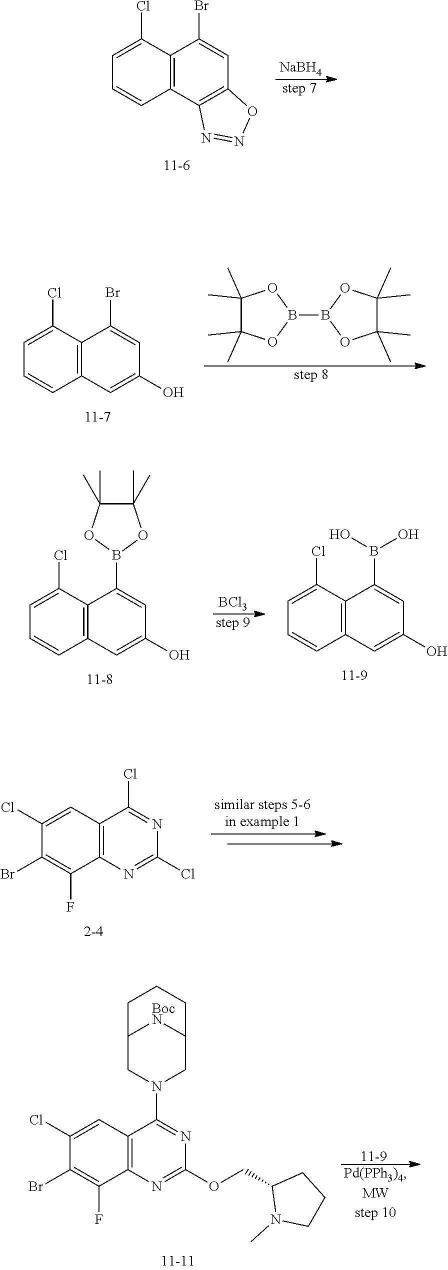

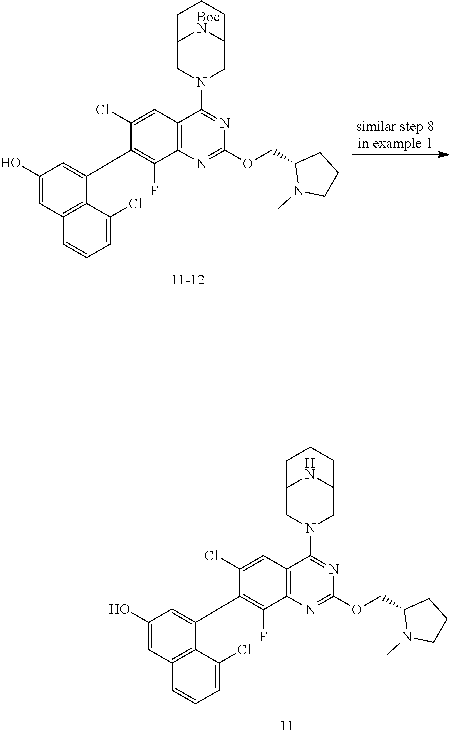 [SMILES: CN1CCC[C@H]1COc1nc(N2CC3CCCC(C2)N3C(=O)OC(C)(C)C)c2cc(Cl)c(-c3cc(O)cc4cccc(Cl)c34)c(F)c2n1 ; "similar step 8in example 1" ; CN1CCC[C@H]1COc1nc(N2CC3CCCC(C2)N3)c2cc(Cl)c(-c3cc(O)cc4cccc(Cl)c34)c(F)c2n1]

Step 1: A mixture of 5-bromo-1-nitro-naphthalene (25 g, 100 mmol), benzophenone imine (24 g, 130 mmol), Pd₂(dba)₃ (4.6 g, 5 mmol), XantPhos (2.9 g, 5 mmol) and Cs₂CO₃ (49 g, 150 mmol) in DMF (250 mL) was stirred at 100° C. for 5 h under nitrogen atmosphere. The mixture was filtered, and the filtrate was poured into water. The mixture was filtered and the filter cake was dried to afford 11-1.

Step 2: To a solution of 11-1 (31.3 g, 89 mmol) in dioxane (200 mL) was added 4N HCl (100 mL). The mixture was stirred at room temperature for 1 h. Then the mixture was filtered and dried to afford 11-2.

Step 3: To a suspension of 11-2 (78.8 g, 350 mmol) in conc. HCl (350 mL) and water (175 mL) was added a solution of sodium nitrite (25.4 g, 367.5 mmol) in water (51 mL) at 0° C. over 30 min. The reaction mixture was added to a vigorously stirred solution of CuCl (41.6 g, 420 mmol) in conc. HCl (131 mL) and water (175 mL) at room temperature over 1 h. The mixture was diluted with water and filtered. The filtrate cake was dissolved in dichloromethane, and washed with water, sat. NaHCO₃ solution and brine. The organic layer was dried over anhydrous Na₂SO₄, filtered and concentrated to afford 11-3.

Step 4: A mixture of 11-3 (67.6 g, 327 mmol) and 5% Pd/C (13.5 g) in ethyl acetate (2.37 L) was stirred at room temperature overnight under H₂ atmosphere. The reaction mixture was filtered. The filtrate was concentrated and triturated with n-heptane to afford 11-4.

Step 5: To a solution of bromine (97.9 g, 613.1 mmol) in acetic acid (470 mL) was added a solution of 11-4 (49.5 g, 278.7 mmol) in acetic acid (200 mL) at room temperature. The mixture was stirred at 70° C. for 4 h. The reaction mixture was cooled to room temperature and filtered. The filter cake was washed with acetic acid (120 mL) and then suspended in 20% NaOH (600 mL). The mixture was stirred at room temperature for 20 min and filtered. The solid was dissolved in dichloromethane, washed with brine, dried over anhydrous Na₂SO₄, filtered and concentrated to afford 11-5.

Step 6: To a solution of 11-5 (45.1 g, 134.3 mmol) in acetic acid (870 mL) and propionic acid (145 mL) was added sodium nitrite (13.0 g, 188.1 mmol) portion-wised at 5° C. The mixture was stirred at 5° C. for 1 h. Then the mixture was filtered, and the filtrate was poured into water. The resulting mixture was filtered. The cake was dissolved in dichloromethane, washed with brine, dried over Na₂SO₄, filtered and concentrated to afford 11-6.

Step 7: To a suspension of 11-6 (30.6 g, 108.1 mmol) in ethanol (310 mL) was added sodium borohydride (8.17 g, 216.15 mmol) portion-wise at 5° C. The mixture was stirred at 5° C. for 1 h, quenched with water (300 mL) and adjusted to about pH 5 with 1N HCl. The mixture was concentrated to remove the organic solvent. The resulting mixture was extracted with ethyl acetate. The combined organic layers were washed with brine, dried over anhydrous Na₂SO₄, filtered and concentrated. The residue was purified by column chromatography on silica gel (petroleum ether to petroleum ether/ethyl acetate=4/1) to afford 11-7.

Step 8: A mixture of 11-7 (6 g, 23.3 mmol), bis(pinacolato)diboron (11.84 g, 46.6 mmol), potassium acetate (6.85 g, 69.9 mmol), and Pd(dppf)Cl₂ (1.7 g, 2.33 mmol) in 1,4-dioxane (100 mL) was stirred at 95° C. for 7 h under N₂ atmosphere. Then the mixture was diluted with water and extracted with ethyl acetate. The combined organic layers were washed with brine, dried over Na₂SO₄, filtered and concentrated. The residue was purified by column chromatography on silica gel (petroleum ether to petroleum ether/ethyl acetate=4/1) to afford 11-8.

Step 9: To a solution of 11-8 (13.5 g, 44.4 mmol) in dichloromethane (300 mL) was added boron trichloride (88.8 mL, 88.8 mmol, 1 M in dichloromethane) at 0° C. The mixture was stirred at room temperature for 2 h. The mixture was quenched with water (200 mL) at 0° C. and then filtered. The filter cake was dissolved in ethyl acetate (200 mL). The filtrate was extracted with ethyl acetate. The ethyl acetate layers were combined, dried over sodium sulfate and concentrated to afford 11-9 which was used directly without purification.

Compound 11-11 was prepared following the procedures for the synthesis of compound 2 in example 1.

Step 10: A mixture of 11-11 (90 mg, 0.15 mmol), 11-9 (68 mg, 0.3 mmol), Pd(PPh₃)₄ (35 mg, 0.03 mmol) and Na₂CO₃ (48 mg, 0.45 mmol) in 1,4-dioxane (9 mL) and water (3 mL) was stirred at 105° C. for 1 h under nitrogen atmosphere and microwave condition. The mixture was cooled, poured into water and extracted with ethyl acetate. The combined organic layers were dried over anhydrous sodium sulfate, filtered and concentrated. The residue was purified by reverse phase chromatography (acetonitrile with 0.1% of formic acid in water: 5% to 95%) to afford 11-12.

Compound 11 was prepared following the procedures for the synthesis of compound 2 in example 1 as a 0.55 eq of formic acid salt. LCMS (ESI, m/z): [M+H]⁺=596.1; HNMR (400 MHz, methanol-d₄, ppm): δ 8.51 (brs, 0.55H), 7.94 (d, J=1.4 Hz, 1H), 7.75 (dd, J=8.0, 1.4 Hz, 1H), 7.37-7.30 (m, 3H), 6.98 (d, J=2.6 Hz, 1H), 4.75 (dd, J=12.4, 3.2 Hz, 2H), 4.69-4.58 (m, 2H), 3.99-3.80 (m, 2H), 3.56-3.48 (m, 4H), 3.93-2.98 (m, 1H), 2.89 (s, 3H), 2.30 (dd, J=15.0, 7.8 Hz, 1H), 2.15-1.95 (m, 8H), 1.71-1.64 (m, 1H).

### Example 4 Synthesis of Compound 60

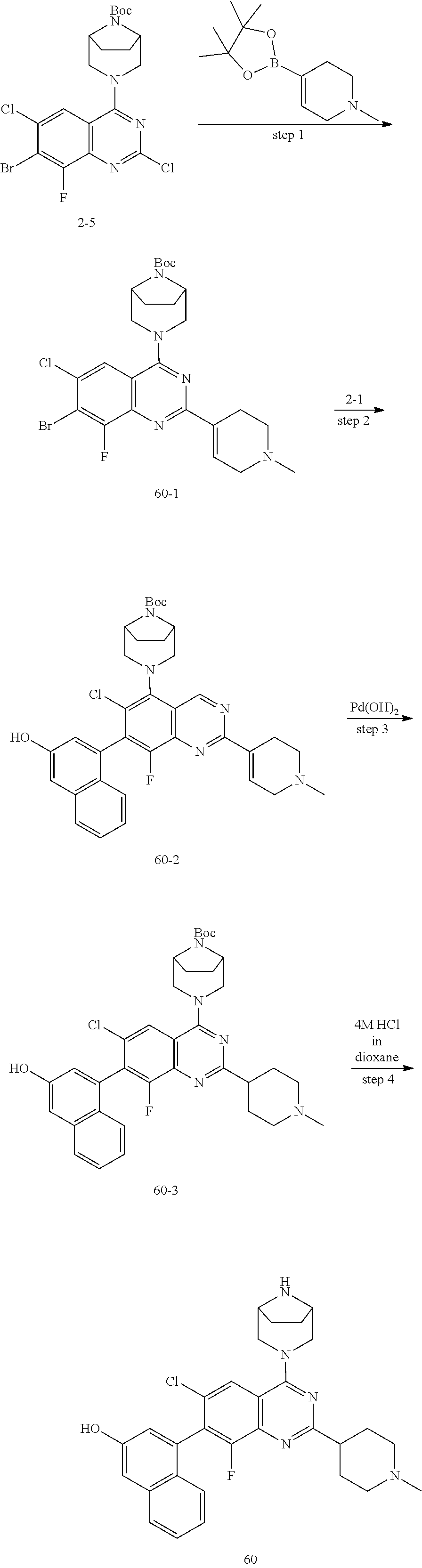

Step 1: A mixture of 2-5 (400 mg, 0.79 mmol), 1-methyl-4-(4,4,5,5-tetramethyl-1,3,2-dioxaborolan-2-yl)-1,2,3,6-tetrahydropyridine (264 mg, 1.2 mmol), Xantphos Pd G2 (60 mg, 0.079 mmol) and Na₂CO₃ (251 mg, 2.4 mmol) in water (2.0 mL) and 1,4-dioxane (20.0 mL) was stirred at 30° C. overnight under nitrogen atmosphere. The mixture was poured into water. The resulting solution was extracted with ethyl acetate. The combined organic layers were dried over Na₂SO₄, filtered and concentrated. The residue was purified by reverse phase flash chromatography (acetonitrile with 0.1% of formic acid in water: 5% to 95%) to afford 60-1.

Step 2: A mixture of 60-1 (150 mg, 0.26 mmol), 2-1 (121 mg, 0.47 mmol), Pd(PPh₃)₄ (30 mg, 0.026 mmol) and Na₂CO₃ (84 mg, 0.79 mmol) in water (2 mL) and 1,4-dioxane (10 mL) was stirred at 90° C. for 3 h under nitrogen atmosphere. The mixture was cooled down to room temperature and poured into water. The resulting solution was extracted with ethyl acetate. The combined organic layers were dried over Na₂SO₄, filtered and concentrated. The residue was purified by reverse phase flash chromatography (acetonitrile with 0.1% of formic acid in water: 5% to 95%) to afford 60-2.

Step 3: To a solution of 60-2 (80 mg, 0.12 mmol) in propan-2-ol (5 mL) was added Pd(OH)₂ (20 mg). The resulting solution was stirred at room temperature for 8 h under hydrogen atmosphere. The mixture was filtered and the filter cake was washed with ethyl acetate. The combined organic layers were dried over Na₂SO₄, filtered and concentrated. The residue was purified by reverse phase flash chromatography (acetonitrile with 0.1% of formic acid in water: 5% to 95%) to afford 60-3.

Step 4: To a solution of 60-3 (40 mg, 0.063 mmol) in 1,4-dioxane (3 mL) was added 4M HCl in 1,4-dioxane (3 mL) at 0° C. The mixture was stirred at room temperature for 6 h. Concentrated and the residue was purified by prep-HPLC to afford 60 (acetonitrile with 0.1% of formic acid in water: 5% to 35%). LCMS (ESI, m/z): [M+H]⁺=532.1; HNMR (400 MHz, methanol-d₄, ppm): δ 8.02 (d, J=1.4 Hz, 1H), 7.77 (d, J=8.4 Hz, 1H), 7.42 (dd, J=8.4, 2.8 Hz, 1H), 7.28 (d, J=2.4 Hz, 1H), 7.19 (d, J=4.8 Hz, 2H), 7.03 (d, J=2.4 Hz, 1H), 4.75 (d, J=13.6 Hz, 2H), 4.26-4.21 (m, 2H), 3.90 (d, J=14.2 Hz, 2H), 3.66 (d, J=12.8 Hz, 2H), 3.20 (t, J=11.8 Hz, 3H), 2.89 (s, 3H), 2.38-2.35 (m, 2H), 2.29-2.22 (m, 2H), 2.18-2.12 (m, 4H).

### Example 5 Synthesis of Compound 81

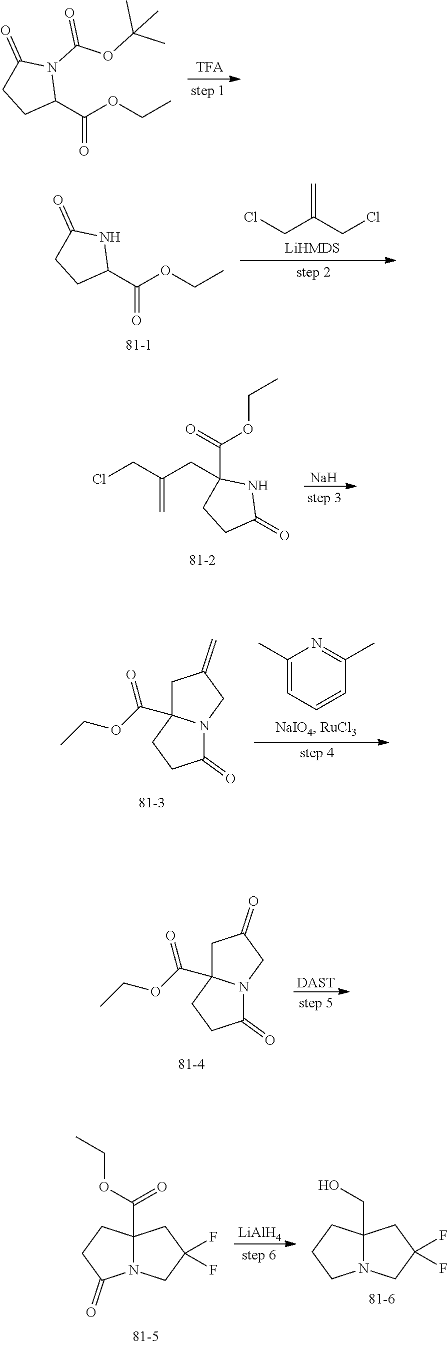

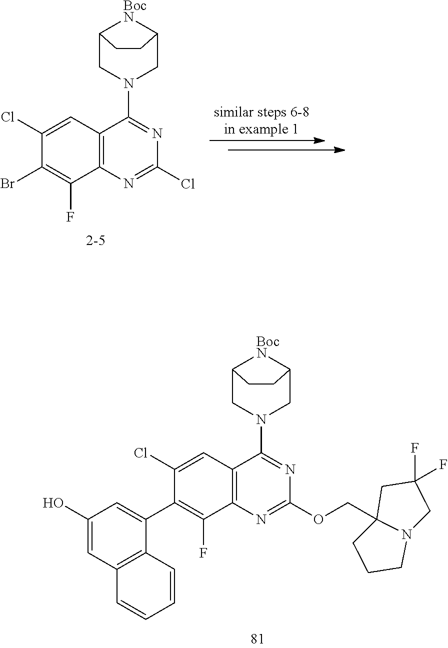 [SMILES: CC(C)(C)OC(=O)N1C2CCC1CN(c1nc(Cl)nc3c(F)c(Br)c(Cl)cc13)C2 ; "similar steps 6-8in example 1" ; CC(C)(C)OC(=O)N1C2CCC1CN(c1nc(OCC34CCCN3CC(F)(F)C4)nc3c(F)c(-c4cc(O)cc5ccccc45)c(Cl)cc13)C2]

Step 1: To a solution of 1-(tert-butyl) 2-ethyl 5-oxopyrrolidine-1,2-dicarboxylate (100 g, 388.7 mmol) in dichloromethane (160 mL) was added trifluoroacetic acid (80 mL) slowly at room temperature. The mixture was stirred at room temperature for 16 h, and then concentrated. The residue was diluted with sat. NaHCO₃ and extracted with ethyl acetate. The combined organic layers were washed with brine, dried over Na₂SO₄, filtered and concentrated to afford 81-1.

Step 2: To a solution of 81-1 (49 g, 311.8 mmol) and 3-chloro-2-(chloromethyl)prop-1-ene (100 g, 800 mmol) in tetrahydrofuran (200 mL) was added LiHMDS (655 mL, 1.0 M in tetrahydrofuran, 655 mmol) at −40° C. under nitrogen atmosphere. The mixture was stirred at room temperature for 2 h. The reaction was quenched with sat. NH₄Cl. The mixture was extracted with ethyl acetate. The combined organic layers were washed with brine, dried over Na₂SO₄, filtered and concentrated. The residue was purified by column chromatography on silica gel (petroleum ether to petroleum ether/ethyl acetate=1/1) to afford 81-2.

Step 3: To a solution of sodium hydride (2.72 g, 68.1 mmol) in tetrahydrofuran (1 L) was added a solution of 81-2 (13.6 g, 55.35 mmol) in tetrahydrofuran (100 mL) dropwise at 0° C. under nitrogen atmosphere. Then the mixture was heated to reflux and stirred for 9 h. The mixture was cooled to 0° C. and quenched with water (500 mL). The mixture was extracted with ethyl acetate. The combined organic layers were washed with brine, dried over Na₂SO₄, filtered and concentrated. The residue was purified by column chromatography on silica gel (petroleum ether to petroleum ether/ethyl acetate=1/1) to afford 81-3.

Step 4: To a solution of 81-3 (9.0 g, 43.15 mmol) in acetonitrile (245 mL) and dichloromethane (245 mL) was added 2,6-dimethylpyridine (9.25 g, 86.3 mmol), water (370 mL), periodate sodium (36.9 g, 172.6 mmol) sequentially. Then a solution of Ruthenium (III) chloride (313 mg, 1.51 mmol) in water (40 mL) was added dropwise to the mixture. The mixture was stirred for 1 h at room temperature. The mixture was diluted with water and extracted with dichloromethane. The combined organic layers were washed with brine, dried over Na₂SO₄, filtered and concentrated. The residue was purified by column chromatography on silica gel (petroleum ether to petroleum ether/ethyl acetate=1/1) to afford 81-4.

Step 5: To a solution of 81-4 (10.55 g, 50 mmol) in dichloromethane (150 mL) was added diethylaminosulfur trifluoride (20.13 g, 125 mmol) at 0° C. under N₂ atmosphere. The mixture was stirred at room temperature for 16 h. The reaction was quenched with ethanol. The mixture was washed with water and brine. The organic layer was dried over Na₂SO₄, filtered and concentrated. The residue was purified by column chromatography on silica gel (petroleum ether to petroleum ether/ethyl acetate=1/1) to afford 81-5.

Step 6: To a solution of LiAlH₄ (3.08 g, 81 mmol) in tetrahydrofuran (60 mL) was added a solution of 81-5 (6.3 g, 27 mmol) in tetrahydrofuran (40 mL) at 0° C. under nitrogen atmosphere. The mixture was stirred at reflux for 1 h. Then the mixture was cooled to 0° C., quenched with sodium sulfate decahydrate and filtered. The filtrate was concentrated to afford 81-6.

Compound 81 was prepared following the procedures for the synthesis of compound 2 in example 1 as a 3 eq of TFA salt. LCMS (ESI, m/z): [M+H]⁺=610.3; HNMR (400 MHz, methanol-d₄, ppm): δ 8.02 (s, 1H), 7.76-7.35 (m, 1H), 7.43-7.48 (m, 1H), 7.37-7.25 (m, 1H), 7.21-7.15 (m, 2H), 7.02-7.00 (m, 1H), 4.78-4.67 (m, 4H), 4.24-4.15 (m, 3H), 3.92-3.80 (m, 4H), 3.46-3.39 (m, 1H), 3.02-2.75 (m, 2H), 2.46-2.13 (m, 8H). FNMR (376 MHz, methanol-d₄, ppm): δ −98.31 (1F), −100.55 (1F), −123.38 (1F).

### Example 6 Synthesis of Compound 73

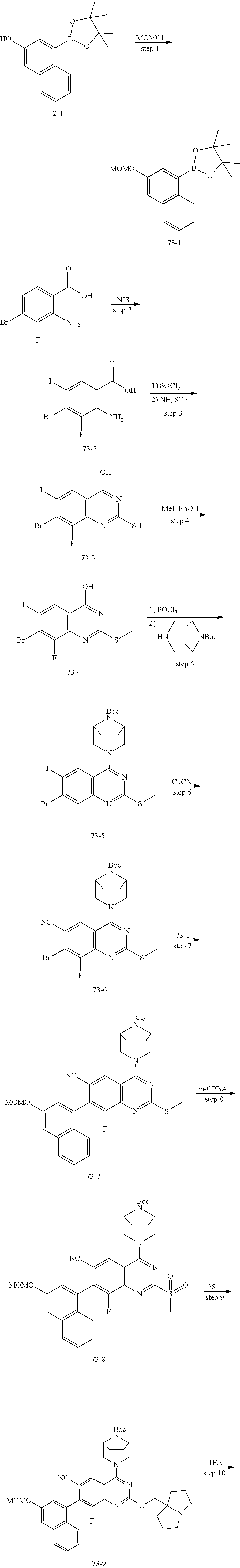

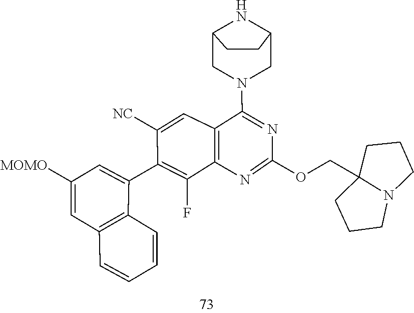

Step 1: A mixture of 2-1 (2.7 g, 10 mmol), N,N-diisopropylethylamine (2.6 g, 20 mmol) and chloro(methoxy)methane (1.21 g, 15 mmol) in dichloromethane (40 mL) was stirred at room temperature overnight. The mixture was diluted with dichloromethane, and washed with water. The organic layer was dried over Na₂SO₄, filtered and concentrated. The residue was purified by column chromatography on silica gel (petroleum ether to petroleum ether/ethyl acetate=9/1) to afford 73-1.

Step 2: To a solution of 2-amino-4-bromo-3-fluorobenzoic acid (4.66 g, 20 mmol) in dimethylformamide (20 mL) was added N-iodosuccinimide (6.75 g, 30 mmol) at room temperature. The mixture was stirred at 80° C. for 2 h, then cooled and poured into water. Then the mixture was filtered and washed with water. The filter cake was triturated with acetonitrile and filtered to afford 73-2.

Step 3: A solution of 73-2 (3.59 g, 10 mmol) in thionyl chloride (60 mL) was stirred at 50° C. for 3 h. Concentrated and the residue was dissolved in acetone (15 mL), which was added into a solution of ammonium thiocyanate (836 mg, 11 mmol) in acetone (40 mL) dropwise. The mixture was stirred at room temperature for 1 h. The mixture was filtered and the filter cake was washed with water and then dissolved in 10% NaOH. The mixture was filtered and the filtrate was adjusted to about pH 2 with 1M HCl. The mixture was filtered again and the filter cake was triturated with methanol to afford 73-3.

Step 4: To a solution of 73-3 (2.3 g, 5.75 mmol) in methanol (60 mL) was added a solution of NaOH (460 mg, 11.5 mmol) in water (46 mL) and iodomethane (1.62 g, 11.5 mmol). The mixture was stirred at room temperature for 2 h. The mixture was poured into water and adjusted to about pH 6 with 1M HCl. Then the mixture was filtered and the cake was triturated with methanol to afford 73-4.

Step 5: To a solution of 73-4 (1 g, 2.4 mmol) in phosphorus oxychloride (8 mL) was added N,N-diisopropylethylamine (1 mL) at room temperature. The mixture was stirred at 100° C. for 2 h, cooled, concentrated, diluted with ethyl acetate, washed with water and brine successively. The organic layer was dried over Na₂SO₄, filtered and concentrated. The residue was dissolved in dimethyl sulfoxide (15 mL), followed by the addition of tert-Butyl 3,8-diazabicyclo[3.2.1]octane-8-carboxylate (636 mg, 3 mmol) and N,N-diisopropylethylamine (645 mg, 5 mmol) at room temperature. The mixture was stirred for 1 h, diluted with ethyl acetate, washed with water and brine. The organic layer was dried over Na₂SO₄, filtered and concentrated. The residue was purified by column chromatography on silica gel (petroleum ether to petroleum ether/ethyl acetate=1/4) to afford 73-5.

Step 6: A mixture of 73-5 (1.22 g, 2 mmol) and copper (I) cyanide (360 mg, 4 mmol) in N,N-dimethylformamide (10 mL) was stirred at 100° C. for 6 h under N₂ atmosphere. The mixture was cooled, diluted with ethyl acetate and washed with water and brine successively. The organic layer was dried over Na₂SO₄, filtered and concentrated. The residue was purified by column chromatography on silica gel (petroleum ether to petroleum ether/ethyl acetate=1/4) to afford 73-6.

Step 7: A mixture of 73-6 (250 mg, 0.5 mmol), 73-1 (188 mg, 0.6 mmol), sodium carbonate (212 mg, 2 mmol) and tetrakis(triphenylphosphine)palladium (58 mg, 0.05 mmol) in 1,4-dioxane/water (4/1, 3 mL) was stirred at 95° C. for 30 min under N₂ atmosphere under microwave condition. The mixture was diluted with ethyl acetate and washed with water and brine successively. The organic layer was dried over Na₂SO₄, filtered and concentrated. The residue was purified by column chromatography on silica gel (petroleum ether to petroleum ether/ethyl acetate=1/4) to afford 73-7.

Step 8 and Step 9: A mixture of 73-7 (215 mg, 0.35 mmol) and 3-chloroperbenzoic acid (71 mg, 0.35 mmol) in dichloromethane (10 mL) was stirred at 0° C. for 0.5 h. The mixture was cooled, diluted with ethyl acetate (50 mL), and washed with water (50 mL) and brine (50 mL) successively. The organic layer was dried over Na₂SO₄, filtered and concentrated to afford 73-8. A solution of 73-8 in toluene (2 mL) was added to a pre-stirred solution of 28-4 (148 mg, 1.05 mmol) and sodium tert-butoxide (58 mg, 0.6 mmol) in toluene (5 mL) at 0° C. under N₂ condition. The reaction was stirred for 0.5 h and quenched with sat. ammonium chloride solution. The mixture was extracted with ethyl acetate. The organic layer was washed with water and brine, dried over Na₂SO₄, filtered and concentrated. The residue was purified by column chromatography on silica gel (dichloromethane to dichloromethane/methanol/ammonia=10/1/0.05) to afford 73-9.

Step 10: To a solution of 73-9 (43 mg, 0.06 mmol) in dichloromethane (1.5 mL) was added trifluoroacetic acid (0.5 mL). The mixture was stirred at room temperature for 1 h. The mixture was diluted with ethyl acetate and washed with sat. NaHCO₃ and brine. The organic layer was dried over Na₂SO₄, filtered and concentrated. The residue was purified by prep-HPLC (acetonitrile with 0.05% of TFA in water: 10% to 95%) to afford 73 as 3 eq of TFA salt. LCMS (ESI, m/z): [M+H]=565.3; HNMR (400 MHz, methanol-d₄, ppm): δ 8.38-8.36 (m, 1H), 7.79-7.76 (m, 1H), 7.46-7.41 (m, 1H), 7.32-7.20 (m, 3H), 7.14-7.12 (m, 1H), 4.82-4.77 (m, 2H), 4.67 (s, 2H), 4.25-4.21 (m, 2H), 3.99-3.93 (m, 2H), 3.72-3.64 (m, 2H), 3.29-3.24 (m, 2H), 2.35-2.05 (m, 12H). FNMR (376 MHz, methanol-d₄, ppm): δ −124.53 (1F).

### Example 7 Synthesis of Compound 71

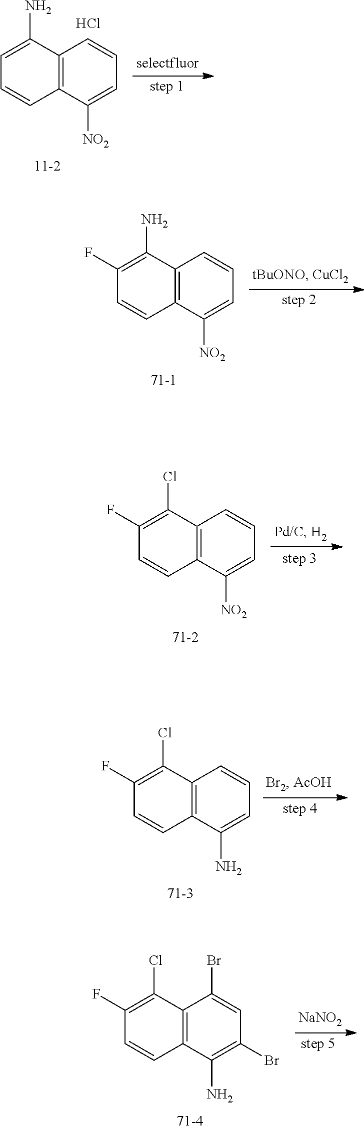

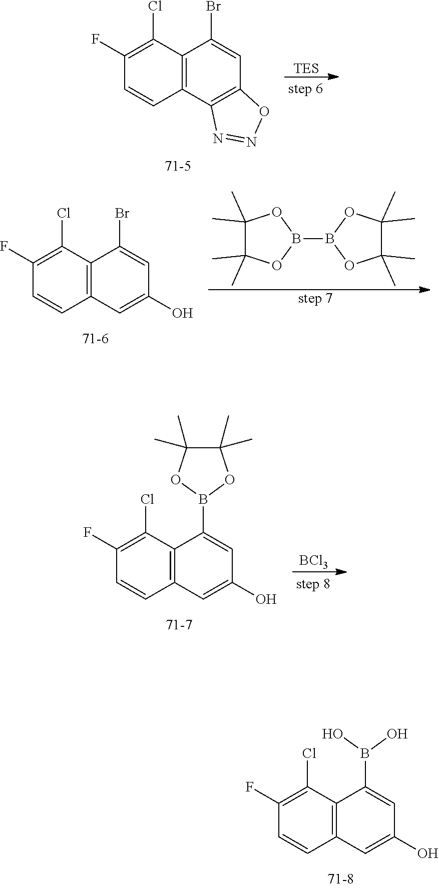

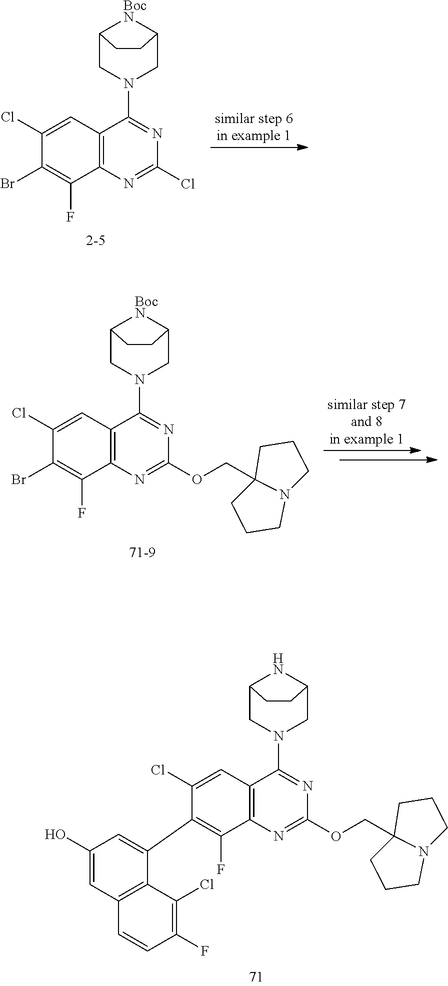 [SMILES: CC(C)(C)OC(=O)N1C2CCC1CN(c1nc(Cl)nc3c(F)c(Br)c(Cl)cc13)C2 ; "similar step 6in example 1" ; CC(C)(C)OC(=O)N1C2CCC1CN(c1nc(OCC34CCCN3CCC4)nc3c(F)c(Br)c(Cl)cc13)C2 ; "similar step 7and 8in example 1" ; Oc1cc(-c2c(Cl)cc3c(N4CC5CCC(C4)N5)nc(OCC45CCCN4CCC5)nc3c2F)c2c(Cl)c(F)ccc2c1]

Step 1: A mixture of 11-2 (19 g, 101 mmol), triethylamine (20.4 g, 202 mmol), selectflour (93 g, 263 mmol) in ethanol/1-Methyl-2-pyrrolidinone (150 mL/150 mL) was stirred at room temperature overnight under N₂ atmosphere. The mixture was diluted with water and extracted with ethyl acetate. The combined organic layers were washed with water, brine, dried over anhydrous Na₂SO₄, filtered and concentrated to afford 71-1.

Step 2: To a mixture of 71-1 (21 g, 105 mmol) and copper chloride (15.5 g, 115.5 mmol) in acetonitrile (200 mL) was added tert-butyl nitrite (16.2 g, 57.5 mmol) under N₂ atmosphere at 0° C. Then the mixture was stirred at room temperature for 2 h. The mixture was diluted with water and extracted with ethyl acetate. The combined organic layers were washed with water, brine, dried over anhydrous Na₂SO₄, filtered and concentrated. The residue was purified by column chromatography on silica gel (petroleum ether to petroleum ether/ethyl acetate=10/1) to afford 71-2.

Step 3: A mixture of 71-2 (18.6 g, 83 mmol) and 5% Pd/C (2.0 g) in ethyl acetate (200 mL) was stirred at room temperature for 24 h under hydrogen atmosphere. Then the mixture was filtered and concentrated to give a residue which was purified by column chromatography on silica gel (petroleum ether to petroleum ether/ethyl acetate=4/1) and prep-HPLC (acetonitrile with 0.05% of TFA in water: 25% to 95%) to afford 71-3.

Step 4: To a mixture of 71-3 (6.6 g, 33.8 mmol) in acetic acid (300 mL) was added bromine (11.9 g, 74.5 mmol) at room temperature. The mixture was stirred at 70° C. for 6 h. Then the mixture was filtered and the filtrate was concentrated to afford 71-4.

Step 5: To a solution of 71-4 (9.1 g, 25.9 mmol) in acetic acid/propionic (100 mL/25 mL) was added sodium nitrite (2.15 g, 31 mmol) at 0° C. The mixture was stirred at 0° C. for 1 h. The mixture was diluted with water and extracted with dichloromethane. The combined organic layers were dried over anhydrous Na₂SO₄, filtered and concentrated to afford 71-5.

Step 6: To a mixture of 71-5 (8.3 g, 27.7 mmol) in isopropyl alcohol (200 mL) was added triethylsilane (6.42 g, 55.3 mmol). The mixture was stirred at 100° C. overnight under N₂ atmosphere. Then concentrated and the residue was purified by column chromatography on silica gel (petroleum ether to petroleum ether/ethyl acetate=4/1) to afford 71-6.

Step 7: To a mixture of 71-6 (2.0 g, 7.3 mmol) in dioxane (30 mL) was added 4,4,4′,4′,5,5,5′,5′-octamethyl-2,2′-bi(1,3,2-dioxaborolane) (2.4 g, 9.5 mmol), potassium acetate (2.15 g, 21.9 mmol) and [1,1′-bis(diphenylphosphino)ferrocene]dichloropalladium(II) (534 mg, 0.73 mmol). The mixture was stirred at 95° C. for 4 h under N₂ atmosphere. The mixture was filtered and the filtrate was diluted with water and extracted with ethyl acetate. The combined organic layers were dried over anhydrous Na₂SO₄, filtered and concentrated. The residue was purified by column chromatography on silica gel (petroleum ether to petroleum ether/ethyl acetate=4/1) to afford 71-7.

Step 8: To a solution of 71-7 (1 g, 3.1 mmol) in dichloromethane (5 mL) was added boron chloride (1.0 M in methylene chloride, 6.2 mL, 6.2 mmol) at room temperature. The mixture was stirred at room temperature for 2 h. The mixture was diluted with ice water and extracted with dichloromethane. The combined organic layers were dried over anhydrous Na₂SO₄, filtered and concentrated. The residue was purified by prep-HPLC (acetonitrile with 0.05% of TFA in water: 5% to 95%) to afford 71-8.

Compound 71-9 was prepared following the procedure for the synthesis of compound 2 in example 1.

Compound 71 was prepared following the procedures for the synthesis of compound 2 in example 1 as a 3 eq of TFA salt. LCMS (ESI, m/z): [M+H]⁺=626.3; HNMR (400 MHz, methanol-d₄, ppm): δ 7.93-7.92 (m, 1H), 7.80 (dd, J=9.2, 5.6 Hz, 1H), 7.40-7.35 (m, 2H), 7.01-7.00 (d, J=2.4 Hz, 1H), 4.77-4.74 (m, 2H), 4.64-4.62 (m, 3H), 4.25-4.22 (m, 2H), 3.93-3.90 (m, 1H), 3.83-3.79 (m, 1H), 3.70-3.62 (m, 2H), 3.26-3.24 (m, 1H), 2.33-2.06 (m, 12H). FNMR (376 MHz, methanol-d₄, ppm): δ −116.5 (1F), −123.7 (1F).

### Example 8 Synthesis of Compound 42

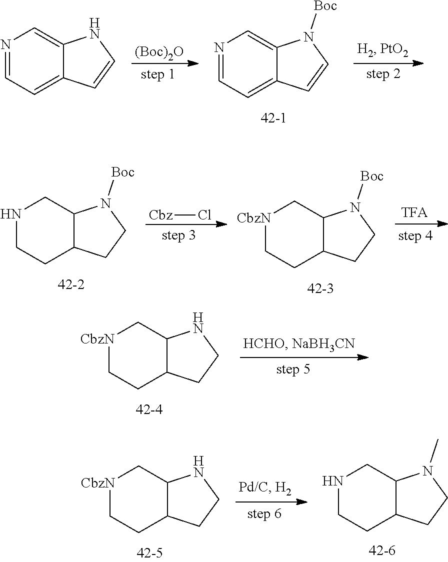

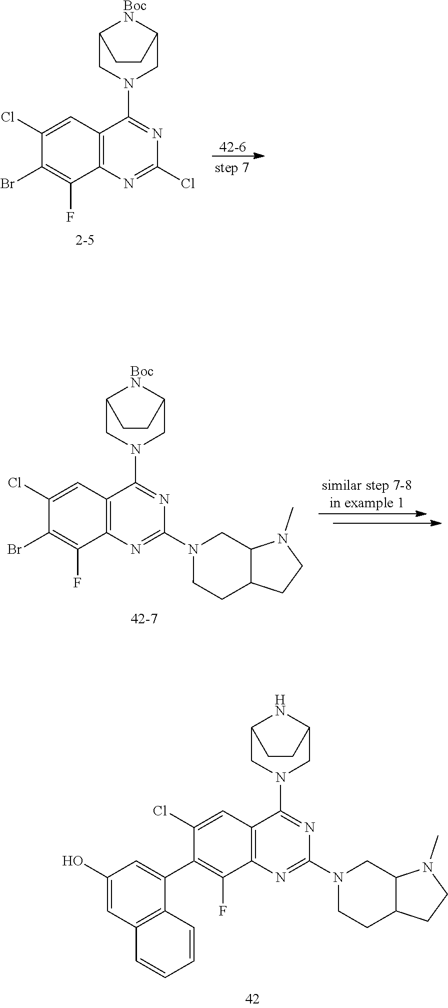 [SMILES: CC(C)(C)OC(=O)N1C2CCC1CN(c1nc(Cl)nc3c(F)c(Br)c(Cl)cc13)C2 ; "42-6" ; CN1CCC2CCN(c3nc(N4CC5CCC(C4)N5C(=O)OC(C)(C)C)c4cc(Cl)c(Br)c(F)c4n3)CC21 ; "similar step 7-8in example 1" ; CN1CCC2CCN(c3nc(N4CC5CCC(C4)N5)c4cc(Cl)c(-c5cc(O)cc6ccccc56)c(F)c4n3)CC21]

Step 1: To a solution of 1H-pyrrolo[2,3-c]pyridine (2.8 g, 23.7 mmol) in DCM (30 mL) were added TEA (3.6 g, 35.6 mmol, 4.96 mL) and di-tert-butyl carbonate (5.69 g, 26.1 mmol). The mixture was stirred at room temperature for 3 h. The mixture was diluted with water and extracted with ethyl acetate. The combined organic layers were washed with water, brine, dried over anhydrous Na₂SO₄, filtered and concentrated. The residue was purified by column chromatography on silica gel (petroleum ether/ethyl acetate=3/1) to afford 42-1.

Step 2: A mixture of 42-1 (1.51 g, 6.92 mmol) and PtO₂ (314 mg, 1.38 mmol) in AcOH (10 mL) was stirred at room temperature for 15 h under 4 atm of H₂. The mixture was filtered and the filtrate was concentrated to afford 42-2 which was used directly in the next step without purification.

Step 3: To a solution of 42-2 (1.56 g, 6.89 mmol) in dichloromethane (20 mL) were added TEA (1.05 g, 10.34 mmol, 1.44 mL) and benzyl chloroformate (1.29 g, 7.58 mmol) at 0° C. The solution was stirred at room temperature for 3 h. The mixture was diluted with water and extracted with dichloromethane. The combined organic layers were washed with water, brine, dried over anhydrous Na₂SO₄, filtered and concentrated. The residue was purified by column chromatography on silica gel (petroleum ether/ethyl acetate=3/1) to afford 42-3.

Step 4: To a solution of 42-3 (507 mg, 1.41 mmol) in dichloromethane (5 mL) was added TFA (801 mg, 7.0 mmol) at 0° C. The resulting solution was stirred at room temperature for 3 h. The solution was concentrated to afford 42-4.

Step 5: To a solution of 42-4 (366 mg, 1.41 mmol) in CH₃OH (5 mL) was added HCHO (324 mg, 3.53 mmol, 37 wt %) and cat. acetic acid at room temperature. The resulting solution was stirred at room temperature for 15 min, followed by addition of NaBH₃CN (265 mg, 4.22 mmol). The resulting solution was stirred at room temperature for 3 h. The mixture was diluted with water and extracted with ethyl acetate. The combined organic layers were washed with water, brine, dried over anhydrous Na₂SO₄, filtered and concentrated. The residue was purified by column chromatography on silica gel (petroleum ether/ethyl acetate=3/1) to afford 42-5.

Step 6: To a solution of 42-5 (302 mg, 1.1 mmol) in CH₃OH (5 mL) was added Pd/C (30 mg). The resulting solution was stirred at room temperature for 15 h under H₂. The mixture was filtered and concentrated to afford 42-6 which was used directly in the next step without purification.

Step 7: A mixture of 42-6 (133 mg, 0.95 mmol), 2-5 (150 mg, 0.3 mmol) and DIEA (230 mg, 1.78 mmol) in dichloromethane (5 mL) was stirred at room temperature for 16 h. The mixture was diluted with dichloromethane and washed with water. The organic layer was dried over anhydrous Na₂SO₄, filtered and concentrated. The residue was purified by column chromatography on silica gel (dichloromethane/methanol=10/1) to afford 42-7.

Compound 42 was prepared following the procedures for the synthesis of compound 2 in example 1 as a 1.5 eq of formic acid salt. LCMS (ESI, m/z): [M+H]⁺=573.2; HNMR (400 MHz, methanol-d₄, ppm): δ 8.44 (s, 1.5H), 7.80-7.73 (m, 2H), 7.41 (t, J=1.2 Hz, 1H), 7.39-7.17 (m, 3H), 7.00 (s, 1H), 5.18-5.06 (m, 2H), 4.67-4.57 (m, 1H), 4.52-4.47 (m, 2H), 4.07-4.00 (m, 2H), 3.71-3.52 (m, 4H), 3.27-3.23 (m, 1H), 3.04-3.01 (m, 1H), 2.98 (s, 3H), 2.68-2.52 (m, 1H), 2.28-2.22 (m, 1H), 2.08-1.99 (m, 4H), 1.98-1.96 (m, 1H), 1.70-1.57 (m, 1H), 1.54-1.50 (m, 1H).

### Example 9 Synthesis of Compound 80

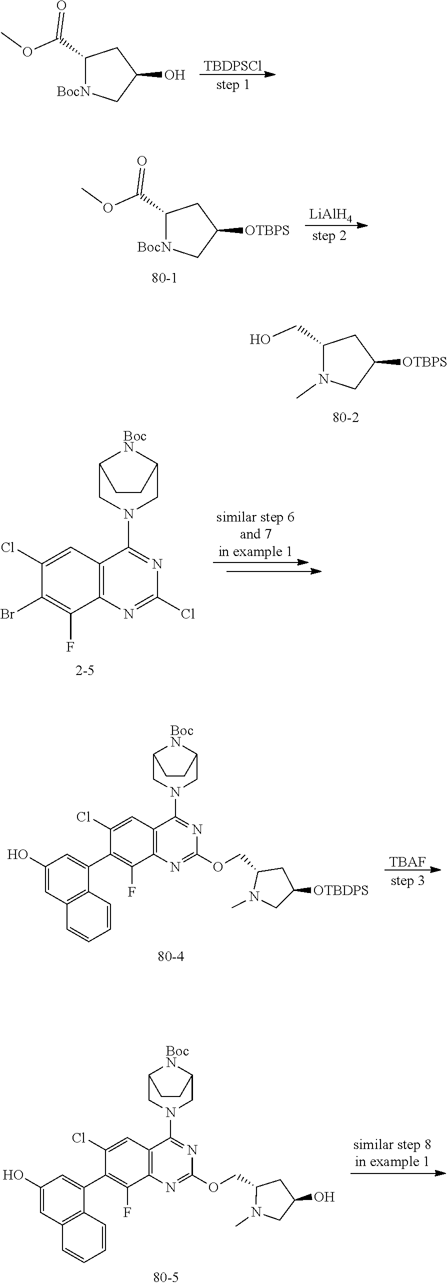

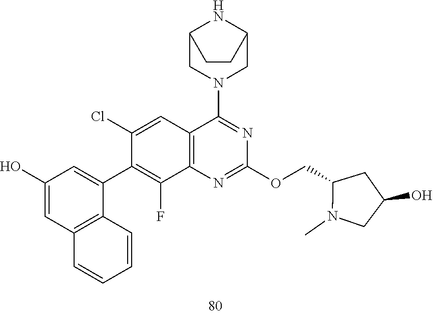 [SMILES: CN1C[C@H](O)C[C@H]1COc1nc(N2CC3CCC(C2)N3)c2cc(Cl)c(-c3cc(O)cc4ccccc34)c(F)c2n1]

Step 1: A mixture of 1-(tert-butyl) 2-methyl (2S,4R)-4-hydroxypyrrolidine-1,2-dicarboxylate (2 g, 8.15 mmol), imidazole (1.67 g, 24.46 mmol), DMAP (49.81 mg, 0.4 mmol), and TBDPSCl (2.69 g, 9.79 mmol) in dichloromethane (40 mL) was stirred at room temperature for 16 h. The mixture was diluted with water and extracted with dichloromethane. The combined organic layers were washed with water, brine, dried over anhydrous Na₂SO₄, filtered and concentrated. The residue was purified by a reverse phase HPLC (acetonitrile with 0.05% of TFA in water: 5% to 95%) to afford 80-1.

Step 2: A mixture of 80-1 (2 g, 4.14 mmol) and LiAlH₄ (1 M in THF, 16 mL, 16 mmol) in dry THF (40 mL) was stirred at 70° C. for 3 h. The reaction was cooled to 0° C. and quenched by addition of potassium bisulfate (2 M, 5 mL). The resulting slurry was filtered and washed with THF. The filtrate was concentrated. The residue was purified by reverse phase HPLC (acetonitrile with 0.05% of TFA in water: 5% to 95%) to afford 80-2.

Followed similar steps in example 1 to synthesize 80-4.

Step 3: To a solution of 80-4 (100 mg, 0.11 mmol) in TH (5 mL) was added TBAF (1 M in THF, 2 mL) at 0° C. The mixture was stirred at room temperature for 6 h. The mixture was diluted with water and extracted with ethyl acetate. The combined organic layers were washed with water, brine, dried over anhydrous Na₂SO₄, filtered and concentrated. The residue was purified by a reverse phase HPLC (acetonitrile with 0.05% of TFA in water: 5% to 95%) to afford 80-5.

Compound 80 was prepared following the procedures for the synthesis of compound 2 in example 1. LCMS (ESI, m/z): [M+H]⁺=564.1; HNMR (400 MHz, DMSO-d₆, ppm): δ 10.00 (s, 1H), 7.94 (s, 1H), 7.80 (d, J=8.4 Hz, 1H), 7.46-7.42 (m, 1H), 7.27 (d, J=2.4 Hz, 1H), 7.21 (d, J=4.2 Hz, 2H), 7.05 (d, J=2.4 Hz, 1H), 4.76 (d, J=4.4 Hz, 1H), 4.36-4.31 (m, 3H), 4.19-4.14 (m, 2H), 3.55-3.50 (m, 4H), 3.18 (dd, J=9.4, 6.0 Hz, 1H), 2.85-2.78 (m, 1H), 2.34 (s, 3H), 2.12 (dd, J=9.4, 6.2 Hz, 1H), 1.87-1.74 (m, 2H), 1.65-1.63 (m, 4H).

### Example 10 Synthesis of Compound 77

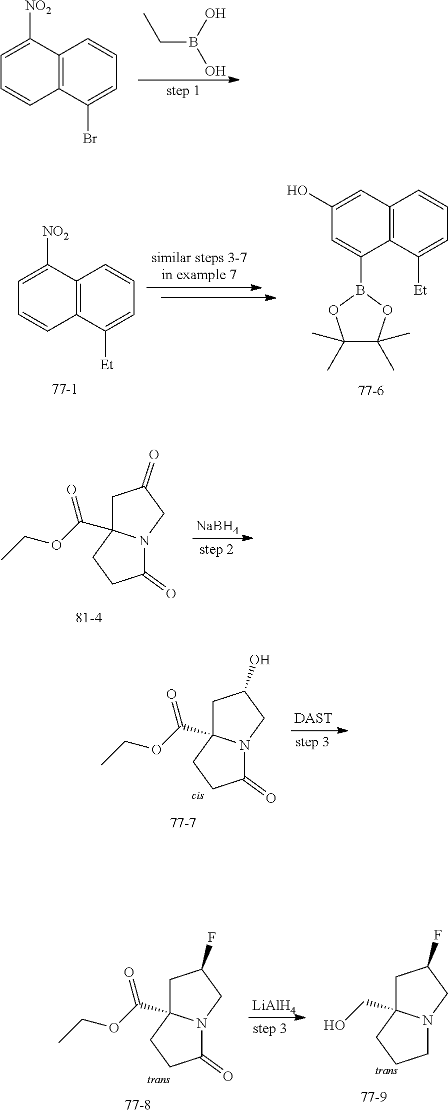

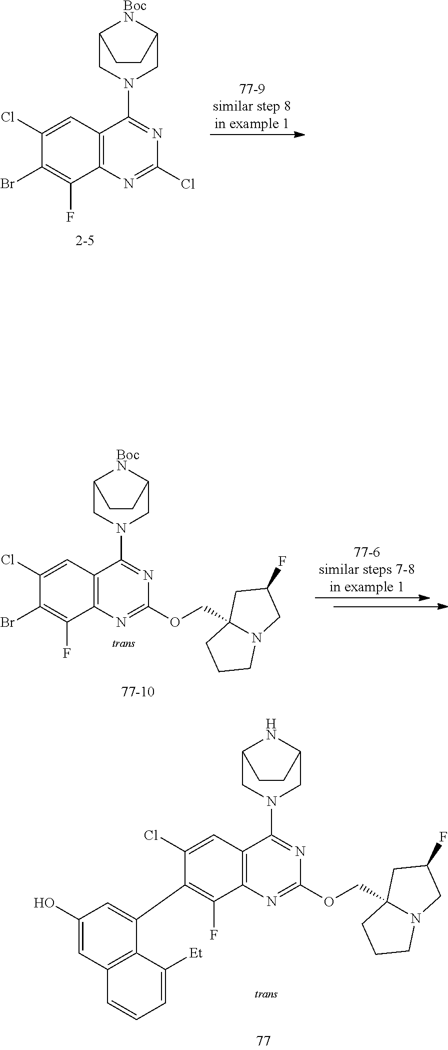 [SMILES: CC(C)(C)OC(=O)N1C2CCC1CN(c1nc(Cl)nc3c(F)c(Br)c(Cl)cc13)C2 ; "77-9similar step 8in example 1" ; CC(C)(C)OC(=O)N1C2CCC1CN(c1nc(OC[C@@]34CCCN3C[C@H](F)C4)nc3c(F)c(Br)c(Cl)cc13)C2 ; "77-6similar steps 7-8in example 1" ; CCc1cccc2cc(O)cc(-c3c(Cl)cc4c(N5CC6CCC(C5)N6)nc(OC[C@@]56CCCN5C[C@H](F)C6)nc4c3F)c12]

Step 1: To a mixture of potassium phosphate (176 g, 714 mmol) in toluene/water (896 mL/112 mL) was added 5-bromo-1-nitro-naphthalene (70 g, 278 mmol), ethylboronic acid (41.15 g, 556 mmol), and [1,1′-bis(diphenylphosphino)ferrocene]dichloropalladium(II) (10.1 g, 13.9 mmol) under nitrogen atmosphere. The mixture was stirred at 100° C. for 16 h. The mixture was filtered and the filtrate was washed with water and brine. The organic layer was dried over sodium sulfate, filtered and concentrated. The residue was purified by column chromatography on silica gel (petroleum ether to petroleum ether/ethyl acetate=95/5) to afford 77-1.

Compound 77-6 was prepared following the procedures for the synthesis of compound 71-7 in example 7.

Step 2: To a solution of 81-4 (10.6 g, 50.2 mmol) in methanol (100 mL) was added sodium borohydride (475 mg, 12.55 mmol) in portions at 0° C. under nitrogen atmosphere, and the mixture was stirred at 0° C. for 5 min. The mixture was concentrated and purified by column chromatography on silica gel (petroleum ether to ethyl acetate) to afford 77-7.

Step 3: To a solution of 77-7 (4.8 g, 22.6 mmol) in dichloromethane (50 mL) was added diethylaminosulfur trifluoride (4.1 g, 2.35 mmol) at −78° C. The mixture was stirred for 5 h at room temperature. Then the mixture was quenched with methanol, diluted with water, and extracted with dichloromethane. The combined organic layers were washed with brine, dried over sodium sulfate, filtered and concentrated. The residue was purified by column chromatography on silica gel (petroleum ether to petroleum ether/ethyl acetate=1/1) to afford 77-8.

Step 4: To a solution of lithium aluminium hydride (1.25 g, 33 mmol) in tetrahydrofuran (33 mL) was added a solution of 77-8 (2.36 g, 11 mmol) in tetrahydrofuran (10 mL) at 0° C. under nitrogen atmosphere. The mixture was stirred at reflux for 2 h, and then cooled to 0° C. Water (1.3 mL), 15% aqueous NaOH solution (1.3 mL) and water (3.9 mL) was added. The mixture was dried over sodium sulfate and filtered. The filtrate was concentrated to afford 77-9.

Compound 77-10, a racemic mixture of the trans isomer, was prepared following the procedures for the synthesis of compound 2-6 in example 1.

Compound 77 was prepared following the procedures for the synthesis of compound 2 in example 1 as a 3 eq of TFA salt. LCMS (ESI, m/z): [M+H]⁺=620.4; HNMR (400 MHz, methanol-d₄, ppm): δ 8.00-7.96 (m, 1H), 7.61 (d, J=8.0 Hz, 1H), 7.34 (t, J=7.6 Hz, 1H), 7.26 (d, J=2.4 Hz, 1H), 7.13-7.11 (m, 1H), 6.80 (d, J=2.4 Hz, 1H), 5.61-5.48 (m, 1H), 4.80-4.60 (m, 5H), 4.26-4.21 (m, 2H), 4.05-3.80 (m, 4H), 3.47-3.44 (m, 1H), 2.80-2.03 (m, 12H), 0.92-0.87 (m, 3H). FNMR (376 MHz, methanol-d₄, ppm): δ −122.65 (1F), −174.3 (1F).

### Example 11 Synthesis of Compounds 105 and 106

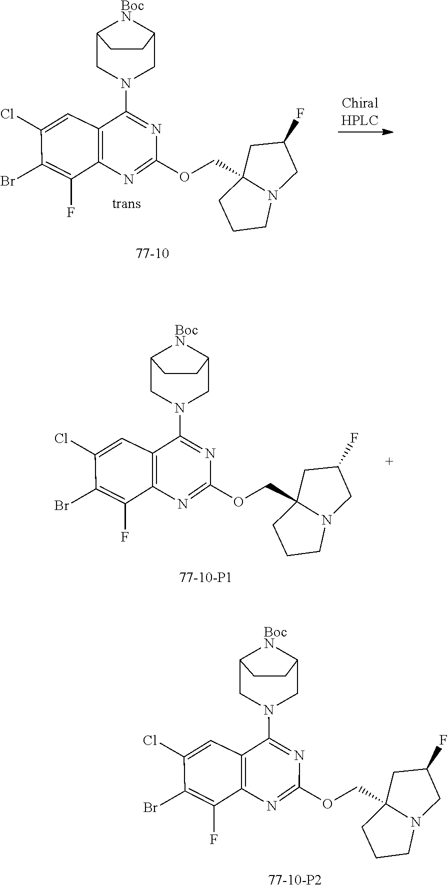 [SMILES: CC(C)(C)OC(=O)N1C2CCC1CN(c1nc(OC[C@@]34CCCN3C[C@H](F)C4)nc3c(F)c(Br)c(Cl)cc13)C2 ; CC(C)(C)OC(=O)N1C2CCC1CN(c1nc(OC[C@]34CCCN3C[C@@H](F)C4)nc3c(F)c(Br)c(Cl)cc13)C2 ; CC(C)(C)OC(=O)N1C2CCC1CN(c1nc(OC[C@@]34CCCN3C[C@H](F)C4)nc3c(F)c(Br)c(Cl)cc13)C2]

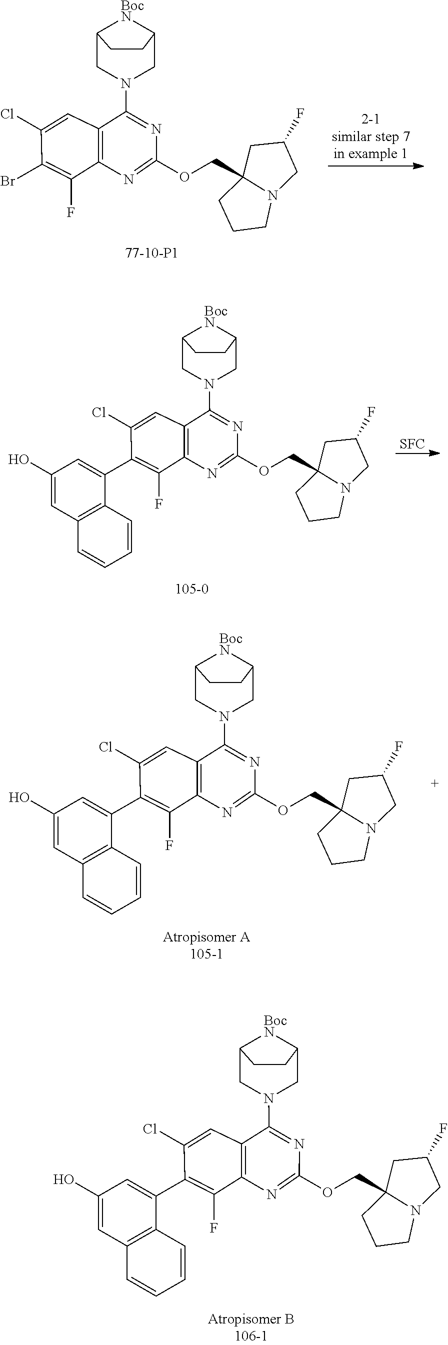

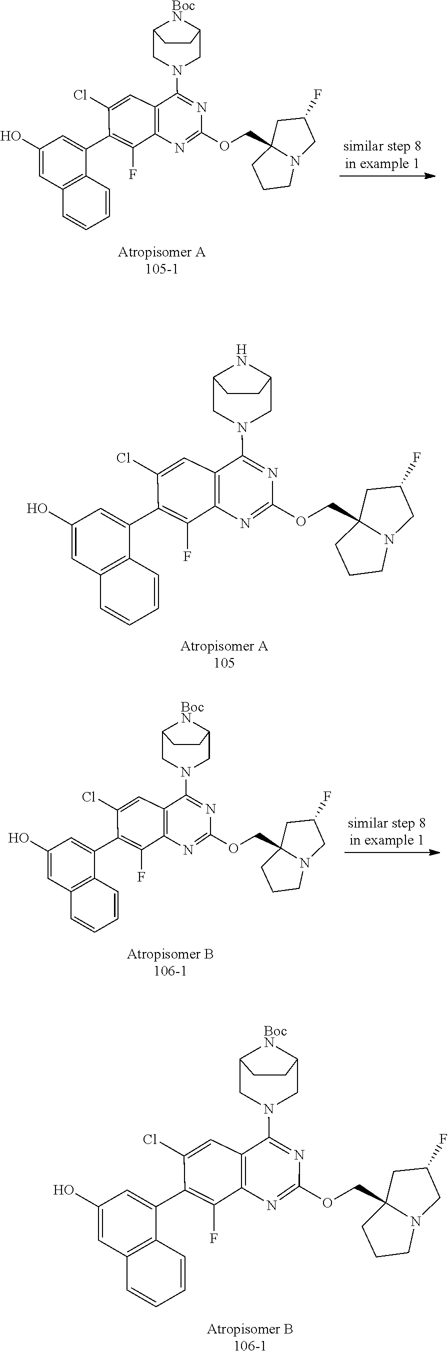 [SMILES: CC(C)(C)OC(=O)N1C2CCC1CN(c1nc(OC[C@]34CCCN3C[C@@H](F)C4)nc3c(F)c(-c4cc(O)cc5ccccc45)c(Cl)cc13)C2 ; Oc1cc(-c2c(Cl)cc3c(N4CC5CCC(C4)N5)nc(OC[C@]45CCCN4C[C@@H](F)C5)nc3c2F)c2ccccc2c1 ; CC(C)(C)OC(=O)N1C2CCC1CN(c1nc(OC[C@]34CCCN3C[C@@H](F)C4)nc3c(F)c(-c4cc(O)cc5ccccc45)c(Cl)cc13)C2 ; CC(C)(C)OC(=O)N1C2CCC1CN(c1nc(OC[C@]34CCCN3C[C@@H](F)C4)nc3c(F)c(-c4cc(O)cc5ccccc45)c(Cl)cc13)C2]

Compound 77-10 (2.3 g) was purified by chiral prep-HPLC (column: CHIRALPAK®IA, 30% IPA in hexane) to afford 77-10-P1 (900 mg, yield: 38%) and 77-10-P2 (820 mg, yield: 34%), respectively.

77-10-P1: Chiral HPLC analysis: >99% ee; Retention time: 4.873 min; column: CHIRALPAK®IA, 30% IPA in hexane; flow rate: 1 mL/min.
77-10-P2: Chiral HPLC analysis: >99% ee; Retention time: 6.710 min; column: CHIRALPAK®IA, 30% IPA in hexane; flow rate: 1 mL/min.

Compound 105-0 was prepared from 77-10-P1 following the procedure for the synthesis of compound 2 in example 1.

Compound 105-0 (430 mg) was purified by SFC (column: Chiral-OM, MeOH (0.1% DEA)/CO₂=45/55) to afford 105-1 (110 mg) and 106-1 (225 mg), respectively.

105-1: SFC analysis: >99% ee; Retention time: 4.92 min; column: Chiral-OM, MeOH (0.1% DEA) in CO₂, 5% to 40%; pressure: 100 bar; flow rate: 1.5 mL/min.
106-1: SFC analysis: >99% ee; Retention time: 5.24 min; column: Chiral-OM, MeOH (0.1% DEA) in CO₂, 5% to 40%; pressure: 100 bar; flow rate: 1.5 mL/min.

Compound 105 was prepared from 105-1 following the procedure for the synthesis of compound 2 in example 1 as a 3 eq of TFA salt. LCMS (ESI, m/z): [M+H]⁺=592.3; HNMR (400 MHz, methanol-d₄, ppm): δ 8.02-8.00 (m, 1H), 7.74 (d, J=8.4 Hz, 1H), 7.43-7.38 (m, 1H), 7.26 (d, J=2.4 Hz, 1H), 7.21-7.15 (m, 2H), 7.01 (d, J=2.4 Hz, 1H), 5.62-5.47 (m, 1H), 4.76-4.67 (m, 4H), 4.23 (s, 2H), 4.03-3.80 (m, 5H), 3.47-3.40 (m, 1H), 2.74-2.51 (m, 2H), 2.44-2.28 (m, 3H), 2.19-2.10 (m, 5H). FNMR (376 MHz, methanol-d₄, ppm): δ −123.44 (1F), −174.28 (1F).

Compound 106 was prepared from 106-1 following the procedure for the synthesis of compound 2 in example 1 as a 3 eq of TFA salt. LCMS (ESI, m/z): [M+H]⁺=592.3; HNMR (400 MHz, methanol-d₄, ppm): δ 8.01-8.00 (m, 1H), 7.74 (d, J=8.0 Hz, 1H), 7.43-7.38 (m, 1H), 7.26 (d, J=2.4 Hz, 1H), 7.21-7.16 (m, 2H), 7.02 (d, J=2.4 Hz, 1H), 5.62-5.47 (m, 1H), 4.80-4.66 (m, 4H), 4.23 (s, 2H), 4.03-3.79 (m, 5H), 3.47-3.38 (m, 1H), 2.74-2.52 (m, 2H), 2.44-2.28 (m, 3H), 2.19-2.08 (m, 5H). FNMR (376 MHz, methanol-d₄, ppm): δ −123.43 (1F), −174.27 (1F).

### Example 12 Synthesis of Compounds 107 and 108

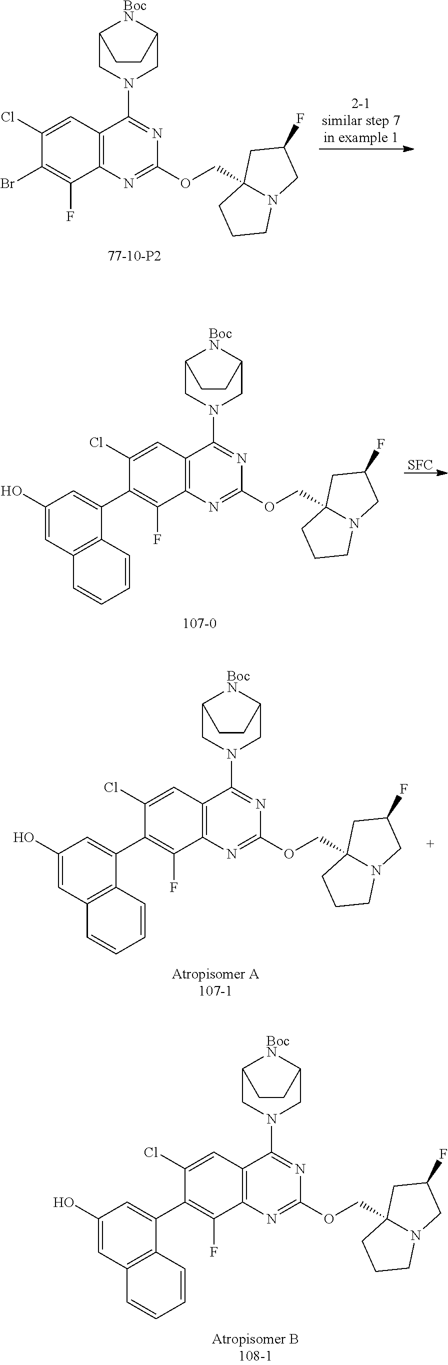

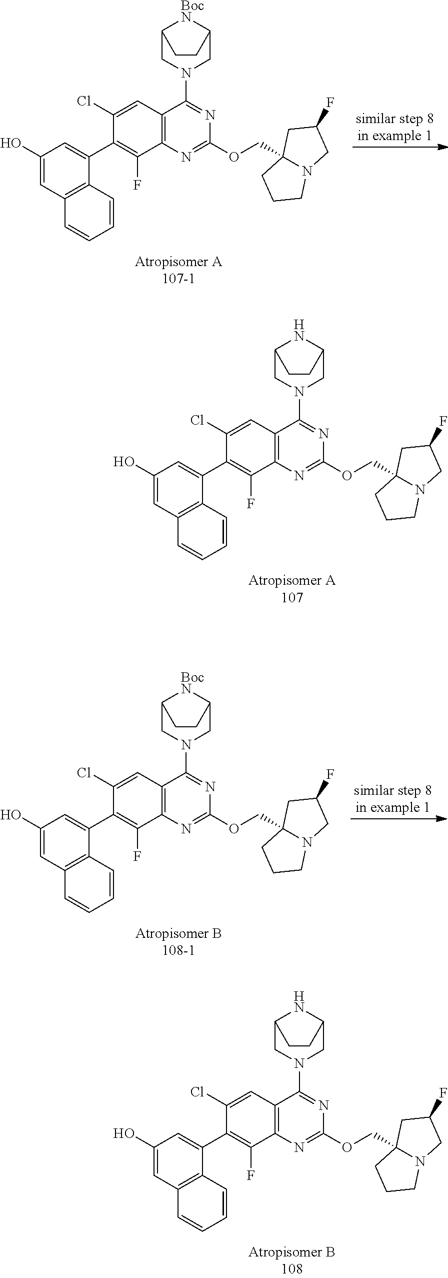 [SMILES: CC(C)(C)OC(=O)N1C2CCC1CN(c1nc(OC[C@@]34CCCN3C[C@H](F)C4)nc3c(F)c(-c4cc(O)cc5ccccc45)c(Cl)cc13)C2 ; "similar step 8in example 1" ; Oc1cc(-c2c(Cl)cc3c(N4CC5CCC(C4)N5)nc(OC[C@@]45CCCN4C[C@H](F)C5)nc3c2F)c2ccccc2c1 ; CC(C)(C)OC(=O)N1C2CCC1CN(c1nc(OC[C@@]34CCCN3C[C@H](F)C4)nc3c(F)c(-c4cc(O)cc5ccccc45)c(Cl)cc13)C2 ; "similar step 8in example 1" ; Oc1cc(-c2c(Cl)cc3c(N4CC5CCC(C4)N5)nc(OC[C@@]45CCCN4C[C@H](F)C5)nc3c2F)c2ccccc2c1]

Compound 107-0 was prepared from 77-10-P2 following the procedure for the synthesis of compound 2 in example 1.

Compound 107-0 (269 mg) was purified by SFC (column: Chiral-OZ, EtOH (0.1% DEA)/CO₂=60/40) to afford 107-1 (101 mg) and 108-1 (140 mg), respectively.

107-1: SFC analysis: >99% ee; Retention time: 4.46 min; column: CHIRALCEL® OZ, 40% MeOH (0.1% of DEA) in CO₂; pressure: 100 bar; flow rate: 3.0 mL/min.
108-1: SFC analysis: >99% ee; Retention time: 6.46 min; column: CHIRALCEL® OZ, 40% MeOH (0.1% of DEA) in CO₂; pressure: 100 bar; flow rate: 3.0 mL/min.

Compound 107 was prepared from 107-1 following the procedure for the synthesis of compound 2 in example 1 as a 3 eq of TFA salt. LCMS (ESI, m/z): [M+H]⁺=592.3; HNMR (400 MHz, methanol-d₄, ppm): δ 8.02-8.00 (m, 1H), 7.74 (d, J=8.4 Hz, 1H), 7.43-7.38 (m, 1H), 7.26 (d, J=2.4 Hz, 1H), 7.20-7.15 (m, 2H), 7.01 (d, J=2.4 Hz, 1H), 5.62-5.47 (m, 1H), 4.79-4.65 (m, 4H), 4.23 (s, 2H), 4.04-3.80 (m, 5H), 3.47-3.40 (m, 1H), 2.74-2.51 (m, 2H), 2.44-2.28 (m, 3H), 2.19-2.09 (m, 5H). FNMR (376 MHz, methanol-d₄, ppm): δ −123.46 (1F), −174.30 (1F).

Compound 108 was prepared from 108-1 following the procedure for the synthesis of compound 2 in example 1 as a 3 eq of TFA salt. LCMS (ESI, m/z): [M+H]⁺=592.3; HNMR (400 MHz, methanol-d₄, ppm): δ 8.01-8.00 (m, 1H), 7.74 (d, J=8.4 Hz, 1H), 7.42-7.38 (m, 1H), 7.26 (d, J=2.4 Hz, 1H), 7.21-7.16 (m, 2H), 7.01 (d, J=2.4 Hz, 1H), 5.62-5.47 (m, 1H), 4.78-4.67 (m, 4H), 4.23 (s, 2H), 4.05-3.81 (m, 5H), 3.47-3.39 (m, 1H), 2.74-2.50 (m, 2H), 2.44-2.28 (m, 3H), 2.19-2.08 (m, 5H). FNMR (376 MHz, methanol-d₄, ppm): δ −123.42 (1F), −174.26 (1F).

### Example 13 Synthesis of Compounds 101 and 102

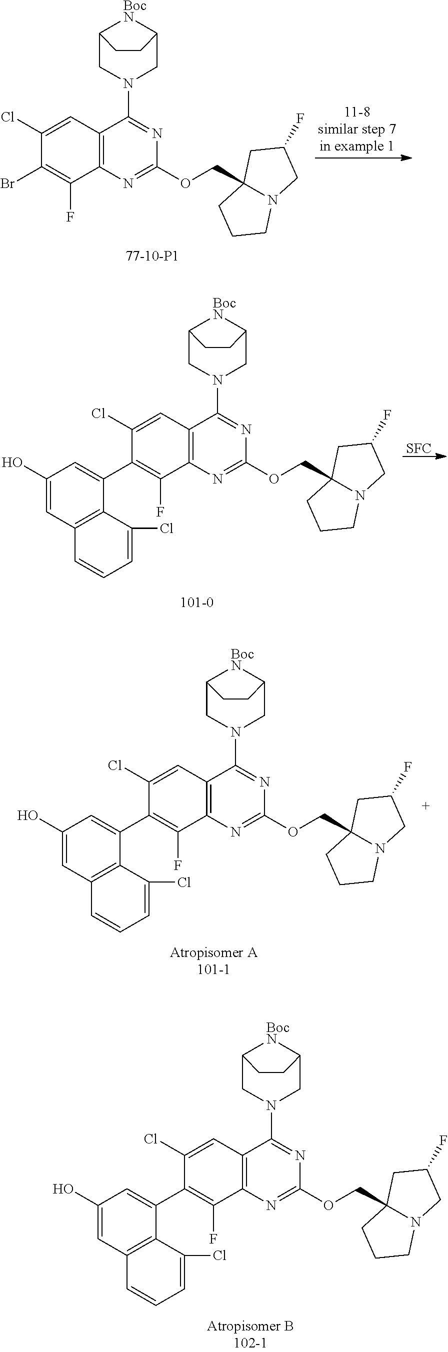

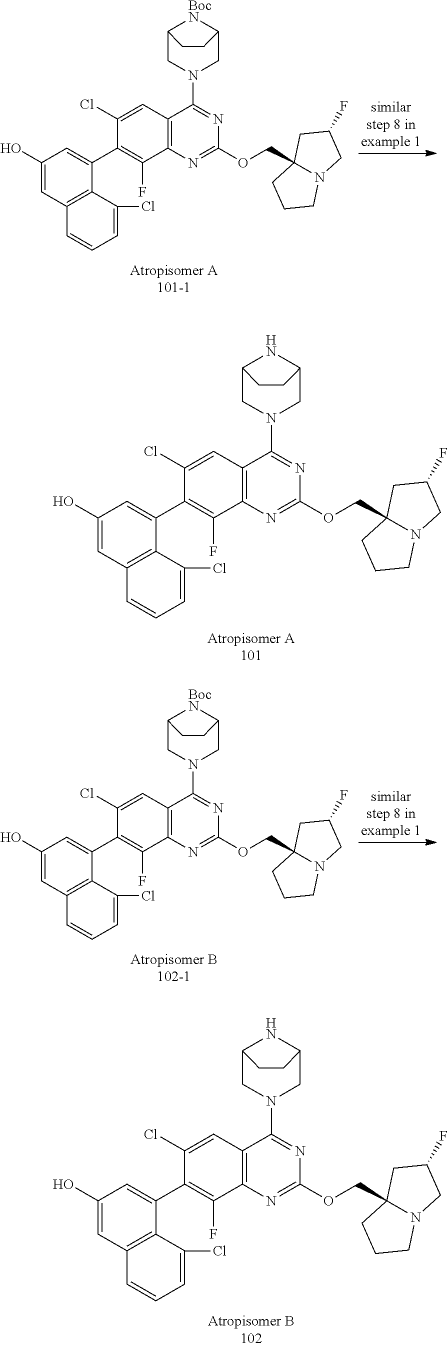 [SMILES: CC(C)(C)OC(=O)N1C2CCC1CN(c1nc(OC[C@]34CCCN3C[C@@H](F)C4)nc3c(F)c(-c4cc(O)cc5cccc(Cl)c45)c(Cl)cc13)C2 ; Oc1cc(-c2c(Cl)cc3c(N4CC5CCC(C4)N5)nc(OC[C@]45CCCN4C[C@@H](F)C5)nc3c2F)c2c(Cl)cccc2c1 ; CC(C)(C)OC(=O)N1C2CCC1CN(c1nc(OC[C@]34CCCN3C[C@@H](F)C4)nc3c(F)c(-c4cc(O)cc5cccc(Cl)c45)c(Cl)cc13)C2 ; Oc1cc(-c2c(Cl)cc3c(N4CC5CCC(C4)N5)nc(OC[C@]45CCCN4C[C@@H](F)C5)nc3c2F)c2c(Cl)cccc2c1]

Compound 101-0 was prepared from 77-10-Pt following the procedure for the synthesis of compound 2 in example 1.

Compound 101-0 (382 mg) was purified by SFC (column: Chiral-OZ, MeOH (0.1% DEA)/CO₂=60/40) to afford 101-1 (187 mg) and 102-1 (170 mg), respectively.

101-1: SFC analysis: >9900 ee; Retention time: 4.82 min; column: CHIRALCEL® OZ-H, 4000 MeOH (0.1% of DEA) in CO₂; pressure: 100 bar; flow rate: 3.0 mL/min.
102-1: SFC analysis: >9900 ee; Retention time: 6.22 min; column: CHIRALCEL® OZ-H, 400% MeOH (0.1) DEA) in CO₂ pressure: 100 bar; flow rate: 3.0 mL/min.

Compound 101 was prepared from 101-1 following the procedure for the synthesis of compound 2 in example 1 as a 3 eq of TFA salt. LCMS (ESI, m/z): [M+H]⁺=626.2; HNMR (400 MHz, methanol-d₄, ppm): δ 7.93-7.90 (m, 1H), 7.75-7.72 (m, 1H), 7.37-7.28 (m, 3H), 6.95 (d, J=2.4 Hz, 1H), 5.62-5.47 (i, 1H), 4.87-4.60 (m, 4H), 4.27-4.18 (i, 2H), 4.04-3.79 (m, 5H), 3.49-3.40 (m, 1H), 2.75-2.10 (in, 10H). FNMR (376 MHz, methanol-d₄, ppm): δ −123.79 (1F), −174.28 (F). (1F).

Compound 102 was prepared from 102-1 following the procedure for the synthesis of compound 2 in example 1 as a 3 eq of TFA salt. LCMS (ESI, m/z): [M+H]⁺=626.3; HNMR (400 MHz, methanol-d₄, ppm): δ 7.92-7.90 (m, 1H), 7.73 (dd, J=8.0, 1.2 Hz, 1H), 7.36-7.28 (m, 3H), 6.96 (d, J=2.4 Hz, 1H), 5.62-5.46 (m, 1H), 4.87-4.60 (m, 4H), 4.27-4.17 (m, 2H), 4.04-3.80 (m, 5H), 3.47-3.39 (m, 1H), 2.75-2.05 (in, 10H). FNMR (376 MHz, methanol-d₄, ppm): δ −123.74 (1F), −174.17 (1F).

### Example 14 Synthesis of Compounds 103 and 104

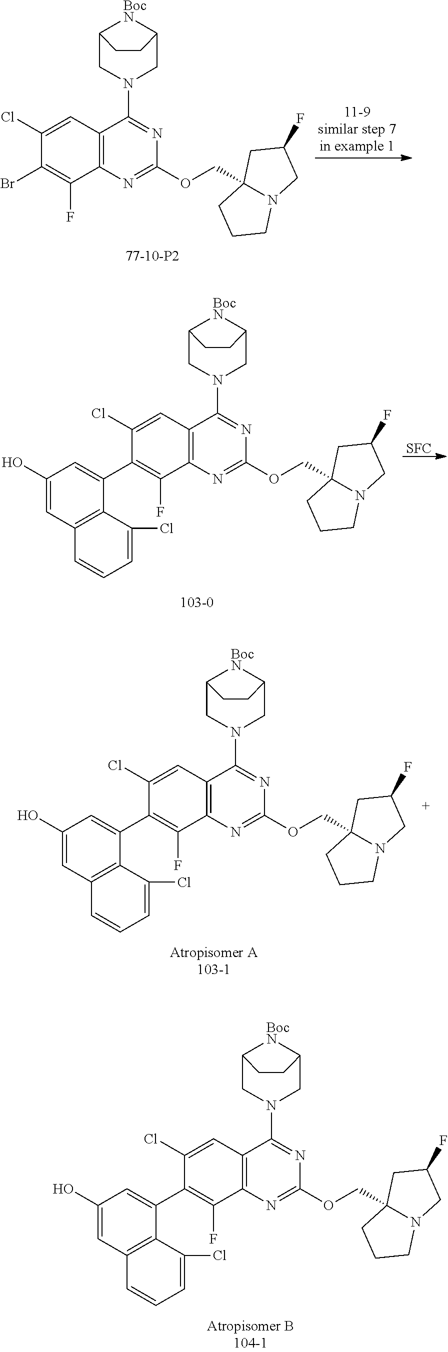

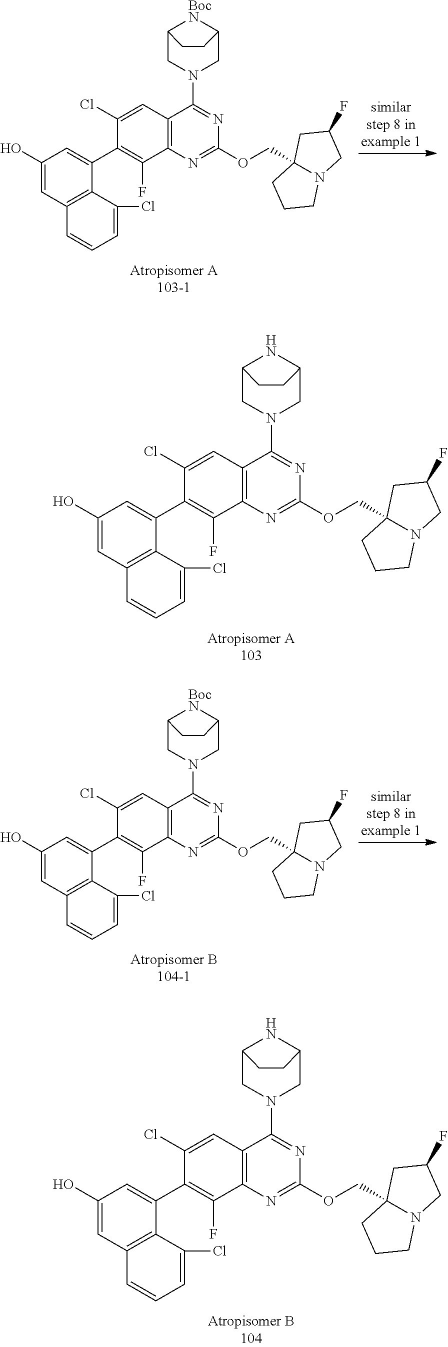 [SMILES: CC(C)(C)OC(=O)N1C2CCC1CN(c1nc(OC[C@@]34CCCN3C[C@H](F)C4)nc3c(F)c(-c4cc(O)cc5cccc(Cl)c45)c(Cl)cc13)C2 ; Oc1cc(-c2c(Cl)cc3c(N4CC5CCC(C4)N5)nc(OC[C@@]45CCCN4C[C@H](F)C5)nc3c2F)c2c(Cl)cccc2c1 ; CC(C)(C)OC(=O)N1C2CCC1CN(c1nc(OC[C@@]34CCCN3C[C@H](F)C4)nc3c(F)c(-c4cc(O)cc5cccc(Cl)c45)c(Cl)cc13)C2 ; Oc1cc(-c2c(Cl)cc3c(N4CC5CCC(C4)N5)nc(OC[C@@]45CCCN4C[C@H](F)C5)nc3c2F)c2c(Cl)cccc2c1]

Compound 103-0 was prepared from 77-10-P2 following the procedure for the synthesis of compound 2 in example 1.

Compound 103-0 was purified by SFC (column: Chiral-MIC, EtOH (0.1% of DEA)/C₂=55/45) to afford 103-1 and 104-1, respectively.

103-1: SFC analysis: >99% ee; Retention time: 1.04 min; column: Chiral-MIC, EtOH (0.7 of DEA) in CO₂, 5% to 40%; pressure: 100 bar; flow rate: 1.5 mL/min.
104-1: SFC analysis: >99% ee; Retention time: 1.62 min; column: Chiral-MIC, EtOH (0.1% of DEA) in CO₂, 5% to 40%; pressure: 100 bar; flow rate: 1.5 mL/min.

Compound 103 was prepared from 103-1 following the procedure for the synthesis of compound 2 in example 1 as a 3 eq of TFA salt. LCMS (ESI, m/z): [M+H]⁺=626.3; HNMR (400 MHz, methanol-d₄, ppm): δ 7.92-7.90 (m, 1H), 7.74 (dd, J=8.0, 1.6 Hz, 1H), 7.37-7.28 (m, 3H), 6.95 (d, J=2.8 Hz, 1H), 5.62-5.47 (m, 1H), 4.87-4.60 (m, 4H), 4.26-4.19 (m, 2H), 4.04-3.76 (m, 5H), 3.45-3.40 (m, 1H), 2.75-2.08 (m, 10H). FNMR (376 MHz, methanol-d₄, ppm): δ −123.79 (1F), −174.24 (1F).

Compound 104 was prepared from 104-1 following the procedure for the synthesis of compound 2 in example 1 as a 3 eq of TFA salt. LCMS (ESI, m/z): [M+H]⁺=626.3; HNMR (400 MHz, methanol-d₄, ppm): δ 7.93-7.90 (m, 1H), 7.74 (dd, J=8.4, 1.6 Hz, 1H), 7.36-7.29 (m, 3H), 6.95 (d, J=2.4 Hz, 1H), 5.62-5.47 (m, 1H), 4.87-4.60 (m, 4H), 4.27-4.20 (m, 2H), 4.04-3.78 (m, 5H), 3.47-3.40 (m, 1H), 2.75-2.07 (in, 10H). FNMR (376 MHz, methanol-d₄, ppm): δ −123.80 (1F), −174.29 (1F).

### Example 15 Synthesis of Compounds 45

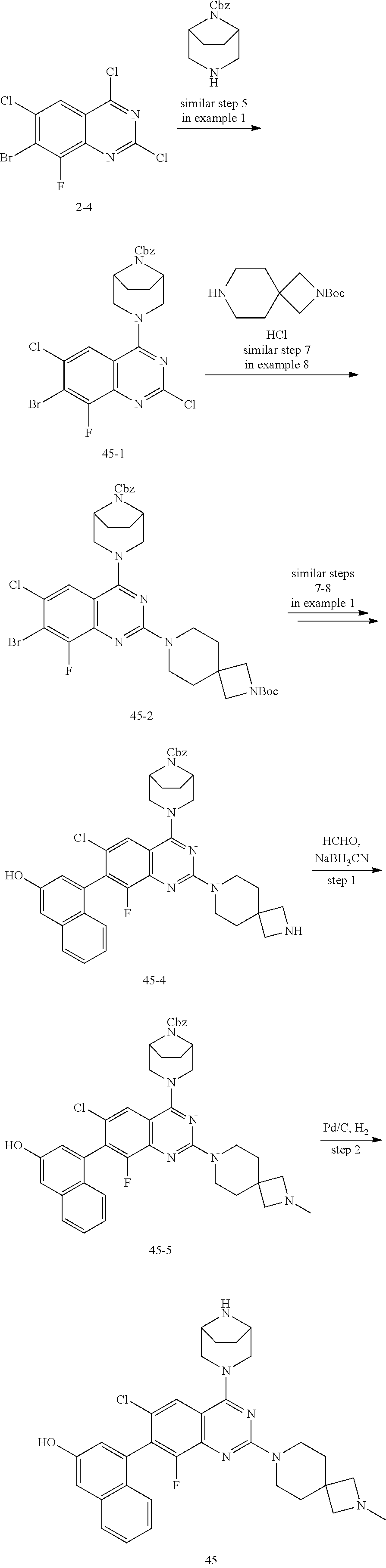

Compound 45-1 was prepared following the procedure for the synthesis of compound 2 in example 1.

Compound 45-2 was prepared following the procedure for the synthesis of compound 42 in example 8.

Compound 45-4 was prepared following the procedure for the synthesis of compound 2 in example 1.

Step 1: To a solution of 45-4 (174 mg, 0.25 mmol) in CH₃OH (3 mL) was added HCHO (37 wt % in water, 325 mg, 3.53 mmol) and cat. acetic acid at room temperature. The solution was stirred for 15 min at room temperature followed by addition of NaBH₃CN (48 mg, 0.75 mmol). The resulting mixture was stirred at room temperature for 3 h. The mixture was quenched with water and extracted with ethyl acetate. The combined organic layers were dried over anhydrous Na₂SO₄, filtered and concentrated. The residue was purified by column chromatography on silica gel (dichloromethane/methanol=10/1) to afford 45-5.

Step 2: A mixture of 45-5 (20 mg, 0.028 mmol) and 10% Pd/C (15 mg) in CH₃OH (5 mL) was stirred at room temperature for 2 h under hydrogen atmosphere. The mixture was filtered and concentrated. The residue was purified by prep-HPLC (acetonitrile with 0.05% of NH₃·H₂O in water: 5% to 95%) to afford 45. LCMS (ESI, m/z): [M+H]⁺=573.2; HNMR (400 MHz, methanol-d₄, ppm): δ 7.74 (d, J=1.2 Hz, 2H), 7.41 (t, J=1.2 Hz, 1H), 7.38-7.26 (m, 3H), 7.01 (s, 1H), 4.27-4.25 (m, 2H), 3.85-3.82 (m, 4H), 3.60-3.56 (m, 2H), 3.51-3.49 (m, 2H), 3.25-3.21 (m, 4H), 2.45 (s, 3H), 1.89-1.79 (m, 8H).

### Example 16 Synthesis of Compounds 116

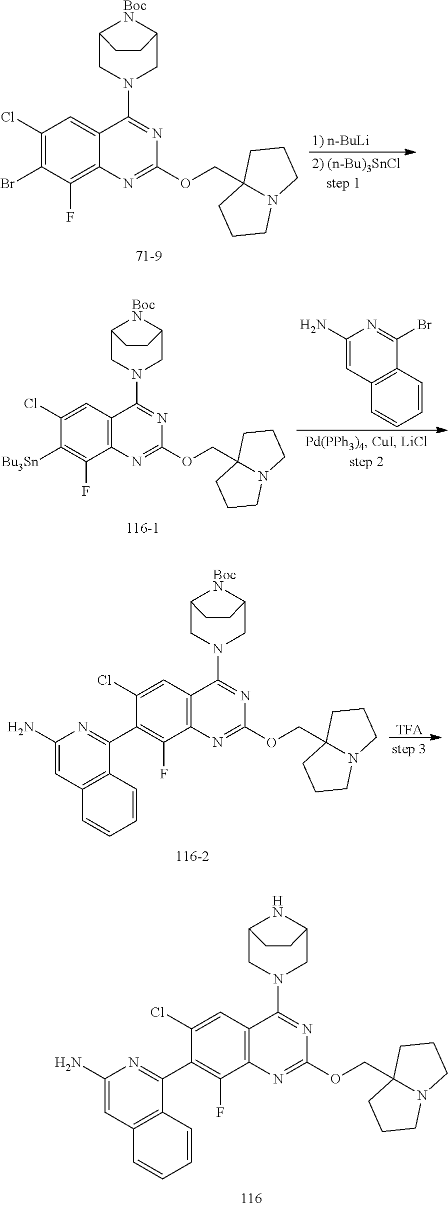

Step 1: To a solution of 71-9 (426 mg, 0.7 mmol) in tetrahydrofuran (10 mL) was added n-butyllithium (0.34 mL, 0.84 mmol) dropwise at −78° C. under N₂ atmosphere. The mixture was stirred at −78° C. for 1 h. To above mixture was added a solution of chlorotributyltin (455 mg, 1.4 mmol) in tetrahydrofuran (5 mL) dropwise. The mixture was allowed to warm to 0° C. and stirred for 1 h. The mixture was quenched with sat. ammonium chloride solution, diluted with water and extracted with ethyl acetate. The combined organic layers were dried over anhydrous Na₂SO₄, filtered and concentrated. The residue was purified by column chromatography on silica gel (dichloromethane to methanol/dichloromethane=1/10) to afford 116-1.

Step 2: A mixture of 116-1 (246 mg, 0.3 mmol), 1-bromoisoquinolin-3-amine (67 mg, 0.3 mmol), CuI (29 mg, 0.15 mmol), lithium chloride (32 mg, 0.75 mmol) and tetrakis(triphenylphosphine)palladium (173 mg, 0.15 mmol) in dimethyl formamide (5 mL) was stirred at 105° C. for 3 h under N₂ atmosphere. The mixture was cooled, diluted with water and extracted with ethyl acetate. The combined organic layers were dried over anhydrous Na₂SO₄, filtered and concentrated. The residue was purified by column chromatography on silica gel (dichloromethane to methanol/dichloromethane/ammonia=1/10/0.005) to afford 116-2.

Step 3: A solution of 116-2 (35 mg, 0.05 mmol) in trifluoroacetic acid (0.5 mL) and dichloromethane (1.5 mL) was stirred at room temperature for 1 h. The mixture was concentrated and the residue was purified by prep-HPLC (acetonitrile with 0.05% of TFA in water: 5% to 95%) to afford 116 as a 3 eq of TFA salt. LCMS (ESI, m/z): [M+H]⁺=574.3; HNMR (400 MHz, methanol-d₄, ppm): δ 8.02-8.01 (m, 1H), 7.70-7.67 (m, 1H), 7.58-7.53 (m, 1H), 7.32-7.29 (m, 1H), 7.20-7.15 (m, 1H), 7.06 (s, 1H), 4.78-4.62 (m, 4H), 4.24 (s, 2H), 3.95-3.88 (m, 2H), 3.71-3.64 (m, 2H), 3.29-3.23 (m, 2H), 2.35-2.05 (m, 12H). FNMR (376 MHz, methanol-d₄, ppm): δ −124.78 (1F).

### Example 17 Synthesis of Compounds 30

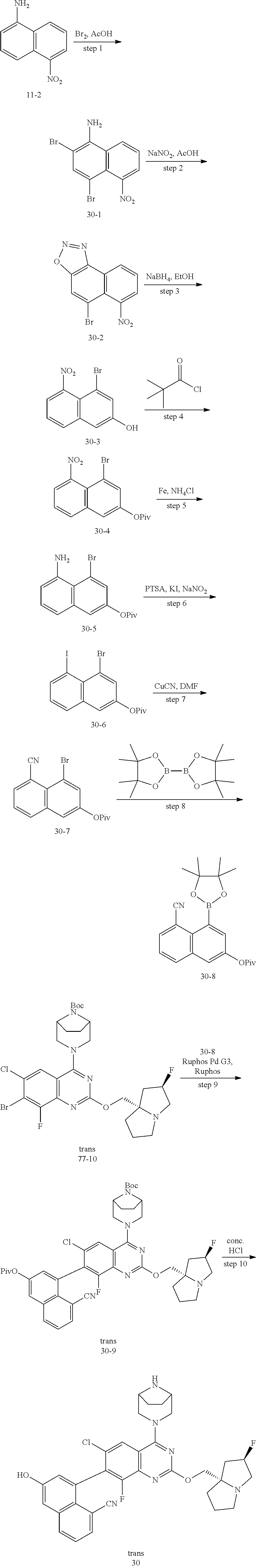

Step 1: To a mixture of 11-2 (80 g, 425 mmol) in acetic acid (2.5 L) was added Br₂ (150 g, 851 mmol) dropwise at room temperature. The mixture was stirred at 70° C. for 2 h, cooled and filtered. The filter cake was suspended in 20% NaOH. The mixture was stirred at room temperature for 20 min and filtered. The solid was slurried with ethanol, filtered and the filter cake was dried to afford 30-1.

Step 2: To a mixture of 30-1 (54 g, 157 mmol) in acetic acid (600 mL) and propionic acid (150 mL) was added sodium nitrite (13 g, 188 mmol) in portions at 5° C. The mixture was stirred for 0.5 h at 5° C. Then the mixture was poured into water and filtered. The filter cake (30-2) was used directly without purification.

Step 3: To a mixture of 30-2 (20 g, crude, ca. 73 mmol) in ethanol (250 mL) was added sodium borohydride (5.5 g, 146 mmol) at 5° C. The mixture was stirred for 0.5 h at 5° C. and then quenched with water (20 mL). The mixture was adjusted to pH=5 with 1N hydrochloric acid. The organic solvent was removed in vacuo. The aqueous layer was extracted with ethyl acetate. The combined organic layers were dried over anhydrous Na₂SO₄, filtered and concentrated. The residue was purified by column chromatography on silica gel (petroleum ether to petroleum ether/ethyl acetate=4/1) to afford 30-3.

Step 4: To a solution of 30-3 (10.7 g, 40 mmol) and triethylamine (6.06 g, 60 mmol) in dichloromethane (100 mL) was added pivaloyl chloride (5.76 g, 48 mmol) dropwise at 0° C. The mixture was stirred at room temperature for 1 h. The mixture was washed with water and brine. The organic layer was dried over anhydrous Na₂SO₄, filtered and concentrated to afford 30-4 which was used directly without purification.

Step 5: A mixture of 30-4 (8.1 g, 23 mmol), iron powder (6.5 g, 115 mmol) and ammonium chloride (12.2 g, 230 mmol) in ethanol (40 mL) and water (10 mL) was stirred at 80° C. for 10 min under N₂ atmosphere. The mixture was cooled, diluted with water and extracted with ethyl acetate. The combined organic layers were washed with brine, dried over sodium sulfate, filtered and concentrated. The residue was purified by silica gel column chromatograph (petroleum ether to petroleum ether/ethyl acetate=4/1) to afford 30-5.

Step 6: A mixture of 30-5 (5.06 g, 15.76 mmol) and p-toluenesulfonic acid (8.13 g, 47.29 mmol) in acetonitrile (126 mL) was stirred at room temperature for 30 min. To above mixture was added a solution of sodium nitrite (2.17 g, 31.52 mmol) and potassium iodide (5.23 g, 31.52 mmol) in water (19 mL) at 0° C. over 30 min. The resulting mixture was allowed to warm to 30° C. and stirred for 2 h. The mixture was diluted with dichloromethane and washed with water, saturated sodium bicarbonate solution and brine successively. The organic layer was dried over sodium sulfate, filtered and concentrated. The residue was purified by silica gel column chromatograph (petroleum ether to petroleum ether/ethyl acetate=10/1) to afford 30-6.

Step 7: A mixture of 30-6 (3.4 g, 7.87 mmol) and copper (I) cyanide (744 mg, 8.26 mmol) in N,N-dimethylformamide (34 mL) was stirred at 80° C. for 0.5 h under N₂ atmosphere. The mixture was cooled, diluted with water and extracted with ethyl acetate. The combined organic layers were dried over sodium sulfate, filtered and concentrated. The residue was triturated with n-hexane to afford 30-7 which was used directly without purification.

Step 8: A mixture of 30-7 (1.16 g, 3.5 mmol), bis(pinacolato)diboron (1.33 g, 5.25 mmol), potassium acetate (1.05 g, 10.5 mmol) and [1,1′-bis(diphenylphosphino)-ferrocene]dichloropalladium(II) (205 mg, 0.28 mmol) in 1,4-dioxane (20 mL) was stirred at 95° C. for 6 h under N₂ atmosphere. The mixture was cooled, diluted with water and extracted with ethyl acetate. The combined organic layers were dried over sodium sulfate, filtered and concentrated. The residue was purified by silica gel column chromatograph (petroleum ether to petroleum ether/ethyl acetate=10/1) to afford 30-8.

Step 9: A mixture of 77-10 (50 mg, 0.08 mmol), 30-8 (90 mg, 0.24 mmol), sodium carbonate (25 mg, 0.24 mmol), 2-dicyclohexylphosphino-2′,6′-diisopropoxybiphenyl (3.6 mg, 0.008 mmol) and methanesulfonato(2-dicyclohexylphosphino-2′,6′-diisopropoxybiphenyl)(2′-amino-1,1′-biphenyl-2-yl)palladium(II) (4.3 mg, 0.008 mmol) in 1,4-dioxane/water (5/1, 4.8 mL) was stirred at 80° C. for 1 h under N₂ atmosphere. The mixture was cooled, diluted with water and extracted with ethyl acetate. The combined organic layers were dried over Na₂SO₄, filtered and concentrated. The residue was purified by prep-HPLC (acetonitrile with 0.05% of TFA in water: 20% to 95%) to afford 30-9.

Step 10: To a solution of 30-9 (10 mg, 0.013 mmol) in ethanol (0.5 mL) was added water (0.25 mL) and concentrated hydrochloric acid (0.25 mL). The mixture was stirred at 70° C. for 5 h under N₂ atmosphere. The mixture was purified by prep-HPLC (acetonitrile with 0.05% of TFA in water: 5% to 95%) to afford 30 as a 3 eq of TFA salt. LCMS (ESI, m/z): [M+H]⁺=617.3; HNMR (400 MHz, methanol-d₄, ppm): δ 8.14-8.11 (m, 1H), 7.97 (s, 1H), 7.76 (d, J=7.2 Hz, 1H), 7.54 (t, J=8.0 Hz, 1H), 7.42 (d, J=2.4 Hz, 1H), 7.16 (d, J=2.8 Hz, 1H), 5.61-5.48 (m, 1H), 4.80-4.60 (m, 4H), 4.26-4.20 (m, 2H), 4.00-3.86 (m, 5H), 3.49-3.42 (m, 1H), 2.75-2.55 (m, 2H), 2.46-2.26 (m, 3H), 2.20-1.97 (m, 5H).

### Example 18 Synthesis of Compound 25

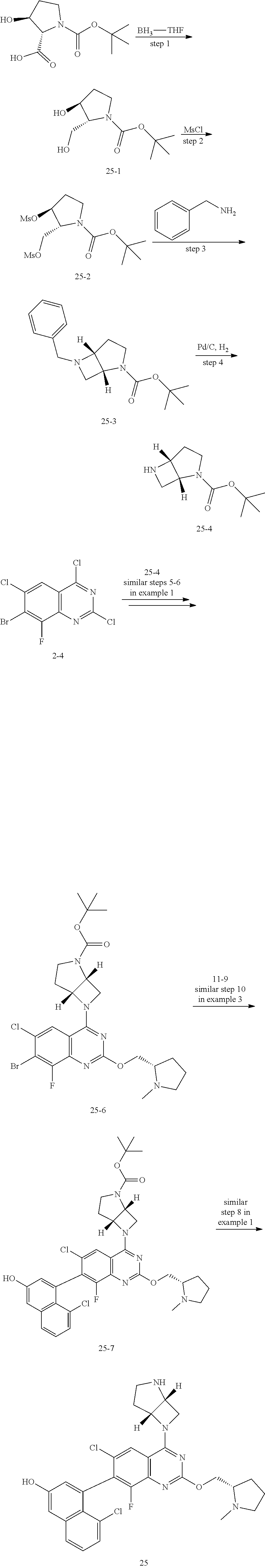

Step 1: To a solution of 1-tert-butoxycarbonyl-3-hydroxy-pyrrolidine-2-carboxylic acid (2 g, 8.65 mmol) in THF (20 mL) was added borane-tetrahydrofuran complex (1 M in THF, 19.03 mL, 19.03 mmol) at 0° C. The resulting solution was stirred at 65° C. for 2 h. The mixture was cooled, quenched with methanol and concentrated. The residue was partitioned between ethyl acetate and aqueous NaHCO₃. The organic layer was separated, dried over Na₂SO₄, filtered and concentrated to afford 25-1 which was used directly in the next step without purification.

Step 2: To a solution of 25-1 (1.69 g, 7.8 mmol) in dichloromethane (20 mL) was added TEA (3.31 g, 32 mmol) and methanesulfonyl chloride (2.67 g, 23.3 mmol) at 0° C. The resulting solution was stirred at room temperature for 3 h. The mixture was quenched with water and extracted with ethyl acetate. The combined organic layers were dried over sodium sulfate, filtered and concentrated. The residue was purified by silica gel column chromatograph (petroleum ether/ethyl acetate=2/1) to afford 25-2.

Step 3: To a solution of 25-2 (2.39 g, 6.4 mmol) in toluene (50 mL) was added phenylmethanamine (2.06 g, 19.2 mmol) at room temperature. The resulting solution was stirred at 110° C. for 15 h. The solution was concentrated. The residue was purified by silica gel column chromatograph (petroleum ether/ethyl acetate=1/2) to afford 25-3.

Step 4: A solution of 25-3 (1.1 g, 3.8 mmol) and 10% Pd/C (0.5 g) in THF (15 mL) was stirred at 50° C. for 8 h under 4 atm of H₂. The mixture was filtered and the filtrate was concentrated to afford 25-4 which was used directly in the next step without purification.

Compound 25-6 was prepared following the procedure for the synthesis of compound 2 in example 1.

Compound 25-7 was prepared following the procedure for the synthesis of compound 11 in example 3.

Compound 25 was prepared following the procedure for the synthesis of compound 2 in example 1 as a 3 eq of TFA salt. LCMS (ESI, m/z): [M+H]⁺=568.1; HNMR (400 MHz, methanol-d₄, ppm): δ 7.83 (s, 1H), 7.73 (d, J=1.2 Hz, 1H), 7.35-7.29 (m, 3H), 6.96 (s, 1H), 5.49-5.47 (m, 1H), 4.49-4.32 (m, 4H), 3.37-3.36 (m, 2H), 3.30-3.26 (m, 2H), 2.98-2.96 (m, 1H), 2.51 (s, 3H), 2.49-2.46 (m, 2H), 2.20-2.16 (m, 1H), 1.87-1.70 (m, 4H).

### Example 19 Synthesis of Compounds 119 and 120

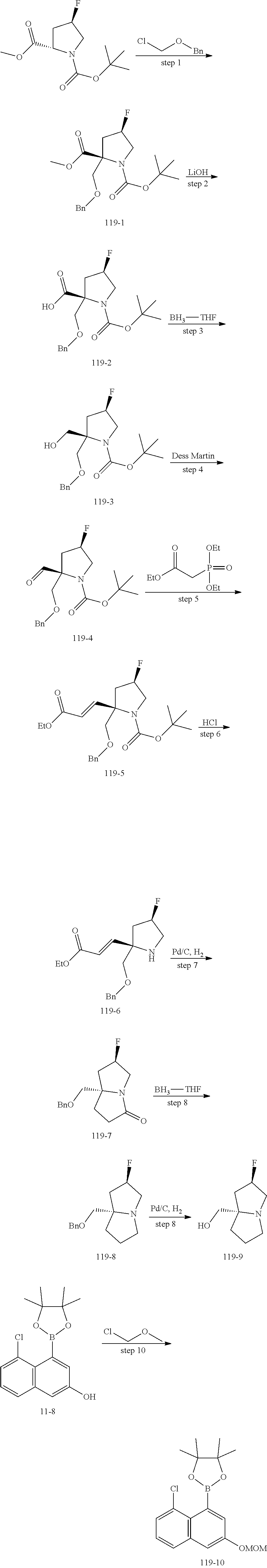

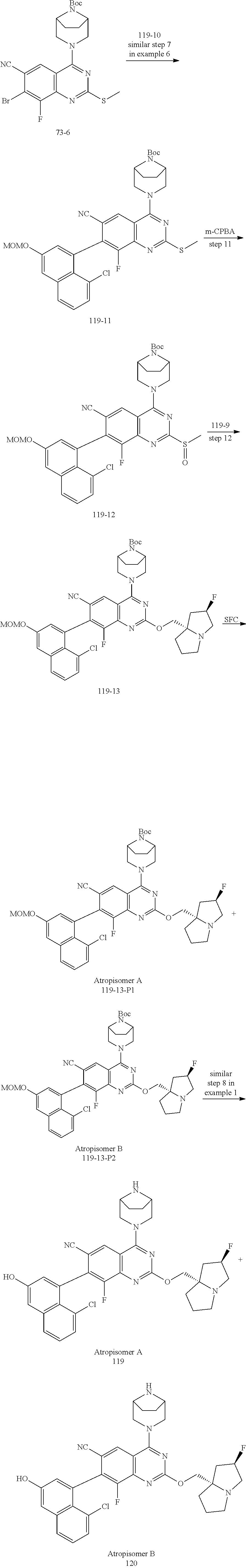

Step 1: To a solution of 1-(tert-butyl) 2-methyl (2S,4R)-4-fluoropyrrolidine-1,2-dicarboxylate (247 g, 1 mol) in tetrahydrofuran (2 L) was added dropwise lithium bis(trimethylsilyl)amide (1.2 L, 1.2 mol, 1.0 M in tetrahydrofuran) at −70° C. under nitrogen atmosphere. The mixture was stirred at −70° C. for 1 h. Then a solution of ((chloromethoxy)methyl)benzene (172 g, 1.1 mol) in tetrahydrofuran (300 mL) was added dropwise at −70° C. The mixture was stirred at −30° C. for 5 h, quenched with sat. aqueous ammonium chloride and extracted with ethyl acetate. The combined organic layers were washed with brine, dried over sodium sulfate, filtered and concentrated to afford 119-1 which was used in the next step directly without purification.

Step 2: To a solution of 119-1 (367 g, 1 mol) in tetrahydrofuran (2 L) and water (600 mL) was added lithium hydroxide monohydrate (114 g, 3 mol) at room temperature. The mixture was stirred at 60° C. overnight. The mixture was concentrated, diluted with water and tert-butyl methyl ether. After being stirred for 30 min, the aqueous phase was separated, adjusted to around pH 3 with 1 N HCl and extracted with ethyl acetate. The combined organic layers were washed with brine, dried over sodium sulfate, filtered and concentrated to afford 119-2 which was used in the next step directly without purification.

Step 3: To a solution of 119-2 (320 g, 906 mmol) in tetrahydrofuran (2.5 L) was added borane tetrahydrofuran complex solution (1.36 L, 1.36 mol, 1.0 M in tetrahydrofuran) dropwise at 0° C. under nitrogen atmosphere. The mixture was stirred at room temperature for 4 h, quenched with methanol (500 mL) and stirred at reflux for 3 h. Then the mixture was diluted with water and extracted with ethyl acetate. The combined organic layers were washed with brine, dried over sodium sulfate, filtered and concentrated to afford 119-3 which was used in the next step directly without purification.

Step 4: To a solution of 119-3 (285 g, 840 mmol) in dichloromethane (3.5 L) was added Dess Martin periodinane (445 g, 1.05 mol) at 0° C. The mixture was stirred at room temperature overnight, quenched with sat. aqueous sodium hyposulfite solution and stirred at room temperature for 3 h. The mixture was filtered and the aqueous layer was extracted with dichloromethane. The combined organic layers were washed with sat. aqueous sodium bicarbonate aqueous, brine, dried over sodium sulfate, filtered and concentrated to afford 119-4 which was used in the next step directly without purification.

Step 5: To a solution of ethyl 2-(diethoxyphosphoryl)acetate (211 g, 944 mmol) in tetrahydrofuran (1.5 L) was added dropwise lithium bis(trimethylsilyl)amide (944 mL, 944 mmol, 1.0 M in tetrahydrofuran) at −40° C. under nitrogen atmosphere. The mixture was stirred at −40° C. for 1 h. Then a solution of 119-4 (265 g, 786 mmol) in tetrahydrofuran (500 mL) was added dropwise to the reaction mixture at −40° C. The resulting mixture was stirred at room temperature for 3 h, quenched with sat. aqueous ammonium chloride and extracted with ethyl acetate. The combined organic layers were washed with brine, dried over sodium sulfate, filtered and concentrated to afford 119-5 which was used in the next step without purification.

Step 6: To a solution of 119-5 (320 g, 786 mmol) in ethyl acetate (500 mL) was added hydrochloric acid (800 mL, 2.8 mol, 3.5M in ethyl acetate) at room temperature. After being stirred at room temperature for 3 h, the mixture was concentrated, diluted with water and tert-butyl methyl ether. The mixture was stirred at room temperature for 30 min. The aqueous phase was separated, adjusted to around pH 10 with sat. aqueous sodium carbonate solution and extracted with ethyl acetate. The combined organic layers were washed with brine, dried over sodium sulfate, filtered and concentrated to afford 119-6 which was used in the next step without purification.

Step 7: A mixture of 119-6 (225 g, 733 mmol) and 10% Pd/C (11 g) in ethyl acetate (1.2 L) was stirred at room temperature overnight under hydrogen atmosphere, then heated to reflux and stirred overnight. The mixture was filtered and the filtrate was concentrated. The residue was purified by column chromatography on silica gel (petroleum ether to petroleum ether/ethyl acetate=1/4) to afford 119-7.

Step 8: To a solution of 119-7 (130 g, 494 mmol) in tetrahydrofuran (1.5 L) was added borane tetrahydrofuran complex solution (740 mL, 740 mmol, 1.0 M in tetrahydrofuran) dropwise at 0° C. under nitrogen atmosphere. Then the mixture was stirred at room temperature for 4 h, quenched with methanol and stirred at reflux for 3 h. The mixture was cooled, diluted with water and extracted with ethyl acetate. The combined organic layers were washed with brine, dried over sodium sulfate, filtered and concentrated to afford 119-8 which was used in the next step without purification.

Step 9: A mixture of 119-8 (2.5 g, 10 mmol) and 10% Pd/C (200 mg) in methanol (30 mL) was stirred at 45° C. overnight under hydrogen atmosphere. The mixture was filtered and the filtrate was concentrated. The residue was purified by column chromatography on silica gel (dichloromethane to dichloromethane/methanol=10/1) to afford 119-9.

Step 10: A mixture of 11-8 (500 mg, 1.64 mmol), N,N-diisopropylethylamine (636 mg, 4.92 mmol) and chloro(methoxy)methane (265 mg, 3.28 mmol) in dichloromethane (5 mL) was stirred at room temperature for 2 h. The mixture was diluted with dichloromethane (100 mL), washed with water and brine. The organic layer was dried over sodium sulfate, filtered and concentrated. The residue was purified by column chromatography on silica gel (petroleum ether to petroleum ether/ethyl acetate=9/1) to afford 119-10.

Compound 119-11 was prepared from compound 73-6 following the procedure for the synthesis of compound 73-7 in example 6.

Step 11: To a solution of 119-11 (910 mg, 1.4 mmol) in dichloromethane (20 mL) was added 3-chloroperoxybenzoic acid (314 mg, 1.82 mmol) in portions at −5° C. The mixture was stirred at −5° C. for 0.5 hour, diluted with dichloromethane (50 mL), washed with sat. aqueous sodium bicarbonate solution and brine, dried over sodium sulfate, filtered and concentrated to afford 119-12 which was used directly in the next step without purification.

Step 12: To a solution of 119-9 (325 mg, 2.04 mmol) in tetrahydrofuran (20 mL) was added lithium bis(trimethylsilyl)amide (1.8 mL, 1.0 M in tetrahydrofuran, 1.8 mmol) at −5° C., then stirred for 5 min. A solution of 119-12 (909 mg, 1.36 mmol) in tetrahydrofuran (5 mL) was added to above mixture dropwise at −5° C. The mixture was stirred at −5° C. for 5 min. The mixture was quenched with aqueous ammonium chloride and extracted with ethyl acetate. The combined organic layers were washed with brine, dried over sodium sulfate, filtered and concentrated. The residue was purified by prep-HPLC (acetonitrile with 0.05% of TFA in water: 15% to 95%) to afford 119-13.

119-13 (421 mg) was purified by SFC (column: REGIS (S,S)WHELK-O1, EtOH/CO₂=55/45) to afford 119-13-P1 (179 mg) and 119-13-P2 (200 mg), respectively. 119-13-P1: SFC analysis: 99.5% ee. Retention time 6.05 min; column: REGIS (S,S)WHELK-O1, IPA (0.1% of DEA) in CO₂; pressure: 100 bar; flow rate: 1.5 mL/min. 119-13-P2: SFC analysis: 98.3% ee. Retention time 7.87 min; column: REGIS (S,S)WHELK-O1, IPA (0.1% of DEA) in CO₂; pressure: 100 bar; flow rate: 1.5 mL/min.

Compound 119 was prepared from compound 119-13-P1 following the procedure for the synthesis of compound 2 in example 1 as a 2 eq of TFA salt. LCMS (ESI, m/z): [M+H]⁺=617.3; HNMR (400 MHz, methanol-d₄, ppm): δ 8.31 (s, 1H), 7.77 (dd, J=8.0, 1.2 Hz, 1H), 7.40-7.32 (m, 3H), 7.07 (d, J=2.4 Hz, 1H), 5.63-5.48 (m, 1H), 4.83-4.80 (m, 1H), 4.73-4.64 (m, 3H), 4.27-4.20 (m, 2H), 4.05-3.80 (m, 5H), 3.50-3.40 (m, 1H), 2.77-2.00 (m, 10H). FNMR (376 MHz, methanol-d₄, ppm): δ −125.07 (1F), −174.24 (1F).

Compound 120 was prepared from compound 119-13-P2 following the procedure for the synthesis of compound 2 in example 1 as a 2 eq of TFA salt. LCMS (ESI, m/z): [M+H]⁺=617.3; HNMR (400 MHz, methanol-d₄, ppm): δ 8.31 (s, 1H), 7.77 (dd, J=8.0, 1.6 Hz, 1H), 7.41-7.32 (m, 3H), 7.07 (d, J=2.4 Hz, 1H), 5.63-5.48 (m, 1H), 4.83-4.78 (m, 1H), 4.76-4.64 (m, 3H), 4.27-4.20 (m, 2H), 4.05-3.81 (m, 5H), 3.50-3.39 (m, 1H), 2.77-2.01 (m, 10H). FNMR (376 MHz, methanol-d₄, ppm): δ −125.09 (1F), −174.22 (1F).

### Example 20 Synthesis of Compound 50

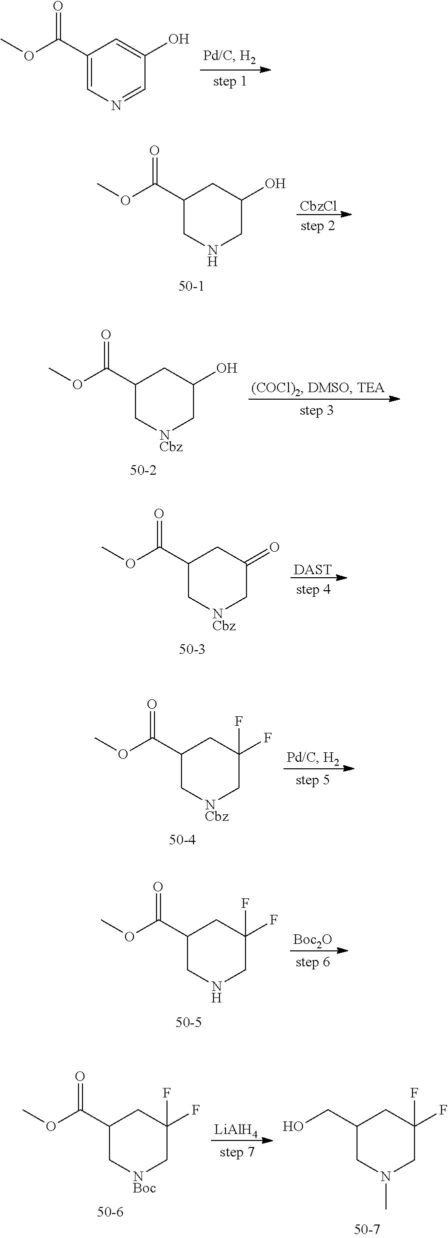

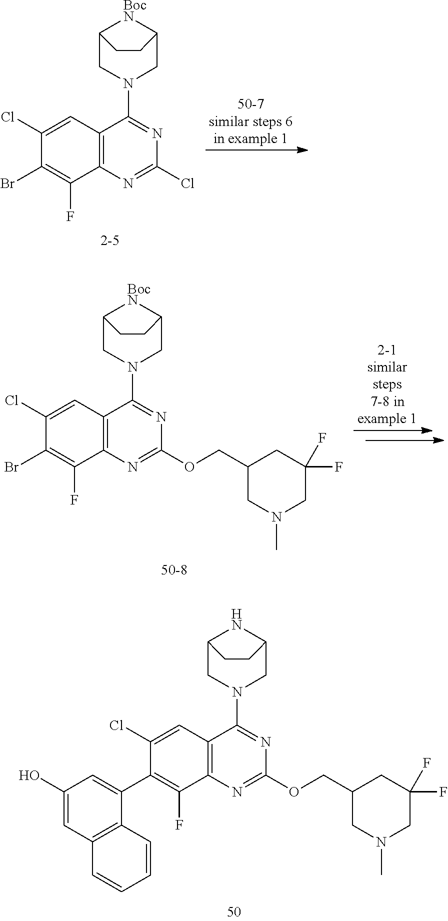

Step 1: To a solution of methyl 5-hydroxypyridine-3-carboxylate (100 g, 653 mmol) in AcOH (1 L) was added Pd/C (10%, 20 g). The reaction mixture was stirred at 70° C. for 72 h under 50 psi H₂. The reaction mixture was filtered with Celite and the filtrate was concentrated to afford 50-1 which was used directly in the next step without purification.

Step 2: To a solution of 50-1 (104 g, 653 mmol) in dichloromethane (1 L) was added N-ethyl-N-isopropyl-propan-2-amine (253 g, 1.96 mol) and benzyl chloroformate (167 g, 1.3 mol). The mixture was stirred at room temperature overnight. The mixture was diluted with water and extracted with dichloromethane. The combined organic layers were washed with brine, dried over anhydrous sodium sulfate, filtered and concentrated. The residue was purified by silica gel column chromatography (petroleum ether/ethyl acetate=5/1 to 1/1) to afford 50-2.

Step 3: To a solution of oxalyl dichloride (10.8 g, 85.2 mmol) in DCM (50 mL) was added DMSO (13.3 g, 170.5 mmol, 12.1 mL) dropwise at −78° C. The mixture was stirred at −78° C. for 0.5 h. 50-2 (5 g, 17.1 mmol) in dichloromethane (20 mL) was added to the mixture at −78° C. and the resulting mixture was stirred at −78° C. for 2 h. Then TEA (25.9 g, 255.7 mmol, 35.7 mL) was added, and the mixture was stirred at −78° C. for another 0.5 h. The mixture was allowed to warm to room temperature and stirred overnight. The mixture was diluted with water and extracted with dichloromethane. The combined organic layers were washed with brine, dried over anhydrous sodium sulfate, filtered and concentrated. The residue was purified by silica gel column chromatography (petroleum ether/ethyl acetate=4/1 to 2/1) to afford 50-3.

Step 4: To a solution of 50-3 (2.9 g, 9.96 mmol) in dichloromethane (30 mL) was added N-ethyl-N-(trifluoro-sulfanyl)ethanamine (4.81 g, 29.9 mmol) at 0° C. The reaction mixture was stirred at room temperature overnight. The reaction mixture was poured into ice water and extracted with dichloromethane. The combined organic layers were washed with brine, dried over anhydrous sodium sulfate, filtered and concentrated. The residue was purified by silica gel column chromatography (petroleum ether/ethyl acetate=5/1 to 3/1) to afford 50-4.

Step 5: To a solution of 50-4 (1.7 g, 5.4 mmol) in MeOH (20 mL) was added Pd/C (10%, 340 mg) and Pd(OH)₂ (20%, 170 mg). The mixture was stirred at room temperature overnight under H₂. The reaction mixture was filtered and concentrated to afford 50-5 which was used directly in the next step without purification.

Step 6: A mixture of 50-5 (0.9 g, 5.0 mmol), TEA (1.52 g, 15.1 mmol) and Boc₂O (1.6 g, 7.5 mmol) in dichloromethane (10 mL) was stirred at room temperature overnight. The reaction mixture was diluted with water and extracted with dichloromethane. The combined organic layers were washed with brine, dried over anhydrous sodium sulfate, filtered and concentrated. The residue was purified by silica gel column chromatography (petroleum ether/ethyl acetate=5/1 to 2/1) to afford 50-6.

Step 7: To a solution of 50-6 (1 g, 3.6 mmol) in THF (10 mL) was added LiAlH₄ (679 mg, 17.9 mmol). The reaction mixture was stirred at 70° C. for 2 h. The reaction mixture was quenched with water, filtered and concentrated. The residue was purified by silica gel column chromatography (petroleum ether/ethyl acetate=1/2 to ethyl acetate) to afford 50-7.

Compound 50-8 was prepared from compound 50-7 and compound 2-5 following the procedure for the synthesis of compound 2-6 in example 1.

Compound 50 was prepared from compound 50-8 following the procedure for the synthesis of compound 2 in example 1. LCMS (ESI, m/z): [M+H]⁺=598.2; HNMR (400 MHz, methanol-d₄, ppm): δ 7.95 (s, 1H), 7.74 (d, J=8.3 Hz, 1H), 7.40 (t, J=7.3 Hz, 1H), 7.28-7.15 (m, 3H), 7.02 (d, J=2.4 Hz, 1H), 4.57-4.30 (m, 4H), 3.69-3.58 (m, 4H), 3.05-2.94 (m, 2H), 2.50-2.18 (m, 6H), 2.15-2.10 (m, 1H), 1.93-1.63 (m, 5H).

### Example 21 Synthesis of Compound 125

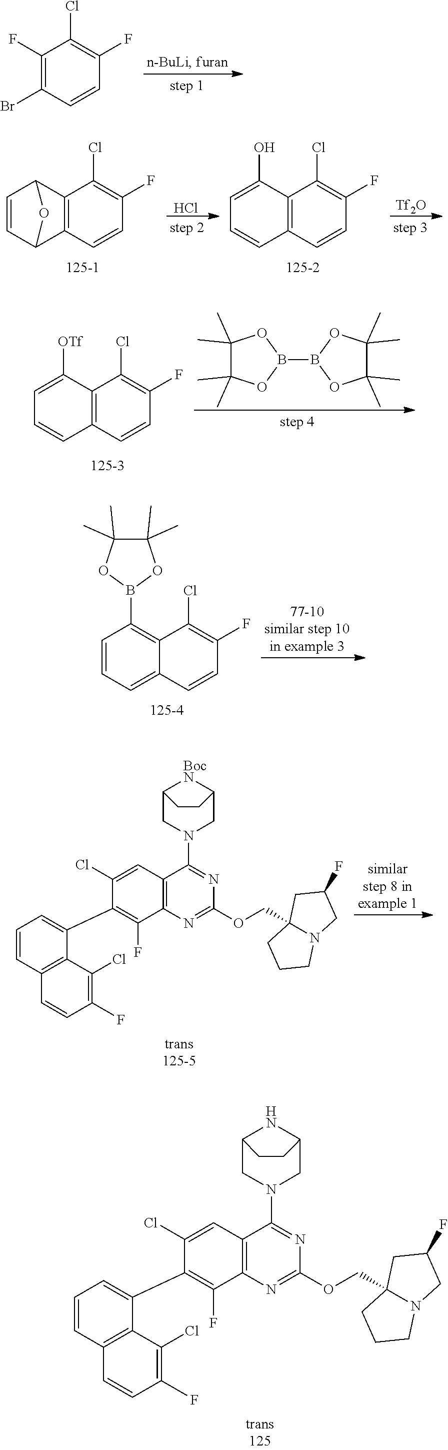

Step 1: To a mixture of 1-bromo-3-chloro-2,4-difluorobenzene (11.35 g, 50 mmol) and furan (6.8 g, 100 mmol) in toluene (200 mL) was added n-butyllithium (38 mL, 60 mmol, 1.6 M in hexane) dropwise at −15° C. over 0.5 h under nitrogen atmosphere. The mixture was warmed to room temperature and stirred for 16 h. The reaction mixture was quenched with water and filtered. The aqueous layer was extracted with ethyl acetate. The combined organic layers were washed with brine, dried over Na₂SO₄, filtered and concentrated. The residue was purified by reverse phase HPLC (acetonitrile with 0.1% of FA in water: 10% to 95%) to afford 125-1.

Step 2: A solution of 125-1 (3.5 g, 17.8 mmol) in conc. HCl (500 mL) and ethanol (40 mL) was stirred at 80° C. for 2 h. The mixture was concentrated and purified by column chromatography on silica gel (petroleum ether to petroleum ether/ethyl acetate=50/1) to afford 125-2.

Step 3: A mixture of 125-2 (1.2 g, 6.1 mmol), N,N-diisopropylethylamine (3.93 g, 30.5 mmol) and 4 Å molecular sieves (1.2 g) in dichloromethane (25 mL) was stirred for 10 min at room temperature under nitrogen atmosphere. Then trifluoroacetic anhydride (2.1 g, 7.3 mmol) was added at −40° C. and the mixture was stirred at −40° C. for 10 min. The reaction mixture was quenched with water and filtered. The aqueous layer was extracted with dichloromethane. The combined organic layers were washed with brine, dried over sodium sulfate, filtered and concentrated. The residue was purified by column chromatography on silica gel (petroleum ether to petroleum ether/ethyl acetate=50/1) to afford 125-3.

Step 4: A mixture of 125-3 (1.9 g, 5.8 mmol), bis(pinacolato)diboron (2.2 g, 8.7 mmol), potassium acetate (2.26 g, 23 mmol) and [1,1′-bis(diphenylphosphino)ferrocene]dichloropalladium (II) (844 mg, 1.15 mmol) in dimethyl sulfoxide (40 mL) was stirred at 80° C. for 2 h. Then the mixture was filtered, diluted with water and extracted with ethyl acetate. The combined organic layers were washed with brine, dried over sodium sulfate, filtered and concentrated. The residue was purified by reverse phase HPLC (acetonitrile with 0.05% of TFA in water: 10% to 95%) to afford 125-4.

Compound 125-5 was prepared following the procedure for the synthesis of compound 11 in example 3.

Compound 125 was prepared following the procedure for the synthesis of compound 2 in example 1 as a 3 eq of TFA salt. LCMS (ESI, m/z): [M+H]⁺=628.2; HNMR (400 MHz, methanol-d₄, ppm): δ 8.17-8.11 (m, 1H), 8.07 (dd, J=9.2, 5.6 Hz, 1H), 7.94 (d, J=1.6 Hz, 1H), 7.68-7.63 (m, 1H), 7.51 (t, J=8.8 Hz, 1H), 7.45 (d, J=7.2 Hz, 1H), 5.67-5.44 (m, 1H), 4.79-4.60 (m, 4H), 4.28-4.19 (m, 2H), 4.04-3.79 (m, 5H), 3.49-3.40 (m, 1H), 2.76-2.51 (m, 2H), 2.45-2.06 (m, 8H). FNMR (376 MHz, methanol-d₄, ppm): δ −111.22 (1F), −123.64 (1F).

### Example 22 Synthesis of Compound 112

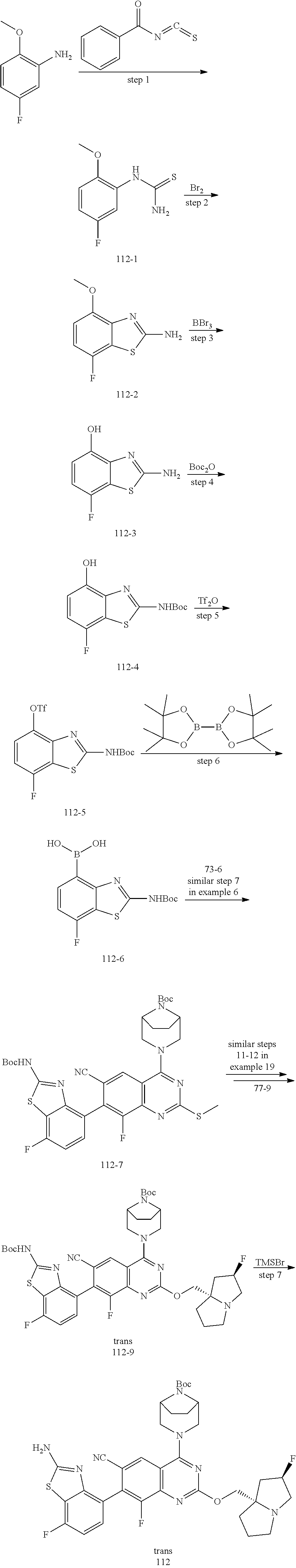

Step 1: To a solution of benzoyl isothiocyanate (36.4 g, 223.2 mmol) in anhydrous THF (150 mL) was added a solution of 5-fluoro-2-methoxy-aniline (30.0 g, 212.5 mmol) in anhydrous THF (150 mL) at 0° C. under nitrogen atmosphere. After addition, the mixture was allowed to warm to room temperature and stirred for 3 h. Then NaOH (1 M, 216.8 mL) solution was added and the resulting mixture was stirred at 80° C. overnight. The mixture was concentrated and filtered. The filter cake was washed with cold hexane to afford 112-1 which was used directly in the next step without purification.

Step 2: To a solution of 112-1 (43.0 g, 214.7 mmol) in CHCl₃ (900 mL) was added Br₂ (35.0 g, 219.1 mmol) dropwise at 0° C. After being stirred at 0° C. for 0.5 h, the mixture was heated at reflux for 2 h. Then the mixture was cooled, filtered and the filter cake was washed with cold hexane to afford 112-2 which was used directly in the next step without purification.

Step 3: To a solution of 112-2 (20.0 g, 100.9 mmol) in dichloromethane was added BBr₃ (1 M in dichloromethane, 312.8 mL) dropwise at 0° C. The mixture was warmed to room temperature and stirred overnight. The reaction was quenched with methanol at 0° C. Then the mixture was filtered and the filter cake was washed with cold dichloromethane to afford 112-3 which was used directly in the next step without purification.

Step 4: To a mixture of 112-3 (16.8 g, 91.2 mmol), Et₃N (19.4 g, 191.5 mmol) and DMAP (557.2 mg, 4.6 mmol) in dichloromethane (280 mL) was added Boc₂O (45.8 g, 209.8 mmol) at room temperature. The mixture was stirred at room temperature overnight. The mixture was diluted with water and extracted with ethyl acetate. The organic layer was concentrated and re-dissolved in methanol (180 mL). MONa (5.4 M in MeOH, 25 mL) was added and the mixture was stirred at room temperature overnight. The mixture was diluted with water and extracted with ethyl acetate. The combined organic layers were washed with brine, dried over sodium sulfate, filtered and concentrated to afford 112-4 which was used directly in the next step without purification.

Step 5: To a solution of 112-4 (23.0 g, 80.9 mmol) and pyridine (12.8 g, 161.8 mmol, 13.0 mL) in dichloromethane (60 mL) was added Tf₂O (27.4 g, 97.1 mmol) at 0° C. The mixture was stirred at 0° C. for 1 h. The mixture was diluted with water and extracted with dichloromethane. The combined organic layers were washed with brine, dried over sodium sulfate, filtered and concentrated. The residue was purified by column chromatography on silica gel (petroleum ether/ethyl acetate=20/1) to afford 112-5.

Step 6: A mixture of 112-5 (18.0 g, 43.2 mmol), 4,4,5,5-tetramethyl-2-(4,4,5,5-tetramethyl-1,3,2-dioxaborolan-2-yl)-1,3,2-dioxaborolane (87.8 g, 345.8 mmol), KOAc (12.7 g, 129.7 mmol) and Pd(PPh₃)₄ (10.0 g, 8.65 mmol) in 1,4-dixoxane (240 mL) was stirred at 80° C. overnight. The mixture was diluted with water and extracted with ethyl acetate. The combined organic layers were washed with brine, dried over sodium sulfate, filtered and concentrated. The residue was purified by reverse phase HPLC (acetonitrile with 0.05% of TFA in water: 10% to 95%) to afford 112-6.

Compound 112-7 was prepared from compound 73-6 following the procedure for the synthesis of compound 73-7 in example 6.

Compound 112-9 was prepared following the procedure for the synthesis of compound 119-13 in example 19.

Step 7: To a solution of 112-9 (60 mg, 0.074 mmol) in acetonitrile/N,N-dimethylacetamide (1 mL/0.5 mL) was added bromo(trimethyl)silane (0.2 mL). The mixture was stirred at room temperature for 6 h. Then the mixture was diluted with dichloromethane, washed with saturated sodium bicarbonate aqueous, water and brine successively. The organic layer was dried over sodium sulfate, filtered and concentrated. The residue was purified by prep-TLC (dichloromethane/methanol=10/1) and prep-HPLC (acetonitrile with 0.1% of FA in water: 5% to 95%) to afford 112 as a 3 eq of FA salt. LCMS (ESI, m/z): [M+H]⁺=607.3; HNMR (400 MHz, methanol-d₄, ppm): δ 8.47 (s, 3H), 8.26 (s, 1H), 7.33 (dd, J=8.8, 5.6 Hz, 1H), 7.02 (t, J=8.8 Hz, 1H), 5.51-5.37 (m, 1H), 4.53-4.44 (m, 4H), 4.03-3.95 (m, 2H), 3.90-3.45 (m, 5H), 3.28-3.22 (m, 1H), 2.60-1.86 (m, 10H).

### Example 23 Synthesis of Compound 143

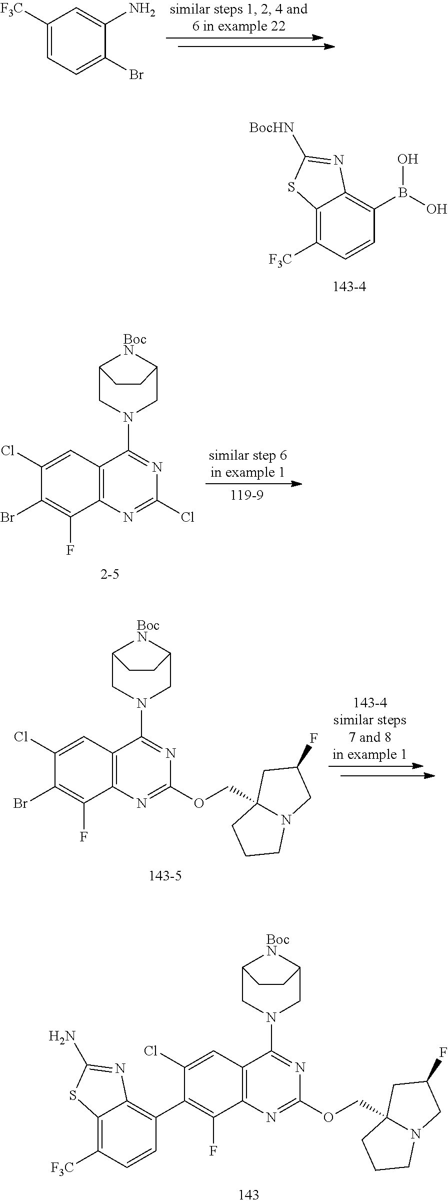

Compound 143-4 was prepared following the procedure for the synthesis of compound 112-6 in example 22.

Compound 143-5 was prepared from compound 2-5 and compound 119-9 following the procedure for the synthesis of compound 2-6 in example 1.

Compound 143 was prepared from compound 143-5 following the procedure for the synthesis of compound 2 in example 1 as a 0.29 eq of FA salt. LCMS (ESI, m/z): [M+H]⁺=666.1; HNMR (400 MHz, methanol-d₄, ppm): δ 8.34 (s, 0.29H), 7.96 (s, 1H), 7.54-7.48 (m, 1H), 7.39-7.34 (m, 1H), 5.61-5.40 (m, 1H), 4.74-4.64 (m, 2H), 4.61-4.54 (m, 2H), 4.19-4.10 (m, 2H), 3.92-3.71 (m, 5H), 3.42-3.35 (m, 1H), 2.70-2.46 (m, 2H), 2.43-2.34 (m, 1H), 2.33-2.22 (m, 2H), 2.17-2.01 (m, 5H).

### Example 24 Synthesis of Compound 121

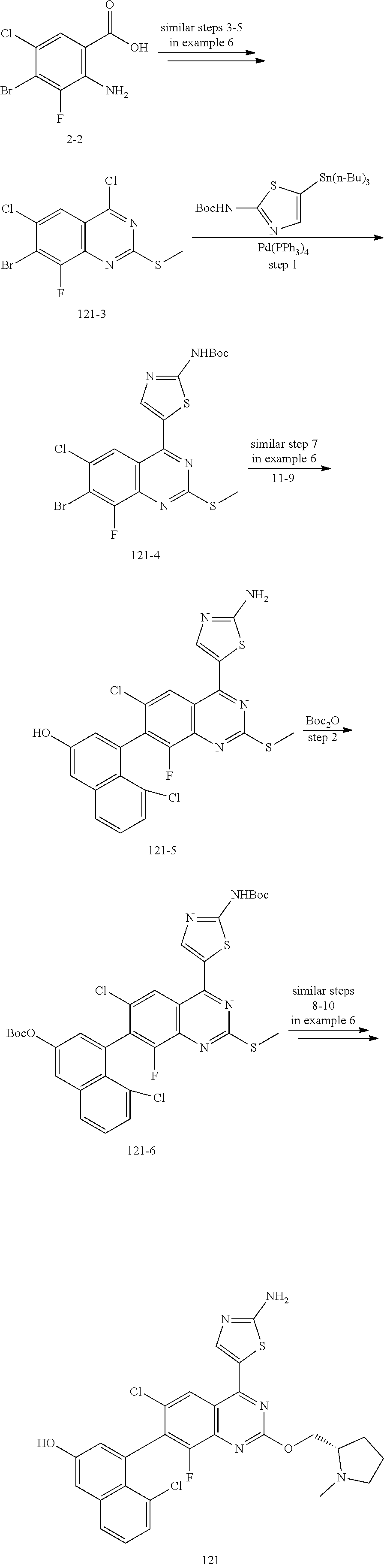

Compound 121-3 was prepared from compound 2-2 following the procedure for the synthesis of compound 73-5 in example 6.

Step 1: To a stirred mixture of 121-3 (1 g, 2.92 mmol) and tert-butyl (5-(tributylstannyl) thiazol-2-yl) carbamate (1.43 g, 2.92 mmol) in 1,4-dioxane (30 mL) was added tetrakis(triphenylphosphine) palladium (337 mg, 0.29 mmol) under nitrogen. The resulting mixture was stirred at 85° C. for 16 h. After being cooled to room temperature, the mixture was filtered and the filtered cake was washed with 1,4-dioxane. The combined organic layers were concentrated to afford 121-4.

Compound 121-5 was prepared from compound 121-4 and compound 11-9 following the procedure for the synthesis of compound 73-7 in example 6.

Step 2: To a stirred mixture of 121-5 (10 mg, 0.020 mmol) and DMAP (2.7 mg, 0.022 mmol) in THF (1 mL) was added TEA (13 mg, 0.13 mmol) and Boc₂O (24 mg, 0.11 mmol). The resulting mixture was stirred at room temperature for 1 h. The mixture was cooled and concentrated. The residue was purified by silica gel column chromatography (petroleum ether/ethyl acetate=3/1) to afford 121-6.

Compound 121 was prepared from compound 121-6 following the procedure for the synthesis of compound 73 in example 6. LCMS (ESI, m/z): [M+H]⁺=570.1; HNMR (400 MHz, methanol-d₄, ppm): δ 8.35 (s, 1H), 8.21 (s, 1H), 7.75 (d, J=8.0 Hz, 1H), 7.37-7.29 (m, 3H), 6.99 (s, 1H), 3.96-3.88 (m, 1H), 3.75-3.70 (m, 1H), 3.08 (s, 3H), 2.80-2.73 (m, 2H), 2.19-2.09 (m, 2H), 2.09-1.95 (m, 2H), 1.60-1.56 (m, 1H). FNMR (376 MHz, methanol-d₄, ppm): δ −123.65 (1F).

### Example 25 Synthesis of Compound 122

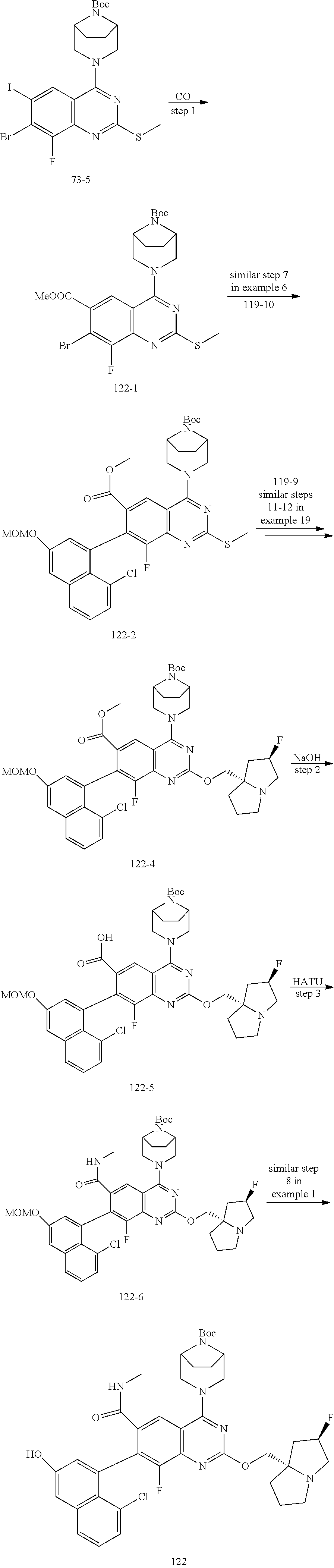

A mixture of 73-5 (500.00 mg, 0.82 mmmol), TEA (249.12 mg, 2.46 mmol, 0.34 mL) and Pd(dppf)Cl₂ (120.14 mg, 0.16 mmol) in methanol (15 mL) were stirred at room temperature under a balloon of carbon monoxide for 5 h. The mixture was filtered and the filtrate was concentrated. The residue was purified by silica gel column chromatography (petroleum ether/ethyl acetate=10/1) to afford 122-1.

Compound 122-2 was prepared from compound 122-1 and 119-10 following the procedure for the synthesis of compound 73-7 in example 6.

Compound 122-4 was prepared from compound 122-2 following the procedure for the synthesis of compound 119-13 in example 19.

Step 2: To a solution of 122-4 (40 mg, 0.05 mmol) in tetrahydrofuran/methanol (3 mL/1 mL) was added sodium hydroxide solution (1 mL, 2 mmol, 2M). The reaction was stirred at room temperature for 16 h. The mixture was acidified by 1M hydrochloric acid to pH 4-5 and extracted with dichloromethane. The combined organic layers were concentrated to afford 122-5.

Step 3: To a solution of 122-5 (35 mg, 0.045 mmol) in dimethylformamide (2 mL) was added 2-(7-aza-1H-benzotriazole-1-yl)-1,1,3,3-tetramethyluronium hexafluorophosphate (25 mg, 0.067 mmol), DIPEA (17 mg, 0.14 mmol) and methylamine hydrochloride (5 mg, 0.067 mmol). The reaction was stirred at room temperature for an hour. The mixture was purified by prep-HPLC (acetonitrile with 0.05% of TFA in water: 10% to 60%) to afford 122-6.

Compound 122 was prepared following the procedure for the synthesis of compound 2 in example 1 as a 3 eq of TFA salt. LCMS (ESI, m/z): [M+H]⁺=649.2; HNMR (400 MHz, methanol-d₄, ppm): δ 7.95 (s, 1H), 7.72-7.70 (m, 1H), 7.35-7.27 (m, 3H), 6.96 (d, J=2.4 Hz, 1H), 5.60-5.47 (m, 1H), 4.79-4.62 (m, 4H), 4.24 (s, 2H), 4.02-3.81 (m, 5H), 3.48-3.41 (m, 1H), 2.72-2.56 (m, 5H), 2.44-2.29 (m, 3H), 2.19-2.08 (m, 5H). FNMR (376 MHz, methanol-d₄, ppm): δ −126.92 (1F), −174.36 (1F).

### Example 26 Synthesis of Compound 142

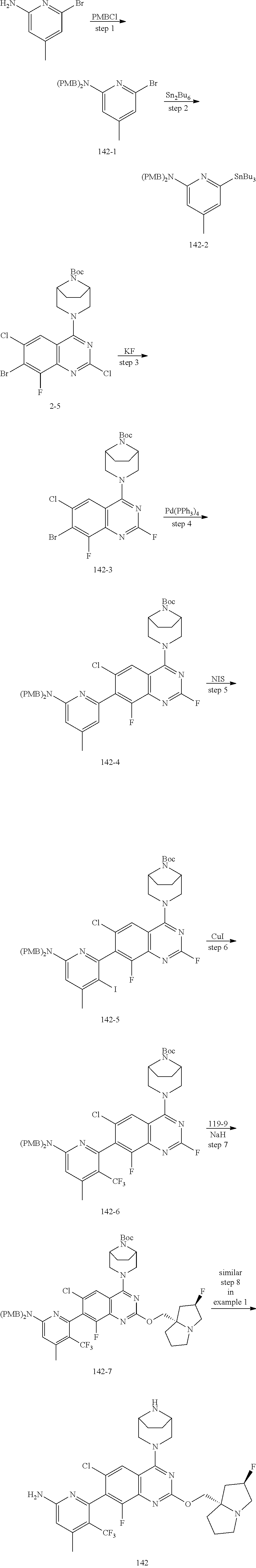

Step 1: To a solution of 6-bromo-4-methylpyridin-2-amine (10 g, 53 mmol) in DMF (150 mL) was added 60% wt. NaH in mineral oil (8.13 g, 203 mmol) in portions at 0° C. The resulting mixture was stirred at room temperature for 1 h. Then 4-methoxybenzylchloride (18.3 g, 117 mmol) was added and the mixture was stirred at this temperature for 2 h. After being quenched with saturated NH₄Cl solution, the mixture was extracted with ethyl acetate. The combined organic layers were washed with brine, dried over anhydrous Na₂SO₄, filtered and concentrated. The residue was purified by silica gel column chromatography (petroleum ether/ethyl acetate=10/1) to afford 142-1.

Step 2: A mixture of 142-1 (1 g, 2.3 mmol), hexabutylditin (4.1 g, 7.1 mmol), Pd₂(dba)₃ (215 mg, 0.23 mmol), tricyclohexyl phosphine (131 mg, 0.46 mmol) and lithium chloride (492 mg, 11.7 mmol) in 1,4-dioxane (20 mL) was stirred at 110° C. for 5 h under nitrogen atmosphere. The reaction mixture was concentrated and the residue was purified by silica gel column chromatography (petroleum ether/ethyl acetate=10/1) to afford 142-2.

Step 3: To a solution of 2-5 (4.08 g, 8.06 mmol) in DMA (120 mL) was added KF (11.27 g, 194.01 mmol). The mixture was stirred at 120° C. for 12 h. The mixture was poured into H₂O and extracted with ethyl acetate. The combined organic layers were dried over sodium sulfate, filtered and concentrated. The residue was purified by silica gel column chromatography (petroleum ether/ethyl acetate=10/1) to afford 142-3.

Step 4: To a solution of 142-3 (500 mg, 1.02 mmol) and 142-2 (1.04 g, 1.63 mmol) in dioxane (10 mL) was added LiCl (108.19 mg, 2.55 mmol), CuI (61.7 mg, 0.32 mmol) and Pd(PPh₃)₄ (235.84 mg, 0.20 mmol) under N₂. The solution was stirred at 120° C. for 10 h and concentrated. The residue was purified by silica gel column chromatography (petroleum ether/ethyl acetate=4/1) to afford 142-4.

Step 5: To a solution 142-4 (410 mg, 0.54 mmol) in DMF (10 mL) was added TsOH·H₂O (108 mg, 0.56 mmol) and N-iodosuccinimide (609 mg, 2.71 mmol). The resulting solution was stirred at 0° C. for 3 h. The mixture was diluted with water and extracted with ethyl acetate. The combined organic layers were dried over sodium sulfate, filtered and concentrated. The residue was purified by silica gel column chromatography (petroleum ether/ethyl acetate=4/1) to afford 142-5.

Step 6: To a solution of 142-5 (130 mg, 0.15 mmol) and CuI (336.41 mg, 1.77 mmol) in DMA (5 mL) was added methyl 2,2-difluoro-2-fluorosulfonyl-acetate (706.96 mg, 3.68 mmol) under N₂. The solution was stirred at 90° C. for 18 h. The mixture was diluted with water and extracted with ethyl acetate. The combined organic layers were dried over sodium sulfate, filtered and concentrated. The residue was purified by silica gel column chromatography (petroleum ether/ethyl acetate=3/1) to afford 142-6.

Step 7: To a solution of 119-9 (48.7 mg, 0.3 mmol) in THF (5 mL) was added NaH (60% in oil, 8.5 mg, 0.35 mmol) at 0° C. under N₂. The solution was stirred at 25° C. for 1 h and a solution of 142-6 (101 mg, 0.12 mmol) in 2 mL of THF was added. The solution was stirred for 1 h at 25° C. The mixture was diluted with water and extracted with dichloromethane. The combined organic layers were dried over sodium sulfate, filtered and concentrated. The residue was purified by silica gel column chromatography (dichloromethane/methanol=20/1) to afford 142-7.

Compound 142 was prepared following the procedure for the synthesis of compound 2 in example 1 as a 0.46 eq of FA salt. LCMS (ESI, m/z): [M+H]⁺=624.0; HNMR (400 MHz, methanol-d₄, ppm): δ 8.41 (s, 0.46H), 7.90 (s, 1H), 6.61 (s, 1H), 5.65-5.45 (m, 1H), 4.64-4.59 (m, 4H), 4.16-4.01 (m, 2H), 3.98-3.81 (m, 5H), 3.49-3.46 (m, 1H), 2.69 (s, 3H), 2.56-2.21 (m, 5H), 2.16-1.99 (m, 5H).

### Example 27 Synthesis of Compound 137

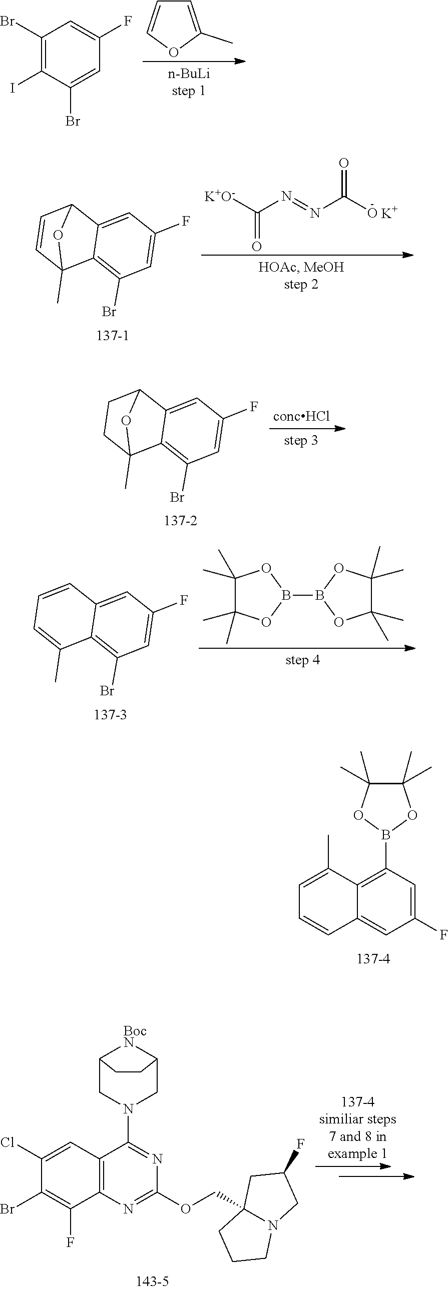

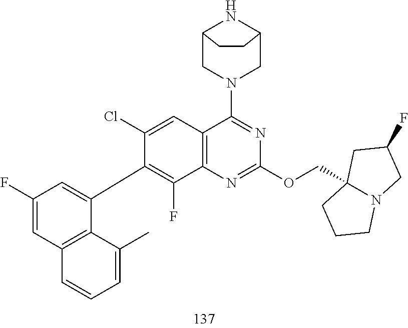 [SMILES: Cc1cccc2cc(F)cc(-c3c(Cl)cc4c(N5CC6CCC(C5)N6)nc(OC[C@@]56CCCN5C[C@H](F)C6)nc4c3F)c12]

Step 1: To a solution of 1,3-dibromo-5-fluoro-2-iodobenzene (5 g, 13 mmol) and 2-methylfuran (3.2 g, 39 mmol) in toluene (50 mL) was added 2.5 M n-BuLi solution in THF (5.7 mL, 14 mmol) dropwise at −50° C. The resulting solution was warmed slowly to room temperature and stirred for 1 h. After being quenched with water, the mixture was extracted with ethyl acetate. The combined organic layers were washed with brine, dried over anhydrous Na₂SO₄, filtered and concentrated. The residue was purified by silica gel column chromatography (petroleum ether) to afford 137-1.

Step 2: To a solution of 137-1 (1.03 g, 4.02 mmol) in MeOH (50 mL) was added potassium azodicarboxylate (2.34 g, 12.06 mmol) at room temperature in the dark. The mixture was stirred while a solution of glacial acetic acid (1.82 mL) in MeOH (30 mL) was added dropwise. The resulting mixture was stirred at room temperature for 15 min. The mixture was diluted with water and extracted with ethyl acetate. The combined organic layers were washed with brine, dried over anhydrous Na₂SO₄, filtered and concentrated to afford 137-2 which was used directly in next step without purification.

Step 3: A mixture of 137-2 (800 mg crude) in 12 N aqueous HCl solution (20 mL) was stirred at 95° C. for 16 h in a sealed tube. After being cooled to room temperature, the mixture was diluted with water and extracted with dichloromethane. The combined organic layers were washed with brine, dried over anhydrous Na₂SO₄, filtered and concentrated. The residue was purified by silica gel column chromatography (petroleum ether) to afford 137-3.

Step 4: A mixture of 137-3 (600 mg, 2.52 mmol), 4,4,4′,4′,5,5,5′,5′-octamethyl-2,2′-bi(1,3,2-dioxaborolane) (960 mg, 3.78 mmol), Pd(dppf)Cl₂ (187 mg, 0.25 mmol) and KOAc (750 mg, 7.65 mmol) in 1,4-dioxane (15 mL) was degassed three times under N₂ and stirred at 90° C. for 5 h. The mixture was cooled and concentrated. The residue was purified by silica gel column chromatography (petroleum ether) to afford 137-4.

Compound 137 was prepared from compound 137-4 and compound 143-5 following the procedure for the synthesis of compound 2 in example 1 as a 3 eq of TFA salt. LCMS (ESI, m/z): [M+H]⁺=608.3; HNMR (400 MHz, methanol-d₄, ppm): δ 7.98 (s, 1H), 7.82 (d, J=8.4 Hz, 1H), 7.70 (dd, J=9.2, 2.4 Hz, 1H), 7.43 (t, J=7.6 Hz, 1H), 7.25 (d, J=7.2 Hz, 1H), 7.15-7.12 (m, 1H), 5.60-5.50 (m, 1H), 4.79-4.73 (m, 2H), 4.68-4.65 (m, 3H), 4.28-4.19 (m, 2H), 3.95-3.81 (m, 4H), 3.46-3.43 (m, 1H), 2.75-2.50 (m, 2H), 2.41-2.28 (m, 3H), 2.19-2.00 (m, 8H). FNMR (376 MHz, methanol-d₄, ppm): δ −119.34 (1F), −123.09 (1F), −174.26 (1F).

### Example 28 Synthesis of Compound 123

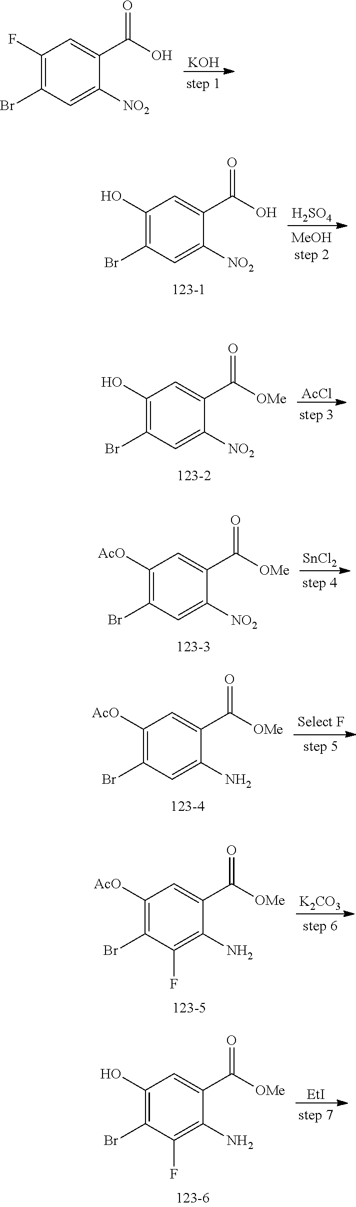

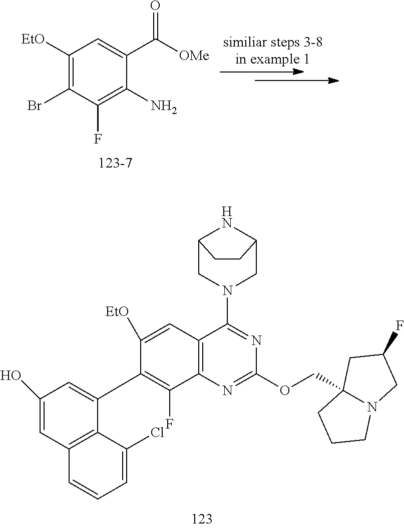 [SMILES: CCOc1cc(C(=O)OC)c(N)c(F)c1Br ; CCOc1cc2c(N3CC4CCC(C3)N4)nc(OC[C@@]34CCCN3C[C@H](F)C4)nc2c(F)c1-c1cc(O)cc2cccc(Cl)c12]

Step 1: To a solution of 4-bromo-5-fluoro-2-nitrobenzoic acid (2.6 g, 10 mmol) in water (16 mL) was added potassium hydroxide solution (12 M, 3 mL, 36 mmol). The reaction was stirred at 80° C. for 1.5 h. The mixture was acidified with 1 M hydrochloric acid to pH=3 and extracted with ethyl acetate. The combined organic layers were dried over anhydrous sodium sulfate, filtered and concentrated to afford 123-1 which was used directly in the next step without purification.

Step 2: To a solution of 123-1 (2.5 g, 10 mmol) in methanol (30 mL) was added conc. H₂SO₄ (2.6 mL). The reaction was stirred at 70° C. for 16 h. The mixture partitioned between ethyl acetate and water. The organic layer was dried over anhydrous sodium sulfate, filtered and concentrated to afford 123-2 which was used directly in the next step without purification.

Step 3: To a solution of 123-2 (1.9 g, 6.9 mmol) and triethylamine (2.1 g, 20.6 mmol) in dichloromethane (60 mL) was added acetyl chloride (0.78 g, 10 mmol) at 0° C. and. The mixture was stirred at 0° C. for 2 h. The mixture partitioned between ethyl acetate and water. The organic layer was dried over anhydrous sodium sulfate, filtered and concentrated to afford 123-3 which was used directly in the next step without purification.

Step 4: To a solution of 123-3 (2.1 g, 69 mmol) in ethyl acetate (60 mL) was added stannous chloride (5.3 g, 28 mmol). The reaction was stirred at 60° C. for 3 h. The mixture was basified with aqeuous sodium bicarbonate to pH=8 and then filtered. The filtrate was dried over anhydrous sodium sulfate, filtered and concentrated to afford 123-4 which was used directly in the next step without purification.

Step 5: To a solution of 123-4 (1.8 g, 6.25 mmol) in acetonitrile (50 mL) was added Selectfluor (2.43 g, 6.8 mmol). The reaction was stirred at room temperature for 16 h. The mixture was basified with aqueous sodium bicarbonate to pH=8 and then extracted with ethyl acetate. The combined organic layers were dried over anhydrous sodium sulfate, filtered and concentrated. The residue was purified by column chromatography on silica gel (petroleum ether/ethyl acetate=4/1) to afford 123-5.

Step 6: To a solution of 123-5 (550 mg, 1.8 mmol) in methanol (10 mL) was added potassium carbonate (496 mg, 3.6 mmol). The reaction was stirred at room temperature for 2 h. The mixture was acidified with 1 M hydrochloric acid to pH=5 and extracted with ethyl acetate. The mixture was concentrated and the residue was purified by column chromatography on silica gel (petroleum ether/ethyl acetate=4/1) to afford 123-6.

Step 7: To a solution of 123-6 (450 mg, 1.7 mmol) in N,N-dimethylformamide (15 mL) was added cesium carbonate (1.1 g, 3.4 mmol). The reaction was stirred at room temperature for 10 min, followed by addition of iodoethane (265 mg, 1.7 mmol). The mixture was stirred at 0° C. for 2 h. The mixture was diluted with water and extracted with ethyl acetate. The combined organic layers were dried over anhydrous sodium sulfate, filtered and concentrated. The residue was purified by column chromatography on silica gel (petroleum ether/ethyl acetate=4/1) to afford 123-7.

Compound 123 was prepared from compound 123-7 following the procedure for the synthesis of compound 2 in example 1. LCMS (ESI, m/z): [M+H]⁺=636.3; HNMR (400 MHz, methanol-d₄, ppm): δ 7.72-7.69 (m, 1H), 7.33-7.25 (m, 3H), 7.06 (s, 1H), 6.94 (t, J=2.0 Hz, 1H), 5.60-5.45 (m, 1H), 4.69-4.53 (m, 4H), 4.24-3.22 (m, 2H), 4.15-3.70 (m, 7H), 3.47-3.42 (m, 1H), 2.67-2.18 (m, 10H), 1.12 (t, J=7.2 Hz, 3H).

### Example 29 Synthesis of Compound 146

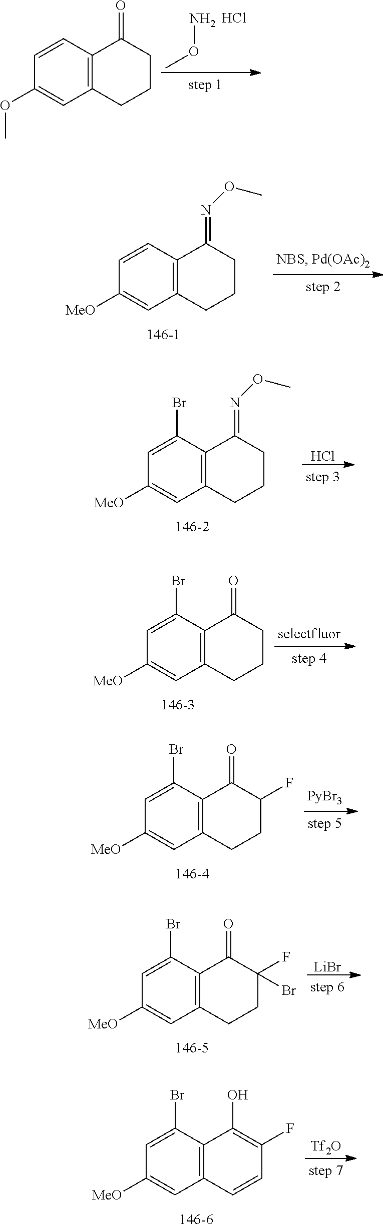

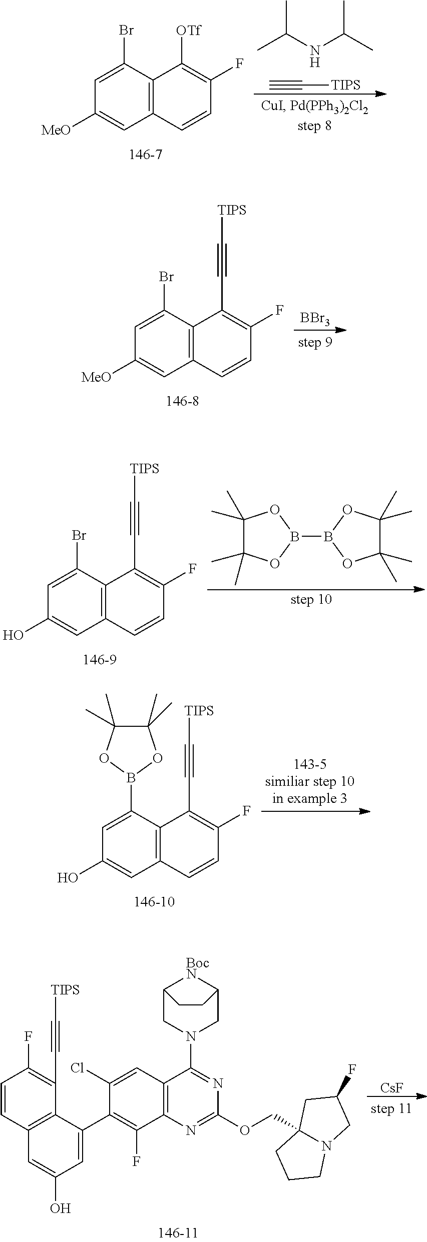

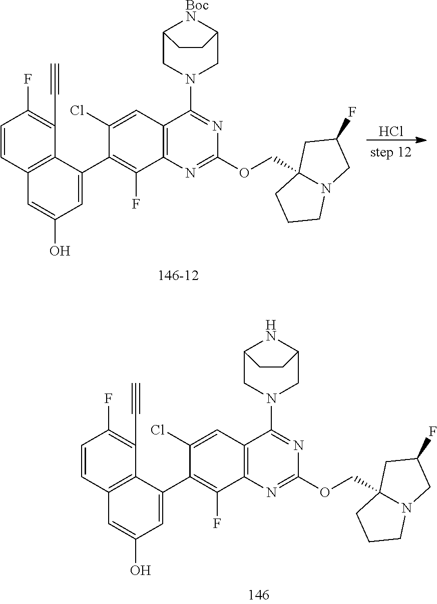 [SMILES: C#Cc1c(F)ccc2cc(O)cc(-c3c(Cl)cc4c(N5CC6CCC(C5)N6C(=O)OC(C)(C)C)nc(OC[C@@]56CCCN5C[C@H](F)C6)nc4c3F)c12 ; Cl ; C#Cc1c(F)ccc2cc(O)cc(-c3c(Cl)cc4c(N5CC6CCC(C5)N6)nc(OC[C@@]56CCCN5C[C@H](F)C6)nc4c3F)c12]

Step 1: A mixture of 6-methoxy-3,4-dihydronaphthalen-1(2H)-one (50 g, 280 mmol), O-methylhydroxylamine hydrochloride (28 g, 336 mmol) in ethanol (500 mL) and pyridine (33 g, 420 mmol) was stirred at room temperature for 2 h. The mixture was concentrated to give an oil. The oil was dissolved in dichloromethane, washed with 2N hydrochloric acid, saturated aqueous sodium bicarbonate, brine, dried over sodium sulfate, filtered and concentrated to afford 146-1 which was used directly in the next step without purification.

Step 2: A mixture of 146-1 (25 g, 120 mmol), palladium(II) acetate (1.3 g, 6 mmol), N-bromosuccinimide (21 g, 120 mmol) in acetic acid (400 mL) was stirred at 80° C. for 1 hour. The solution was poured into water and filtered. The cake was dried to afford 146-2 which was used directly in the next step without purification.

Step 3: A suspension of 146-2 (18 g, 80 mmol) in concentrated hydrochloric acid (100 mL) and dioxane (150 mL) was stirred at reflux for 1 h. The mixture was concentrated, and the residue was dissolved in ethyl acetate, washed with 1 N NaOH, water, brine (150 mL), and concentrated to afford the crude product. The product was purified by column chromatography on silica gel (petroleum ether to petroleum ether/ethyl acetate=4/1) to afford 146-3.

Step 4: To a mixture of 1-chloromethyl-4-fluoro-1,4-diazoniabicyclo[2.2.2]octane bis(tetrafluoroborate) (8.14 g, 23 mmol) and 146-3 (5.1 g, 20 mmol) in methanol (80 mL) was added concentrated sulfuric acid (0.1 mL). The mixture was stirred at 50° C. for 5 h under N₂ atmosphere. The mixture was concentrated, diluted with ethyl acetate, washed with water and brine. The organic layer was dried over sodium sulfate, filtered and concentrated. The residue was triturated with petroleum ether/ethyl acetate (10/1) to afford 146-4.

Step 5: The mixture of 146-4 (4.63 g, 16.96 mmol) and pyridinium tribromide (5.97 g, 18.66 mmol) in acetonitrile (46 mL) was stirred at 60° C. for 30 min under N₂ atmosphere. The mixture was diluted with ethyl acetate, washed with water and brine. The organic layer was dried over sodium sulfate, filtered and concentrated. The residue was triturated with petroleum ether/ethyl acetate (10/1) to afford 146-5.

Step 6: A mixture of 146-5 (5.4 g, 15.38 mmol), lithium bromide (2.94 g, 33.85 mmol) in N,N-dimethylformamide (15 mL) was stirred at 100° C. for 30 min under N₂ atmosphere. After being cooled to room temperature, the mixture was diluted with ethyl acetate, washed with water and brine. The organic layer was dried over sodium sulfate, filtered and concentrated. The residue was triturated with petroleum ether/ethyl acetate (10/1) to afford 146-6.

Step 7: To a mixture of 146-6 (12.96 g, 48 mmol) and pyridine (11.4 g, 144 mmol) in dichloromethane (150 mL) was added triflic anhydride (16.2 g, 57.6 mmol) dropwise at 0° C. under N₂ atmosphere. The mixture was stirred at room temperature for 1 h. The reaction mixture was washed with water, brine, dried over sodium sulfate, filtered and concentrated. The residue was purified by column chromatography on silica gel (petroleum ether to petroleum ether/ethyl acetate=8/1) to afford 146-7.

Step 8: To a mixture of 146-7 (18 g, 45 mmol) in N,N-dimethylformamide (300 mL) were added triisopropylsilylacetylene (12.3 g, 67.5 mmol), diisopropylamine (45.5 g, 450 mmol), CuI (855 mg, 4.5 mmol) and bis(triphenylphosphine)palladium(II) chloride (1.58 g, 2.25 mmol) under N₂ atmosphere. The mixture was stirred at 50° C. for 16 h. The mixture was diluted with ethyl acetate, washed with water and brine. The organic layer was dried over sodium sulfate, filtered and concentrated. The residue was purified by column chromatography on silica gel (petroleum ether to petroleum ether/ethyl acetate=10/1) to afford 146-8.

Step 9: To a mixture of 146-8 (10.6 g, 24.4 mmol) in dichloromethane (150 mL) was added boron tribromide (14.6 mL, 29.2 mmol, 2 M in dichloromethane) dropwise at −78° C. under N₂ atmosphere. The mixture was stirred at 0° C. for 3 h. The reaction was quenched with ice-water. The organic layer was washed with saturated aqueous sodium hydrogencarbonate and brine, dried over sodium sulfate, filtered and concentrated. The residue was purified by column chromatography on silica gel (petroleum ether to petroleum ether/ethyl acetate=6/1) to afford 146-9.

Step 10: A mixture of 146-9 (8.89 g, 19 mmol), bis(pinacolato)diboron (9.65 g, 38 mmol), potassium acetate (5.59 g, 57 mmol), tris(dibenzylideneacetone)dipalladium (870 mg, 0.95 mmol) and tricyclohexyl phosphine (532 mg, 1.9 mmol) in dioxane (100 mL) was stirred at 105° C. for 10 h under N₂ atmosphere. The mixture was diluted with ethyl acetate, washed with water and brine. The organic layer was dried over sodium sulfate, filtered and concentrated. The residue was purified by column chromatography on silica gel (petroleum ether to petroleum ether/ethyl acetate=8/1) to afford 146-10.

Compound 146-11 was prepared from compound 146-10 and compound 143-5 following the procedure for the synthesis of compound 11-12 in example 3.

Step 11: To a solution of 146-11 (18 mg, 0.02 mmol) in N,N-dimethylformamide (5 mL) was added caesium fluoride (31 mg, 0.2 mmol) at room temperature. The mixture was stirred at 50° C. for 1 h under N₂ atmosphere. The mixture was diluted with ethyl acetate, washed with water and brine. The organic layer was dried over sodium sulfate, filtered and concentrated to afford 146-12 which was used directly in the next step without purification.

Step 12: 146-12 obtained in previous step was dissolved in a 0.75 M HCl in ethylacetate (2.7 mL) at room temperature. The mixture was stirred at 50° C. for 1 h under N₂ atmosphere. The mixture was concentrated and the residue was purified by prep-HPLC (acetonitrile with 0.05% of TFA in water: 5% to 95%) to afford 146 as a 3 eq of TFA salt. LCMS (ESI, m/z): [M+H]⁺=634.3; HNMR (400 MHz, methanol-d₄, ppm): δ 7.90-7.84 (m, 2H), 7.34-7.30 (m, 2H), 7.03 (s, 1H), 5.62-5.48 (m, 1H), 4.80-4.73 (m, 1H), 4.71-4.62 (m, 3H), 4.27-4.24 (m, 2H), 4.03-3.81 (m, 5H), 3.47-3.44 (m, 1H), 3.28 (s, 1H), 2.73-2.55 (m, 2H), 2.45-2.34 (m, 3H), 2.24-2.09 (m, 5H). FNMR (376 MHz, methanol-d₄, ppm): δ −115.53 (1F), −123.83 (1F).

### Example 30 Synthesis of Compounds 154 and 155

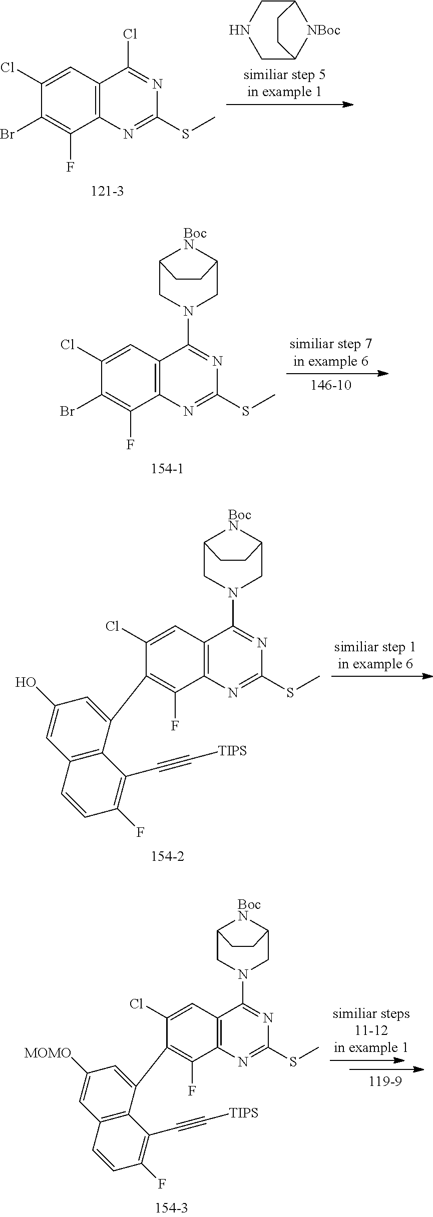

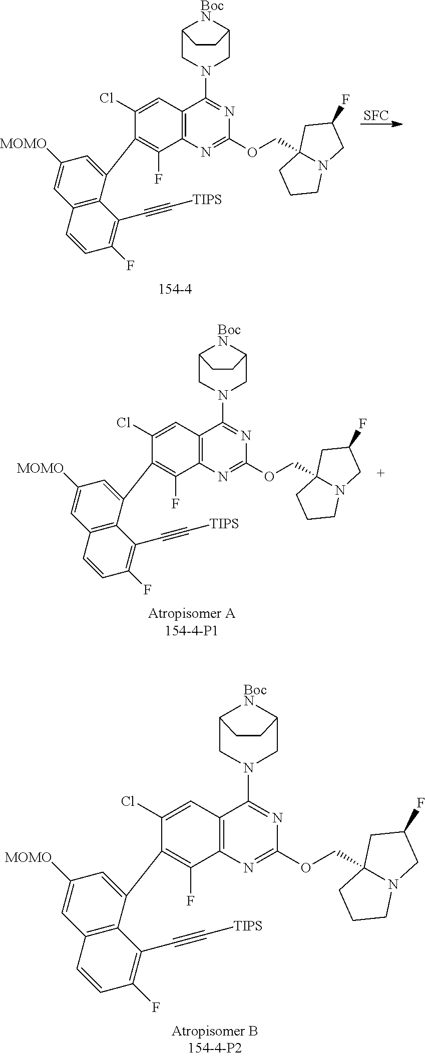

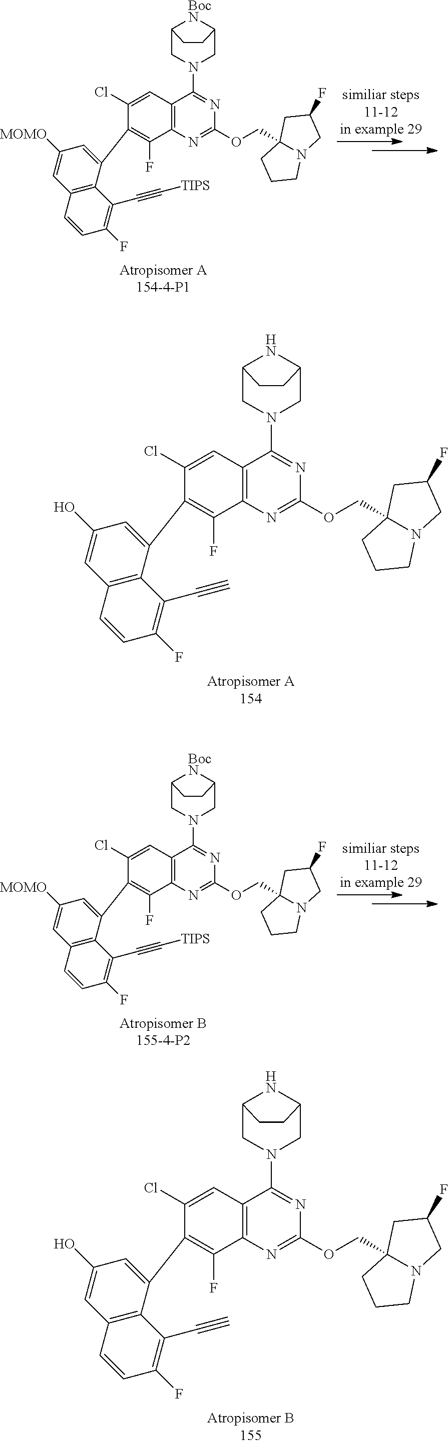

Compound 154-1 was prepared from compound 121-3 following the procedure for the synthesis of compound 2-5 in example 1.

Compound 154-2 was prepared from compound 154-1 and 146-10 following the procedure for the synthesis of compound 73-7 in example 6.

Compound 154-3 was prepared from compound 154-2 following the procedure for the synthesis of compound 73-1 in example 6.

Compound 154-4 was prepared from compound 154-3 following the procedure for the synthesis of compound 119-13 in example 19.

Compound 154-4 (646 mg) was purified by SFC (column: DAICEL CHIRALPAK IC, EtOH/n-Hexane/CO₂) to afford 154-4-P1 (275 mg) and 154-4-P2 (318 mg), respectively.

154-4-P1: SFC analysis: >99% ee; Retention time: 4.91 min; column: Daicel CHIRALPAK®IC, n-Hexane/EtOH (0.2% of DEA) in CO₂; pressure: 100 bar; flow rate: 1.0 mL/min.
154-4-P2: SFC analysis: >99% ee; Retention time: 5.73 min; column: Daicel CHIRALPAK®IC, n-Hexane/EtOH (0.2% DEA) in CO₂; pressure: 100 bar; flow rate: 1.0 mL/min.

Compound 154 was prepared from compound 154-4-P1 following the procedure for the synthesis of compound 146 in example 29. LCMS (ESI, m/z): [M+H]⁺=634.3; HNMR (400 MHz, methanol-d₄, ppm): δ 7.90-7.84 (m, 2H), 7.35-7.30 (m, 2H), 7.04-7.03 (m, 1H), 5.61-5.49 (m, 1H), 4.77-4.64 (m, 4H), 4.26-4.24 (m, 2H), 4.03-3.83 (m, 5H), 3.49-3.42 (m, 1H), 3.28-3.27 (m, 1H), 2.74-2.10 (m, 10H).

Compound 155 was prepared from compound 154-4-P2 following the procedure for the synthesis of compound 146 in example 29. LCMS (ESI, m/z): [M+H]⁺=634.3; HNMR (400 MHz, methanol-d₄, ppm): δ 7.90-7.84 (m, 2H), 7.35-7.30 (m, 2H), 7.03 (d, J=2.4 Hz, 1H), 5.63-5.49 (m, 1H), 4.83-4.74 (m, 1H), 4.73-4.61 (m, 3H), 4.30-4.21 (m, 2H), 4.05-3.81 (m, 5H), 3.48-3.41 (m, 1H), 3.27 (s, 1H), 2.74-2.53 (m, 2H), 2.45-2.29 (m, 3H), 2.23-2.02 (m, 5H).

### Example 31 Synthesis of Compounds 152 and 153

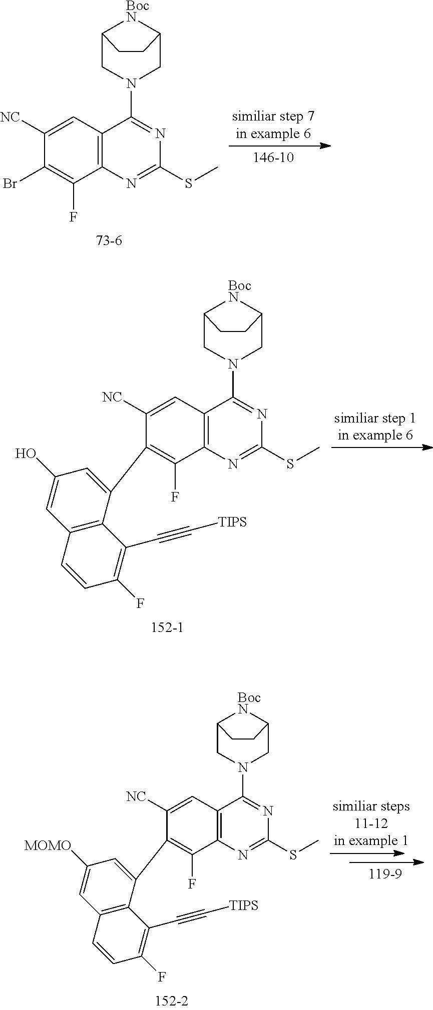

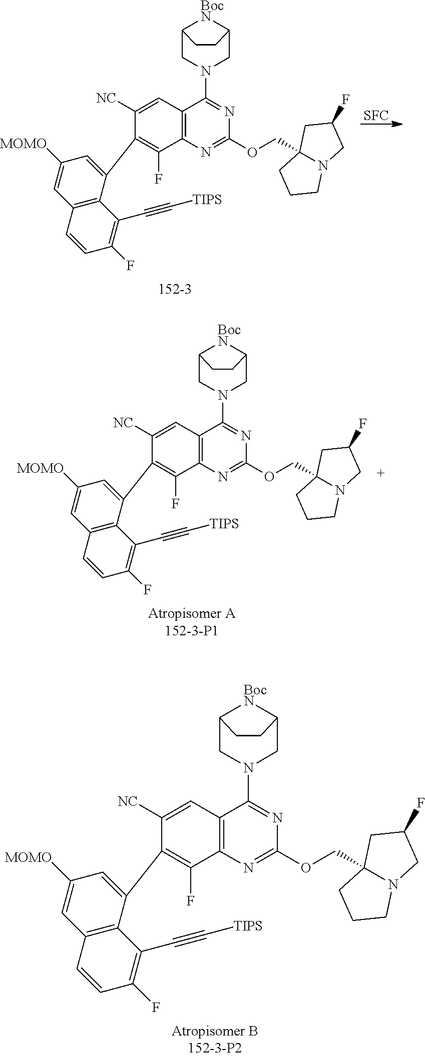

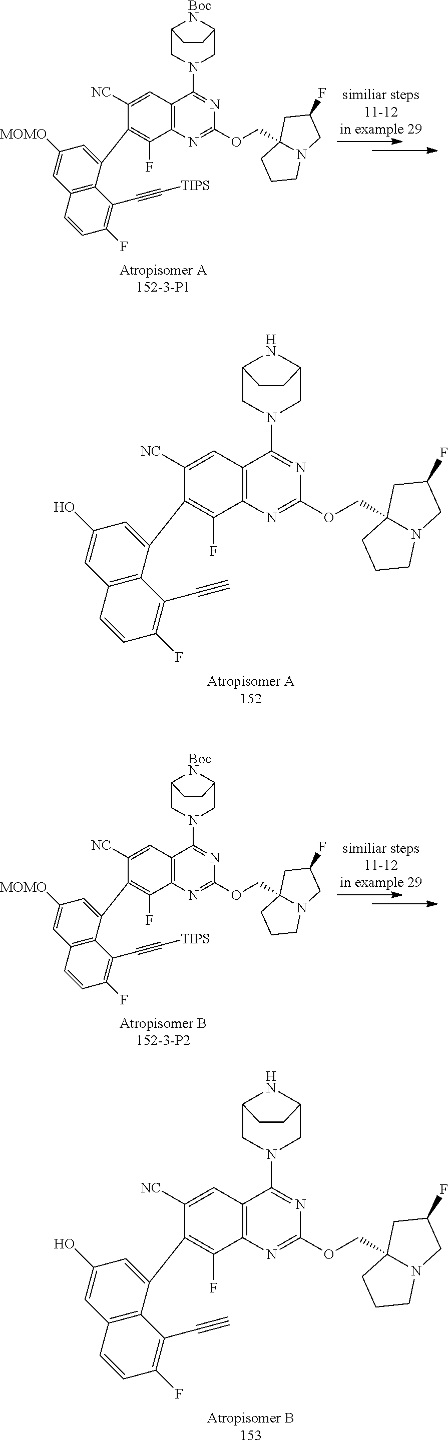

Compound 152-1 was prepared from compound 73-6 and 146-10 following the procedure for the synthesis of compound 73-7 in example 6.

Compound 152-2 was prepared from compound 152-1 following the procedure for the synthesis of compound 73-1 in example 6.

Compound 152-3 was prepared from compound 152-2 following the procedure for the synthesis of compound 119-13 in example 19.

Compound 152-3 (441 mg) was purified by SFC (column: DAICELCHIRALPAK®MIC, MeOH (0.2% of DEA)/CO₂) to afford 152-3-P1 (221 mg) and 152-3-P2 (206 mg), respectively.

152-3-P1: SFC analysis: >99% ee; Retention time: 1.68 min; column: DAICELCHIRALPAK®IC, MeOH (0.1% of DEA) in CO₂; pressure: 100 bar; flow rate: 1.5 mL/min.
152-3-P2: SFC analysis: >99% ee; Retention time: 2.20 min; column: DAICELCHIRALPAK®IC, MeOH (0.1% of DEA) in CO₂; pressure: 100 bar; flow rate: 1.5 mL/min.

Compound 152 was prepared from compound 152-3-P1 following the procedure for the synthesis of compound 146 in example 29 as a 3 eq. of TFA salt. LCMS (ESI, m/z): [M+H]⁺=625.3; HNMR (400 MHz, methanol-d₄, ppm): δ 8.30 (s, 1H), 7.92-7.88 (m, 1H), 7.40-7.33 (m, 2H), 7.14 (d, J=2.4 Hz, 1H), 5.63-5.50 (m, 1H), 4.83-4.66 (m, 4H), 4.32-4.21 (m, 2H), 4.05-3.83 (m, 5H), 3.49-3.42 (m, 1H), 3.37-3.34 (m, 1H), 2.78-2.53 (m, 2H), 2.49-2.28 (m, 3H), 2.25-2.03 (m, 5H). FNMR (376 MHz, methanol-d₄, ppm): δ −111.06 (1F), −124.88 (1F), −174.27 (1F).

Compound 153 was prepared from compound 152-3-P2 following the procedure for the synthesis of compound 146 in example 29 as a 3 eq. of TFA salt. LCMS (ESI, m/z): [M+H]⁺=625.3; HNMR (400 MHz, methanol-d₄, ppm): δ 8.29 (s, 1H), 7.92-7.88 (m, 1H), 7.40-7.33 (m, 2H), 7.15 (d, J=2.4 Hz, 1H), 5.63-5.50 (m, 1H), 4.85-4.67 (m, 4H), 4.32-4.21 (m, 2H), 4.08-3.82 (m, 5H), 3.53-3.42 (m, 1H), 3.37-3.34 (m, 1H), 2.78-2.53 (m, 2H), 2.49-2.28 (m, 3H), 2.25-2.03 (m, 5H). FNMR (376 MHz, methanol-d₄, ppm): δ −111.09 (1F), −124.84 (1F), −174.25 (1F).

### Example 32 Synthesis of Compounds 167

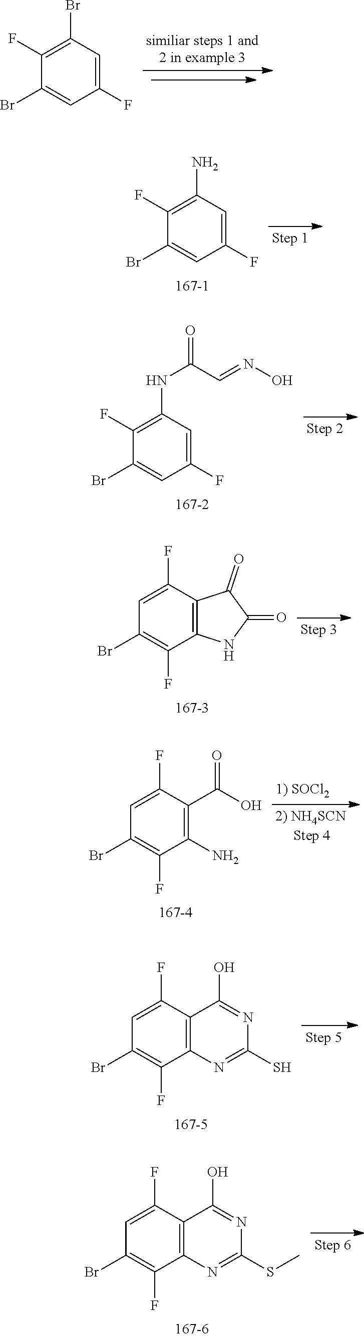

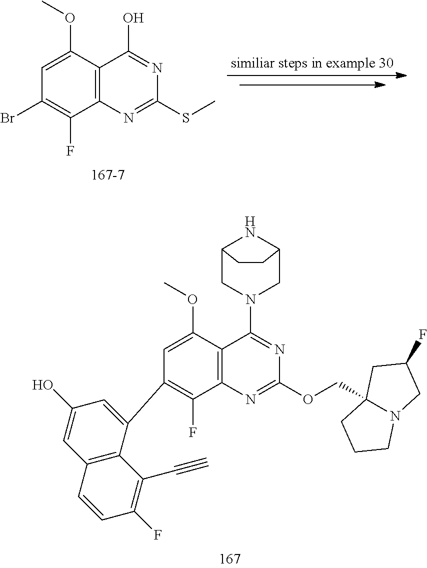 [SMILES: COc1cc(Br)c(F)c2nc(SC)nc(O)c12 ; C#Cc1c(F)ccc2cc(O)cc(-c3cc(OC)c4c(N5CC6CCC(C5)N6)nc(OC[C@@]56CCCN5C[C@H](F)C6)nc4c3F)c12]

Compound 167-1 was prepared from 1,3-dibromo-2,5-difluorobenzene and benzophenone imine following the procedure for the synthesis of compound 11-2 in example 3.

Step 1: A mixture of sodium sulfate (46.3 g, 326.16 mmol), hydroxylamine hydrochloride (9.92 g, 142.70 mmol) and chloral hydrate (10.12 g, 61.16 mmol) in water (200 mL) was stirred at room temperature for 0.5 hour. Then a solution of 167-1 (16 g, −40.77 mmol) in ethanol (28 mL), water (16 mL) and concentrated hydrochloric acid (7 mL) was added to above mixture. The reaction mixture was stirred at 60° C. for 16 hours with mechanical stirring. The mixture was cooled to room temperature and filtered. The cake was slurried with petroleum ether/ethyl acetate (240 mL/40 mL) to afford 167-2.

Step 2: 167-2 (7.75 g, 27.88 mmol) was dissolved in sulfuric acid (70 mL) at 60° C. Then the reaction mixture was stirred at 90° C. for 1 hour. The reaction mixture was cooled down to room temperature and poured to ice water slowly. The resulting precipitate was collected by filtration, washed with water and dried under vacuum. The cake was purified by column chromatography on silica gel (petroleum ether to petroleum ether/ethyl acetate=2/1) to give 167-3.

Step 3: To a solution of 167-3 (5.46 g, 20.84 mmol) in 2N sodium hydroxide aqueous (94 mL) was added 30% hydrogen peroxide aqueous (11.81 g, 104.20 mmol) at 0° C., then stirred at room temperature for 4 hours. The mixture was adjusted to pH-8 with concentrated hydrochloric acid. The resulting cream precipitate was filtered to afford 167-4.

Step 4: A solution of 167-4 (4.07 g, 16.15 mmol) in thionyl chloride (50 mL) was stirred for 1 hour at 45° C. The mixture was concentrated and dissolved in acetone (50 mL). The mixture was treated with ammonium thiocyanate (1.35 g, 17.77 mmol), then stirred for 1 hour at room temperature. The reaction mixture was diluted with water and filtered to give 167-5.

Step 5: The mixture of 167-5 (4.32 g, 14.75 mmol) in methanol (60 mL) was added a solution of sodium hydroxide (1.18 g, 29.5 mmol) in water (45 mL) and iodomethane (4.19 g, 29.5 mmol) at room temperature, then stirred for 1 hour. Reaction mixture was poured into water, adjusted to pH-6 with 2N hydrochloride aqueous, filtered and washed with water. The cake was made a slurry with methanol (20 mL) to give 167-6.

Step 6: To a solution of methanol (313 mg, 9.78 mmol) in N,N-dimethylformamide (10 mL) was added sodium hydride (456 mg, 60%, 11.41 mmol) at 0° C., and the reaction was stirred at 0° C. for 0.5 hour. Then the reaction mixture was treated with 167-6 (1 g, 3.26 mmol) in portions and stirred at room temperature for 16 hours. The mixture was diluted with water, and adjusted to pH-3 with 2N hydrochloric acid. The mixture was filtered to give 167-7.

Compound 167 was prepared from compound 167-7 and tert-butyl 3,8-diazabicyclo[3.2.1]octane-8-carboxylate following the procedure for the synthesis of compound 154 in example 30 as a 3 eq. of TFA salt. LCMS (ESI, m/z): [M+H]⁺=630.3; HNMR (400 MHz, methanol-d₄, ppm): δ 7.87-7.83 (m, 1H), 7.34-7.29 (m, 2H), 7.13 (d, J=2.4 Hz, 1H), 6.90 (d, J=4.8 Hz, 1H), 5.60-5.46 (m, 1H), 4.74-4.62 (m, 2H), 4.57-4.29 (m, 2H), 4.22-4.18 (m, 2H), 4.04-3.64 (m, 8H), 3.50-3.37 (m, 1H), 3.35-3.31 (m, 1H), 2.75-2.53 (m, 2H), 2.51-2.26 (m, 3H), 2.22-1.98 (m, 5H). FNMR (400 MHz, methanol-d₄, ppm): δ −111.51 (1F), −140.39 (1F), −174.26 (1F).

Compounds of the present disclosure can be synthesized by those skilled in the art in view of the present disclosure. Representative further compounds synthesized by following similar procedures/methods described herein in the Examples section and their characterization data are shown in Table 1 below.

TABLE 1
Characterization of representative compounds of the present disclosure

| Com- | | | |
|---|---|---|---|
| pound | | [M + | |
| No. | Structure | H]⁺ | ¹H-NMR and ¹⁵F-NMR |
| 1 | 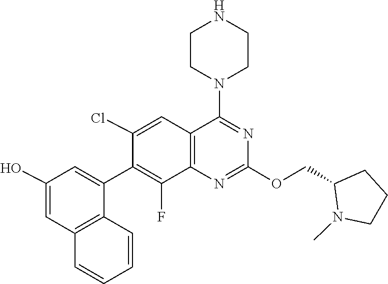 [SMILES: CN1CCC[C@H]1COc1nc(N2CCNCC2)c2cc(Cl)c(-c3cc(O)cc4ccccc34)c(F)c2n1] | 522.1 | HNMR (400 MHz, DMSO-d₆, ppm): δ 10.03 (brs, 1H), 7.93 (s, 1H), 7.81 (d, J = 8.4 Hz, 1H), 7.50-7.40 (m, 1H), 7.29 (d, J = 2.4 Hz, 1H), 7.22 (d, J = 2.4 Hz, 1H), 7.07 (d, J = 2.4 Hz, 1H), 4.40-4.36 (m, 1H), 4.19-4.15 (m, 1H), 3.78 (d, J = 2.4 Hz, 4H), 2.96-2.90 (m, 5H), 2.60-2.57 (m, 1H), 2.35 (d, J = 1.2 Hz, 3H), 2.25-2.15 (m, 1H), 2.01-1.90 (m, 1H), 1.70-1.65 (m, 3H). FNMR (376 MHz, DMSO-d₆, ppm): δ −122.42 (1F). |
| 3 | 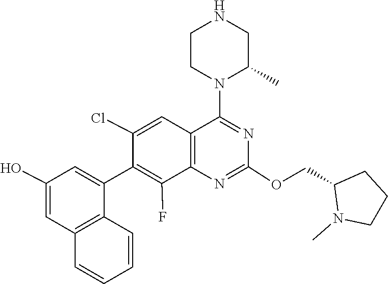 [SMILES: C[C@H]1CNCCN1c1nc(OC[C@@H]2CCCN2C)nc2c(F)c(-c3cc(O)cc4ccccc34)c(Cl)cc12] | 536.2 | FA salt, HNMR (300 MHz, DMSO-d₆, ppm): δ 8.22 (s, 1H), 7.87 (s, 1H), 7.83 (d, J = 8.4 Hz, 1H), 7.48-7.39 (m, 1H), 7.32-7.18 (m, 3H), 7.07 (s, 1H), 4.80-4.67 (m, 1H), 4.54-4.38 (m, 1H), 4.19-4.02 (m, 2H), 3.75-3.50 (m, 1H), 3.10-2.80 (m, 5H), 2.65-2.60 (m, 1H), 2.38 (s, 3H), 2.27- 2.13 (m, 1H), 2.05-1.88 (m, 1H), 1.80-1.60 (m, 3H), 1.55- 1.42 (m, 3H). FNMR (282 MHz, DMSO-d₆, ppm): δ −122.31 (1F). |
| 4 | 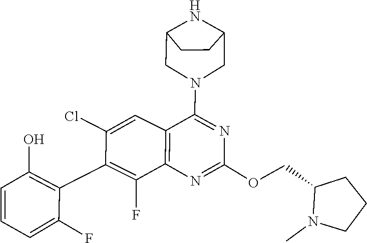 [SMILES: CN1CCC[C@H]1COc1nc(N2CC3CCC(C2)N3)c2cc(Cl)c(-c3c(O)cccc3F)c(F)c2n1] | 516.1 | HNMR (300 MHz, DMSO-d₆, ppm): δ 8.21 (s, 2H), 7.88 (s, 1H), 7.41-7.29 (m, 1H), 6.88-6.74 (m, 2H), 4.40-4.28 (m, 3H), 4.19-4.13 (m, 1H), 3.65-3.54 (m, 3H), 3.02-2.92 (m, 2H), 2.65-2.59 (m, 1H), 2.38 (s, 3H), 2.30-2.15 (m, 1H), 2.01-1.88 (m, 1H), 1.69-1.59 (m, 7H). FNMR (282 MHz, DMSO-d₆, ppm): δ −113.65 (1F), −121.06 (1F). |
| 5 | 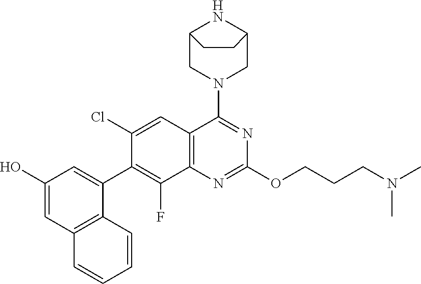 [SMILES: CN(C)CCCOc1nc(N2CC3CCC(C2)N3)c2cc(Cl)c(-c3cc(O)cc4ccccc34)c(F)c2n1] | 536.3 | HNMR (400 MHz, methanol-d₄, ppm): δ 7.97 (d, J = 1.6 Hz, 1H), 7.74 (d, J = 8.0 Hz, 1H), 7.42-7.38 (m, 1H), 7.26 (d, J = 2.4 Hz, 1H), 7.21-7.15 (m, 2H), 7.01 (d, J = 2.4 Hz, 1H), 4.69 (d, J = 13.6 Hz, 2H), 4.56 (t, J = 6.0 Hz, 2H), 4.28-4.22 (m, 2H), 3.86 (d, J = 14.0 Hz, 2H), 3.38-3.34 (m, 2H), 2.92 (s, 6H), 2.27-2.24 (m, 2H), 2.20-2.10 (m, 4H). FNMR (376 MHz, methanol-d₄, ppm): δ −123.17 (1F). |
| 6 | 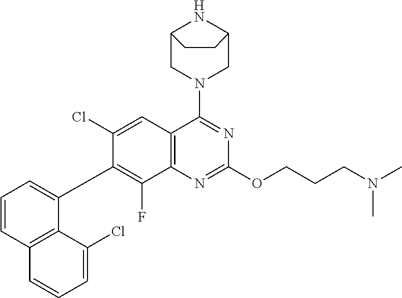 [SMILES: CN(C)CCCOc1nc(N2CC3CCC(C2)N3)c2cc(Cl)c(-c3cccc4cccc(Cl)c34)c(F)c2n1] | 554.3 | 3TFA salt, HNMR (400 MHz, DMSO-d₆, ppm): δ 9.68 (brs, 1H), 9.48-9.38 (m, 1H), 9.20 (brs, 1H), 8.23 (dd, J = 8.4, 1.2 Hz, 1H), 8.12 (dd, J = 8.4, 1.2 Hz, 1H), 7.93 (d, J = 1.6 Hz, 1H), 7.75 (dd, J = 8.4, 7.2 Hz, 1H), 7.67 (dd, J = 7.8, 1.4 Hz, 1H), 7.61-7.55 (m, 1H), 7.48 (dd, J = 7.2, 1.2 Hz, 1H), 4.54 (d, J = 13.8 Hz, 1H), 4.47-4.34 (m, 3H), 4.20 (d, J = 13.8 Hz, 2H), 3.95-3.65 (m, 2H), 3.30-3.20 (m, 2H), 2.91-2.78 (m, 6H), 2.22-2.10 (m, 2H), 2.02-1.93 (m, 4H). FNMR (376 MHz, DMSO-d₆, ppm): δ −121.94 (1F). |
| 7 | 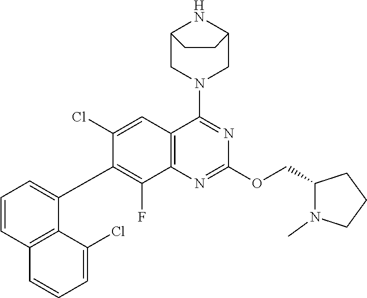 [SMILES: CN1CCC[C@H]1COc1nc(N2CC3CCC(C2)N3)c2cc(Cl)c(-c3cccc4cccc(Cl)c34)c(F)c2n1] | 566.3 | 3HCl salt, HNMR (300 MHz, DMSO-d₆, ppm): δ 10.91 (brs, 1H), 10.12-9.99 (m, 1H), 9.89-9.77 (m, 1H), 8.22 (d, J = 8.1 Hz, 1H), 8.12 (d, J = 8.1 Hz, 1H), 7.94 (d, J = 1.6 Hz, 1H), 7.74 (t, J = 7.8 Hz, 1H), 7.67 (d, J = 7.2 Hz, 1H), 7.57 (t, J = 7.8 Hz, 1H), 7.49 (d, J = 7.2 Hz, 1H), 4.80-4.64 (m, 2H), 4.55 (d, J = 13.8 Hz, 1H), 4.43 (d, J = 13.8 Hz, 1H), 4.25-4.12 (m, 2H), 4.10-3.99 (m, 1H), 3.95-3.79 (m, 3H), 3.20-3.05 (m, 1H), 2.93 (d, J = 4.8 Hz, 3H), 2.35-2.19 (m, 1H), 2.15-1.78 (m, 7H). FNMR (282 MHz, DMSO-d₆, ppm): δ −121.77 (1F). |
| 8 | 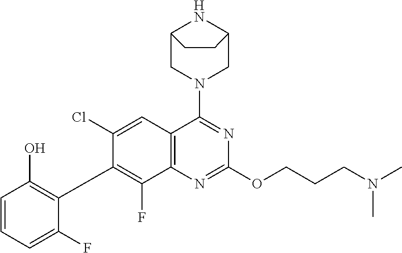 [SMILES: CN(C)CCCOc1nc(N2CC3CCC(C2)N3)c2cc(Cl)c(-c3c(O)cccc3F)c(F)c2n1] | 504.2 | 2FA salt, HNMR (300 MHz, DMSO-d₆, ppm): δ 8.26 (brs, 2H), 7.88 (s, 1H), 7.40-7.30 (m, 1H), 6.87 (d, J = 8.4 Hz, 1H), 6.79 (t, J = 8.7 Hz, 1H), 4.40-4.28 (m, 4H), 3.82-3.72 (m, 2H), 3.70-3.56 (m, 2H), 2.60-2.50 (m, 2H), 2.27 (s, 6H), 2.01-1.88 (m, 2H), 1.85-1.65 (m, 4H). FNMR (376 MHz, DMSO-d₆, ppm): δ −113.50 (1F), −120.99 (1F). |
| 9 | 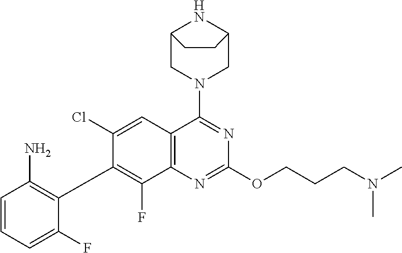 [SMILES: CN(C)CCCOc1nc(N2CC3CCC(C2)N3)c2cc(Cl)c(-c3c(N)cccc3F)c(F)c2n1] | 503.3 | HNMR (400 MHz, methanol-d₄, ppm): δ 7.97 (s, 1H), 7.25-7.15 (m, 1H), 6.67 (d, J = 8.0 Hz, 1H), 6.49 (t, J = 8.4 Hz, 1H), 4.70 (t, J = 12.8 Hz, 2H), 4.60 (t, J = 6.0 Hz, 2H), 4.23 (s, 2H), 3.94 (dd, J = 14.0, 4.8 Hz, 2H), 3.36 (t, J = 7.6 Hz, 2H), 2.92 (s, 6H), 2.31-2.23 (m, 2H), 2.14-2.04 (m, 4H). |
| 10 | 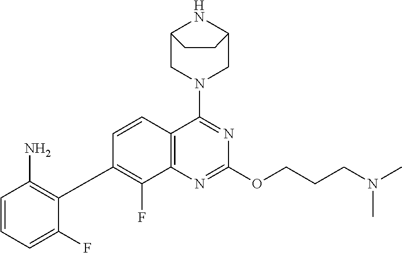 [SMILES: CN(C)CCCOc1nc(N2CC3CCC(C2)N3)c2ccc(-c3c(N)cccc3F)c(F)c2n1] | 469.3 | HNMR (400 MHz, methanol-d₄, ppm): δ 7.88 (d, J = 8.8 Hz, 1H), 7.35 (dd, J = 6.4, 8.8 Hz, 1H), 7.19-7.13 (m, 1H), 6.67 (d, J = 8.4 Hz, 1H), 6.50 (t, J = 9.2 Hz, 1H), 4.70 (d, J = 14.0 Hz, 2H), 4.61 (t, J = 5.6 Hz, 2H), 4.22 (s, 2H), 3.85 (d, J = 14.0 Hz, 2H), 3.37 (t, J = 7.6 Hz, 2H), 2.95 (s, 6H), 2.31-2.23 (m, 2H), 2.13-2.09 (m, 4H). |
| 12 | 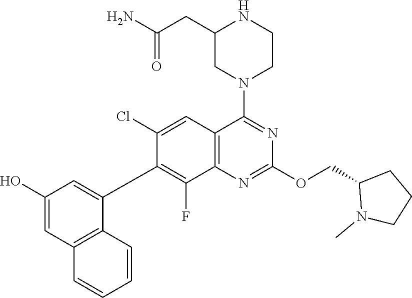 [SMILES: CN1CCC[C@H]1COc1nc(N2CCNC(CC(N)=O)C2)c2cc(Cl)c(-c3cc(O)cc4ccccc34)c(F)c2n1] | 579.1 | HNMR (400 MHz, methanol-d₄, ppm): δ 7.98 (s, 1H), 7.75-7.73 (m, 1H), 7.44-7.38 (m, 1H), 7.28-7.16 (m, 3H), 7.04-7.03 (m, 1H), 4.54-4.42 (m, 3H), 4.39-4.36 (m, 1H), 3.47-3.37 (m, 2H), 3.19-3.01 (m, 4H), 2.84-2.79 (m, 1H), 2.53 (s, 3H), 2.44-2.42 (m, 2H), 2.39-2.35 (m, 1H), 2.16- 2.07 (m, 1H), 1.83-1.73 (m, 3H). |
| 14 | 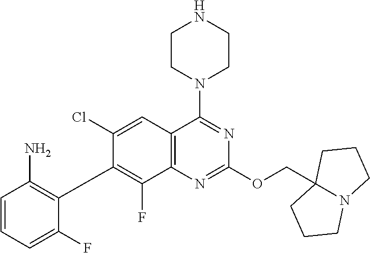 [SMILES: Nc1cccc(F)c1-c1c(Cl)cc2c(N3CCNCC3)nc(OCC34CCCN3CCC4)nc2c1F] | 515.3 | HNMR (400 MHz, methanol-d₄, ppm): δ 8.00 (s, 1H), 7.19 (dd, J = 14.8, 8.4 Hz, 1H), 6.65 (d, J = 8.4 Hz, 1H), 6.47 (t, J = 8.4 Hz, 1H), 4.65 (s, 2H), 4.12-4.09 (m, 4H), 3.71-3.64 (m, 2H), 3.50-3.45 (m, 4H), 3.35-3.25 (m, 2H), 2.35-2.10 (m, 8H). |
| 15 | 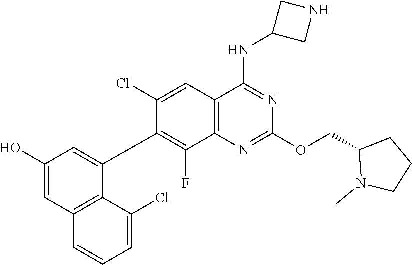 [SMILES: CN1CCC[C@H]1COc1nc(NC2CNC2)c2cc(Cl)c(-c3cc(O)cc4cccc(Cl)c34)c(F)c2n1] | 542.0 | HNMR (400 MHz, methanol-d₄, ppm): δ 8.20 (s, 1H), 7.75 (d, J = 7.4 Hz, 1H), 7.38-7.30 (m, 3H), 6.98 (d, J = 2.4 Hz, 1H), 5.23-5.16 (m, 2H), 4.66-4.61 (m, 1H), 4.52-4.46 (m, 2H), 4.39-4.35 (m, 2H), 3.86 (d, J = 8.6 Hz, 1H), 3.75-3.65 (m, 1H), 3.26-3.17 (m, 1H), 3.08 (s, 3H), 2.42-2.26 (m, 1H), 2.24-2.02 (m, 3H). |
| 16 | 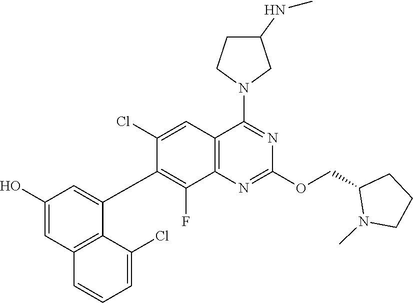 [SMILES: CNC1CCN(c2nc(OC[C@@H]3CCCN3C)nc3c(F)c(-c4cc(O)cc5cccc(Cl)c45)c(Cl)cc23)C1] | 570.1 | 1.66FA salt, HNMR (400 MHz, methanol-d₄, ppm): δ 8.29 (brs, 1.66H), 8.20 (s, 1H), 7.78-7.76 (d, J = 7.8 Hz, 1H), 7.39-7.32 (m, 3H), 7.00 (s, 1H), 4.67-4.64 (m, 2H), 4.32- 4.26 (m, 2H), 4.20-4.13 (m, 2H), 3.82-3.80 (d, J = 7.4 Hz, 2H), 3.66-3.63 (d, J = 11.4 Hz, 1H), 3.20-3.13 (m, 1H), 3.03 (s, 3H), 2.72 (s, 3H), 2.53-2.48 (m, 1H), 2.34-2.29 (m, 2H), 2.18-2.06 (m, 3H). |
| 17 | 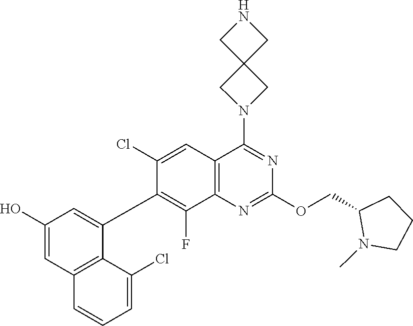 [SMILES: CN1CCC[C@H]1COc1nc(N2CC3(CNC3)C2)c2cc(Cl)c(-c3cc(O)cc4cccc(Cl)c34)c(F)c2n1] | 568.0 | 1.69FA salt, HNMR (400 MHz, methanol-d₄, ppm): δ 8.55 (brs, 1.69H), 7.79-7.73 (m, 2H), 7.37-7.29 (m, 3H), 6.95 (d, J = 2.4 Hz, 1H), 4.84-4.72 (m, 4H), 4.66-4.63 (m, 1H), 4.55-4.50 (m, 1H), 4.35-4.25 (m, 4H), 3.43-3.32 (m, 2H), 2.87-2.75 (m, 4H), 2.26-2.21 (d, J = 8.2 Hz, 1H), 2.01-1.92 (m, 3H). |
| 18 | 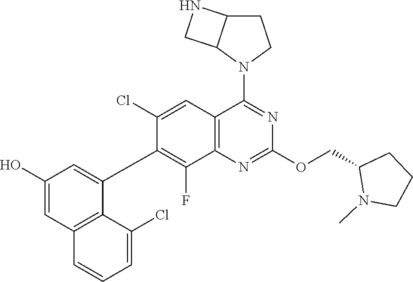 [SMILES: CN1CCC[C@H]1COc1nc(N2CCC3NCC32)c2cc(Cl)c(-c3cc(O)cc4cccc(Cl)c34)c(F)c2n1] | 568.1 | HNMR (400 MHz, methanol-d₄, ppm): δ 7.91 (s, 1H), 7.73 (d, J = 7.8 Hz, 1H), 7.36-7.29 (m, 3H), 6.97 (d, J = 2.6 Hz, 1H), 5.26-5.19 (m, 1H), 4.84-4.77 (m, 1H), 4.71-4.66 (t, J = 10.4 Hz, 1H), 4.53-4.33 (m, 3H), 4.18 (dd, J = 9.4, 5.8 Hz, 1H), 3.14-3.12 (m, 1H), 2.89-2.80 (m, 1H), 2.57-2.51 (m, 3H), 2.45-2.36 (m, 1H), 2.21-2.05 (m, 3H), 1.91-1.68 (m, 4H). |
| 19 | 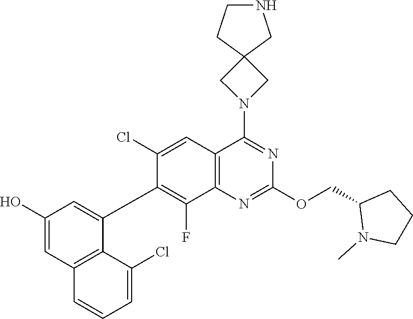 [SMILES: CN1CCC[C@H]1COc1nc(N2CC3(CCNC3)C2)c2cc(Cl)c(-c3cc(O)cc4cccc(Cl)c34)c(F)c2n1] | 582.1 | HNMR (400 MHz, methanol-d₄, ppm): δ 7.80 (d, J = 1.6 Hz, 1H), 7.72 (dd, J = 7.8, 1.6 Hz, 1H), 7.35-7.28 (m, 3H), 6.97-6.94 (d, J = 2.6 Hz, 1H), 4.58-4.37 (m, 6H), 3.22 (s, 2H), 3.15-3.04 (m, 3H), 2.84-2.77 (m, 1H), 2.53 (s, 3H), 2.41-2.34 (m, 1H), 2.24-2.20 (m, 2H), 2.16-2.06 (m, 1H), 1.86-1.89 (m, 3H). |
| 20 | 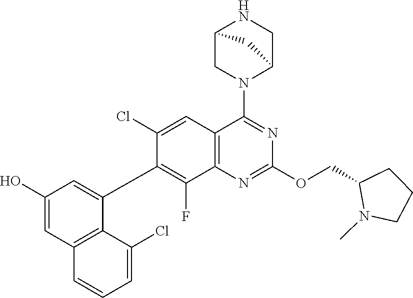 [SMILES: CN1CCC[C@H]1COc1nc(N2C[C@@H]3C[C@H]2CN3)c2cc(Cl)c(-c3cc(O)cc4cccc(Cl)c34)c(F)c2n1] | 568.1 | 0.74FA salt, HNMR (400 MHz, methanol-d₄, ppm): δ 8.81 (s, 1H), 871-8.35 (m, 0.74H), 7.75 (d, J = 7.8 Hz, 1H), 7.37-7.30 (m, 3H), 6.99 (s, 1H), 4.86-4.78 (m, 1H), 4.64- 4.57 (m, 1H), 4.43-4.39 (m, 1H), 3.79-3.69 (m, 1H), 3.63- 3.60 (m, 1H), 3.41-3.36 (m, 1H), 3.25-3.20 (m, 1H), 3.17- 3.11 (m, 1H), 3.05-2.99 (m, 6H), 2.37-2.31 (m, 1H), 2.20- 1.97 (m, 3H), 1.29-1.24 (m, 1H), 1.11-1.05 (m, 1H). |
| 21 | 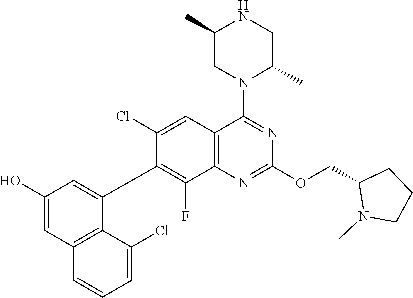 [SMILES: C[C@@H]1CN(c2nc(OC[C@@H]3CCCN3C)nc3c(F)c(-c4cc(O)cc5cccc(Cl)c45)c(Cl)cc23)[C@@H](C)CN1] | 584.0 | Mono FA salt, HNMR (400 MHz, methanol-d₄, ppm): δ 8.52 (brs, 1H), 7.96 (s, 1H), 7.75 (d, J = 7.8 Hz, 1H), 7.39- 7.28 (m, 3H), 6.98 (s, 1H), 4.75-4.71 (m, 1H), 4.62-4.57 (m, 1H), 4.33-4.22 (m, 1H), 3.87-3.84 (m, 1H), 3.57-3.39 (m, 5H), 3.02-2.99 (m, 1H), 2.91-2.86 (m, 4H), 2.29 (d, J = 7.4 Hz, 1H), 2.04-1.97 (m, 3H), 1.44-1.42 (m, 3H), 1.35- 1.20 (m, 3H). |
| 22 | 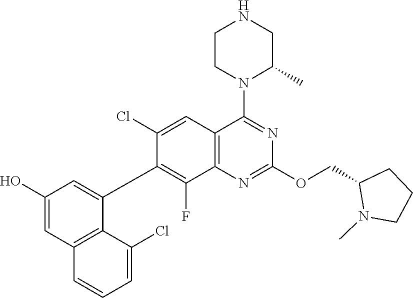 [SMILES: C[C@H]1CNCCN1c1nc(OC[C@@H]2CCCN2C)nc2c(F)c(-c3cc(O)cc4cccc(Cl)c34)c(Cl)cc12] | 570.1 | HNMR (400 MHz, methanol-d₄, ppm): δ 7.79 (s, 1H), 7.74 (d, J = 1.2 Hz, 1H), 7.39-7.30 (m, 3H), 6.98 (d, J = 2.5 Hz, 1H), 4.50-4.47 (m, 2H), 4.16-4.01 (m, 1H), 3.58-3.52 (m, 1H), 3.21-2.76 (m, 7H), 2.51 (s, 3H), 2.46-2.40 (m, 1H), 2.20-2.08 (m, 1H), 1.80-1.70 (m, 3H), 1.55-1.51 (m, 3H). |
| 23 | 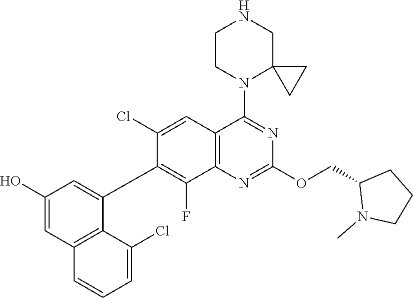 [SMILES: CN1CCC[C@H]1COc1nc(N2CCNCC23CC3)c2cc(Cl)c(-c3cc(O)cc4cccc(Cl)c34)c(F)c2n1] | 582.1 | HNMR (400 MHz, methanol-d₄, ppm): δ 7.85 (s, 1H), 7.73 (d, J = 1.2 Hz, 1H), 7.43-7.36 (m, 3H), 6.98 (s, 1H), 4.49- 4.45 (m, 2H), 4.43-3.76 (m, 3H), 3.08-2.68 (m, 5H), 2.52 (s, 3H), 2.48-2.45 (m, 1H), 2.18-2.06 (m, 1H), 1.90-1.70 (m, 3H), 1.07-1.00 (m, 2H), 0.99-0.96 (m, 2H). |
| 24 | 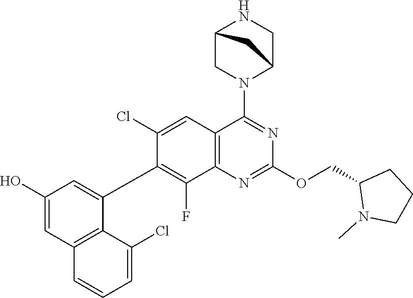 [SMILES: CN1CCC[C@H]1COc1nc(N2C[C@H]3C[C@@H]2CN3)c2cc(Cl)c(-c3cc(O)cc4cccc(Cl)c34)c(F)c2n1] | 568.1 | 0.36FA salt, HNMR (400 MHz, methanol-d₄, ppm): δ 8.79 (s, 1H), 8.67-8.42 (m, 0.36H), 7.74 (d, J = 7.8 Hz, 1H), 7.36-7.30 (m, 3H), 7.02-6.98 (m, 1H), 4.64-4.51 (m, 2H), 4.42-4.38 (m, 1H), 3.37-3.22 (m, 4H), 3.07-2.97 (m, 3H), 2.81-2.69 (m, 4H), 2.24-2.19 (m, 1H), 2.05-1.85 (m, 3H), 1.27-1.22 (m, 1H), 1.10-1.04 (m, 1H). |
| 27 | 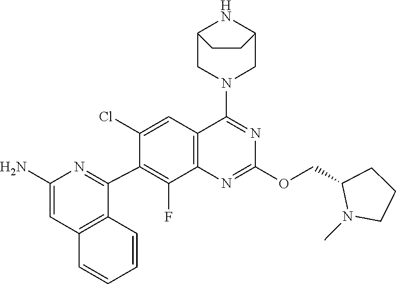 [SMILES: CN1CCC[C@H]1COc1nc(N2CC3CCC(C2)N3)c2cc(Cl)c(-c3nc(N)cc4ccccc34)c(F)c2n1] | 548.3 | HNMR (400 MHz, methanol-d₄, ppm): δ 8.03-8.01 (m, 1H), 7.73-7.69 (m, 1H), 7.61-7.56 (m, 1H), 7.35-7.32 (m, 1H), 7.24-7.19 (m, 1H), 7.13 (s, 1H), 4.98-4.88 (m, 1H), 4.74-4.62 (m, 3H), 4.24 (s, 2H), 3.96-3.83 (m, 3H), 3.77- 3.69 (m, 1H), 3.26-3.18 (m, 1H), 3.08 (s, 3H), 2.43-2.33 (m, 1H), 2.25-2.02 (m, 7H). |
| 29 | 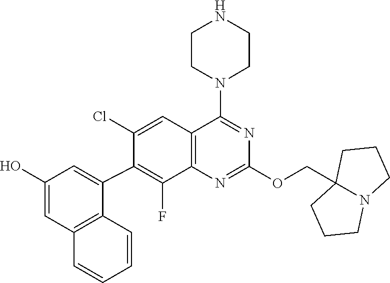 [SMILES: Oc1cc(-c2c(Cl)cc3c(N4CCNCC4)nc(OCC45CCCN4CCC5)nc3c2F)c2ccccc2c1] | 548.3 | 2TFA salt, HNMR (400 MHz, DMSO-d₆, ppm): δ 10.37 (s, 1H), 9.15 (s, 2H), 8.08 (s, 1H), 7.79 (d, J = 8.0 Hz, 1H), 7.44-7.40 (m, 1H), 7.27 (d, J = 2.0 Hz, 1H), 7.22-7.18 (m, 1H), 7.14-7.12 (m, 1H), 7.04 (d, J = 2.4 Hz, 1H), 4.53 (s, 2H), 4.05-3.95 (m, 4H), 3.52-3.46 (m, 2H), 3.40-3.25 (m, 4H), 3.21-3.17 (m, 2H), 2.20-1.94 (m, 8H). |
| 31 | 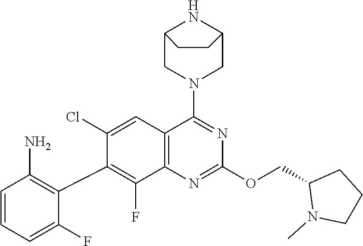 [SMILES: CN1CCC[C@H]1COc1nc(N2CC3CCC(C2)N3)c2cc(Cl)c(-c3c(N)cccc3F)c(F)c2n1] | 515.3 | HNMR (400 MHz, methanol-d₄, ppm): δ 7.95 (d, J = 1.2 Hz, 1H), 7.22-7.16 (m, 1H), 6.65 (d, J = 8.4 Hz, 1H), 6.48- 6.44 (m, 1H), 4.89-4.87 (m, 1H), 4.70-4.62 (m, 3H), 4.21 (s, 2H), 3.88-3.83 (m, 3H), 3.73-3.71 (m, 1H), 3.22-3.19 (m, 1H), 3.08 (s, 3H), 2.42-2.36 (m, 1H), 2.21-2.04 (m, 7H). FNMR (376 MHz, methanol-d₄, ppm): δ −116.64 (1F), −122.13 (1F). |
| 32 | 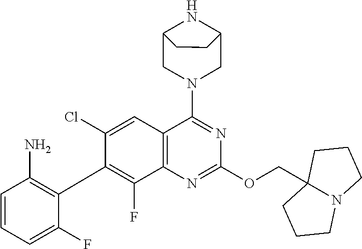 [SMILES: Nc1cccc(F)c1-c1c(Cl)cc2c(N3CC4CCC(C3)N4)nc(OCC34CCCN3CCC4)nc2c1F] | 541.3 | HNMR (400 MHz, methanol-d₄, ppm): δ 7.96 (d, J = 1.2 Hz, 1H), 7.22-7.16 (m, 1H), 6.65 (d, J = 8.4 Hz, 1H), 6.49- 6.44 (m, 1H), 4.73-4.64 (m, 4H), 4.21 (s, 2H), 3.90-3.85 (m, 2H), 3.71-3.64 (m, 2H), 3.29-3.25 (m, 2H), 2.33-2.28 (m, 2H), 2.25-2.15 (m, 10H). FNMR (376 MHz, methanol- d₄, ppm): δ −116.61 (1F), −122.29 (1F). |
| 33 | 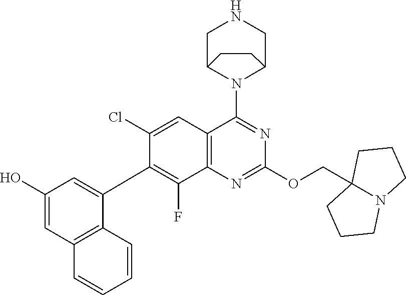 [SMILES: Oc1cc(-c2c(Cl)cc3c(N4C5CCC4CNC5)nc(OCC45CCCN4CCC5)nc3c2F)c2ccccc2c1] | 574.3 | 3TFA salt, HNMR (400 MHz, DMSO-d₆, ppm): δ 10.40 (s, 1H), 10.10 (s, 1H), 9.66 (s, 1H), 9.07 (s, 1H), 8.07 (s, 1H), 7.79 (d, J = 8.0 Hz, 1H), 7.44-7.40 (m, 1H), 7.28 (d, J = 2.4 Hz, 1H), 7.22-7.20 (m, 1H), 7.18-7.13 (m, 1H), 7.04 (d, J = 2.4 Hz, 1H), 5.06 (s, 2H), 4.53 (s, 2H), 3.50-3.47 (m, 4H), 3.35-3.33 (m, 2H), 3.21-3.17 (m, 2H), 2.15-1.94 (m, 12H). |
| 34 | 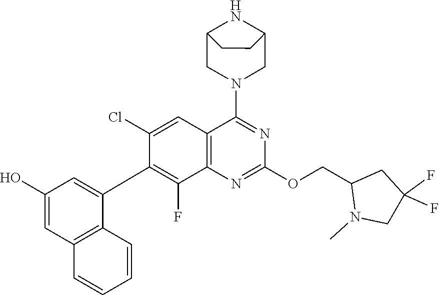 [SMILES: CN1CC(F)(F)CC1COc1nc(N2CC3CCC(C2)N3)c2cc(Cl)c(-c3cc(O)cc4ccccc34)c(F)c2n1] | 584.3 | HNMR (400 MHz, methanol-d₄, ppm): δ 8.02-7.97 (m, 1H), 7.75-7.72 (m, 1H), 7.42-7.38 (m, 1H), 7.26 (d, J = 2.4 Hz, 1H), 7.21-7.14 (m, 2H), 7.02-7.01 (m, 1H), 4.95-4.92 (m, 1H), 4.74-4.69 (m, 3H), 4.23-4.02 (m, 4H), 3.91-3.87 (m, 2H), 3.76-3.65 (m, 1H), 3.08 (s, 3H), 2.97-2.84 (m, 1H), 2.76-2.62 (m, 1H), 2.21-2.01 (m, 4H). FNMR (376 MHz, methanol-d₄, ppm): δ −98.01 (2F), −123.09 (1F). |
| 35 | 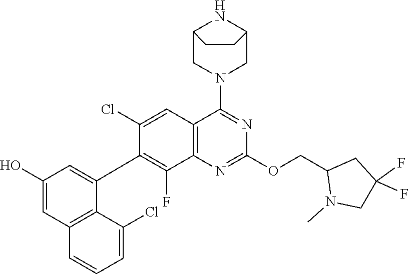 [SMILES: CN1CC(F)(F)CC1COc1nc(N2CC3CCC(C2)N3)c2cc(Cl)c(-c3cc(O)cc4cccc(Cl)c34)c(F)c2n1] | 618.2 | HNMR (400 MHz, methanol-d₄, ppm): δ 7.90 (d, J = 0.8 Hz, 1H), 7.73 (dd, J = 8.4, 1.2 Hz, 1H), 7.36-7.28 (m, 3H), 6.96-6.94 (m, 1H), 5.00-4.92 (m, 1H), 4.73-4.59 (m, 3H), 4.24-4.21 (m, 2H), 4.15-3.95 (m, 2H), 3.91-3.79 (m, 2H), 3.71-3.50 (m, 1H), 3.08-2.98 (m, 3H), 2.91-2.81 (m, 1H), 2.74-2.55 (m, 1H), 2.23-2.07 (m, 4H). FNMR (376 MHz, methanol-d₄, ppm): δ −98.05 (2F), −123.38 (1F). |
| 36 | 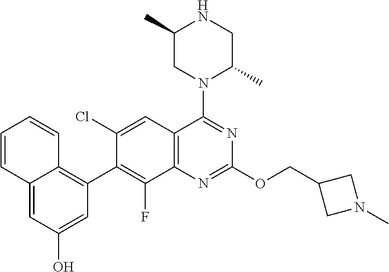 [SMILES: C[C@@H]1CN(c2nc(OCC3CN(C)C3)nc3c(F)c(-c4cc(O)cc5ccccc45)c(Cl)cc23)[C@@H](C)CN1] | 536.2 | 1.78FA salt, HNMR (400 MHz, methanol-d₄, ppm): δ 8.53 (brs, 1.78H), 8.08 (s, 1H), 7.77 (d, J = 8.4 Hz, 1H), 7.46- 7.37 (m, 1H), 7.29 (s, 1H), 7.22-7.19 (m, 2H), 7.07-7.00 (m, 1H), 4.46-4.34 (m, 3H), 4.31-4.21 (m, 5H), 3.97-3.94 (m, 1H), 3.66-3.63 (m, 2H), 3.54-.351 (m, 1H), 3.18-3.13 (m, 1H), 2.93 (s, 3H), 1.49-1.47 (m, 3H), 1.35-1.33 (m, 3H). |
| 37 | 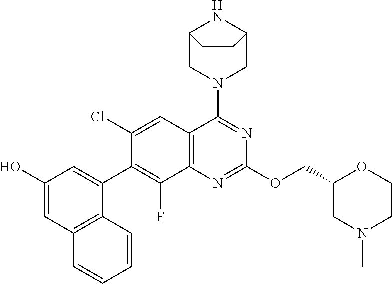 [SMILES: CN1CCO[C@@H](COc2nc(N3CC4CCC(C3)N4)c3cc(Cl)c(-c4cc(O)cc5ccccc45)c(F)c3n2)C1] | 564.3 | HNMR (400 MHz, methanol-d₄, ppm): δ 7.96-7.95 (m, 1H), 7.75-7.72 (m, 1H), 7.42-7.37 (m, 1H), 7.25 (d, J = 2.4 Hz, 1H), 7.21-7.14 (m, 2H), 7.01-7.00 (m, 1H), 4.69-4.52 (m, 4H), 4.22-4.12 (m, 4H), 3.89-3.81 (m, 3H), 3.66-3.43 (m, 2H), 3.22-3.08 (m, 2H), 2.93 (s, 3H), 2.20-2.06 (m, 4H). FNMR (376 MHz, methanol-d₄, ppm): δ −122.70 (1F). |
| 38 | 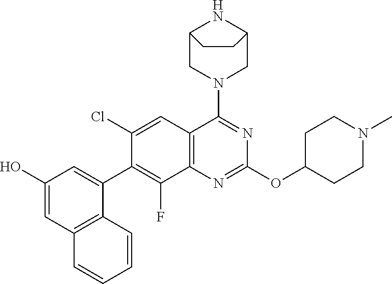 [SMILES: CN1CCC(Oc2nc(N3CC4CCC(C3)N4)c3cc(Cl)c(-c4cc(O)cc5ccccc45)c(F)c3n2)CC1] | 548.2 | HNMR (400 MHz, methanol-d₄, ppm): δ 7.94 (s, 1H), 7.74 (d, J = 8.4 Hz, 1H), 7.40 (t, J = 6.8 Hz, 1H), 7.26-7.16 (m, 3H), 7.02 (d, J = 2.4 Hz, 1H), 5.25-5.13 (m, 1H), 4.48 (d, J = 11.2 Hz, 2H), 3.70-3.60 (m, 4H), 2.88-2.68 (m, 2H), 2.56-2.40 (m, 2H), 2.33 (s, 3H), 2.20-2.05 (m, 2H), 2.00- 1.78 (m, 6H). |
| 39 | 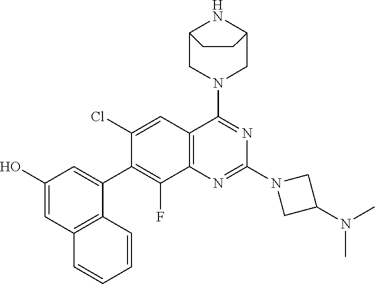 [SMILES: CN(C)C1CN(c2nc(N3CC4CCC(C3)N4)c3cc(Cl)c(-c4cc(O)cc5ccccc45)c(F)c3n2)C1] | 533.2 | 2FA salt, HNMR (400 MHz, methanol-d₄, ppm): δ 8.56- 8.38 (m, 2H), 7.80 (s, 1H), 7.76-7.74 (m, 1H), 7.43-7.39 (m, 1H), 7.26-7.17 (m, 3H), 7.01-6.98 (m, 1H), 4.56-4.52 (m, 2H), 4.34-4.30 (m, 2H), 4.17-4.08 (m, 4H), 3.79-3.75 (m, 2H), 3.48-3.43 (m, 1H), 2.38 (s, 6H), 2.19-2.04 (m, 4H). |
| 40 | 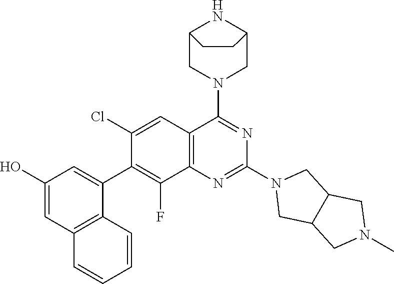 [SMILES: CN1CC2CN(c3nc(N4CC5CCC(C4)N5)c4cc(Cl)c(-c5cc(O)cc6ccccc56)c(F)c4n3)CC2C1] | 559.1 | HNMR (400 MHz, methanol-d₄, ppm): δ 7.90 (s, 1H), 7.76 (d, J = 8.3 Hz, 1H), 7.40-7.44 (m, 1H), 7.28-7.17 (m, 3H), 7.01 (d, J = 2.4 Hz, 1H), 4.71 (d, J = 13.0 Hz, 2H), 4.29- 4.21 (m, 2H), 4.07-3.67 (m, 8H), 3.53-3.36 (m, 3H), 3.08- 2.87 (m, 4H), 2.18-2.14 (m, 4H). |
| 41 | 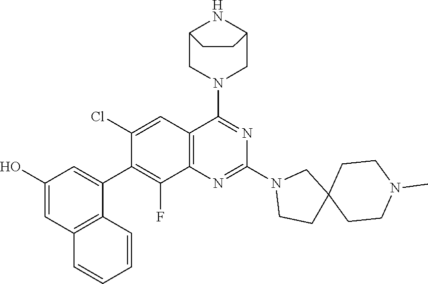 [SMILES: CN1CCC2(CC1)CCN(c1nc(N3CC4CCC(C3)N4)c3cc(Cl)c(-c4cc(O)cc5ccccc45)c(F)c3n1)C2] | 587.2 | HNMR (400 MHz, methanol-d₄, ppm): δ 7.75-7.72 (m, 2H), 7.40 (t, J = 1.2 Hz, 1H), 7.38-7.17 (m, 3H), 7.01 (s, 1H), 4.47-4.37 (m, 2H), 3.75-3.64 (m, 4H), 3.60-3.51 (m, 4H), 2.74-2.50 (m, 4H), 2.48 (s, 3H), 1.99-1.81 (m, 6H), 1.70-1.53 (m, 4H). |
| 43 | 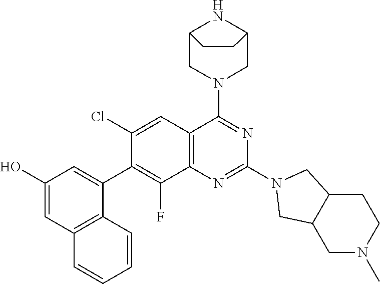 [SMILES: CN1CCC2CN(c3nc(N4CC5CCC(C4)N5)c4cc(Cl)c(-c5cc(O)cc6ccccc56)c(F)c4n3)CC2C1] | 573.1 | HNMR (400 MHz, methanol-d₄, ppm): δ 7.73 (d, J = 8.6 Hz, 2H), 7.40 (t, J = 8.1 Hz, 1H), 7.24 (d, J = 2.5 Hz, 2H), 7.21-7.16 (m, 1H), 7.02-6.99 (m, 1H), 4.45-4.38 (m, 2H), 3.80-3.49 (m, 9H), 2.61-2.30 (m, 8H), 1.95-1.85 (m, 5H). |
| 44 | 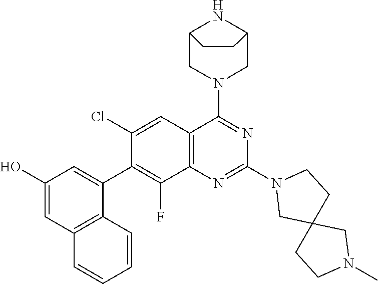 [SMILES: CN1CCC2(CCN(c3nc(N4CC5CCC(C4)N5)c4cc(Cl)c(-c5cc(O)cc6ccccc56)c(F)c4n3)C2)C1] | 573.2 | 4FA salt, HNMR (400 MHz, methanol-d₄, ppm): δ 8.44 (s, 4H), 7.78 (s, 1H), 7.75 (t, J = 1.2 Hz, 1H), 7.40 (t, J = 1.2 Hz, 1H), 7.25-7.18 (m, 3H), 7.00 (s, 1H), 4.49-4.37 (m, 2H), 4.17-4.06 (m, 2H), 3.75-3.61 (m, 6H), 3.50-3.31 (m, 4H), 2.90 (s, 3H), 2.16-2.01 (m, 8H). |
| 46 | 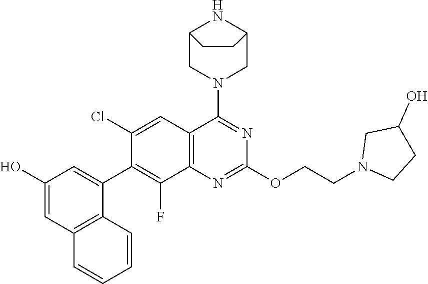 [SMILES: Oc1cc(-c2c(Cl)cc3c(N4CC5CCC(C4)N5)nc(OCCN4CCC(O)C4)nc3c2F)c2ccccc2c1] | 564.1 | HNMR (400 MHz, methanol-d₄, ppm): δ 7.95 (s, 1H), 7.74 (d, J = 8.4 Hz, 1H), 7.40 (t, J = 7.5 Hz, 1H), 7.25 (d, J = 2.4 Hz, 1H), 7.20 (dd, J = 13.4, 7.0 Hz, 2H), 7.02 (d, J = 2.4 Hz, 1H), 4.58 (t, J = 5.8 Hz, 2H), 4.51 (d, J = 10.7 Hz, 2H), 4.39-4.30 (m, 1H), 3.64 (d, J = 9.9 Hz, 4H), 3.02-2.77 (m, 3H), 2.87 (dd, J = 16.3, 7.8 Hz, 1H), 2.75-2.64 (m, 2H), 2.20-2.10 (m, 1H), 1.87-1.65 (m, 5H). |
| 47 | 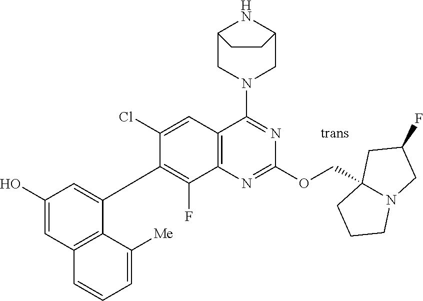 [SMILES: Cc1cccc2cc(O)cc(-c3c(Cl)cc4c(N5CC6CCC(C5)N6)nc(OC[C@@]56CCCN5C[C@H](F)C6)nc4c3F)c12] | 606.3 | HNMR (400 MHz, methanol-d₄, ppm): δ 7.96-7.95 (m, 1H), 7.62 (d, J = 8.0 Hz, 1H), 7.29-7.25 (m, 2H), 7.03 (d, J = 7.2 Hz, 1H), 6.83-6.82 (m, 1H), 5.61-5.48 (m, 1H), 4.79- 4.74 (m, 1H), 4.70-4.62 (m, 3H), 4.24-4.23 (m, 2H), 4.03- 3.81 (m, 5H), 3.48-3.41 (m, 1H), 2.75-2.52 (m, 2H), 2.41- 2.29 (m, 3H), 2.18-2.09 (m, 5H), 1.99 (s, 3H). FNMR (376 MHz, methanol-d₄, ppm): δ −123.22 (1F), −174.30 (1F). |
| 48 | 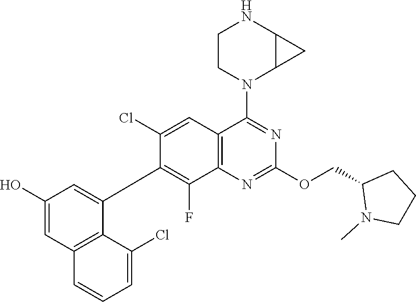 [SMILES: CN1CCC[C@H]1COc1nc(N2CCNC3CC32)c2cc(Cl)c(-c3cc(O)cc4cccc(Cl)c34)c(F)c2n1] | 568.1 | HNMR (400 MHz, methanol-d₄, ppm): δ 8.02 (s, 1H), 7.75 (d, J = 7.9 Hz, 1H), 7.34 (dd, J = 14.3, 4.7 Hz, 3H), 7.01- 6.93 (m, 1H), 5.35-5.30 (m, 1H), 4.52-4.42 (m, 2H), 4.38- 4.27 (m, 1H), 3.99-3.85 (m, 2H), 3.35-3.25 (m, 1H), 3.17- 3.06 (m, 2H), 2.89-2.80 (m, 1H), 2.56 (s, 3H), 2.49-2.38 (m, 1H), 2.25-1.70 (m, 6H). |
| 49 | 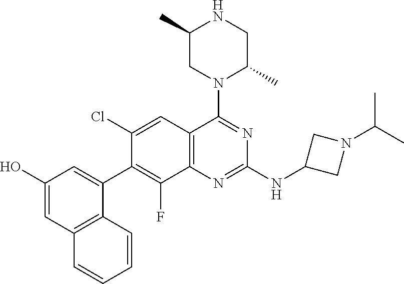 [SMILES: CC(C)N1CC(Nc2nc(N3C[C@@H](C)NC[C@@H]3C)c3cc(Cl)c(-c4cc(O)cc5ccccc45)c(F)c3n2)C1] | 549.2 | HNMR (400 MHz, methanol-d₄, ppm): δ 8.03 (s, 1H), 7.74 (d, J = 8.0 Hz, 1H), 7.41 (t, J = 8.0 Hz, 1H), 7.28-7.09 (m, 3H), 6.98 (d, J = 2.0 Hz, 1H), 4.73 (s, 1H), 4.46-4.38 (m, 2H), 4.24 (t, J = 5.0 Hz, 1H), 4.06-3.98 (m, 1H), 3.80-3.73 (m, 1H), 3.58-3.49 (m, 1H), 3.48-3.40 (m, 2H), 3.25-3.15 (m, 2H), 3.13-3.06 (m, 1H), 1.45-1.32 (m, 12H). |
| 51 | 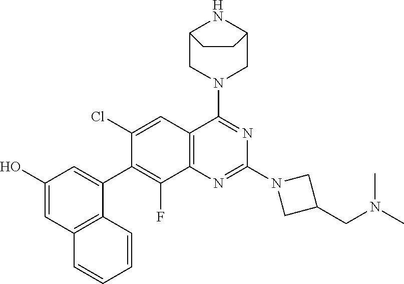 [SMILES: CN(C)CC1CN(c2nc(N3CC4CCC(C3)N4)c3cc(Cl)c(-c4cc(O)cc5ccccc45)c(F)c3n2)C1] | 547.2 | HNMR (400 MHz, methanol-d₄, ppm): δ 7.76-7.72 (m, 2H), 7.43-7.36 (m, 1H), 7.25-7.23 (m, 2H), 7.20-7.18 (m, 1H), 7.00 (d, J = 4 Hz, 1H), 4.39-4.26 (m, 4H), 3.87-3.84 (m, 2H), 3.61 (m, 2H), 3.54-3.51 (m, 2H), 2.95-2.92 (m, 1H), 2.67 (d, J = 8 Hz, 2H), 2.29 (s, 6H), 1.94-1.81 (m, 4H). |
| 52 | 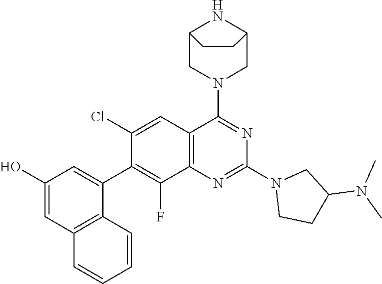 [SMILES: CN(C)C1CCN(c2nc(N3CC4CCC(C3)N4)c3cc(Cl)c(-c4cc(O)cc5ccccc45)c(F)c3n2)C1] | 547.3 | HNMR (400 MHz, methanol-d₄, ppm): δ 7.75-7.72 (m, 2H), 7.41-7.37 (m, 1H), 7.27-7.23 (m, 2H), 7.21-7.15 (m, 1H), 7.00 (d, J = 4 Hz, 1H), 4.39-4.31 (m, 2H), 4.05-3.89 (m, 2H), 3.65-3.48 (m, 5H), 3.42-3.38 (m, 1H), 2.95-2.88 (m, 1H), 2.35 (s, 6H), 2.30-2.24 (m, 1H), 1.95-1.84 (m, 5H). |
| 53 | 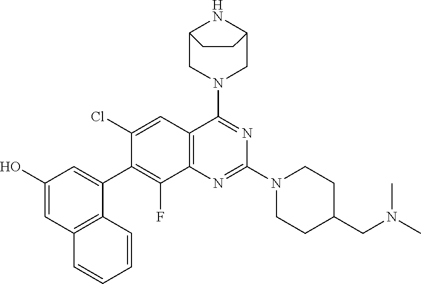 [SMILES: CN(C)CC1CCN(c2nc(N3CC4CCC(C3)N4)c3cc(Cl)c(-c4cc(O)cc5ccccc45)c(F)c3n2)CC1] | 575.1 | FA salt, HNMR (400 MHz, methanol-d₄, ppm): δ 8.53 (s, 1H), 7.75-7.73 (m, 2H), 7.42-7.38 (m, 1H), 7.25-7.24 (m, 2H), 7.20-7.17 (m, 1H), 7.01-6.99 (m, 1H), 4.95-4.91 (m, 2H), 4.40-4.37 (m, 2H), 3.99-3.93 (m, 2H), 3.64-3.61 (m, 2H), 3.03-2.97 (m, 2H), 2.81-2.79 (m, 2H), 2.71 (s, 6H), 2.13-1.98 (m, 5H), 1.86-1.83 (m, 2H), 1.30-1.22 (m, 2H). |
| 54 | 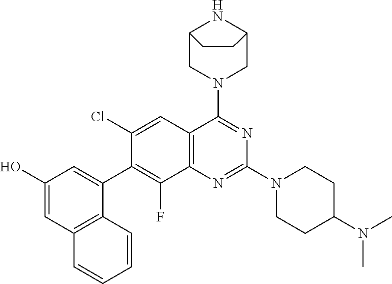 [SMILES: CN(C)C1CCN(c2nc(N3CC4CCC(C3)N4)c3cc(Cl)c(-c4cc(O)cc5ccccc45)c(F)c3n2)CC1] | 561.1 | 2FA salt, HNMR (400 MHz, methanol-d₄, ppm): δ 8.50 (s, 2H), 7.76 (dd, J = 13.6, 4.8 Hz, 2H), 7.47-7.36 (m, 1H), 7.28-7.15 (m, 3H), 7.00 (d, J = 2.4 Hz, 1H), 5.10 (d, J = 13.6 Hz, 2H), 4.45 (d, J = 13.8 Hz, 2H), 4.12 (s, 2H), 3.71 (d, J = 13.6 Hz, 2H), 3.50-3.35 (m, 1H), 2.99 (t, J = 12.2 Hz, 2H), 2.81 (s, 6H), 2.24-2.05 (m, 6H), 1.80-1.58 (m, 2H). |
| 55 | 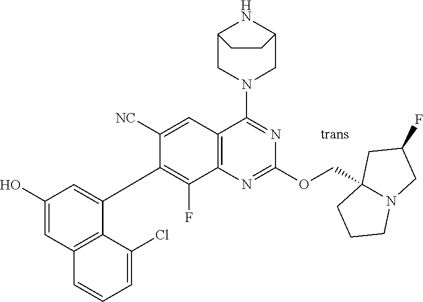 [SMILES: N#Cc1cc2c(N3CC4CCC(C3)N4)nc(OC[C@@]34CCCN3C[C@H](F)C4)nc2c(F)c1-c1cc(O)cc2cccc(Cl)c12] | 617.3 | HNMR (400 MHz, methanol-d₄, ppm): δ 8.31-8.29 (m, 1H), 7.77 (d, J = 8.0 Hz, 1.6 Hz, 1H), 7.40-7.32 (m, 3H), 7.07 (d, J = 2.4 Hz, 1H), 5.62-5.48 (m, 1H), 4.76-4.65 (m, 3H), 4.30-4.16 (m, 2H), 4.06-3.80 (m, 5H), 3.50-3.40 (m, 1H), 2.80-2.00 (m, 11H). FNMR (376 MHz, methanol-d₄, ppm): δ −125.06 (1F), −174.20 (1F). |
| 56 | 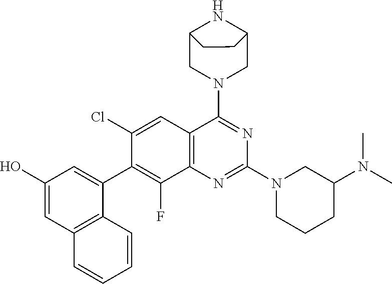 [SMILES: CN(C)C1CCCN(c2nc(N3CC4CCC(C3)N4)c3cc(Cl)c(-c4cc(O)cc5ccccc45)c(F)c3n2)C1] | 561.1 | HNMR (400 MHz, methanol-d₄, ppm): δ 7.73 (d, J = 8.2 Hz, 2H), 7.39 (t, J = 7.6 Hz, 1H), 7.28-7.15 (m, 3H), 7.01 (d, J = 2.4 Hz, 1H), 4.99-4.70 (m, 2H), 4.31 (t, J = 11.4 Hz, 2H), 3.65-3.60 (m, 2H), 3.51 (dd, J = 12.2, 8.4 Hz, 2H), 2.99-2.90 (m, 2H), 2.38-2.32 (m, 7H), 2.08 (s, 1H), 1.95-1.79 (m, 5H), 1.61-1.54 (m, 2H). |
| 57 | 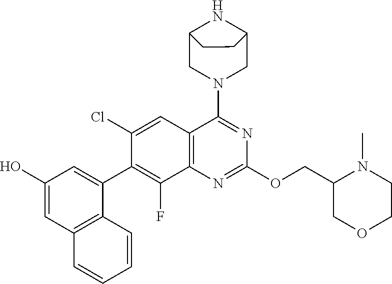 [SMILES: CN1CCOCC1COc1nc(N2CC3CCC(C2)N3)c2cc(Cl)c(-c3cc(O)cc4ccccc34)c(F)c2n1] | 564.1 | HNMR (400 MHz, methanol-d₄, ppm): δ 7.94-7.95 (d, J = 1.6 Hz, 1H), 7.73-7.75 (d, J = 8.4 Hz, 1H), 7.42-7.38 (m, 1H), 7.26-7.17 (m, 3H), 7.02-7.03 (d, J = 2.4 Hz, 1H), 4.63-4.58 (m, 1H), 4.51 (d, J = 11.4 Hz, 2H), 4.45-4.40 (m, 1H), 3.99 (dd, J = 11.4, 3.2 Hz, 1H), 3.79 (d, J = 11.6 Hz, 1H), 3.67-3.61 (m, 5H), 3.54-3.49 (m, 1H), 2.76 (dd, J = 9.6, 2.4 Hz, 1H), 2.64-2.59 (m, 1H), 2.45-2.36 (m, 4H), 1.90-1.83 (m, 4H). |
| 58 | 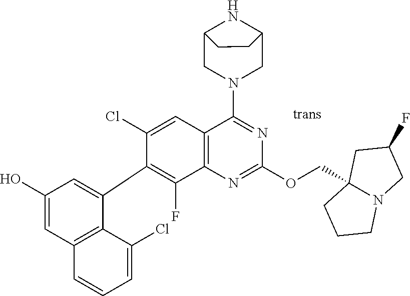 [SMILES: Oc1cc(-c2c(Cl)cc3c(N4CC5CCC(C4)N5)nc(OC[C@@]45CCCN4C[C@H](F)C5)nc3c2F)c2c(Cl)cccc2c1] | 626.3 | HNMR (400 MHz, methanol-d₄, ppm): δ 7.91 (d, J = 1.6 Hz, 1H), 7.73 (dd, J = 8.0, 1.2 Hz, 1H), 7.36-7.28 (m, 3H), 6.95 (d, J = 2.4 Hz, 1H), 5.61-5.47 (m, 1H), 4.87-4.60 (m, 4H), 4.27-4.16 (m, 2H), 4.04-3.80 (m, 5H), 3.47-3.40 (m, 1H), 2.75-2.12 (m, 10H). FNMR (376 MHz, methanol-d₄, ppm): δ −123.79 (1F), −174.30 (1F). |
| 59 | 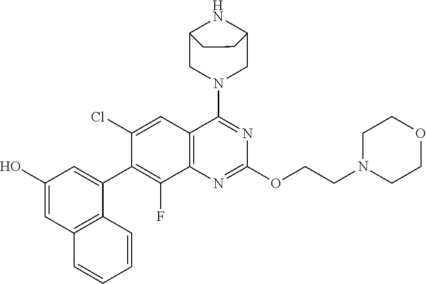 [SMILES: Oc1cc(-c2c(Cl)cc3c(N4CC5CCC(C4)N5)nc(OCCN4CCOCC4)nc3c2F)c2ccccc2c1] | 564.1 | HNMR (400 MHz, methanol-d₄, ppm): δ 7.95 (d, J = 1.6 Hz, 1H), 7.74 (d, J = 8.4 Hz, 1H), 7.42-7.38 (m, 1H), 7.25- 7.17 (m, 3H), 7.02 (d, J = 2.4 Hz, 1H), 4.60 (t, J = 5.7 Hz, 2H), 4.51 (d, J = 11.5 Hz, 2H), 3.75-3.65 (m, 8H), 2.84 (t, J = 5.7 Hz, 2H), 2.67-2.58 (m, 4H), 1.87-1.83 (m, 4H). |
| 61 | 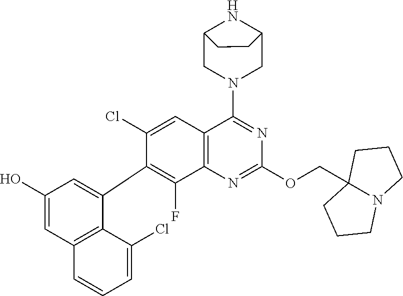 [SMILES: Oc1cc(-c2c(Cl)cc3c(N4CC5CCC(C4)N5)nc(OCC45CCCN4CCC5)nc3c2F)c2c(Cl)cccc2c1] | 608.3 | 3TFA salt, HNMR (400 MHz, DMSO-d₆, ppm): δ 10.36 (s, 1H), 10.28-10.18 (m, 1H), 9.38-9.28 (m, 1H), 9.12-9.02 (m, 1H), 7.90 (s, 1H), 7.83-7.81 (m, 1H), 7.40-7.31 (m, 3H), 6.95 (d, J = 2.4 Hz, 1H), 4.57-4.49 (m, 3H), 4.38 (d, J = 13.2 Hz, 1H), 4.16 (d, J = 16.0 Hz, 2H), 3.83 (d, J = 13.2 Hz, 1H), 3.67 (d, J = 13.6 Hz, 1H), 3.47-3.44 (m, 2H), 3.20-3.16 (m, 2H), 2.15-2.05 (m, 4H), 2.03-1.87 (m, 8H). FNMR (376 MHz, DMSO-d₆, ppm): δ −122.12 (1F). |
| 63 | 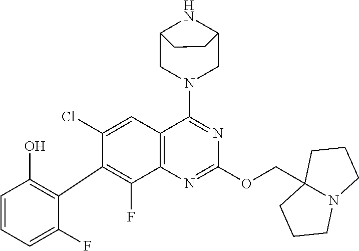 [SMILES: Oc1cccc(F)c1-c1c(Cl)cc2c(N3CC4CCC(C3)N4)nc(OCC34CCCN3CCC4)nc2c1F] | 542.2 | HNMR (400 MHz, DMSO-d₆, ppm): δ 10.29 (brs, 1H), 7.88 (d, J = 1.6 Hz, 1H), 7.35 (td, J = 8.4, 6.8 Hz, 1H), 6.89-6.76 (m, 2H), 4.31 (t, J = 10.0 Hz, 2H), 4.11 (s, 2H), 3.72-3.45 (m, 4H), 3.10-3.01 (m, 2H), 2.72-2.62 (m, 2H), 2.00-1.74 (m, 6H), 1.73-1.59 (m, 6H). FNMR (376 MHz, DMSO-d₆, ppm): δ −113.65 (1F), −121.10 (1F). |
| 64 | 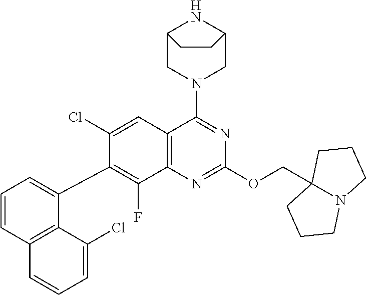 [SMILES: Fc1c(-c2cccc3cccc(Cl)c23)c(Cl)cc2c(N3CC4CCC(C3)N4)nc(OCC34CCCN3CCC4)nc12] | 592.3 | 3TFA salt, HNMR (300 MHz, DMSO-d₆, ppm): δ 10.33 (brs, 1H), 9.43 (m, 1H), 9.25-9.12 (m, 1H), 8.23 (dd, J = 8.4, 1.2 Hz, 1H), 8.12 (dd, J = 8.4, 1.2 Hz, 1H), 7.95 (d, J = 1.8 Hz, 1H), 7.75 (dd, J = 8.4, 7.2 Hz, 1H), 7.67 (dd, J = 7.5, 1.5 Hz, 1H), 7.58 (t, J = 7.8 Hz, 1H), 7.48 (dd, J = 7.2, 1.2 Hz, 1H), 4.70-4.50 (m, 3H), 4.42 (d, J = 13.8 Hz, 1H), 4.21 (d, J = 12.0 Hz, 2H), 3.89 (d, J = 13.8 Hz, 1H), 3.72 (d, J = 13.8 Hz, 1H), 3.60-3.40 (m, 2H), 3.21 (d, J = 12.0 Hz, 2H), 2.24-1.96 (m, 12H). FNMR (282 MHz, DMSO-d₆, ppm): δ −121.80 (1F). |
| 65 | 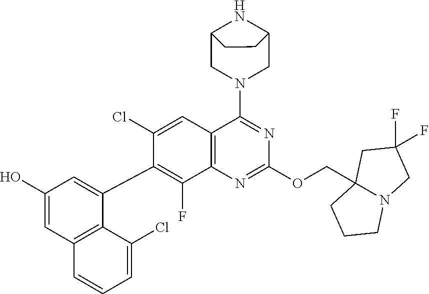 [SMILES: Oc1cc(-c2c(Cl)cc3c(N4CC5CCC(C4)N5)nc(OCC45CCCN4CC(F)(F)C5)nc3c2F)c2c(Cl)cccc2c1] | 644.2 | HNMR (400 MHz, methanol-d₄, ppm): δ 7.92 (d, J = 1.2 Hz, 1H), 7.75-7.72 (m, 1H), 7.35-7.28 (m, 3H), 6.97-6.95 (m, 1H), 4.79-4.62 (m, 4H), 4.24-4.13 (m, 3H), 3.95-3.80 (m, 4H), 3.46-3.39 (m, 1H), 3.02-2.74 (m, 2H), 2.47-2.07 (m, 8H). FNMR (376 MHz, methanol-d₄, ppm): δ −98.33 (2F), −123.40 (1F). |
| 66 | 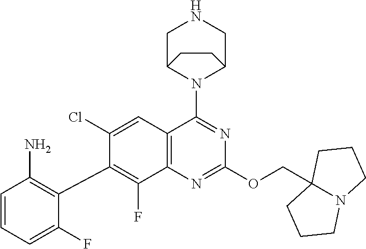 [SMILES: Nc1cccc(F)c1-c1c(Cl)cc2c(N3C4CCC3CNC4)nc(OCC34CCCN3CCC4)nc2c1F] | 541.3 | HNMR (400 MHz, methanol-d₄, ppm): δ 8.02 (d, J = 1.6 Hz, 1H), 7.22-7.17 (m, 1H), 6.66 (d, J = 8.0 Hz, 1H), 6.47 (t, J = 8.4 Hz, 1H), 5.17-5.13 (m, 2H), 4.64 (s, 2H), 3.69- 3.63 (m, 4H), 3.43 (d, J = 12.8 Hz, 2H), 3.27-3.26 (m, 2H), 2.32-2.07 (m, 12H). |
| 67 | 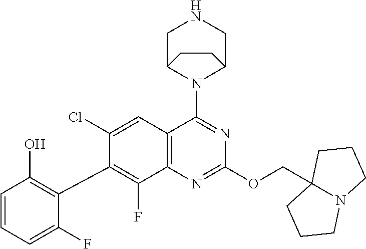 [SMILES: Oc1cccc(F)c1-c1c(Cl)cc2c(N3C4CCC3CNC4)nc(OCC34CCCN3CCC4)nc2c1F] | 542.3 | 3TFA salt, HNMR (400 MHz, DMSO-d₆, ppm): δ 10.34 (s, 1H), 10.25 (s, 1H), 9.52-9.42 (m, 1H), 8.88 (s, 1H), 7.99 (s, 1H), 7.36-7.31 (m, 1H), 6.84-6.76 (m, 2H), 5.01 (s, 2H), 4.60-4.48 (m, 2H), 3.55-3.15 (m, 8H), 2.00-1.94 (m, 12H). |
| 68 | 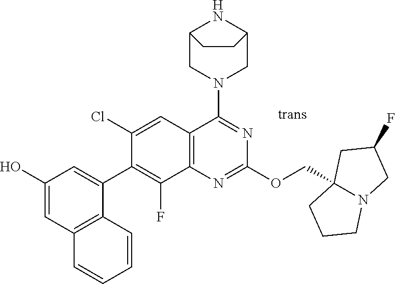 [SMILES: Oc1cc(-c2c(Cl)cc3c(N4CC5CCC(C4)N5)nc(OC[C@@]45CCCN4C[C@H](F)C5)nc3c2F)c2ccccc2c1] | 592.3 | HNMR (400 MHz, methanol-d₄, ppm): δ 8.01 (d, J = 1.2 Hz, 1H), 7.74 (d, J = 8.4 Hz, 1H), 7.43-7.39 (m, 1H), 7.26 (d, J = 2.4 Hz, 1H), 7.22-7.17 (m, 2H), 7.01-7.00 (m, 1H), 5.61-5.47 (m, 1H), 4.80-4.62 (m, 4H), 4.25-4.21 (m, 2H), 4.03-3.81 (m, 5H), 3.48-3.42 (m, 1H), 2.73-2.56 (m, 2H), 2.52-2.29 (m, 3H), 2.19-2.03 (m, 5H). FNMR (376 MHz, methanol-d₄, ppm): δ −123.49 (1F), −174.35 (1F). |
| 69 | 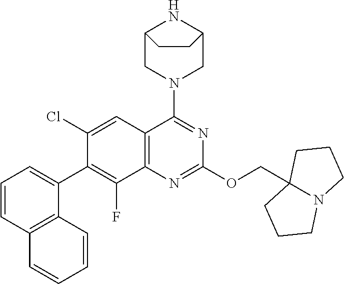 [SMILES: Fc1c(-c2cccc3ccccc23)c(Cl)cc2c(N3CC4CCC(C3)N4)nc(OCC34CCCN3CCC4)nc12] | 558.3 | 3TFA salt, HNMR (400 MHz, DMSO-d₆, ppm): δ 10.35 (s, 1H), 9.45-9.38 (m, 1H), 9.22-9.12 (m, 1H), 8.09-8.01 (m, 3H), 7.66 (t, J = 7.2 Hz, 1H), 7.56 (t, J = 6.8 Hz, 1H), 7.49-7.45 (m, 2H), 7.32 (d, J = 8.4 Hz, 1H), 4.55-4.51 (m, 4H), 4.18 (s, 2H), 3.79 (t, J = 12.8 Hz, 2H), 3.51-3.45 (m, 2H), 3.21-3.17 (m, 2H), 2.15-1.93 (m, 12H). FNMR (376 MHz, DMSO-d₆, ppm): δ −121.85 (1F). |
| 70 | 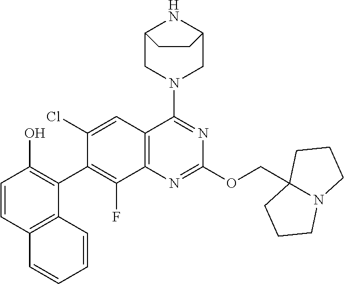 [SMILES: Oc1ccc2ccccc2c1-c1c(Cl)cc2c(N3CC4CCC(C3)N4)nc(OCC34CCCN3CCC4)nc2c1F] | 574.3 | HNMR (400 MHz, methanol-d₄, ppm): δ 8.00 (d, J = 1.2 Hz, 1H), 7.89-7.82 (m, 2H), 7.32-7.28 (m, 2H), 7.23 (d, J = 9.2 Hz, 1H), 7.12-7.09 (m, 1H), 4.78-4.71 (m, 2H), 4.64 (s, 2H), 4.23 (s, 2H), 3.91-3.85 (m, 2H), 3.68-3.63 (m, 2H), 3.26-3.24 (m, 2H), 2.35-2.05 (m, 12H). FNMR (376 MHz, methanol-d₄, ppm): δ −122.98 (1F). |
| 72 | 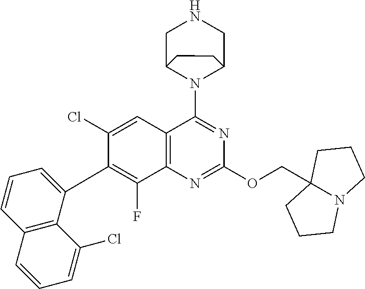 [SMILES: Fc1c(-c2cccc3cccc(Cl)c23)c(Cl)cc2c(N3C4CCC3CNC4)nc(OCC34CCCN3CCC4)nc12] | 592.3 | HNMR (400 MHz, methanol-d₄, ppm): δ 8.12 (dd, J = 8.0, 0.8 Hz, 1H), 8.01-7.98 (m, 2H), 7.67-7.64 (m, 1H), 7.57- 7.55 (m, 1H), 7.48-7.06 (m, 1H), 7.41-7.39 (m, 1H), 5.21- 5.14 (m, 2H), 4.64 (s, 2H), 3.73-3.65 (m, 4H), 3.45-3.42 (m, 2H), 3.40-3.20 (m, 2H), 2.34-2.05 (m, 12H). |
| 74 | 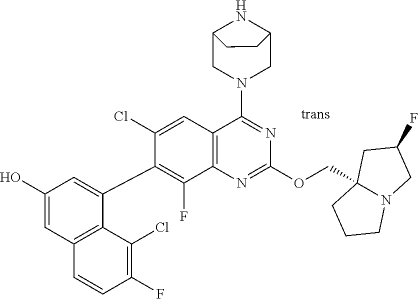 [SMILES: Oc1cc(-c2c(Cl)cc3c(N4CC5CCC(C4)N5)nc(OC[C@@]45CCCN4C[C@H](F)C5)nc3c2F)c2c(Cl)c(F)ccc2c1] | 644.2 | HNMR (400 MHz, methanol-d₄, ppm): δ 7.93-7.92 (m, 1H), 7.80 (dd, J = 9.2, 5.6 Hz, 1H), 7.40-7.35 (m, 2H), 7.01 (d, J = 2.4 Hz, 1H), 5.61-5.48 (m, 1H), 4.79-4.74 (m, 1H), 4.70-4.62 (m, 3H), 4.24-4.22 (m, 2H), 4.04-3.81 (m, 5H), 3.48-3.41 (m, 1H), 2.75-2.08 (m, 10H). FNMR (376 MHz, methanol-d₄, ppm): δ −116.5 (1F), −123.7 (1F), −174.3 (1F). |
| 75 | 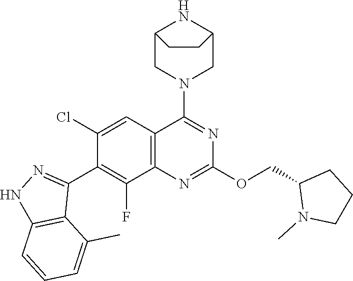 [SMILES: Cc1cccc2[nH]nc(-c3c(Cl)cc4c(N5CC6CCC(C5)N6)nc(OC[C@@H]5CCCN5C)nc4c3F)c12] | 536.3 | HNMR (400 MHz, methanol-d₄, ppm): δ 8.00-7.99 (m, 1H), 7.46-7.43 (m, 1H), 7.36-7.31 (m, 1H), 6.94-6.91 (m, 1H), 4.91-4.85 (m, 1H), 4.74-4.62 (m, 3H), 4.23 (s, 2H), 3.92-3.81 (m, 3H), 3.74-3.69 (m, 1H), 3.25-3.19 (m, 1H), 3.08 (s, 3H), 2.43-2.33 (m, 1H), 2.25-2.02 (m, 10H). FNMR (376 MHz, methanol-d₄, ppm): δ −122.35 (1F). |
| 76 | 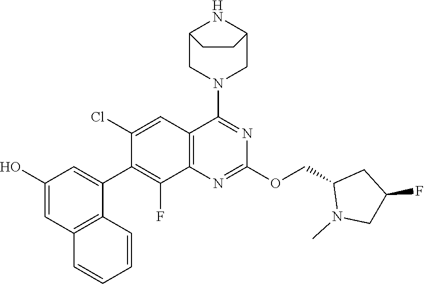 [SMILES: CN1C[C@H](F)C[C@H]1COc1nc(N2CC3CCC(C2)N3)c2cc(Cl)c(-c3cc(O)cc4ccccc34)c(F)c2n1] | 566.3 | HNMR (400 MHz, methanol-d₄, ppm): δ 8.00 (d, J = 1.2 Hz, 1H), 7.74 (d, J = 8.4 Hz, 1H), 7.42-7.38 (m, 1H), 7.26 (d, J = 2.4 Hz, 1H), 7.21-7.15 (m, 2H), 7.01 (d, J = 2.4 Hz, 1H), 5.51-5.38 (m, 1H), 4.98-4.94 (m, 1H), 4.75-4.68 (m, 3H) 4.25-4.21 (m, 3H), 4.07-3.99 (m, 1H), 3.91-3.88 (m, 2H), 3.66-3.57 (m, 1H), 3.16 (s, 3H), 2.69-2.60 (m, 1H), 2.47-2.31 (m, 1H), 2.15-2.11 (m, 4H). FNMR (376 MHz, methanol-d₄, ppm): δ −123.54 (1F), −174.17 (1F). |
| 78 | 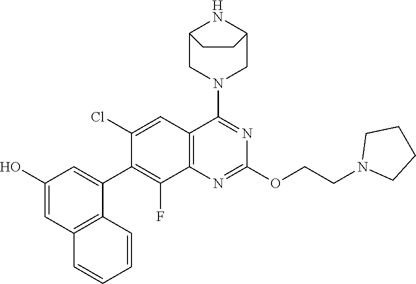 [SMILES: Oc1cc(-c2c(Cl)cc3c(N4CC5CCC(C4)N5)nc(OCCN4CCCC4)nc3c2F)c2ccccc2c1] | 548.0 | HNMR (400 MHz, methanol-d₄, ppm): δ 7.95 (d, J = 1.6 Hz, 1H), 7.74 (d, J = 8.3 Hz, 1H), 7.45-7.35 (m, 1H), 7.30- 7.15 (m, 3H), 7.02 (d, J = 2.4 Hz, 1H), 4.61 (t, J = 5.8 Hz, 2H), 4.51 (d, J = 11.0 Hz, 2H), 3.75-3.58 (m, 4H), 3.03 (t, J = 5.8 Hz, 2H), 2.80-2.76 (m, 4H), 1.91-1.77 (m, 8H). |
| 79 | 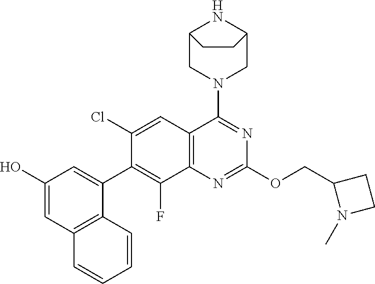 [SMILES: CN1CCC1COc1nc(N2CC3CCC(C2)N3)c2cc(Cl)c(-c3cc(O)cc4ccccc34)c(F)c2n1] | 534.3 | HNMR (400 MHz, methanol-d₄, ppm): δ 8.00 (s, 1H), 7.74 (d, J = 8.4 Hz, 1H), 7.42-7.38 (m, 1H), 7.26 (d, J = 2.4 Hz, 1H), 7.21-7.15 (m, 2H), 7.01 (d, J = 2.4 Hz, 1H), 4.82-4.70 (m, 5H), 4.23-4.17 (m, 3H), 3.99-3.87 (m, 3H), 2.99 (s, 3H), 2.64-2.55 (m, 2H), 2.17-2.12 (m, 4H). FNMR (376 MHz, methanol-d₄, ppm): δ −123.10 (1F). |
| 82 | 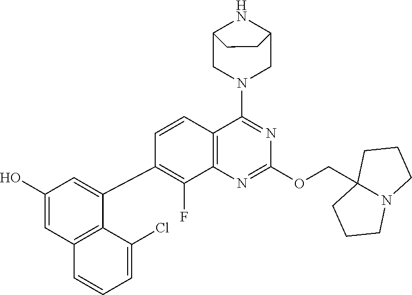 [SMILES: Oc1cc(-c2ccc3c(N4CC5CCC(C4)N5)nc(OCC45CCCN4CCC5)nc3c2F)c2c(Cl)cccc2c1] | 574.3 | HNMR (400 MHz, methanol-d₄, ppm): δ 7.93-7.85 (m, 1H), 7.71-7.69 (m, 1H), 7.57-7.43 (m, 3H), 7.24-7.17 (m, 2H), 4.75-4.61 (m, 2H), 4.39-4.37 (m, 1H), 4.24-4.20 (m, 2H), 3.86-3.60 (m, 4H), 3.47-3.41 (m, 2H), 3.29-3.27 (m, 1H), 2.42-2.31 (m, 2H), 2.27-2.03 (m, 10H). FNMR (376 MHz, methanol-d₄, ppm): δ −134.11 (1F). |
| 83 | 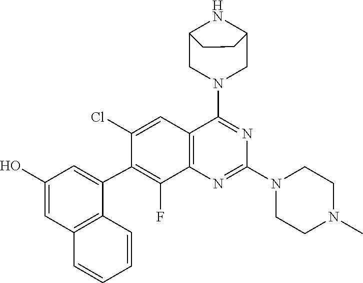 [SMILES: CN1CCN(c2nc(N3CC4CCC(C3)N4)c3cc(Cl)c(-c4cc(O)cc5ccccc45)c(F)c3n2)CC1] | 533.2 | HNMR (400 MHz, methanol-d₄, ppm): δ 7.76-7.72 (m, 2H), 7.41-7.39 (m, 1H), 7.26-7.19 (m, 3H), 7.01 (d, J = 4 Hz, 1H), 4.32-4.31 (m, 2H), 3.97-3.88 (m, 4H), 3.62 (m, 2H), 3.53-3.50 (m, 2H), 2.53-2.48 (m, 4H), 2.34 (s, 3H), 1.93-1.88 (m, 4H). |
| 84 | 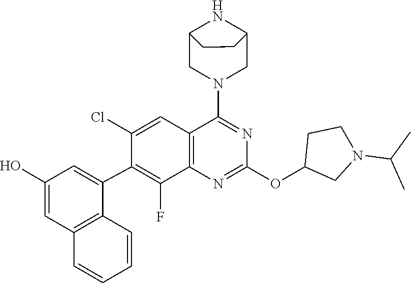 [SMILES: CC(C)N1CCC(Oc2nc(N3CC4CCC(C3)N4)c3cc(Cl)c(-c4cc(O)cc5ccccc45)c(F)c3n2)C1] | 562.1 | HNMR (400 MHz, methanol-d₄, ppm): δ 7.95 (d, J = 1.6 Hz, 1H), 7.74 (d, J = 8.4 Hz, 1H), 7.45-7.35 (m, 1H), 7.29- 7.15 (m, 3H), 7.02 (dd, J = 2.4, 0.9 Hz, 1H), 5.60-5.50 (m, 1H), 4.60-4.45 (m, 2H), 3.79-3.57 (m, 4H), 3.30-3.16 (m, 1H), 3.01-2.88 (m, 2H), 2.86-2.65 (m, 1H), 2.62-2.54 (m, 1H), 2.48-2.35 (m, 1H), 2.20-2.05 (m, 1H), 1.95-1.76 (m, 4H), 1.16 (d, J = 6.3 Hz, 6H). |
| 85 | 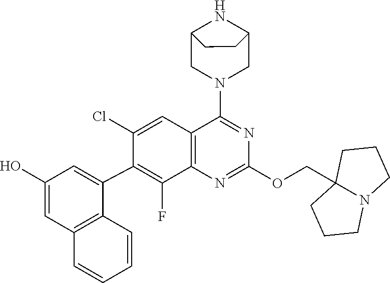 [SMILES: Oc1cc(-c2c(Cl)cc3c(N4CC5CCC(C4)N5)nc(OCC45CCCN4CCC5)nc3c2F)c2ccccc2c1] | 574.3 | 2TFA salt, HNMR (400 MHz, methanol-d₄, ppm): δ 8.01 (d, J = 1.2 Hz, 1H), 7.75 (d, J = 8.4 Hz, 1H), 7.43-7.38 (m, 1H), 7.26 (d, J = 2.4 Hz, 1H), 7.21-7.15 (m, 2H), 7.01 (d, J = 2.4 Hz, 1H), 4.74 (d, J = 13.6 Hz, 2H), 4.70-4.62 (m, 2H), 4.28-4.19 (m, 2H), 3.89 (d, J = 14.0 Hz, 2H), 3.69- 3.63 (m, 2H), 3.25-3.24 (m, 2H), 2.32-2.05 (m, 12H). FNMR (376 MHz, methanol-d₄, ppm): δ −123.40 (1F). |
| 86 | 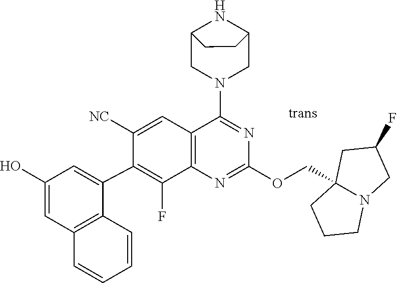 [SMILES: N#Cc1cc2c(N3CC4CCC(C3)N4)nc(OC[C@@]34CCCN3C[C@H](F)C4)nc2c(F)c1-c1cc(O)cc2ccccc12] | 583.4 | HNMR (400 MHz, methanol-d₄, ppm): δ 8.38 (s, 1H), 7.77 (d, J = 8.4 Hz, 1H), 7.45 (d, J = 7.2 Hz, 1H), 7.31 (d, J = 2.4 Hz, 1H), 7.28-7.20 (m, 2H), 7.13 (d, J = 2.4 Hz, 1H), 5.67-5.46 (m, 1H), 4.81-4.69 (m, 4H), 4.26-4.19 (m, 2H), 4.04-3.80 (m, 5H), 3.50-3.40 (m, 1H), 2.80-2.06 (m, 10H). FNMR (376 MHz, methanol-d₄, ppm): δ −124.17 (1F), −174.23 (1F). |
| 87 | 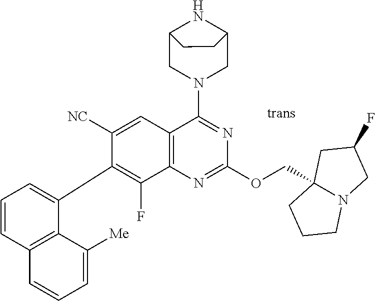 [SMILES: Cc1cccc2cccc(-c3c(C#N)cc4c(N5CC6CCC(C5)N6)nc(OC[C@@]56CCCN5C[C@H](F)C6)nc4c3F)c12] | 581.3 | 4TFA salt, HNMR (400 MHz, methanol-d₄, ppm): δ 8.35 (s, 1H), 8.07 (d, J = 8.0 Hz, 1H), 7.89 (d, J = 8.0 Hz, 1H), 7.58 (t, J = 7.6 Hz, 1H), 7.44 (t, J = 7.6 Hz, 1H), 7.37 (d, J = 7.2 Hz, 1H), 7.33 (d, J = 17.6, 7.2 Hz, 1H), 5.62-5.49 (m, 1H), 4.76-4.68 (m, 4H), 4.27-4.21 (m, 2H), 4.04-3.82 (m, 5H), 3.48-3.42 (m, 1H), 2.76-2.53 (m, 2H), 2.46-2.29 (m, 3H), 2.23-2.03 (m, 8H). FNMR (376 MHz, methanol- d₄, ppm): δ −124.73 (1F), −174.20 (1F). |
| 88 | 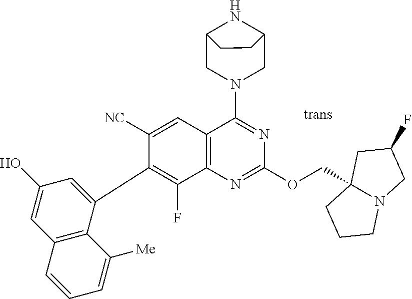 [SMILES: Cc1cccc2cc(O)cc(-c3c(C#N)cc4c(N5CC6CCC(C5)N6)nc(OC[C@@]56CCCN5C[C@H](F)C6)nc4c3F)c12] | 597.4 | HNMR (400 MHz, methanol-d₄, ppm): δ 8.34 (s, 1H), 7.65 (d, J = 8.0 Hz, 1H), 7.33-7.30 (m, 2H), 7.08 (d, J = 7.2 Hz, 1H), 6.94 (d, J = 2.4 Hz, 1H), 5.62-5.49 (m, 1H), 4.78-4.64 (m, 4H), 4.27-4.20 (m, 2H), 4.02-3.82 (m, 5H), 3.49-3.42 (m, 1H), 2.76-2.72 (m, 1H), 2.63-2.56 (m, 1H), 2.45-2.29 (m, 3H), 2.19-1.92 (m, 8H). FNMR (376 MHz, methanol- d₄, ppm): δ −124.81 (1F), −174.23 (1F). |
| 89 | 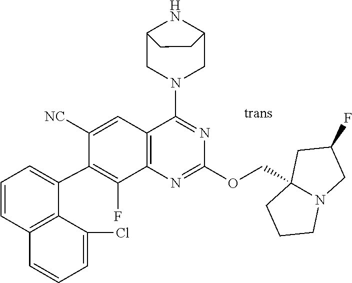 [SMILES: N#Cc1cc2c(N3CC4CCC(C3)N4)nc(OC[C@@]34CCCN3C[C@H](F)C4)nc2c(F)c1-c1cccc2cccc(Cl)c12] | 601.3 | HNMR (400 MHz, methanol-d₄, ppm): δ 8.32-8.31 (m, 1H), 8.18-8.16 (m, 1H), 8.05-8.02 (m, 1H), 7.70 (t, J = 7.2 Hz, 1H), 7.62-7.60 (m, 1H), 7.54-7.51 (m, 2H), 5.62-5.49 (m, 1H), 4.81-4.65 (m, 4H), 4.27-4.21 (m, 2H), 4.05-3.80 (m, 5H), 3.49-3.42 (m, 1H), 2.76-2.53 (m, 2H), 2.45-2.29 (m, 3H), 2.23-2.06 (m, 5H). FNMR (376 MHz, methanol- d₄, ppm): δ −124.98 (1F), −174.23. |
| 90 | 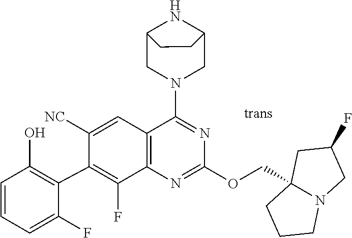 [SMILES: N#Cc1cc2c(N3CC4CCC(C3)N4)nc(OC[C@@]34CCCN3C[C@H](F)C4)nc2c(F)c1-c1c(O)cccc1F] | 551.3 | HNMR (400 MHz, methanol-d₄, ppm): δ 8.31-8.30 (m, 1H), 7.93-7.33 (m, 1H), 6.82 (d, J = 8.4 Hz, 1H), 6.76 (t, J = 8.4 Hz, 1H), 5.63-5.48 (m, 1H), 4.79-4.65 (m, 4H), 4.24- 4.18 (m, 2H), 4.05-3.83 (m, 5H), 3.50-3.43 (m, 1H), 2.76- 2.53 (m, 2H), 2.45-2.30 (m, 3H), 2.23-1.98 (m, 5H). FNMR (376 MHz, methanol-d₄, ppm): δ −115.40 (1F), −123.51 (1F), −174.21 (1F). |
| 91 | 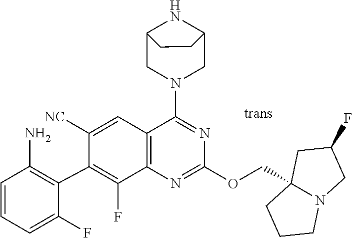 [SMILES: N#Cc1cc2c(N3CC4CCC(C3)N4)nc(OC[C@@]34CCCN3C[C@H](F)C4)nc2c(F)c1-c1c(N)cccc1F] | 550.3 | HNMR (400 MHz, methanol-d₄, ppm): δ 8.33 (s, 1H), 7.25-7.19 (m, 1H), 6.68 (d, J = 8.4 Hz, 1H), 6.49 (t, J = 8.8 Hz, 1H), 5.63-5.50 (m, 1H), 4.80-4.70 (m, 4H), 4.22 (s, 2H), 4.04-3.84 (m, 5H), 3.50-3.43 (m, 1H), 2.75-2.53 (m, 2H), 2.45-2.30 (m, 3H), 2.19-2.02 (m, 5H). FNMR (376 MHz, methanol-d₄, ppm): δ −116.57 (1F), −123.40 (1F), −174.26 (1F). |
| 92 | 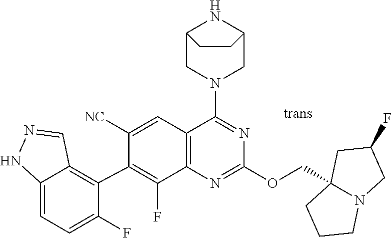 [SMILES: N#Cc1cc2c(N3CC4CCC(C3)N4)nc(OC[C@@]34CCCN3C[C@H](F)C4)nc2c(F)c1-c1c(F)ccc2[nH]ncc12] | 571.3 | HNMR (400 MHz, methanol-d₄, ppm): δ 8.40 (s, 1H), 7.70-7.54 (m, 2H), 7.48-7.45 (m, 1H), 5.63-5.49 (m, 1H), 4.82-4.68 (m, 4H), 4.30-4.20 (m, 2H), 4.06-3.80 (m, 5H), 3.50-3.40 (m, 1H), 2.80-2.00 (m, 13H). |
| 95 | 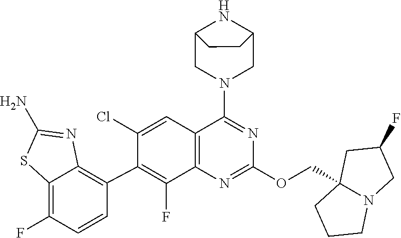 [SMILES: Nc1nc2c(-c3c(Cl)cc4c(N5CC6CCC(C5)N6)nc(OC[C@@]56CCCN5C[C@H](F)C6)nc4c3F)ccc(F)c2s1] | 616.0 | HNMR (400 MHz, methanol-d₄, ppm): δ 7.86 (s, 1H), 7.21 (dd, J = 8.4, 5.5 Hz, 1H), 6.98 (t, J = 8.8 Hz, 1H), 5.40- 5.20 (m, 1H), 4.62-4.35 (m, 2H), 4.25-4.21 (m, 2H), 3.73- 3.52 (m, 4H), 3.25-3.17 (m, 3H), 3.06-2.93 (m, 1H), 2.39- 2.07 (m, 3H), 2.04-1.65 (m, 7H). |
| 96 | 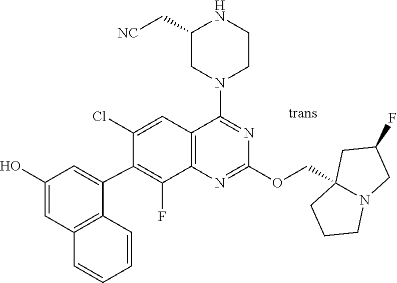 [SMILES: N#CC[C@H]1CN(c2nc(OC[C@@]34CCCN3C[C@H](F)C4)nc3c(F)c(-c4cc(O)cc5ccccc45)c(Cl)cc23)CCN1] | 605.3 | HNMR (400 MHz, DMSO-d₆, ppm): δ 10.02 (s, 1H), 7.92 (s, 1H), 7.78 (d, J = 8.4 Hz, 1H), 7.41 (t, J = 6.4, 1H), 7.25 (s, 1H), 7.20-7.15 (m, 2H), 7.03 (s, 1H), 5.23 (d, J = 53.6 Hz, 1H), 4.26-4.12 (m, 2H), 3.26-2.94 (m, 7H), 2.90-2.65 (m, 6H), 2.12-1.99 (m, 3H), 1.85-1.70 (m, 3H). FNMR (376 MHz, DMSO-d₆, ppm): δ −122.34 (1F), −172.07 (1F). |
| 100 | 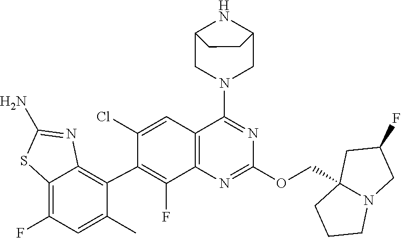 [SMILES: Cc1cc(F)c2sc(N)nc2c1-c1c(Cl)cc2c(N3CC4CCC(C3)N4)nc(OC[C@@]34CCCN3C[C@H](F)C4)nc2c1F] | 630.2 | HNMR (400 MHz, methanol-d₄, ppm): δ 7.98 (s, 1H), 6.93 (d, J = 8.0 Hz, 1H), 5.65-5.45 (m, 1H), 4.85-4.66 (m, 4H), 4.24 (s, 2H), 3.90-3.87 (m, 4H), 3.49-3.35 (m, 2H), 2.69- 2.57 (m, 2H), 2.51-2.40 (m, 1H), 2.36-2.34 (m, 2H), 2.17- 2.08 (m, 8H). |
| 109 | 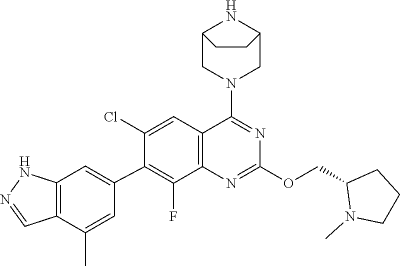 [SMILES: Cc1cc(-c2c(Cl)cc3c(N4CC5CCC(C4)N5)nc(OC[C@@H]4CCCN4C)nc3c2F)cc2[nH]ncc12] | 536.3 | HNMR (400 MHz, methanol-d₄, ppm): δ 8.19-8.18 (m, 1H), 7.96-7.94 (m, 1H), 7.36 (s, 1H), 6.88 (s, 1H), 4.89- 4.80 (m, 1H), 4.70-4.62 (m, 3H), 4.22 (s, 2H), 3.90-3.81 (m, 3H), 3.74-3.69 (m, 1H), 3.25-3.19 (m, 1H), 3.09 (s, 3H), 2.66 (s, 3H), 2.43-2.36 (m, 1H), 2.25-2.02 (m, 7H). FNMR (376 MHz, methanol-d₄, ppm): δ −124.92 (1F). |
| 111 | 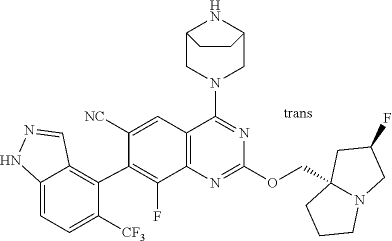 [SMILES: N#Cc1cc2c(N3CC4CCC(C3)N4)nc(OC[C@@]34CCCN3C[C@H](F)C4)nc2c(F)c1-c1c(C(F)(F)F)ccc2[nH]ncc12] | 625.3 | HNMR (400 MHz, methanol-d₄, ppm): δ 8.41 (s, 1H), 7.92-7.87 (m, 2H), 7.73 (s, 1H), 5.62-5.49 (m, 1H), 4.79- 4.68 (m, 4H), 4.26-4.22 (m, 2H), 4.03-3.83 (m, 5H), 3.50- 3.41 (m, 1H), 2.76-2.53 (m, 2H), 2.45-2.29 (m, 3H), 2.23- 2.04 (m, 5H). FNMR (376 MHz, methanol-d₄, ppm): δ −58.83 (3F), −123.52 (1F), −174.22 (1F) |
| 117 | 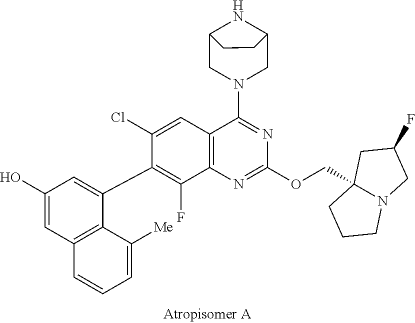 [SMILES: Cc1cccc2cc(O)cc(-c3c(Cl)cc4c(N5CC6CCC(C5)N6)nc(OC[C@@]56CCCN5C[C@H](F)C6)nc4c3F)c12] | 606.3 | HNMR (400 MHz, methanol-d₄, ppm): δ 7.97-7.95 (m, 1H), 7.62 (d, J = 8.4 Hz, 1H), 7.30-7.25 (m, 2H), 7.03 (d, J = 6.8 Hz, 1H) 6.84-6.82 (m, 1H), 5.61-5.47 (m, 1H), 4.77- 4.74 (m, 1H), 4.69-4.61 (m, 3H), 4.25-4.21 (m, 2H), 4.02- 3.82 (m, 5H), 3.47-3.40 (m, 1H), 2.73-2.51 (m, 2H), 2.44- 2.28 (m, 3H), 2.19-2.07 (m, 5H), 1.99 (s, 3H). FNMR (376 MHz, methanol-d₄, ppm): δ −123.26 (1F), −174.3 (1F). |
| 118 | 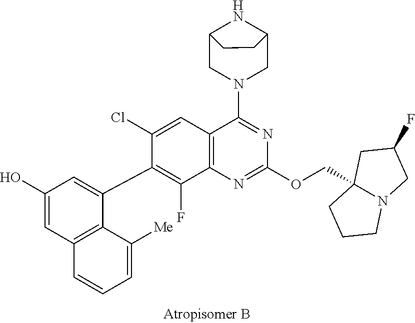 [SMILES: Cc1cccc2cc(O)cc(-c3c(Cl)cc4c(N5CC6CCC(C5)N6)nc(OC[C@@]56CCCN5C[C@H](F)C6)nc4c3F)c12] | 606.3 | HNMR (400 MHz, methanol-d₄, ppm): δ 7.96-7.95 (m, 1H), 7.61 (d, J = 8.0 Hz, 1H), 7.31-7.25 (m, 2H), 7.03 (d, J = 6.8 Hz, 1H), 6.84-6.82 (m, 1H), 5.61-5.48 (m, 1H), 4.85- 4.74 (m, 1H), 4.70-4.62 (m, 3H), 4.25-4.22 (m, 2H), 4.03- 3.81 (m, 5H), 3.48-3.41 (m, 1H), 2.75-2.52 (m, 2H), 2.44- 2.29 (m, 3H), 2.21-2.06 (m, 5H), 1.99 (s, 3H). FNMR (376 MHz, methanol-d₄, ppm): δ −123.23 (1F), −174.29 (1F). |
| 124 | 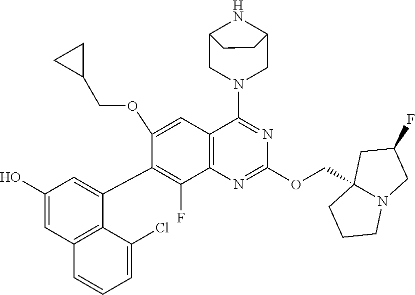 [SMILES: Oc1cc(-c2c(OCC3CC3)cc3c(N4CC5CCC(C4)N5)nc(OC[C@@]45CCCN4C[C@H](F)C5)nc3c2F)c2c(Cl)cccc2c1] | 662.3 | 3TFA salt, HNMR (400 MHz, DMSO-d₆, ppm): δ 10.85 (s, 1H), 10.26 (s, 1H), 9.25 (s, 1H), 9.06 (s, 1H), 7.82-7.79 (m, 1H), 7.37-7.29 (m, 3H), 7.03 (s, 1H), 6.95 (s, 1H), 5.60-5.49 (m, 1H), 4.54 (s, 2H), 4.48-4.41 (m, 1H), 4.35- 4.31 (m, 1H), 4.20-4.15 (m, 2H), 4.14-4.10 (m, 1H), 3.82- 3.58 (m, 7H), 2.56-2.51 (m, 2H), 2.37-1.97 (m, 8H), 0.95- 0.85 (m, 1H), 0.35-0.19 (m, 2H), 0.05-0.11 (m, 2H). |
| 126 | 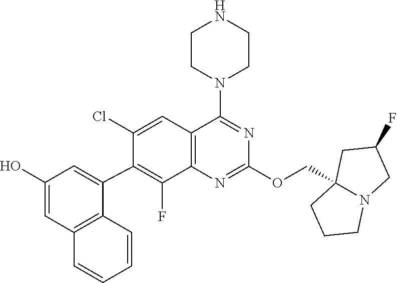 [SMILES: Oc1cc(-c2c(Cl)cc3c(N4CCNCC4)nc(OC[C@@]45CCCN4C[C@H](F)C5)nc3c2F)c2ccccc2c1] | 566.3 | 3TFA salt, HNMR (400 MHz, DMSO-d₆, ppm): δ 10.91 (s, 1H), 10.07 (s, 1H), 9.07 (s, 2H), 8.07 (s, 1H), 7.78 (d, J = 8.4 Hz, 1H), 7.43-7.39 (m, 1H), 7.27 (s, 1H), 7.26-7.21 (m, 1H), 7.19-7.11 (m, 1H), 7.02 (d, J = 2.4 Hz, 1H), 5.60- 5.46 (m, 1H), 4.56-4.55 (m, 2H), 3.98-3.89 (m, 4H), 3.87- 3.67 (m, 4H), 3.38-3.21 (m, 3H), 2.64-2.62 (m, 1H), 2.58- 2.53 (m, 2H), 2.27-2.20 (m, 1H), 2.19-1.99 (m, 3H). |
| 127 | 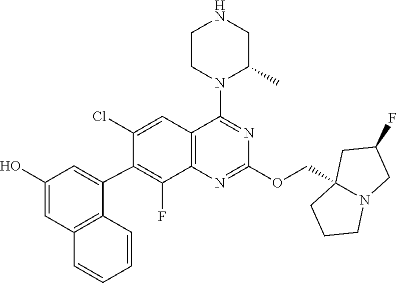 [SMILES: C[C@H]1CNCCN1c1nc(OC[C@@]23CCCN2C[C@H](F)C3)nc2c(F)c(-c3cc(O)cc4ccccc34)c(Cl)cc12] | 580.3 | 3TFA salt, HNMR (400 MHz, DMSO-d₆, ppm): δ 10.82 (s, 1H), 10.06 (s, 1H), 9.23 (s, 1H), 8.83 (s, 1H), 7.98 (s, 1H), 7.79 (d, J = 8.4 Hz, 1H), 7.44-7.39 (m, 1H), 7.27 (d, J = 2.0 Hz, 1H), 7.21-7.17 (m, 1H), 7.14-7.10 (m, 1H), 7.03- 7.01 (m, 1H), 5.58-5.45 (m, 1H), 4.83 (s, 1H), 4.55 (s, 2H), 4.21 (d, J = 12.8 Hz, 1H), 3.83-3.70 (m, 5H), 3.23-3.10 (m, 3H), 2.47-2.46 (m, 2H), 2.29-2.26 (m, 1H), 2.20-2.00 (m, 4H), 1.47-1.45 (m, 3H). |
| 128 | 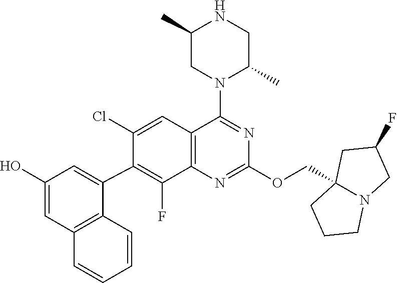 [SMILES: C[C@@H]1CN(c2nc(OC[C@@]34CCCN3C[C@H](F)C4)nc3c(F)c(-c4cc(O)cc5ccccc45)c(Cl)cc23)[C@@H](C)CN1] | 594.3 | 3TFA salt, HNMR (400 MHz, DMSO-d₆, ppm): δ 10.81 (s, 1H), 10.07 (s, 1H), 9.13 (s, 1H), 8.98 (s, 1H), 8.10 (s, 1H), 7.79 (d, J = 8.4 Hz, 1H), 7.44-7.40 (m, 1H), 7.27 (d, J = 2.0 Hz, 1H), 7.20-7.08 (m, 2H), 7.03-7.01 (m, 1H), 5.59- 5.49 (m, 1H), 4.62-4.54 (m, 2H), 4.45-4.42 (m, 1H), 3.86- 3.68 (m, 6H), 3.60-3.50 (m, 1H), 3.22-3.13 (m, 2H), 2.75- 2.54 (m, 2H), 2.31-2.25 (m, 1H), 2.18-2.11 (m, 2H), 2.03- 1.99 (m, 1H), 1.37-1.27 (m, 6H). |
| 129 | 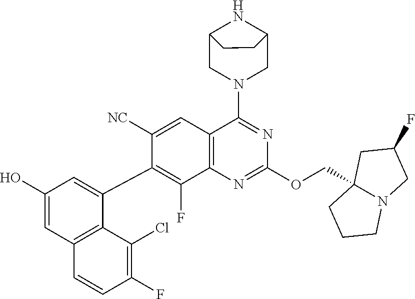 [SMILES: N#Cc1cc2c(N3CC4CCC(C3)N4)nc(OC[C@@]34CCCN3C[C@H](F)C4)nc2c(F)c1-c1cc(O)cc2ccc(F)c(Cl)c12] | 635.2 | 3TFA salt, HNMR (400 MHz, methanol-d₄, ppm): δ 8.32 (s, 1H), 7.83 (dd, J = 9.2, 5.6 Hz, 1H), 7.43-7.39 (m, 2H), 7.13 (d, J = 2.8 Hz, 1H), 5.63-5.47 (m, 1H), 4.75-4.65 (m, 4H), 4.28-4.20 (m, 2H), 4.03-3.81 (m, 5H), 3.48-3.41 (m, 1H), 2.76-2.53 (m, 2H), 2.44-2.29 (m, 3H), 2.21-2.01 (m, 5H). FNMR (376 MHz, methanol-d₄, ppm): δ −116.07 (1F), −125.03 (1F), −174.25 (1F). |
| 130 | 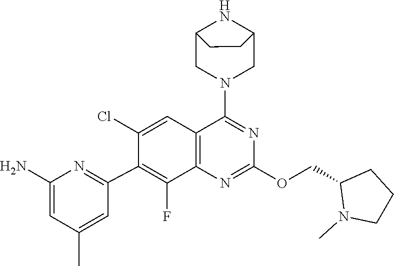 [SMILES: Cc1cc(N)nc(-c2c(Cl)cc3c(N4CC5CCC(C4)N5)nc(OC[C@@H]4CCCN4C)nc3c2F)c1] | 512.3 | HNMR (400 MHz, methanol-d₄, ppm): δ 7.98 (s, 1H), 6.88-6.87 m, 2H), 4.68-4.65 (m, 3H), 4.25-4.23 (m, 2H), 3.91-3.88 (m, 3H), 3.80-3.71 (m, 1H), 3.27-3.19 (m, 2H), 3.10 (s, 3H), 2.46 (s, 3H), 2.42-2.38 (m, 1H), 2.24-2.02 (m, 7H). |
| 131 | 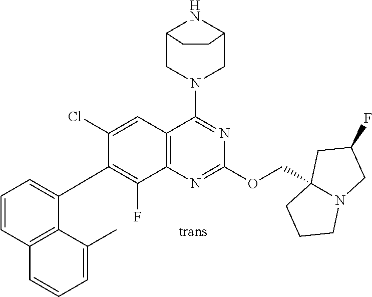 [SMILES: Cc1cccc2cccc(-c3c(Cl)cc4c(N5CC6CCC(C5)N6)nc(OC[C@@]56CCCN5C[C@H](F)C6)nc4c3F)c12] | 590.3 | HNMR (400 MHz, methanol-d₄, ppm): δ 8.01 (d, J = 8.0 Hz, 1H), 7.96 (d, J = 1.2 Hz, 1H), 7.85 (d, J = 8.0 Hz, 1H), 7.54 (t, J = 7.6 Hz, 1H), 7.40 (t, J = 7.6 Hz, 1H), 7.28-7.23 (m, 2H), 5.61-5.47 (m, 1H), 4.81-4.74 (m, 1H), 4.71-4.60 (m, 3H), 4.32-4.19 (m, 2H), 4.05-3.79 (m, 5H), 3.48-3.39 (m, 1H), 2.77-2.52 (m, 2H), 2.47-2.26 (m, 3H), 2.24-2.01 (m, 8H). FNMR (376 MHz, methanol-d₄, ppm): δ −123.05 (1F), −174.25 (1F) |
| 132 | 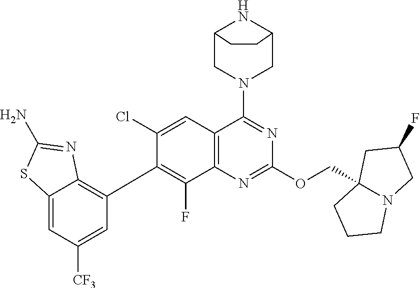 [SMILES: Nc1nc2c(-c3c(Cl)cc4c(N5CC6CCC(C5)N6)nc(OC[C@@]56CCCN5C[C@H](F)C6)nc4c3F)cc(C(F)(F)F)cc2s1] | 666.2 | HNMR (400 MHz, methanol-d₄, ppm): δ 8.06 (s, 1H), 7.88 (s, 1H), 7.46 (s, 1H), 5.29 (m, 1H), 4.60-4.48 (m, 1H), 4.47-4.38 (m, 1H), 4.31-4.12 (m, 2H), 3.69-3.54 (m, 4H), 3.26-3.11 (m, 3H), 3.05-2.95 (m, 1H), 2.39-2.07 (m, 3H), 2.02-1.75 (m, 7H). |
| 133 | 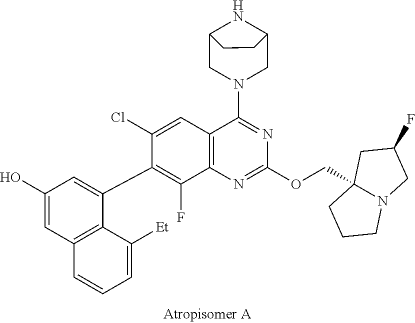 [SMILES: CCc1cccc2cc(O)cc(-c3c(Cl)cc4c(N5CC6CCC(C5)N6)nc(OC[C@@]56CCCN5C[C@H](F)C6)nc4c3F)c12] | 620.3 | HNMR (400 MHz, methanol-d₄, ppm): δ 8.00-7.96 (m, 1H), 7.61 (d, J = 8.0 Hz, 1H), 7.34 (t, J = 7.6 Hz, 1H), 7.26 (d, J = 2.4 Hz, 1H), 7.13-7.11 (m, 1H), 6.81 (d, J = 2.8 Hz, 1H), 5.61-5.47 (m, 1H), 4.76-4.60 (m, 4H), 4.25-4.20 (m, 2H), 4.04-3.80 (m, 5H), 3.50-3.40 (m, 1H), 2.77-2.04 (m, 12H), 0.90 (t, J = 7.2 Hz, 3H). FNMR (376 MHz, methanol-d₄, ppm): δ −122.63 (1F), −174.23 (1F). |
| 134 | 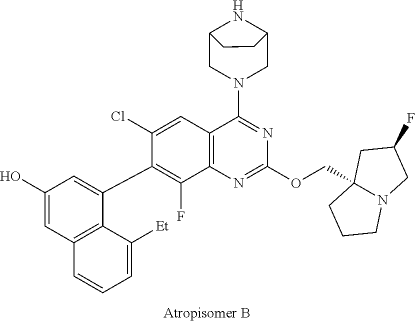 [SMILES: CCc1cccc2cc(O)cc(-c3c(Cl)cc4c(N5CC6CCC(C5)N6)nc(OC[C@@]56CCCN5C[C@H](F)C6)nc4c3F)c12] | 620.3 | HNMR (400 MHz, methanol-d₄, ppm): δ 8.00-7.96 (m, 1H), 7.61 (d, J = 8.0 Hz, 1H), 7.33 (t, J = 8.0 Hz, 1H), 7.26 (d, J = 2.4 Hz, 1H), 7.13-7.11 (m, 1H), 6.81 (d, J = 2.4 Hz, 1H), 5.62-5.47 (m, 1H), 4.79-4.60 (m, 4H), 4.27-4.20 (m, 2H), 4.04-3.81 (m, 5H), 3.49-3.40 (m, 1H), 2.77-2.04 (m, 12H), 0.89 (t, J = 7.2 Hz, 3H). FNMR (376 MHz, methanol-d₄, ppm): δ −122.57 (1F), −174.21 (1F). |
| 135 | 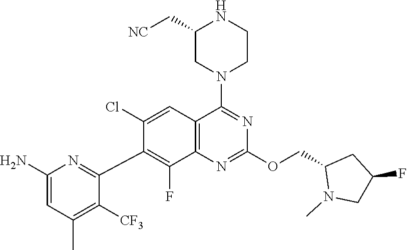 [SMILES: Cc1cc(N)nc(-c2c(Cl)cc3c(N4CCN[C@@H](CC#N)C4)nc(OC[C@@H]4C[C@@H](F)CN4C)nc3c2F)c1C(F)(F)F] | 611.3 | HNMR (400 MHz, methanol-d₄, ppm): δ 7.86 (s, 1H), 6.60 (s, 1H), 5.10 (d, J = 2.0 Hz, 1H), 4.49-4.40 (m, 3H), 4.25- 4.20 (m, 1H), 3.50-3.35 (m, 3H), 3.18-2.94 (m, 4H), 2.72- 2.50 (m, 3H), 2.49 (s, 3H), 2.47 (s, 3H), 2.48-2.37 (m, 1H), 2.19-1.98 (m, 1H). |
| 136 | 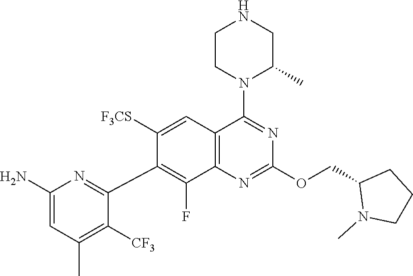 | 634.2 | HNMR (400 MHz, DMSO-d₆, ppm): δ 8.06 (d, J = 7.3 Hz, 1H), 6.84 (s, 2H), 6.50 (s, 1H), 4.45 (m, 2H), 4.27-3.97 (m, 2H), 3.56-3.35 (m, 2H), 2.96-2.92 (m, 3H), 2.79-2.74 (m, 2H), 2.59-2.56 (m, 1H), 2.36-2.35 (m, 6H), 2.19-2.14 (m, 1H), 1.96-1.90 (m, 1H), 1.66-1.56 (m, 3H), 1.50-1.46 (m, 3H). |
| 138 | 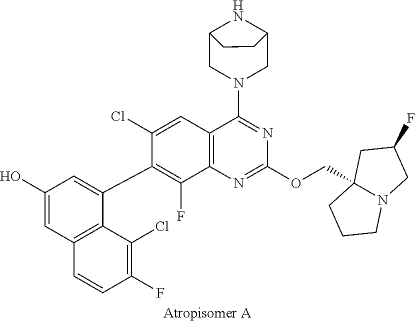 [SMILES: Oc1cc(-c2c(Cl)cc3c(N4CC5CCC(C4)N5)nc(OC[C@@]45CCCN4C[C@H](F)C5)nc3c2F)c2c(Cl)c(F)ccc2c1] | 644.2 | 3TFA salt, HNMR (400 MHz, methanol-d₄, ppm): δ 7.93 (d, J = 1.6 Hz, 1H), 7.79 (dd, J = 9.2, 5.6 Hz, 1H), 7.40- 7.34 (m, 2H), 7.01 (d, J = 2.4 Hz, 1H), 5.62-5.46 (m, 1H), 4.78-4.73 (m, 1H), 4.71-4.63 (m, 3H), 4.25-4.22 (m, 2H), 4.03-3.81 (m, 5H), 3.47-3.41 (m, 1H), 2.74-2.52 (m, 2H), 2.44-2.28 (m, 3H), 2.20-2.08 (m, 5H). FNMR (376 MHz, methanol-d₄, ppm): δ −116.5 (1F), −123.7 (1F), −174.3 (1F). |
| 139 | 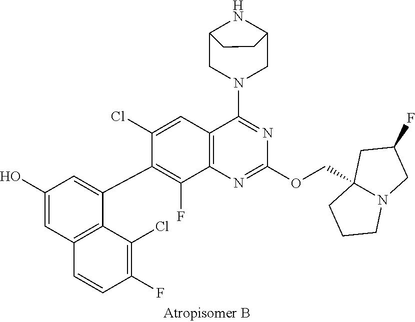 [SMILES: Oc1cc(-c2c(Cl)cc3c(N4CC5CCC(C4)N5)nc(OC[C@@]45CCCN4C[C@H](F)C5)nc3c2F)c2c(Cl)c(F)ccc2c1] | 644.2 | 3TFA salt, HNMR (400 MHz, methanol-d₄, ppm): δ 7.93 (d, J = 1.6 Hz, 1H), 7.79 (dd, J = 9.2, 5.6 Hz, 1H), 7.40- 7.34 (m, 2H), 7.01 (d, J = 2.4 Hz, 1H), 5.62-5.47 (m, 1H), 4.79-4.75 (m, 1H), 4.69-4.62 (m, 3H), 4.25-4.21 (m, 2H), 4.04-3.80 (m, 5H), 3.47-3.41 (m, 1H), 2.75-2.52 (m, 2H), 2.44-2.28 (m, 3H), 2.18-2.04 (m, 5H). FNMR (376 MHz, methanol-d₄, ppm): δ −116.5 (1F), −123.17 (1F), −174.3 (1F). |
| 140 | 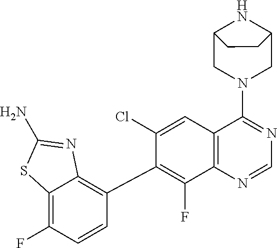 [SMILES: Nc1nc2c(-c3c(Cl)cc4c(N5CC6CCC(C5)N6)ncnc4c3F)ccc(F)c2s1] | 459.0 | HNMR (400 MHz, methanol-d₄, ppm): δ 8.55 (s, 1H), 7.94 (s, 1H), 7.24 (d, J = 1.2 Hz, 1H), 7.00 (d, J = 1.2 Hz, 1H), 4.50 (dd, J = 2.0, 1.2 Hz, 2H), 3.60-3.50 (m, 4H), 1.87- 1.54 (m, 4H). |
| 141 | 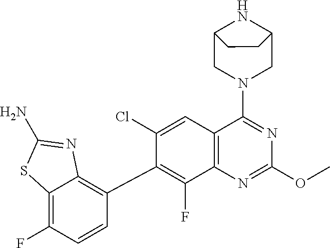 [SMILES: COc1nc(N2CC3CCC(C2)N3)c2cc(Cl)c(-c3ccc(F)c4sc(N)nc34)c(F)c2n1] | 489.2 | HNMR (400 MHz, methanol-d₄, ppm): δ 7.92-7.88 (m, 3H), 7.24-7.20 (m, 1H), 7.09-7.05 (m, 1H), 4.41-4.38 (m, 2H), 4.01-3.95 (m, 2H), 3.93 (s, 3H), 3.73-3.62 (m, 2H), 1.87-1.83 (m, 4H). |
| 143 | 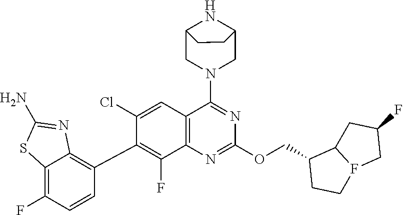 | 666.1 | HNMR (400 MHz, DMSO-d₆, ppm): δ 7.96 (s, 1H), 7.54- 7.48 (m, 1H), 7.39-7.34 (m, 1H), 5.61-5.40 (m, 1H), 4.74- 4.54 (m, 4H), 4.19-4.10 (m, 2H), 3.92-3.71 (m, 5H), 3.42- 3.35 (m, 1H), 2.70-2.46 (m, 2H), 2.43-2.34 (m, 1H), 2.33- 2.22 (m, 2H), 2.17-2.01 (m, 5H). |
| 144 | 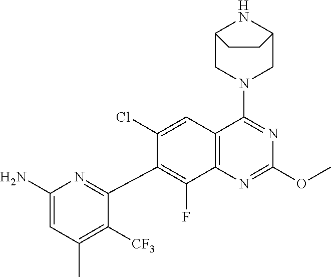 [SMILES: COc1nc(N2CC3CCC(C2)N3)c2cc(Cl)c(-c3nc(N)cc(C)c3C(F)(F)F)c(F)c2n1] | 497.1 | HNMR (400 MHz, methanol-d₄, ppm): δ 7.84-7.82 (m 1H), 6.60 (s, 1H), 4.51-4.47 (m, 2H), 4.03 (s, 3H), 3.82- 3.76 (m, 2H), 3.68-3.64 (m, 2H), 2.45-2.44 (m, 3H), 1.95- 1.89 (m, 4H). |
| 145 | 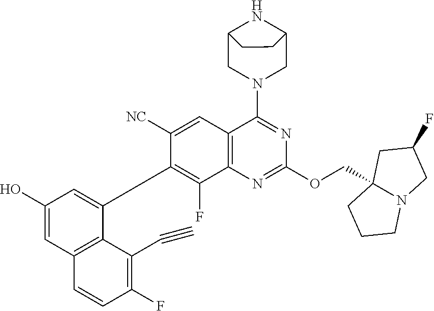 [SMILES: C#Cc1c(F)ccc2cc(O)cc(-c3c(C#N)cc4c(N5CC6CCC(C5)N6)nc(OC[C@@]56CCCN5C[C@H](F)C6)nc4c3F)c12] | 625.3 | 3TFA salt, HNMR (400 MHz, methanol-d₄, ppm): δ 8.30 (s, 1H), 7.92-7.88 (m, 1H), 7.40-7.33 (m, 2H), 7.15 (s, 1H), 5.63-5.50 (m, 1H), 4.83-4.67 (m, 4H), 4.32-4.21 (m, 2H), 4.07-3.83 (m, 5H), 3.53-3.42 (m, 1H), 3.39-3.34 (m, 1H), 2.78-2.53 (m, 2H), 2.51-2.29 (m, 3H), 2.24-2.01 (m, 5H). FNMR (376 MHz, methanol-d₄, ppm): δ −111.10 (1F), −124.89 (1F), −174.28 (1F). |
| 147 | 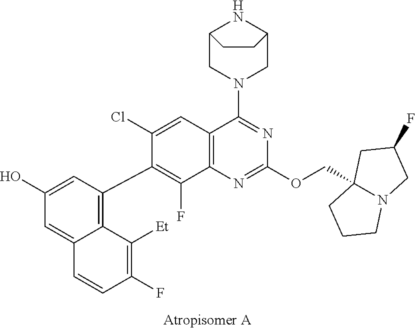 [SMILES: CCc1c(F)ccc2cc(O)cc(-c3c(Cl)cc4c(N5CC6CCC(C5)N6)nc(OC[C@@]56CCCN5C[C@H](F)C6)nc4c3F)c12] | 638.3 | 3TFA salt, HNMR (400 MHz, methanol-d₄, ppm): δ 8.02- 7.99 (m, 1H), 7.70-7.66 (m, 1H), 7.30-7.23 (m, 2H), 6.88- 6.87 (m, 1H), 5.63-5.49 (m, 1H), 4.77-4.64 (m, 4H), 4.29- 4.22 (m, 2H), 4.05-3.83 (m, 5H), 3.49-3.43 (m, 1H), 2.77- 2.54 (m, 3H), 2.46-2.05 (m, 9H), 0.79 (t, J = 7.6 Hz, 3H). |
| 148 | 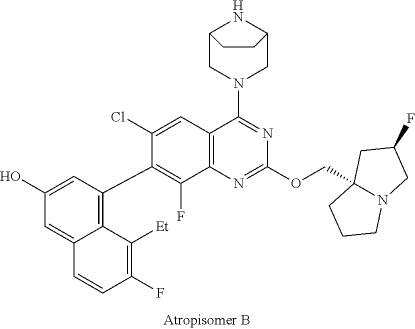 [SMILES: CCc1c(F)ccc2cc(O)cc(-c3c(Cl)cc4c(N5CC6CCC(C5)N6)nc(OC[C@@]56CCCN5C[C@H](F)C6)nc4c3F)c12] | 638.3 | 3TFA salt, HNMR (400 MHz, methanol-d₄, ppm): δ 8.00- 7.98 (m, 1H), 7.70-7.65 (m, 1H), 7.30-7.22 (m, 2H), 6.89- 6.87 (m, 1H), 5.63-5.50 (m, 1H), 4.78-4.67 (m, 4H), 4.26- 4.24 (m, 2H), 4.07-3.82 (m, 5H), 3.49-3.42 (m, 1H), 2.78- 2.54 (m, 3H), 2.51-2.10 (m, 9H), 0.78 (t, J = 7.6 Hz, 3H). |
| 149 | 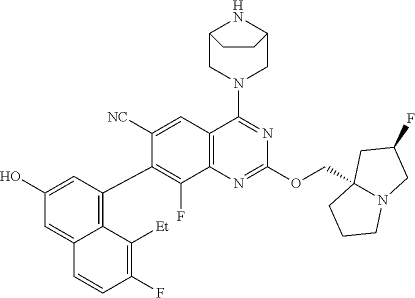 [SMILES: CCc1c(F)ccc2cc(O)cc(-c3c(C#N)cc4c(N5CC6CCC(C5)N6)nc(OC[C@@]56CCCN5C[C@H](F)C6)nc4c3F)c12] | 629.3 | 3TFA salt, HNMR (400 MHz, methanol-d₄, ppm): δ 8.38 (s, 1H), 7.74-7.69 (m, 1H), 7.35-7.34 (m, 1H), 7.31-7.26 (m, 1H), 6.99-6.98 (m, 1H), 5.65-5.50 (m, 1H), 4.83-4.70 (m, 4H), 4.92-4.22 (m, 2H), 4.05-3.84 (m, 5H), 3.50-3.43 (m, 1H), 2.78-2.48 (m, 3H), 2.47-2.06 (m, 9H), 0.83-0.79 (m, 3H). FNMR (376 MHz, methanol-d₄, ppm): δ −120.61 (1F), −123.88 (1F), −174.24 (1F). |
| 150 | 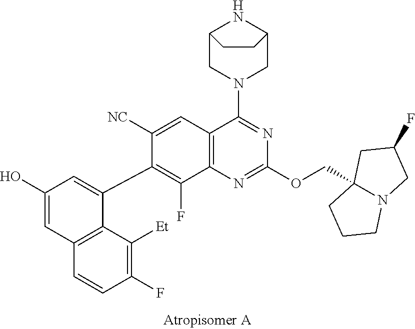 [SMILES: CCc1c(F)ccc2cc(O)cc(-c3c(C#N)cc4c(N5CC6CCC(C5)N6)nc(OC[C@@]56CCCN5C[C@H](F)C6)nc4c3F)c12] | 629.3 | 3TFA salt, HNMR (400 MHz, methanol-d₄, ppm): δ 8.38 (s, 1H), 7.73-7.69 (m, 1H), 7.35-7.34 (m, 1H), 7.31-7.26 (m, 1H), 7.00-6.98 (m, 1H), 5.64-5.50 (m, 1H), 4.83-4.66 (m, 4H), 4.29-4.23 (m, 2H), 4.06-3.84 (m, 5H), 3.50-3.43 (m, 1H), 2.77-2.10 (m, 12H), 0.82 (t, J = 7.6 Hz, 3H). FNMR (376 MHz, methanol-d₄, ppm): δ −120.62 (1F), −123.90 (1F), −174.19 (1F). |
| 151 | 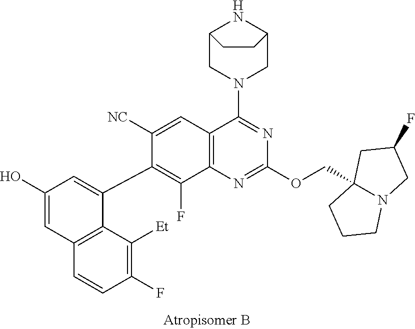 [SMILES: CCc1c(F)ccc2cc(O)cc(-c3c(C#N)cc4c(N5CC6CCC(C5)N6)nc(OC[C@@]56CCCN5C[C@H](F)C6)nc4c3F)c12] | 629.3 | 3TFA salt, HNMR (400 MHz, methanol-d₄, ppm): δ 8.38 (s, 1H), 7.74-7.70 (m, 1H), 7.35-7.34 (m, 1H), 7.31-7.26 (m, 1H), 6.99-6.97 (m, 1H), 5.64-5.51 (m, 1H), 4.82-4.70 (m, 4H), 4.29-4.22 (m, 2H), 4.06-3.83 (m, 5H), 3.50-3.43 (m, 1H), 2.78-2.05 (m, 12H), 0.81 (t, J = 7.2 Hz, 3H). FNMR (376 MHz, methanol-d₄, ppm): δ −120.61 (1F), −123.88 (1F), −174.21 (1F). |
| 156 | 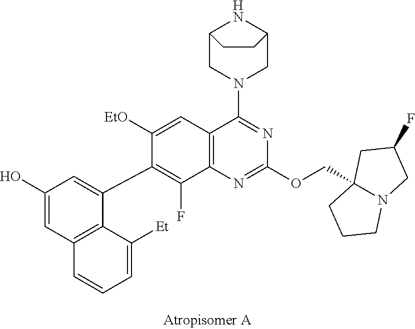 [SMILES: CCOc1cc2c(N3CC4CCC(C3)N4)nc(OC[C@@]34CCCN3C[C@H](F)C4)nc2c(F)c1-c1cc(O)cc2cccc(CC)c12] | 630.4 | 2.7TFA salt, HNMR (400 MHz, methanol-d₄, ppm): δ 7.60 (d, J = 7.6 Hz, 1H), 7.32 (t, J = 7.2 Hz, 1H), 7.23 (d, J = 2.8 Hz, 1H), 7.13-7.09 (m, 2H), 6.82 (d, J = 2.4 Hz, 1H), 5.62-5.48 (m, 1H), 4.70-4.57 (m, 4H), 4.27-3.79 (m, 9H), 3.49-3.42 (m, 1H), 2.75-2.18 (m, 12H), 1.13 (t, J = 6.8 Hz, 3H), 0.86 (t, J = 7.2 Hz, 3H). FNMR (400 MHz, methanol- d₄, ppm): δ −127.18 (1F), −174.30 (1F). |
| 157 | 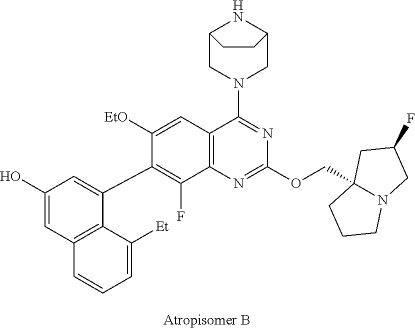 [SMILES: CCOc1cc2c(N3CC4CCC(C3)N4)nc(OC[C@@]34CCCN3C[C@H](F)C4)nc2c(F)c1-c1cc(O)cc2cccc(CC)c12] | 630.4 | 3.7TFA salt, HNMR (400 MHz, methanol-d₄, ppm): δ 7.60 (d, J = 7.6 Hz, 1H), 7.32 (t, J = 8.0 Hz, 1H), 7.23 (d, J = 2.8 Hz, 1H), 7.13-7.09 (m, 2H), 6.82 (d, J = 2.8 Hz, 1H), 5.63-5.48 (m, 1H), 4.71-4.57 (m, 4H), 4.27-3.79 (m, 9H), 3.49-3.42 (m, 1H), 2.76-2.17 (m, 12H), 1.12 (t, J = 6.8 Hz, 3H), 0.85 (t, J = 7.6 Hz, 3H). FNMR (400 MHz, methanol- d₄, ppm): δ −127.13 (1F), −174.28 (1F). |
| 158 | 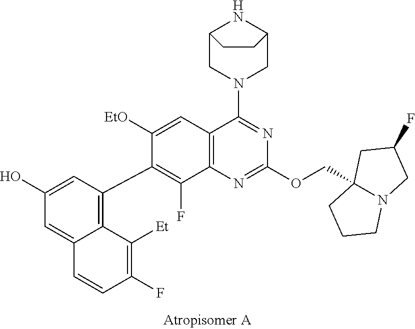 [SMILES: CCOc1cc2c(N3CC4CCC(C3)N4)nc(OC[C@@]34CCCN3C[C@H](F)C4)nc2c(F)c1-c1cc(O)cc2ccc(F)c(CC)c12] | 648.4 | 3TFA salt, HNMR (400 MHz, methanol-d₄, ppm): δ 7.66- 7.63 (m, 1H), 7.25-7.19 (m, 2H), 7.14 (s, 1H), 6.86 (d, J = 2.8 Hz, 1H), 5.62-5.49 (m, 1H), 4.70-4.59 (m, 4H), 4.27- 3.80 (m, 9H), 3.49-3.44 (m, 1H), 2.74-2.18 (m, 12H), 1.15 (t, J = 6.8 Hz, 3H), 0.75 (t, J = 7.2 Hz, 3H). FNMR (400 MHz, methanol-d₄, ppm): δ −121.96 (1F), −126.76 (1F), −174.28 (1F). |
| 159 | 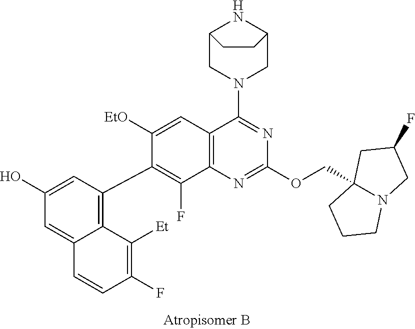 [SMILES: CCOc1cc2c(N3CC4CCC(C3)N4)nc(OC[C@@]34CCCN3C[C@H](F)C4)nc2c(F)c1-c1cc(O)cc2ccc(F)c(CC)c12] | 648.4 | 3TFA salt, HNMR (400 MHz, methanol-d₄, ppm): δ 7.67- 7.63 (m, 1H), 7.25-7.19 (m, 2H), 7.14 (s, 1H), 6.86 (d, J = 2.4 Hz, 1H), 5.62-5.48 (m, 1H), 4.71-4.58 (m, 4H), 4.27- 3.79 (m, 9H), 3.49-3.43 (m, 1H), 2.74-2.17 (m, 12H), 1.15 (t, J = 6.8 Hz, 3H), 0.74 (t, J = 7.2 Hz, 3H). FNMR (400 MHz, methanol-d₄, ppm): δ −121.95 (1F), −126.71 (1F), −174.27 (1F). |
| 160 | 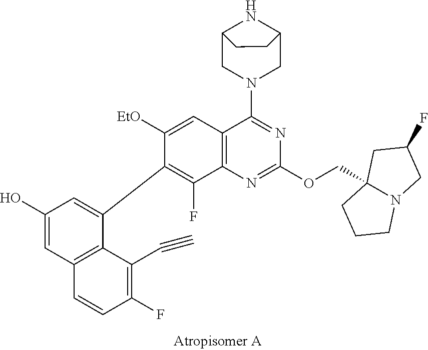 [SMILES: C#Cc1c(F)ccc2cc(O)cc(-c3c(OCC)cc4c(N5CC6CCC(C5)N6)nc(OC[C@@]56CCCN5C[C@H](F)C6)nc4c3F)c12] | 644.3 | 3TFA salt, HNMR (400 MHz, methanol-d₄, ppm): δ 7.85- 7.81 (dd, J = 9.2, 5.6 Hz, 1H), 7.31-7.27 (m, 2H), 7.04- 7.01 (m, 2H), 5.61-5.47 (m, 1H), 4.72-4.55 (m, 4H), 4.27- 3.76 (m, 9H), 3.48-3.41 (m, 1H), 3.20 (s, 1H), 2.72-2.16 (m, 10H), 1.13 (t, J = 7.2 Hz, 3H). FNMR (400 MHz, methanol-d₄, ppm): δ −112.10 (1F), −128.47 (1F), −174.46 (1F). |
| 161 | 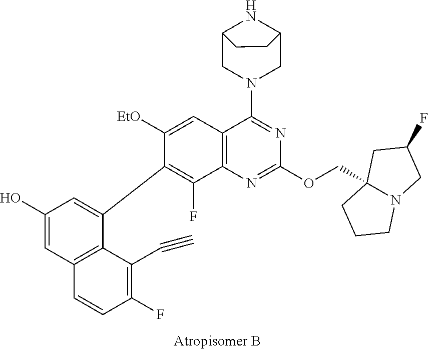 [SMILES: C#Cc1c(F)ccc2cc(O)cc(-c3c(OCC)cc4c(N5CC6CCC(C5)N6)nc(OC[C@@]56CCCN5C[C@H](F)C6)nc4c3F)c12] | 644.3 | 4TFA salt, HNMR (400 MHz, methanol-d₄, ppm): δ 7.85- 7.81 (dd, J = 8.8, 5.6 Hz, 1H), 7.31-7.27 (m, 2H), 7.04- 7.01 (m, 2H), 5.60-5.47 (m, 1H), 4.68-4.56 (m, 4H), 4.27- 3.76 (m, 9H), 3.49-3.42 (m, 1H), 3.19 (s, 1H), 2.72-2.17 (m, 10H), 1.13 (t, J = 6.8 Hz, 3H). FNMR (400 methanol-d₄, ppm): δ −112.14 (1F), −128.43 (1F), −174.44 (1F). |
| 162 | 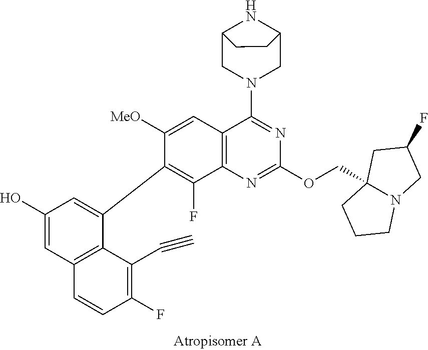 [SMILES: C#Cc1c(F)ccc2cc(O)cc(-c3c(OC)cc4c(N5CC6CCC(C5)N6)nc(OC[C@@]56CCCN5C[C@H](F)C6)nc4c3F)c12] | 630.3 | 3TFA salt, HNMR (400 MHz, methanol-d₄, ppm): δ 7.83 (dd, J = 9.2, 6.0 Hz, 1H), 7.31-7.27 (m, 2H), 7.06 (s, 1H), 7.00 (d, J = 2.4 Hz, 1H), 5.62-5.48 (m, 1H), 4.73-4.57 (m, 4H), 4.28-4.25 (m, 2H), 4.04-3.76 (m, 8H), 3.48-3.42 (m, 1H), 3.20 (s, 1H), 2.72-2.57 (m, 2H), 2.45-2.07 (m, 8H). FNMR (400 MHz, methanol-d₄, ppm): δ −111.97 (1F), −128.42 (1F), −174.46 (1F). |
| 163 | 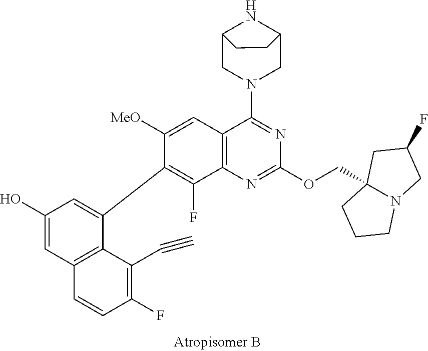 [SMILES: C#Cc1c(F)ccc2cc(O)cc(-c3c(OC)cc4c(N5CC6CCC(C5)N6)nc(OC[C@@]56CCCN5C[C@H](F)C6)nc4c3F)c12] | 630.3 | 3TFA salt, HNMR (400 MHz, methanol-d₄, ppm): δ 7.83 (dd, J = 9.2, 5.6 Hz, 1H), 7.31-7.27 (m, 2H), 7.06 (s, 1H), 7.00 (d, J = 2.4 Hz, 1H), 5.61-5.47 (m, 1H), 4.71-4.59 (m, 4H), 4.27-4.25 (m, 2H), 4.02-3.78 (m, 8H), 3.49-3.42 (m, 1H), 3.19 (s, 1H), 2.72-2.57 (m, 2H), 2.45-2.17 (m, 8H). FNMR (400 MHz, methanol-d₄, ppm): δ −112.02 (1F), −128.35 (1F), −174.43 (1F). |
| 164 | 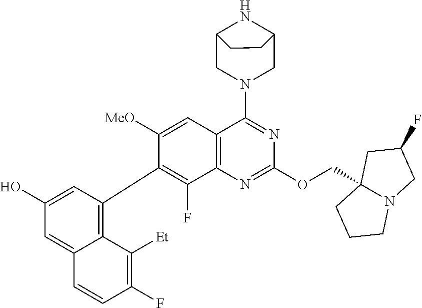 [SMILES: CCc1c(F)ccc2cc(O)cc(-c3c(OC)cc4c(N5CC6CCC(C5)N6)nc(OC[C@@]56CCCN5C[C@H](F)C6)nc4c3F)c12] | 634.3 | 3TFA salt, HNMR (400 MHz, methanol-d₄, ppm): δ 7.65 (dd, J = 8.8, 6.0 Hz, 1H), 7.25-7.16 (m, 3H), 6.85 (d, J = 2.4 Hz, 1H), 5.62-5.49 (m, 1H), 4.72-4.61 (m, 4H), 4.30- 4.24 (m, 2H), 4.06-3.81 (m, 8H), 3.49-3.42 (m, 1H), 2.75- 2.18 (m, 12H), 0.76-0.72 (m, 3H). |
| 165 | 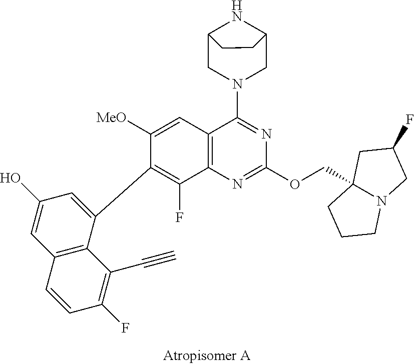 [SMILES: C#Cc1c(F)ccc2cc(O)cc(-c3c(OC)cc4c(N5CC6CCC(C5)N6)nc(OC[C@@]56CCCN5C[C@H](F)C6)nc4c3F)c12] | 618.3 | 3TFA salt, HNMR (400 MHz, methanol-d₄, ppm): δ 7.89- 7.85 (m, 1H), 7.62-7.59 (m, 1H), 7.37-7.31 (m, 2H), 7.11 (d, J = 2.4 Hz, 1H), 5.62-5.49 (m, 1H), 4.81-4.59 (m, 4H), 4.26-4.23 (m, 2H), 4.03-3.76 (m, 5H), 3.49-3.42 (m, 1H) 3.34-3.32 (m, 1H), 2.73-2.58 (m, 2H), 2.45-2.29 (m, 3H), 2.22-2.01 (m, 5H). FNMR (400 MHz, methanol-d₄, ppm): δ −111.35 (1F), −116.51 (1F), −125.54 (1F), −174.50 (1F). |
| 166 | 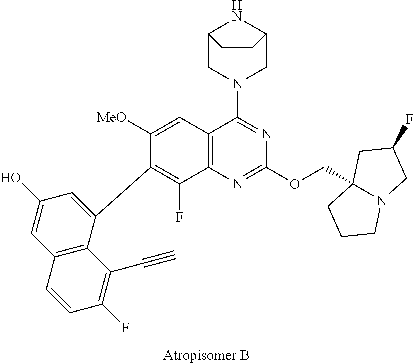 [SMILES: C#Cc1c(F)ccc2cc(O)cc(-c3c(OC)cc4c(N5CC6CCC(C5)N6)nc(OC[C@@]56CCCN5C[C@H](F)C6)nc4c3F)c12] | 618.3 | 3TFA salt, HNMR (400 MHz, methanol-d₄, ppm): δ 7.89- 7.85 (m, 1H), 7.61-7.58 (m, 1H), 7.36-7.30 (m, 2H), 7.12 (d, J = 2.4 Hz, 1H), 5.63-5.49 (m, 1H), 4.72-4.60 (m, 4H), 4.28-4.21 (m, 2H), 4.06-3.77 (m, 5H), 3.50-3.41 (m, 1H), 3.33-3.32 (m, 1H), 2.75-2.53 (m, 2H), 2.46-2.30 (m, 3H), 2.25-2.07 (m, 5H). FNMR (400 MHz, methanol-d₄, ppm): δ −111.40 (1F), −116.64 (1F), −125.41 (1F), −174.30 (1F). |
| 168 | 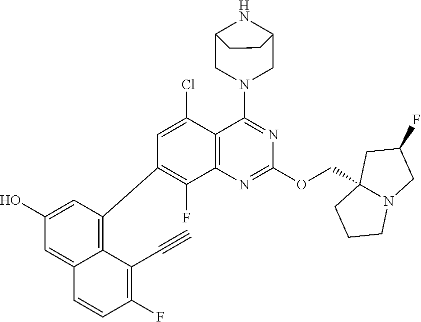 [SMILES: C#Cc1c(F)ccc2cc(O)cc(-c3cc(Cl)c4c(N5CC6CCC(C5)N6)nc(OC[C@@]56CCCN5C[C@H](F)C6)nc4c3F)c12] | 634.3 | 3TFA salt, HNMR (400 MHz, methanol-d₄, ppm): δ 7.88- 7.84 (m, 1H), 7.45-7.42 (m, 1H), 7.35-7.31 (m, 2H), 7.10 (d, J = 2.4 Hz, 1H), 5.63-5.50 (m, 1H), 4.73-4.35 (m, 4H), 4.28-3.83 (m, 7H), 3.49-3.42 (m, 1H), 3.35-3.34 (m, 1H), 2.76-2.54 (m, 2H), 2.46-2.30 (m, 3H), 2.22-1.70 (m, 5H). FNMR (400 MHz, methanol-d₄, ppm): δ −111.31 (1F), −130.40 (1F), −174.25 (1F). |
| 169 | 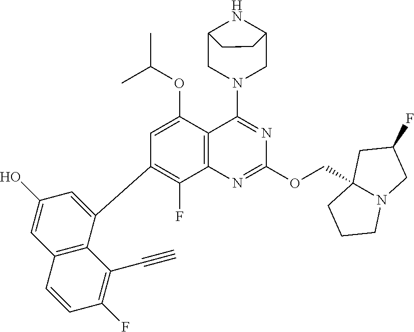 [SMILES: C#Cc1c(F)ccc2cc(O)cc(-c3cc(OC(C)C)c4c(N5CC6CCC(C5)N6)nc(OC[C@@]56CCCN5C[C@H](F)C6)nc4c3F)c12] | 658.3 | 3TFA salt, HNMR (400 MHz, methanol-d₄, ppm): δ 7.87- 7.83 (m, 1H), 7.34-7.29 (m, 2H), 7.10 (d, J = 2.4 Hz, 1H), 6.87 (d, J = 5.6 Hz, 1H), 5.61-5.47 (m, 1H), 4.83-4.62 (m, 4H), 4.56-4.27 (m, 1H), 4.21-4.14 (m, 2H), 4.06-3.78 (m, 5H), 3.51-3.40 (m, 1H), 3.38-3.35 (m, 1H), 2.75-2.52 (m, 2H), 2.49-2.25 (m, 3H), 2.23-1.76 (m, 5H), 1.44 (d, J = 6.0 Hz, 3H), 1.37 (d, J = 6.00 Hz, 3H). FNMR (400 MHz, methanol-d₄, ppm): δ −111.44 (1F), −140.95 (1F), −174.26 (1F). |
| 170 | 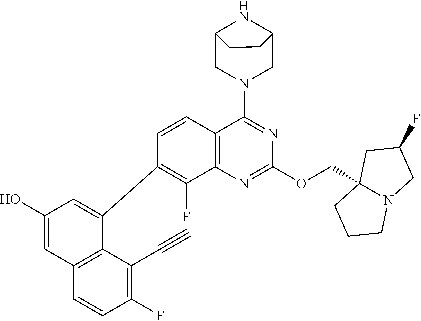 [SMILES: C#Cc1c(F)ccc2cc(O)cc(-c3ccc4c(N5CC6CCC(C5)N6)nc(OC[C@@]56CCCN5C[C@H](F)C6)nc4c3F)c12] | 600.4 | 3TFA salt, HNMR (400 MHz, methanol-d₄, ppm): δ 7.89- 7.82 (m, 2H), 7.45-7.37 (m, 1H), 7.34-7.28 (m, 2H), 7.11- 7.07 (m, 1H), 5.63-5.44 (m, 1H), 4.77-4.65 (m, 4H), 4.31- 4.21 (m, 2H), 4.05-3.79 (m, 5H), 3.49-3.40 (m, 1H), 3.25- 3.21 (m, 1H), 2.76-2.52 (m, 2H), 2.48-2.29 (m, 3H), 2.24- 2.08 (m, 5H). FNMR (400 MHz, methanol-d₄, ppm): δ −111.58 (1F), −129.47 (1F), −174.34 (1F). |

### Biological Example 1. Cell Assay

Ba/F3_KRAS^{G12D} cells (KYinno, China) were generated by transducing Ba/F3 parental cells with the recombinant KRAS^{G12D} lentivirus and followed by 1 ug/mL of puromycin selection and IL3 depletion. Cells were grown in RPMI 1640 medium supplemented with 10% fetal bovine serum, 100 U/mL penicillin and 100 μg/mL streptomycin at 37° C. in an atmosphere of 5% CO₂ in air. Cells were seeded at a density of 5×10³ per well into 96-well plate and incubated overnight. Serial diluted compounds were added to each well. Cells were were treated with the compounds for 3 days, after which cell-titer Glo reagent (Promega #G7572) was used to assess cell proliferation. The luminescence signal was then collected on Tecan Spark plate reader. Inhibition rate is calculated with the formula of % inhibition=100*(Control−well)/(Control−Blank). Cell growth inhibition of IC₅₀ is calculated with the equation of Y=Bottom+(Top−Bottom)/(1+10\hat{ }((Log IC₅₀−X)*Hill Slope))

In Table 2 below, the IC₅₀ levels are described as I, II, or III, wherein I represents IC₅₀ value less than or equal to 500 nM; II represents IC₅₀ value between 500 nM to 5000 nM; and III represents IC₅₀ value more than 5000 nM.

TABLE 2
Inhibition of Ba/F3 KRAS^{G12D}
Cell Proliferation by
Representative Compounds

| | BaF3_KRAS^{G12D} |
|---|---|
| Compound | IC₅₀ (nM) |
| 1 | II |
| 2 | II |
| 3 | II |
| 4 | III |
| 5 | II |
| 6 | II |
| 7 | II |
| 8 | III |
| 9 | III |
| 11 | II |
| 12 | III |
| 14 | III |
| 16 | II |
| 17 | III |
| 18 | II |
| 19 | III |
| 20 | II |
| 21 | II |
| 22 | II |
| 23 | II |
| 24 | II |
| 25 | II |
| 27 | II |
| 28 | I |
| 29 | II |
| 30 | II |
| 31 | II |
| 32 | III |
| 33 | II |
| 34 | II |
| 35 | II |
| 36 | II |
| 37 | II |
| 38 | II |
| 39 | II |
| 40 | II |
| 41 | II |
| 42 | II |
| 43 | II |
| 44 | II |
| 45 | II |
| 46 | III |
| 47 | I |
| 48 | II |
| 49 | III |
| 50 | II |
| 51 | II |
| 52 | II |
| 53 | II |
| 54 | II |
| 55 | I |
| 56 | II |
| 57 | II |
| 58 | I |
| 59 | II |
| 60 | II |
| 61 | II |
| 64 | I |
| 65 | II |
| 66 | III |
| 67 | III |
| 69 | II |
| 70 | III |
| 72 | II |
| 74 | I |
| 75 | II |
| 76 | II |
| 77 | I |
| 78 | II |
| 79 | II |
| 80 | III |
| 81 | II |
| 82 | III |
| 83 | II |
| 85 | I |
| 86 | I |
| 87 | II |
| 88 | I |
| 89 | II |
| 90 | III |
| 91 | II |
| 92 | I |
| 96 | II |
| 100 | II |
| 101 | I |
| 102 | II |
| 103 | I |
| 104 | II |
| 105 | II |
| 106 | I |
| 107 | I |
| 108 | II |
| 109 | II |
| 111 | II |
| 116 | III |
| 117 | I |
| 118 | I |
| 119 | II |
| 120 | I |
| 121 | III |
| 122 | III |
| 123 | III |
| 124 | I |
| 126 | II |
| 127 | II |
| 128 | II |
| 129 | I |
| 130 | III |
| 131 | II |
| 132 | II |
| 133 | I |
| 134 | II |
| 135 | III |
| 137 | II |
| 138 | I |
| 139 | I |
| 140 | II |
| 142 | I |
| 143 | II |
| 144 | II |
| 146 | I |
| 147 | I |
| 148 | I |
| 149 | I |
| 150 | I |
| 151 | I |
| 152 | I |
| 153 | I |
| 154 | I |
| 155 | I |
| 156 | I |
| 157 | II |
| 158 | I |
| 159 | II |
| 160 | I |
| 161 | I |
| 162 | I |
| 163 | I |
| 164 | I |
| 165 | I |
| 166 | I |
| 167 | I |
| 168 | I |
| 169 | I |
| 170 | I |

### Biological Example 2. KRAS^{G12D} Protein Binding Assay

The Temperature-dependent Fluorescence (TdF) assay was used to analyze binding affinity of compound to recombinant human KRAS^{G12D} protein. The TdF assay was conducted in the 96-well-based real-time fluorescence plate reader (ABI 7500 or Roche LightCycler 480). Fluorescent dye Sypro Orange (Sigma) was used to monitor the protein folding-unfolding transition. Protein-compound binding was gauged by the shift in the unfolding transition temperature (ΔTm) acquired with and without compound. Each reaction sample consists of 6 μM KRAS^{G12D} Protein, 10 μM compound, and Sypro Orange dye (in 1% DMSO) in 20 μL reaction buffer (25 mM HEPES pH 7.5, 150 mM NaCl, 10 mM MgCl₂). The sample plate was heated from 30° C. to 95° C. with a thermal ramping rate of 0.5%, taking a fluorescence reading every 0.4° C. using a CY3 channel matching the excitation and emission wavelengths of Sypro Orange (λ ex 470 nm; λ em 570 nm). Binding affinity (K_{d} value) was calculated based on the degree of fluorescent shift of the protein with and without compound.

In Table 3 below, the K_{d} levels are described as I, II, or III, wherein I represents K_{d} value less than or equal to 500 nM; II represents K_{d} value in the range of 500 nM to 5000 nM; and III represents K_{d} value more than 5000 nM.

TABLE 3
Binding Affinity of
Representative Compounds

| | TdF |
|---|---|
| Compound | K_{d} (nM) |
| 1 | III |
| 2 | I |
| 3 | III |
| 4 | II |
| 5 | II |
| 6 | III |
| 7 | III |
| 8 | III |
| 11 | II |
| 12 | III |
| 14 | III |
| 15 | III |
| 16 | III |
| 17 | III |
| 18 | III |
| 19 | III |
| 20 | III |
| 21 | III |
| 22 | III |
| 23 | II |
| 24 | III |
| 25 | III |
| 27 | III |
| 28 | III |
| 29 | III |
| 30 | I |
| 31 | III |
| 32 | II |
| 33 | I |
| 35 | II |
| 36 | III |
| 39 | III |
| 40 | III |
| 41 | III |
| 42 | III |
| 43 | III |
| 44 | III |
| 45 | III |
| 46 | II |
| 47 | I |
| 48 | II |
| 49 | III |
| 50 | II |
| 51 | III |
| 52 | III |
| 53 | III |
| 54 | III |
| 55 | I |
| 56 | III |
| 57 | III |
| 58 | I |
| 59 | III |
| 60 | III |
| 61 | I |
| 63 | II |
| 64 | II |
| 65 | I |
| 66 | III |
| 67 | II |
| 68 | I |
| 69 | II |
| 70 | II |
| 71 | I |
| 72 | II |
| 73 | I |
| 74 | I |
| 75 | III |
| 76 | I |
| 77 | I |
| 78 | III |
| 79 | I |
| 80 | I |
| 81 | I |
| 82 | III |
| 83 | III |
| 84 | III |
| 85 | I |
| 86 | I |
| 87 | II |
| 88 | I |
| 89 | II |
| 91 | III |
| 92 | I |
| 96 | III |
| 100 | I |
| 101 | I |
| 102 | III |
| 103 | I |
| 104 | III |
| 105 | III |
| 106 | I |
| 107 | I |
| 108 | III |
| 109 | III |
| 111 | II |
| 112 | I |
| 116 | II |
| 117 | I |
| 118 | I |
| 119 | III |
| 120 | I |
| 121 | III |
| 122 | I |
| 123 | I |
| 124 | I |
| 126 | II |
| 127 | II |
| 128 | III |
| 129 | I |
| 130 | III |
| 131 | I |
| 132 | I |
| 133 | I |
| 134 | III |
| 135 | III |
| 136 | III |
| 137 | I |
| 138 | I |
| 139 | I |
| 140 | II |
| 141 | II |
| 142 | I |
| 143 | I |
| 144 | III |
| 146 | I |
| 154 | I |
| 163 | I |
| 166 | I |
| 168 | I |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 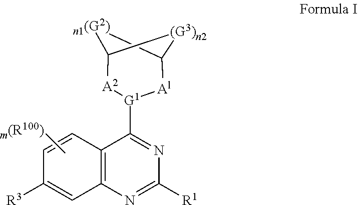
wherein:
G¹ is CR¹⁰ or N;
each occurrence of G² and G³ is independently CR¹¹R¹², O, or NR²⁰, provided that at least one instance of G² and G³ is NR²⁰;
n1 and n2 are each independently an integer of 1, 2, 3, or 4;
A¹ and A² are each independently a bond, CR¹¹R¹², O, or NR²⁰, provided that at least one of A¹ and A² is not O or NR²⁰;
R¹ is hydrogen, -(L¹)ⱼ₁-OR³⁰, halogen, -(L¹)ⱼ₁-NR²¹R²², or an optionally substituted heterocyclic or heteroaryl ring;
R³ is an optionally substituted aryl or an optionally substituted heteroaryl,
R¹⁰⁰ at each occurrence is independently F, Cl, Br, I, CN, —OH, —C(O)NH₂, —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), cyclopropyl, cyclobutyl, optionally substituted C₁₋₄ alkoxy (e.g., methoxy, ethoxy, —O—CH₂-cyclopropyl), cyclopropoxy, or cyclobutoxy; and
m is 0, 1, 2, or 3;
wherein:
j1 is 0 or 1, and when j1 is 1, L¹ is an optionally substituted alkylene, an optionally substituted carbocyclylene, an optionally substituted heterocyclylene;
each occurrence of R¹⁰, R¹¹, or R¹² is independently hydrogen, F, —OH, or an optionally substituted C₁₋₆ alkyl, or R¹¹ and R¹² together with the carbon they are both attached to are joined to form an oxo or imino group or a ring;
R²⁰ at each occurrence is independently hydrogen, a nitrogen protecting group, or an optionally substituted C₁₋₆ alkyl;
R²¹ and R²² are independently hydrogen, a nitrogen protecting group, an optionally substituted C₁₋₆ alkyl, an optionally substituted carbocyclic ring, or an optionally substituted heterocyclic ring; or R²¹ and R²² are joined to form an optionally substituted heterocyclic or heteroaryl ring; and
R³⁰ is hydrogen, an oxygen protecting group, an optionally substituted C₁₋₆ alkyl, an optionally substituted carbocyclic ring, an optionally substituted aryl, an optionally substituted heteroaryl, or an optionally substituted heterocyclic ring.

2. The compound of claim 1, or a pharmaceutically acceptable salt thereof, wherein: G¹ is CH or N.

3. The compound of claim 1 or 2, or a pharmaceutically acceptable salt thereof, wherein A¹ and A² are each independently a bond or CH₂.

4. The compound of claim 1 or 2, or a pharmaceutically acceptable salt thereof, wherein A¹ and A² are both a bond or both CH₂.

5. The compound of any one of claims 1-4, or a pharmaceutically acceptable salt thereof, wherein each occurrence of G² is independently CR¹¹R¹².

6. The compound of any one of claims 1-5, or a pharmaceutically acceptable salt thereof, wherein n1 is 1, 2, or 3.

7. The compound of any one of claims 1-6, or a pharmaceutically acceptable salt thereof, wherein one instance of G³ is NH.

8. The compound of any one of claims 1-7, or a pharmaceutically acceptable salt thereof, wherein n2 is 1, 2, or 3.

9. The compound of claim 1, or a pharmaceutically acceptable salt thereof, wherein the moiety 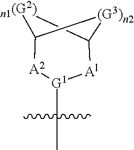
in Formula I is selected from the following:

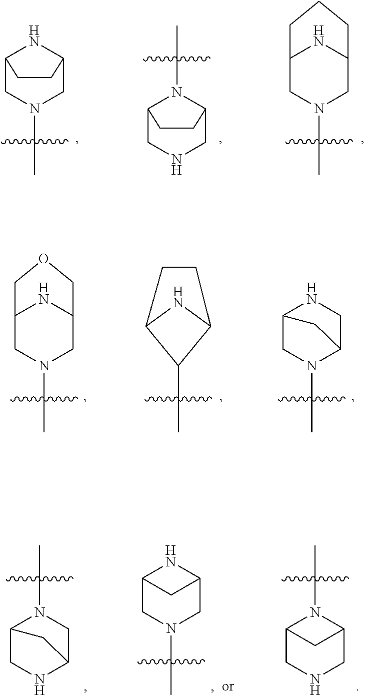

10. The compound of any one of claims 1-9, or a pharmaceutically acceptable salt thereof, wherein R¹ is —OR³⁰, wherein R³⁰ is a —C₁₋₆ alkylene-R¹⁰¹, wherein R¹⁰ is NR²³R²⁴ or an optionally substituted 4-10 membered heterocyclic ring,
wherein the C₁₋₆ alkylene is optionally substituted, e.g., with one or more substituents independently selected from F, OH, NR²⁵R²⁶, and C₁₋₄ alkyl optionally substituted with 1-3 fluorine, or two substituents of the alkylene group are joined to form a ring;
R²³ and R²⁴ are independently hydrogen, a nitrogen protecting group, an optionally substituted C₁₋₆ alkyl, an optionally substituted carbocyclic ring, or an optionally substituted heterocyclic ring; or R²³ and R²⁴ are joined to form an optionally substituted heterocyclic or heteroaryl ring; and
R²⁵ and R²⁶ are independently hydrogen, a nitrogen protecting group, an optionally substituted C₁₋₆ alkyl, an optionally substituted carbocyclic ring, or an optionally substituted heterocyclic ring; or R²⁵ and R²⁶ are joined to form an optionally substituted heterocyclic or heteroaryl ring.

11. The compound of claim 10, or a pharmaceutically acceptable salt thereof, wherein R¹⁰¹ is NR²³R²⁴, wherein R²³ and R²⁴ are independently hydrogen, a C₁₋₄ alkyl, or R²³ and R²⁴ together with the N they are both attached to are joined to form an optionally substituted 4-8 membered monocyclic heterocyclic ring having one or two ring heteroatoms.

12. The compound of claim 10 or 11, or a pharmaceutically acceptable salt thereof, wherein R¹⁰¹ is NR²³R²⁴, wherein R²³ and R²⁴ together with the N they are both attached to are joined to form a ring selected from 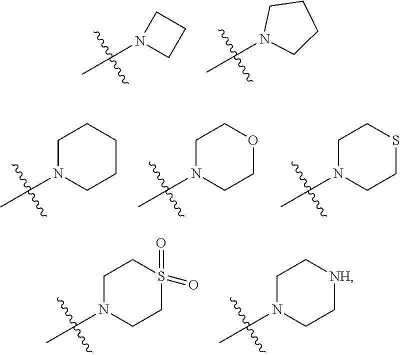
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

13. The compound of claim 10 or 11, or a pharmaceutically acceptable salt thereof, wherein R¹⁰¹ is a monocyclic 4-8 membered heterocyclic ring having 1 or 2 ring heteroatoms independently selected from N, O, and S, or a fused or spiro bicyclic 6-10 membered heterocyclic ring having one to three ring heteroatoms independently selected from N, O, and S, wherein the monocyclic or bicyclic ring is optionally substituted.

14. The compound of claim 13, or a pharmaceutically acceptable salt thereof, wherein R¹⁰¹ is a monocyclic ring selected from the following: 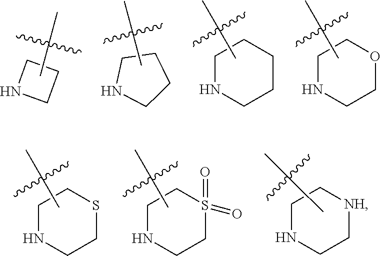
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

15. The compound of claim 13, or a pharmaceutically acceptable salt thereof, wherein R¹⁰¹ is a bicyclic ring selected from the following: 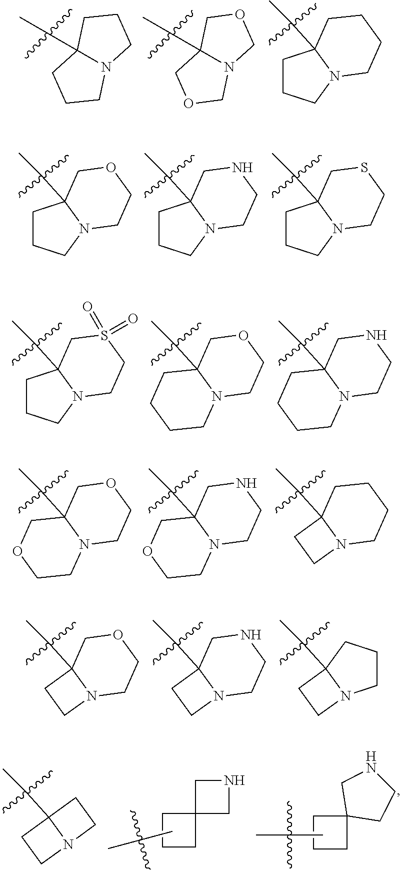
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

16. The compound of any one of claims 10-15, or a pharmaceutically acceptable salt thereof, wherein the —C₁₋₆ alkylene-unit in R³⁰ is selected from —CH₂—, —CH₂—CH₂—, —CH₂—CH₂—CH₂—,

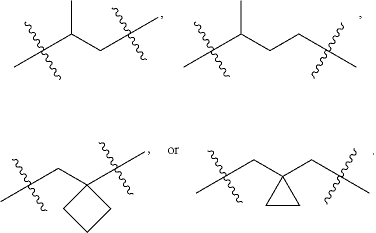

17. The compound of any one of claims 1-9, or a pharmaceutically acceptable salt thereof, wherein R¹ is

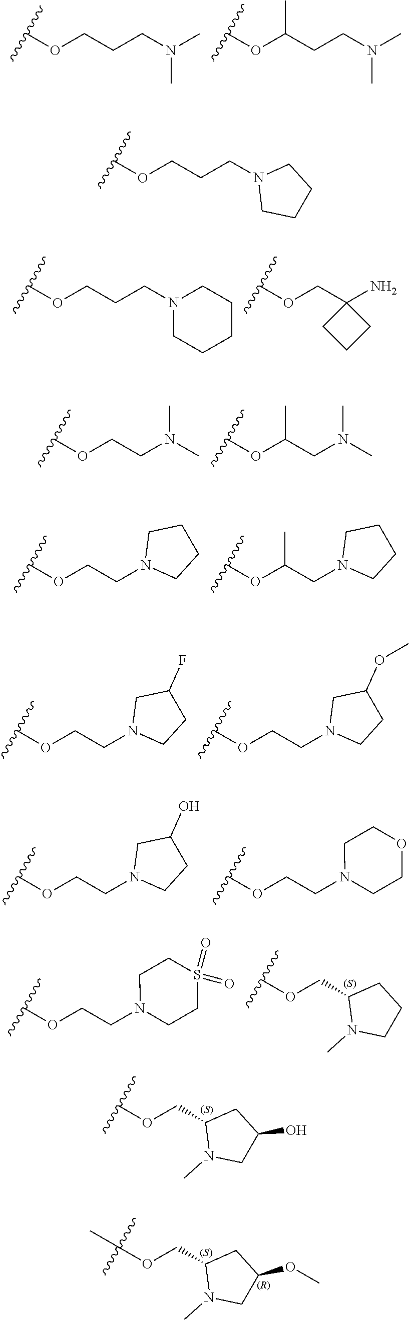

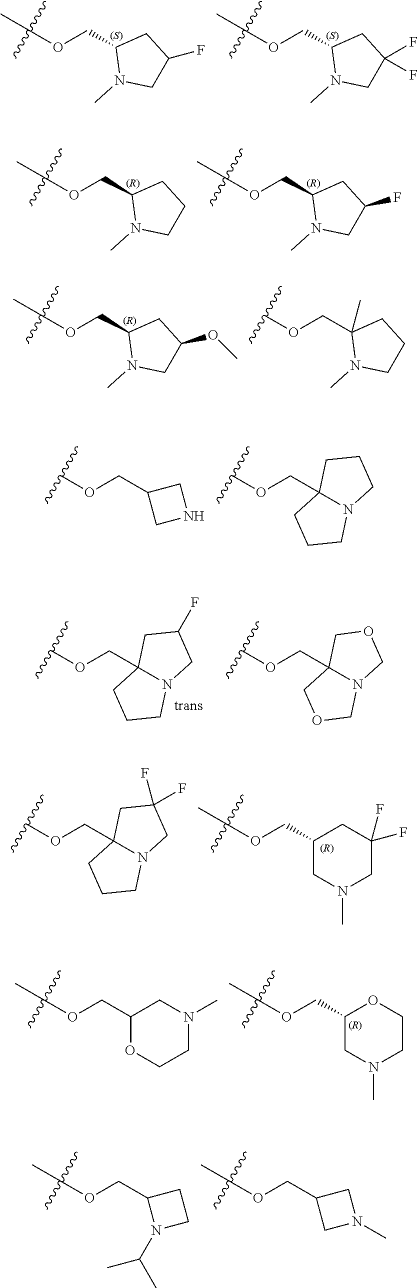

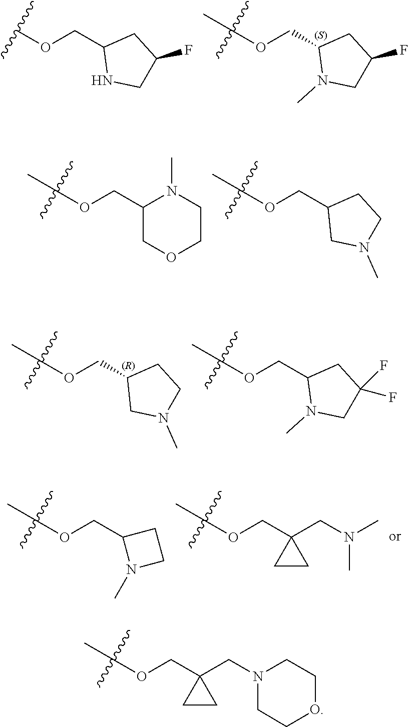

18. The compound of any one of claims 1-9, or a pharmaceutically acceptable salt thereof, wherein R¹ is OR³⁰, wherein R³⁰ is an optionally substituted C₃₋₆ carbocyclic ring or 4-10 membered heterocyclic ring, preferably, a monocyclic 4-8 membered heterocyclic ring having 1 or 2 ring heteroatoms independently selected from N, O, and S, or a fused or spiro bicyclic 6-10 membered heterocyclic ring having one to three ring heteroatoms independently selected from N, O, and S, wherein the monocyclic or bicyclic ring is optionally substituted.

19. The compound of claim 18, or a pharmaceutically acceptable salt thereof, wherein R³⁰ is a monocyclic ring selected from the following: 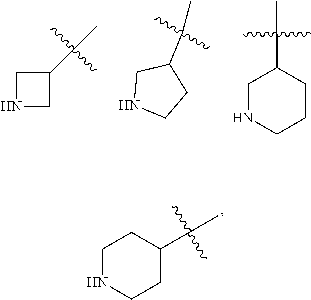 [SMILES: *C1CNC1 ; *C1CCNC1 ; *C1CCCNC1 ; *C1CCNCC1]
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, tetrahydropyranyl, —N(CH₃)₂, —OH, and —OCH₃.

20. The compound of any one of claims 1-9, or a pharmaceutically acceptable salt thereof, wherein R¹ is selected from

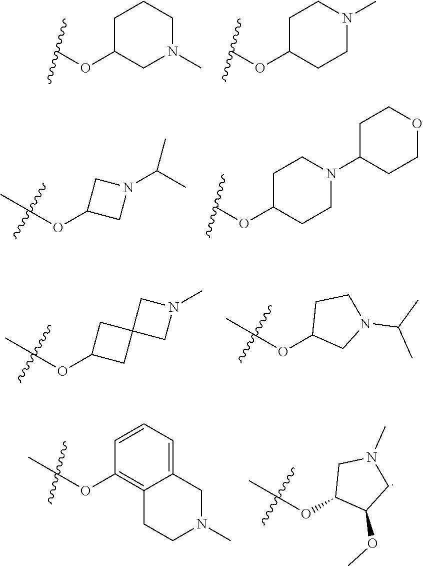

21. The compound of any one of claims 1-9, or a pharmaceutically acceptable salt thereof, wherein R¹ is NR²¹R²² or —C₁₋₆ alkylene-NR²¹R²²,
wherein R²¹ and R²² are independently hydrogen, an optionally substituted C₁₋₆ alkyl, or an optionally substituted heterocyclic ring; or R²¹ and R²² together with the N they are both attached to are joined to form an optionally substituted heterocyclic ring having one or two ring heteroatoms.

22. The compound of claim 21, or a pharmaceutically acceptable salt thereof, wherein R²¹ and R²² together with the N they are both attached to are joined to form a ring selected from 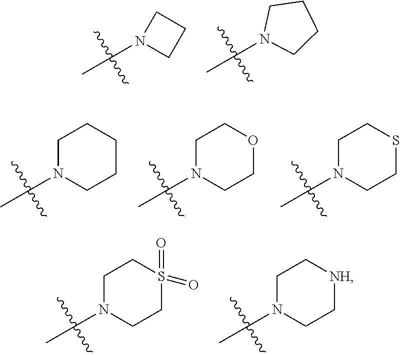
each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

23. The compound of any one of claims 1-9, or a pharmaceutically acceptable salt thereof, wherein R¹ is selected from

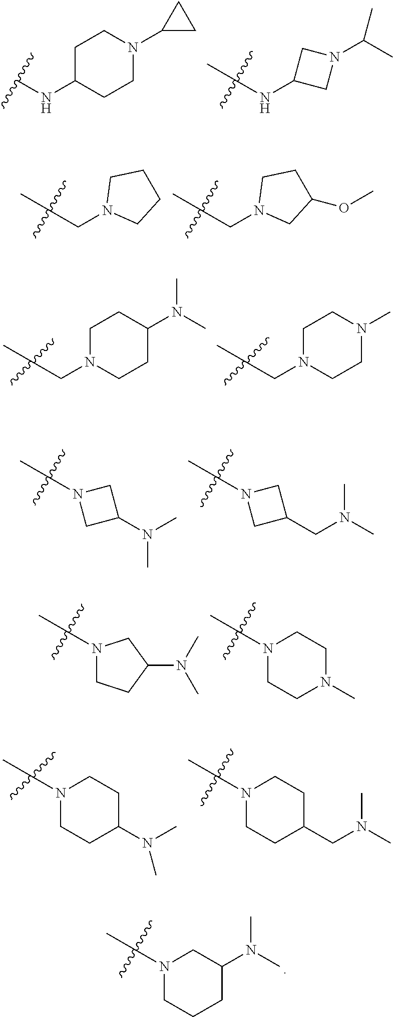

24. The compound of any one of claims 1-9, or a pharmaceutically acceptable salt thereof, wherein R¹ is an optionally substituted heterocyclic ring, preferably, a monocyclic 4-8 membered heterocyclic ring having 1 or 2 ring heteroatoms independently selected from N, O, and S, or a fused or spiro bicyclic 6-10 membered heterocyclic ring having one to three ring heteroatoms independently selected from N, O, and S, wherein the monocyclic or bicyclic ring is optionally substituted.

25. The compound of claim 24, or a pharmaceutically acceptable salt thereof, wherein R¹ is selected from 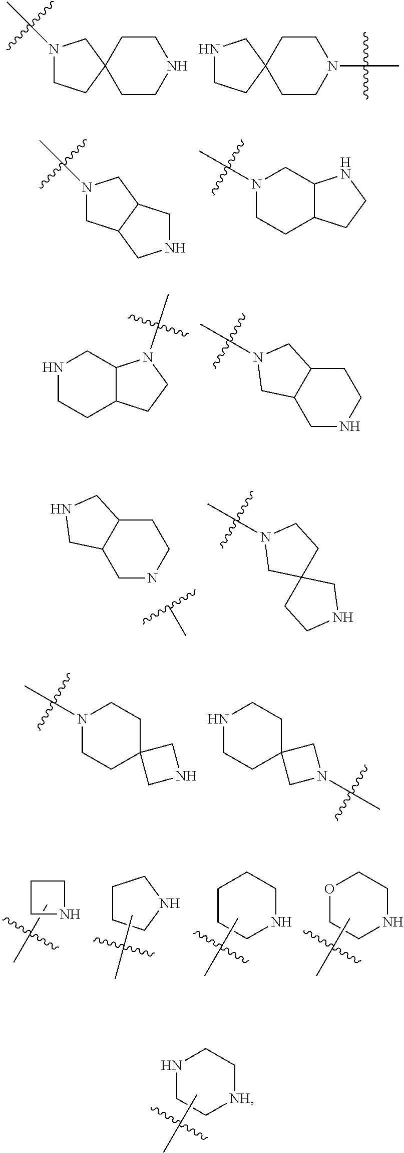
each of which is optionally substituted with one or more (e.g., 1 or 2) substituents independently selected from F, —(CH₂)ₓ—OH, —(CH₂)ₓ—C₁₋₄ alkoxy, optionally substituted with 1-3 fluorine, oxo, C₁₋₄ alkyl optionally substituted with 1-3 fluorine, —(CH₂)ₓ—NH₂, —(CH₂)ₓ—NH(C₁₋₄ alkyl), —(CH₂)ₓ—N(C₁₋₄ alkyl)(C₁₋₄ alkyl), —(CH₂)ₓ-cyclopropyl, —(CH₂)ₓ-cyclobutyl, and —(CH₂)ₓ-(4-6 membered heterocyclic ring having 1 or 2 ring heteroatoms independently selected from O, N, and S), wherein x is 0, 1, 2, or 3, preferably, the substituents are independently selected from F, methyl, ethyl, isopropyl, cyclopropyl, —(CH₂)—N(CH₃)₂, —N(CH₃)₂, —OH, and —OCH₃.

26. The compound of claim 24, or a pharmaceutically acceptable salt thereof, wherein R¹ is selected from

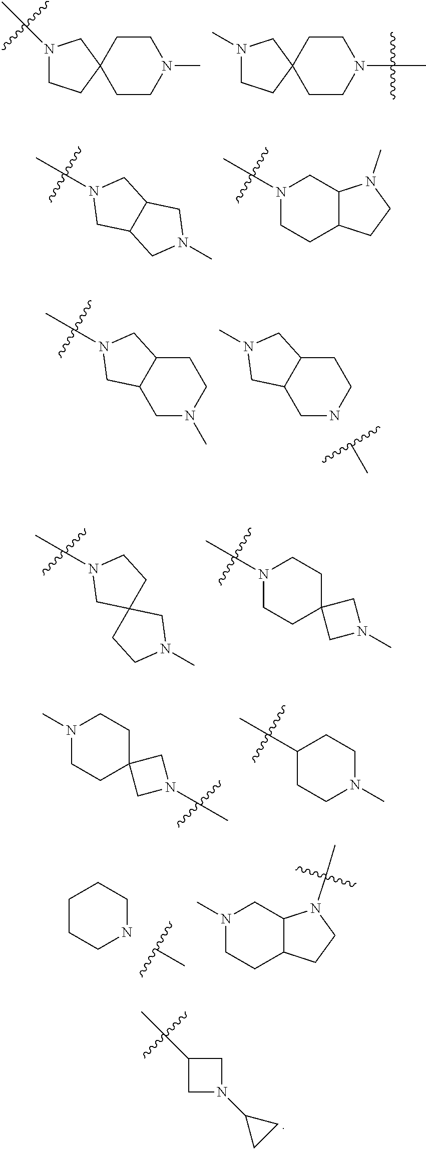

27. The compound of any one of claims 1-26, wherein R¹⁰⁰ at each occurrence is independently F, Cl, —CN, —OH, methoxy, ethoxy, —O—CH₂-cyclopropyl, —C(O)NHMe, CF₃, methyl, ethyl, isopropyl, or cyclopropyl.

28. The compound of any one of claims 1-26, wherein m is 2, and both R¹⁰⁰ are ortho to the R³ group.

29. The compound of any one of claims 1-28, wherein R³ is (1) a phenyl, pyridyl, naphthyl, or bicyclic heteroaryl (e.g., benzothiazolyl, indazolyl, or isoquinolinyl) each of which is optionally substituted, e.g., with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂; or (2) a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂.

30. The compound of any one of claims 1-28, wherein R³ is selected from: 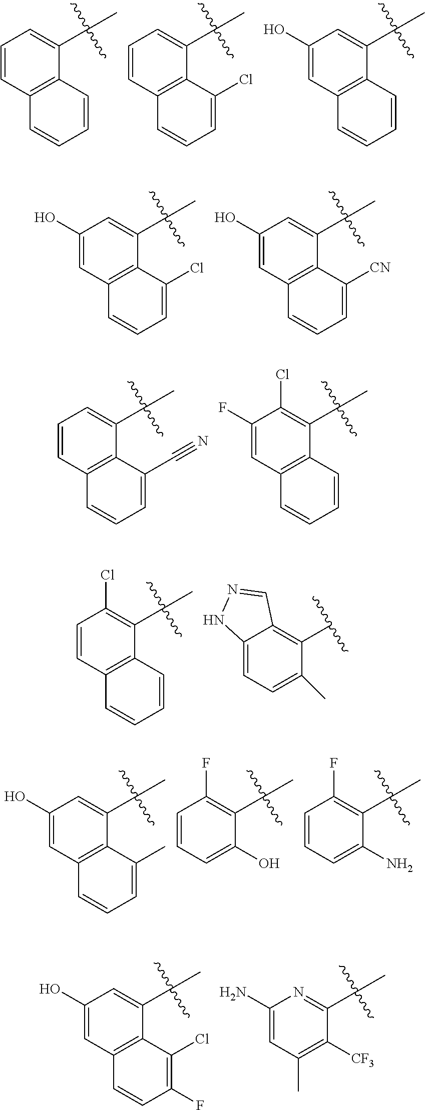 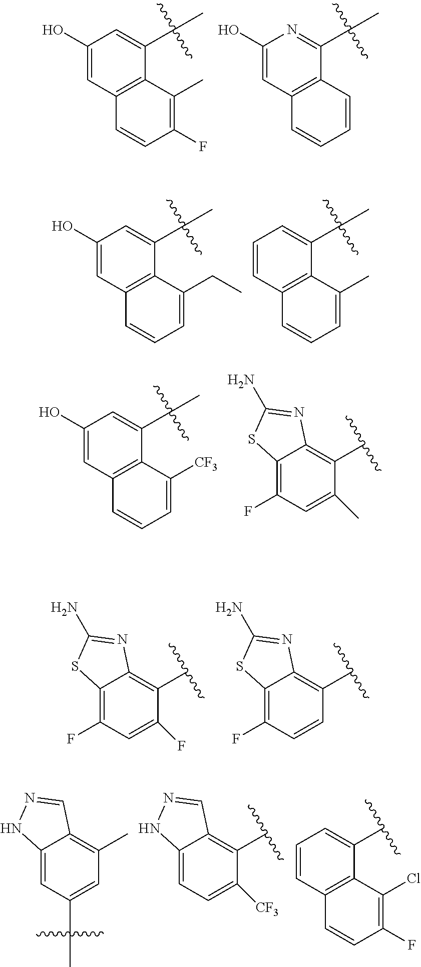
or R³ is selected from

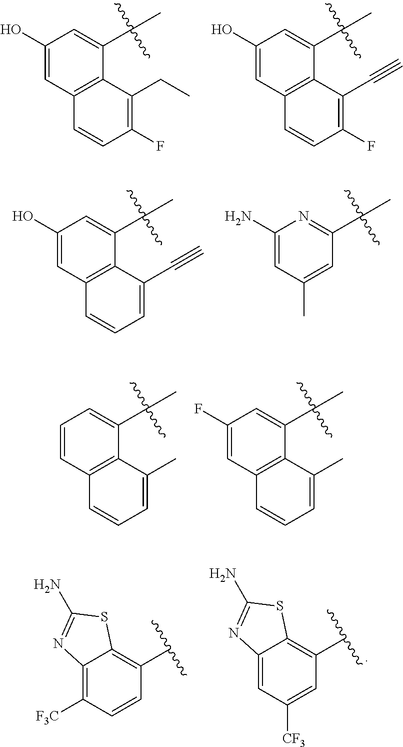

31. A compound of Formula II, or a pharmaceutically acceptable salt thereof: 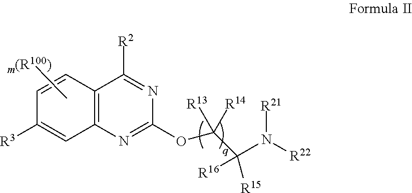
wherein:
R¹³ and R¹⁴ at each occurrence are independently hydrogen or a C₁₋₄ alkyl,
q is an integer of 0-6,
R¹⁵, R¹⁶, R²¹, and R²², together with the intervening carbon and nitrogen atoms, form an optionally substituted 6-10 membered fused bicyclic ring,
R² is a ring or ring-chain structure which has a pKa of about 6 or higher,
R³ is an optionally substituted aryl or an optionally substituted heteroaryl,
R¹⁰⁰ at each occurrence is independently F, Cl, Br, I, —CN, —OH, —C(O)NH₂, —C(O)NH(C₁₋₆ alkyl), —C(O)N(C₁₋₆ alkyl)(C₁₋₆ alkyl), optionally substituted C₁₋₄ alkyl (e.g., methyl, ethyl, CF₃, etc.), cyclopropyl, cyclobutyl, optionally substituted C₁₋₄ alkoxy (e.g., methoxy, ethoxy, —O—CH₂-cyclopropyl), cyclopropoxy, or cyclobutoxy; and
m is 0, 1, 2, or 3.

32. The compound of claim 31, or a pharmaceutically acceptable salt thereof, wherein q is 1.

33. The compound of claim 31, or a pharmaceutically acceptable salt thereof, wherein q is 2.

34. The compound of any one of claims 31-33, or a pharmaceutically acceptable salt thereof, wherein R¹³ and R¹⁴ at each occurrence are independently hydrogen or methyl.

35. The compound of any one of claims 31-34, or a pharmaceutically acceptable salt thereof, wherein R¹⁵, R¹⁶, R²¹, and R²², together with the intervening carbon and nitrogen atoms, form an optionally substituted 6-10 membered fused bicyclic ring 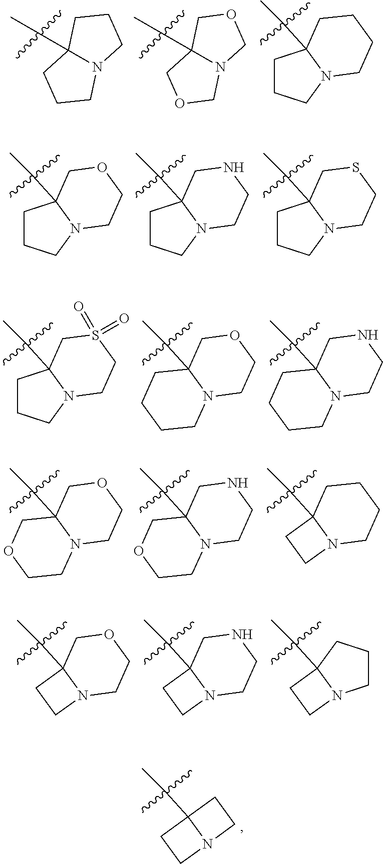
each of which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

36. The compound of any one of claims 31-34, or a pharmaceutically acceptable salt thereof, wherein R¹⁵, R¹⁶, R²¹, and R²², together with the intervening carbon and nitrogen atoms, form 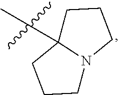 [SMILES: *C12CCCN1CCC2]
which is optionally substituted with one or more (e.g., 1 or 2) substituents independently selected from F, —OH, C₁₋₄ alkoxy optionally substituted with 1-3 fluorine, oxo, C₁₋₄ alkyl optionally substituted with 1-3 fluorine, NH₂, NH(C₁₋₄ alkyl), N(C₁₋₄ alkyl)(C₁₋₄ alkyl), cyclopropyl, cyclobutyl, and a 4-6 membered heterocyclic ring having 1 or 2 ring heteroatoms independently selected from O, N, and S, preferably, the substituents are independently selected from F, methyl, ethyl, isopropyl, cyclopropyl, —N(CH₃)₂, —OH, and —OCH₃.

37. The compound of any one of claims 31-34, or a pharmaceutically acceptable salt thereof, wherein the 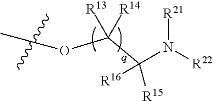
unit in Formula II is selected from

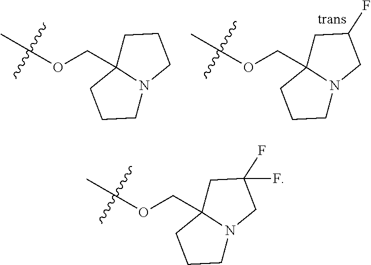

38. The compound of any one of claims 31-37, or a pharmaceutically acceptable salt thereof, wherein R² is -(L²)ⱼ₂-R¹⁰², wherein
j2 is 0 or 1, and when j2 is 1, L² is CH₂, O, NH, or NCH₃,
R¹⁰² is an optionally substituted 4-10 membered heterocyclic or heteroaryl ring having one or two ring nitrogen atoms.

39. The compound of claim 38, or a pharmaceutically acceptable salt thereof, wherein j2 is 0, and R¹⁰² is an optionally substituted 4-10 membered heterocyclic ring having one or two ring nitrogen atoms.

40. The compound of claim 39, or a pharmaceutically acceptable salt thereof, wherein R¹⁰² is selected from the following ring structures: 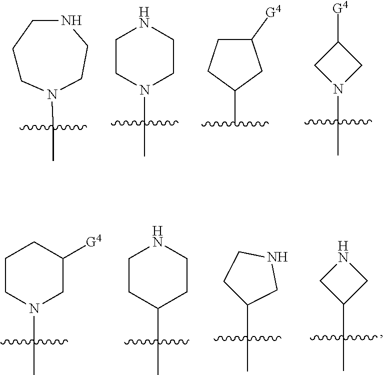
wherein G⁴ is -(L³)ⱼ₃-NH₂, -(L³)ⱼ₃-NH(C₁₋₄ alkyl), wherein j3 is 0 or 1, and when j3 is 1, L³ is C₁₋₄ alkylene, or G⁴ and one substituent on the ring are joined together to form a 4-6 membered heterocyclic ring having one or two ring nitrogen atoms;
and wherein each of the ring structures is optionally substituted with 1-3 (typically 1 or 2) substituents independently selected from C₁₋₄ alkyl, fluorine substituted C₁₋₄ alkyl, hydroxyl substituted C₁₋₄ alkyl, alkoxy substituted C₁₋₄ alkyl, cyano substituted C₁₋₄ alkyl, and CONH₂, or two substituents are combined to form an oxo, imino, or a ring structure.

41. The compound of claim 39, or a pharmaceutically acceptable salt thereof, wherein R¹⁰² is selected from:

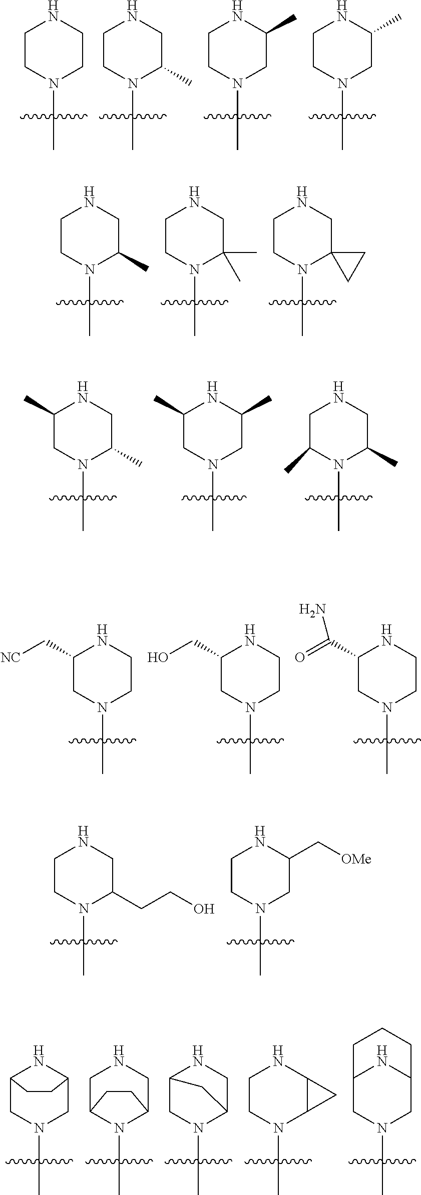

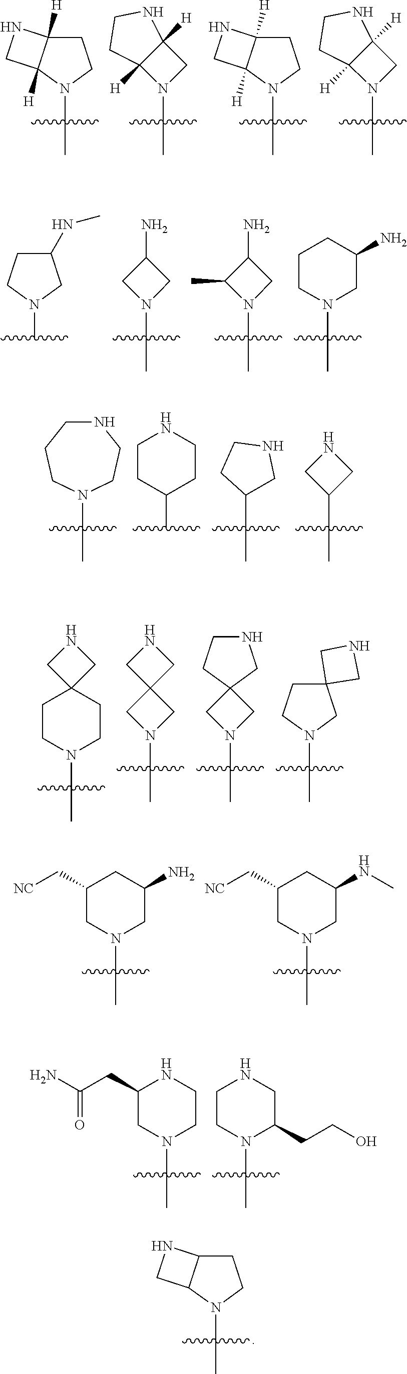

42. The compound of claim 38, or a pharmaceutically acceptable salt thereof, wherein j2 is 1, L² is CH₂ or NHT, and R¹⁰² is an optionally substituted 4-8 membered heterocyclic ring.

43. The compound of claim 42, or a pharmaceutically acceptable salt thereof, wherein R² is selected from

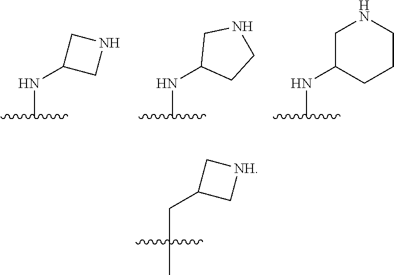 [SMILES: *NC1CNC1 ; *NC1CCNC1 ; *NC1CCCNC1 ; *CC1CNC1]

44. The compound of any one of claims 31-37, or a pharmaceutically acceptable salt thereof, wherein R² is a C₃₋₇ carbocyclic, phenyl, or 5 or 6 membered heteroaryl ring, each of which has at least one nitrogen containing substituent.

45. The compound of claim 44, or a pharmaceutically acceptable salt thereof, wherein R² is selected from

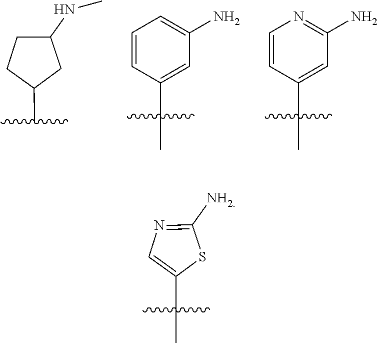

46. The compound of any one of claims 31-45, wherein R¹⁰⁰ at each occurrence is independently F, Cl, —CN, —OH, methoxy, ethoxy, —O—CH₂-cyclopropyl, —C(O)NHMe, CF₃, methyl, ethyl, isopropyl, or cyclopropyl.

47. The compound of any one of claims 31-46, wherein m is 2, and both R¹⁰⁰ are ortho to the R³ group.

48. The compound of any one of claims 31-47, wherein R³ is (1) a phenyl, pyridyl, naphthyl, or bicyclic heteroaryl (e.g., benzothiazolyl, indazolyl, or isoquinolinyl) each of which is optionally substituted, e.g., with 1-3 substituents independently selected from F, Cl, Br, I, —OH, C₁₋₄ alkyl (e.g., methyl, ethyl, propyl, isopropyl, tert-butyl), CF₃, —NH₂, —CN, protected —OH, and a protected —NH₂; or (2) a naphthyl optionally substituted with one or more (typically, 1-3) substituents independently selected from F, Cl, Br, I, —OH, optionally substituted C₁₋₄ alkyl (e.g., methyl, ethyl, propyl, isopropyl, tert-butyl, CH₂CH₂—CN, CF₂H, or CF₃), optionally substituted C₂₋₄ alkenyl, optionally substituted C₂₋₄ alkynyl (e.g., ethynyl), cyclopropyl, —NH₂, —CN, protected —OH, and a protected —NH₂.

49. The compound of any one of claims 31-48, wherein R³ is selected from: 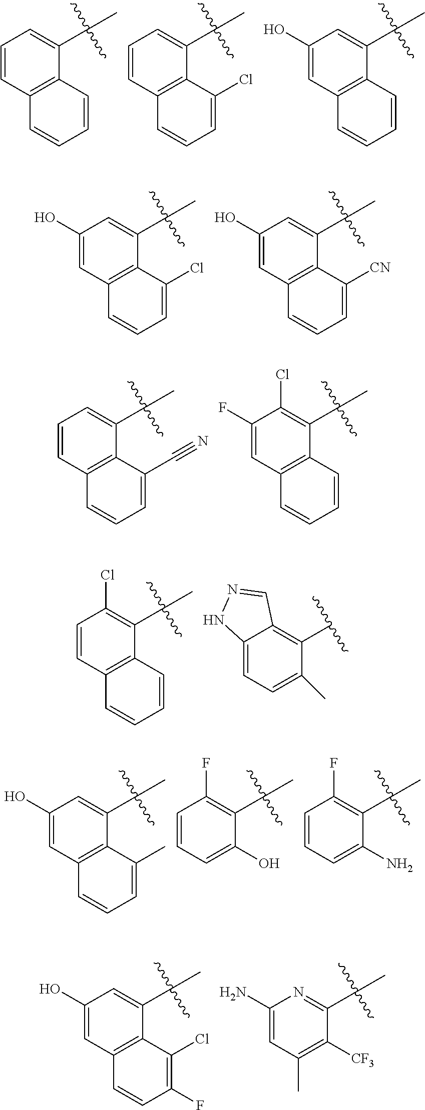 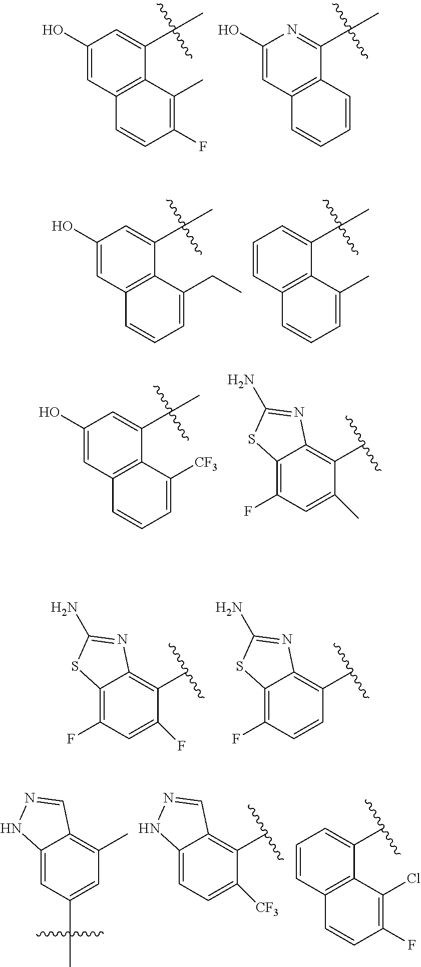
or R³ is selected from

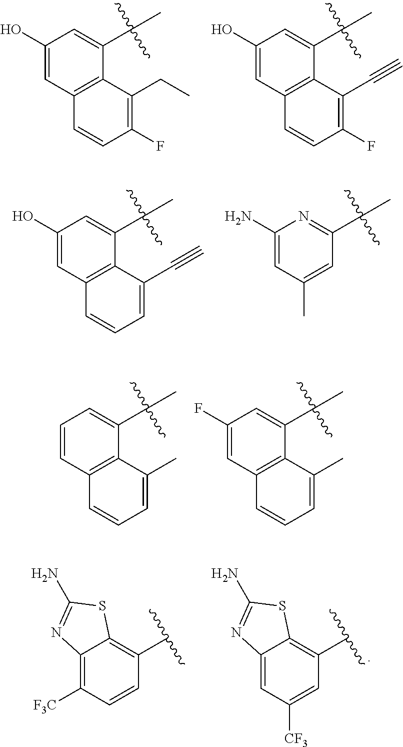

50. A compound selected from the compounds listed in Table A herein, or a pharmaceutically acceptable salt thereof.

51. A pharmaceutical composition comprising the compound of any one of claims 1-50 or a pharmaceutically acceptable salt thereof and a pharmaceutically acceptable excipient.

52. A method of inhibiting KRAS mutant protein in a cancer cell, the method comprising contacting the cancer cell with the compound of any one of claims 1-50 or a pharmaceutically acceptable salt thereof.

53. A method of treating cancer in a subject, the method comprising administering to the subject a therapeutically effective amount of the compound of any one of claims 1-50 or a pharmaceutically acceptable salt thereof, or the pharmaceutical composition of claim 51.

54. The method of claim 53, wherein the cancer is pancreatic cancer, colorectal cancer, lung cancer, endometrial cancer, appendix cancer, cholangiocarcinoma, bladder urothelial cancer, ovarian cancer, gastric cancer, breast cancer, bile duct cancer or a hematologic malignancy.

55. The method of claim 43 or 54, further comprising treating the subject with an additional therapy (combination therapy).

56. The method of claim 55, wherein the additional therapy (combination therapy) is a targeted therapeutic agent, chemotherapeutic agent, therapeutic antibody, radiation, cell therapy, gene therapy, or immunotherapy.

57. The method of any one of claims 53-56, wherein the subject has a mutation of KRAS, HRAS and/or NRAS.

58. A method for inhibiting proliferation of a cell population, the method comprising contacting the cell population with the compound of any one of claims 1-50 or a pharmaceutically acceptable salt thereof.

59. The method of claim 58, wherein inhibition of proliferation is measured as a decrease in cell viability of the cancer cell population.

60. A method for treating a disease or disorder mediated by a Ras (KRAS, HRAS and/or NRAS) mutant protein in a subject in need thereof, the method comprising:
determining if the subject has a KRAS, HRAS and/or NRAS mutation; and if the subject is determined to have the KRAS, HRAS and/or NRAS mutation, then administering to the subject a therapeutically effective amount of the compound of any one of claims 1-50 or a pharmaceutically acceptable salt thereof, or the pharmaceutical composition of claim 51.

61. The method of claim 60, wherein the disease or disorder is cancer, for example pancreatic cancer, colorectal cancer, lung cancer (e.g., non-small cell lung cancer), endometrial cancer, appendix cancer, cholangiocarcinoma, bladder urothelial cancer, ovarian cancer, gastric cancer, breast cancer, bile duct cancer or a hematologic malignancy.

62. A method for inhibiting cancer metastasis or tumor metastasis, the method comprising administering an effective amount of the compound of any one of claims 1-50 or a pharmaceutically acceptable salt thereof, or the pharmaceutical composition of claim 51 to a subject in need thereof.

63. The method of claim 61 or 62, further comprising treating the subject with an additional therapy (combination therapy), wherein the additional therapy is a targeted therapeutic agent, chemotherapeutic agent, therapeutic antibody, radiation, cell therapy, gene therapy, and/or immunotherapy.
